(19) United States
(12) Patent Application Publication
(10) Pub. No.: US 2022/0310929 A1
(43) Pub. Date: Sep. 29, 2022

(54) OPTOELECTRONIC DEVICE

(71) Applicant: OXFORD UNIVERSITY INNOVATION LIMITED, Oxgord (GB)

(72) Inventors: HENRY JAMES SNAITH, Oxford (GB); YEN-HUNG LIN, Oxford (GB)

(21) Appl. No.: 17/618,342

(22) PCT Filed: May 28, 2020
(86) PCT No.: PCT/GB2020/051287

§ 371 Date: Dec. 10, 2021

(30) Foreign Application Priority Data
Jun. 11, 2019 (GB) 1908334.4

(51) Int. Cl. H01L 51/00; C07C 251/00; C07D 233/02; C07D 221/00

(52) U.S. Cl. CPC H01L 51/0047; C07C 251/00; C07D 233/02; C07D 221/00; H01L 51/4213

(57) ABSTRACT

The invention relates to an optoelectronic device comprising: (a) a layer comprising a crystalline A/M/X material, wherein the crystalline A/M/X material comprises a compound of formula: [A]ₐ[M]_{b}[X]_{c} wherein: [A] comprises one or more A cations; [M] comprises one or more M cations which are metal or metalloid cations; [X] comprises one or more X anions; a is a number from 1 to 6; b is a number from 1 to 6; and c is a number from 1 to 18; and (b) an ionic solid which is a salt comprising an organic cation and a counter anion. The invention also provides various processes for producing an ionic solid-modified film of the crystalline A/M/X material.

### FIELD OF THE INVENTION

The invention provides an optoelectronic device comprising a layer of an ionic solid-modified crystalline A/M/X material. Also provided are processes for producing an ionic solid-modified film of a crystalline A/M/X material and a process for producing an optoelectronic device comprising an ionic-solid modified film of a crystalline A/M/X material.

### BACKGROUND TO THE INVENTION

When the first report of a perovskite solar cell was made in 2009, the power conversion efficiency stood at 3%. By 2012, perovskite photovoltaic devices achieving 9.2% and 10.9% had been demonstrated. Since then, there has been burgeoning research into the field of perovskite photovoltaics and photovoltaic devise based on other A/M/X materials, with such materials showing the promise to completely transform the energy landscape. Perovskite-based photovoltaic devices have since achieved certified efficiencies of 23%.

Solar cells based on metal halide perovskites are emerging as one of the most promising future photovoltaic (PV) technologies. The certified power conversion efficiency (PCE) has reached to 23.3 percent within only a few years, surpassing multi-crystalline silicon and all other thin film PV technologies. Previous efforts on composition engineering of perovskites, interface engineering of device structures, and encapsulation techniques have significantly advanced the long-term stability of perovskite solar cells over the last few years. However, operational device stability under combined full spectrum sunlight and heat stressing is still a key challenge for practical applications of perovskite solar cells. Among all the factors that affect the device stability, ion migration in the perovskite active layer poses a unique threat.

The ion migration in metal halide perovskites is related to instabilities in the materials and ensuing solar cells, and the presence of mobile defects represent a unique challenge for stabilizing these photovoltaic materials. Previous investigations have demonstrated that the ion migration is thermally activated, and that the activation energy is further decreased under illumination. Furthermore, it is expected that the mobile ionic species are defects such as vacancies or interstitials, and that these defects, which will be primarily located at the surfaces and grain boundaries, are expected to be the source for the onset of degradation to environmental factors. Hence light and heat, especially in the presence of any air, pose a significant threat to the long-term stability of perovskites. It is therefore very difficult to obtain a perovskite which combines both excellent PCE with good long-term stability for practical applications.

Jung et al. “Efficient, stable and scalable perovskite solar cells using poly(3-hexylthiophene)” describes perovskite solar cells that use P3HT as a hole-transport material (HTM). In those cells, a thin layer is formed on top of the light-absorbing perovskite layer, and beneath the P3HT layer, by an in situ reaction of the quaternary ammonium halide salt n-hexyl trimethyl ammonium bromide on the perovskite surface. This is said to improve interfacial contact between the perovskite and the HTM. An unencapsulated device monitored at 80% humidity and in room temperature showed a deterioration in power conversion efficiency over 1,000 hrs, and the device was said to maintain nearly 80% of its initial efficiency over that period. However, greater device stabilities in ambient conditions are required for viable solar cells, which must maintain high efficiency for as long as possible under ambient conditions.

Zheng et al. “Defect passivation in hybrid perovskite solar cells using quaternary ammonium halide anions and cations” Nature Energy, 2, 17102 (2017) relates to the use of quaternary ammonium halides, and in particular the quaternary ammonium halide salt choline chloride, to passivate ionic defects in perovskite solar cells. The solar cells shown in Zheng et al. exhibited a PCE of about 21-18% for about a month, with gradual deterioration over that period. For real-world applications, however, much longer useable lifetimes for solar cell devices are required, which are also required to be stable under sun light at temperatures much higher than room temperature, such as 85° C.

There therefore exists a need for perovskite materials which can be incorporated into optoelectronic devices such as photovoltaics, that simultaneously exhibit high PCE as well as long device life time in harsh aging conditions, for example full spectrum sunlight with heat stressing.

### SUMMARY OF THE INVENTION

The present invention provides optoelectronic devices comprising crystalline A/M/X materials that simultaneously exhibit improved performance (e.g. improved efficiency) and excellent long-term stability. This is achieved by incorporating ionic solids into the perovskite light-harvesting layer, resulting in improved efficiency and stability.

Ion migration is related to instabilities in the A/M/X materials. Ion migration leads to defects which are thought to be the source for the onset of degradation due to environmental factors.

Ion migration is heat and light activated, therefore it is important to develop materials that are stable and suppress ion migration in response to combined light and heat stress. The inventors have unexpectedly discovered that ionic solids increase the open-circuit voltage of the solar cells, and therefore reduce the unwanted trap assisted recombination in devices, and inhibit degradation of the perovskite material, thereby providing materials that do not rapidly degrade when used in non-ideal, simulated real-world conditions e.g. full spectrum sunlight at elevated temperature and humidity. This represents a key step towards the commercial upscale and deployment of the perovskite photovoltaic technology.

Further, the ionic solid doped A/M/X materials provide improved energy alignment between the A/M/X material and any adjacent charge transporting layers. This results in improved charge extraction and efficiency for optoelectronic devices employing ionic solid doped A/M/X materials. For instance, the efficiency of “positive-intrinsic-negative” (p-i-n) planar heterojunction solar cells employing p-type hole conductors such as PolyTPD:F4-TCNQ and N,N′-bis(1-naphthyl)-N,N′-diphenyl-1,1′-biphenyl-4,4′-diamine (NPD), and an A/M/X material as described herein can be stabilised at over 19 percent efficiency.

Further, the optoelectronic devices according to the present application may be fabricated using either solution-based methods or vacuum based methods. This gives a flexible choice as to the ideal manufacturing methodology for these improved materials.

The advantages described above represent a significant advance towards realizing a commercially viable low-cost PV technology.

Accordingly, the present invention provides an optoelectronic device comprising:

(a) a layer comprising a crystalline A/M/X material, wherein the crystalline A/M/X material comprises a compound of formula:

[A]ₐ[M]_{b}[X]_{c},

wherein:
[A] comprises one or more A cations;
[M] comprises one or more M cations which are metal or metalloid cations;
[X] comprises one or more X anions;
a is a number from 1 to 6;
b is a number from 1 to 6; and
c is a number from 1 to 18; and
   (b) an ionic solid which is a salt comprising an organic cation and a counter anion. Typically, the ionic solid is other than a quaternary ammonium halide salt. The ionic solid is usually other than a primary ammonium halide salt. The ionic solid is often other than a secondary ammonium halide salt. The ionic solid is usually other than a tertiary ammonium halide salt. Thus, typically, the ionic solid is other than a primary, secondary, tertiary or quaternary ammonium halide salt. The ionic solid is typically other than a formamidinium halide salt, and usually other than a guanidinium halide salt. Thus, typically, the ionic solid is other than a primary, secondary, tertiary or quaternary ammonium halide salt and other than a formamidinium or guanidinium halide salt. The ionic solid is typically other than a halide salt of a cation of formula (X) as defined hereinbelow. Often, the ionic solid is other than a primary, secondary, tertiary or quaternary ammonium halide salt and other than a halide salt of a cation of formula (X) as defined hereinbelow. Typically, when the counter-anion of the ionic solid is halide, the organic cation of the ionic solid is other than each of the one or more A cations of the crystalline A/M/X material. Often, the organic cation of the ionic solid is other than each of the one or more A cations of the crystalline A/M/X material.

The invention also provides a process for producing an ionic solid-modified film of a crystalline A/M/X material, wherein the crystalline A/M/X material comprises a compound of formula: [A]ₐ[M]_{b}[X]_{c}, wherein: [A] comprises one or more A cations; [M] comprises one or more M cations which are metal or metalloid cations; [X] comprises one or more X anions; wherein a is a number from 1 to 6; b is a number from 1 to 6; and c is a number from 1 to 18, and the ionic solid is a salt which comprises an organic cation and a counter-anion,

the process comprising: disposing a film-forming solution on a substrate, wherein the film-forming solution comprises a solvent, the one or more A cations, the one or more M cations, the one or more X anions, the organic cation and the counter-anion. Typically, the ionic solid is other than a quaternary ammonium halide salt, i.e. the counter-anion is other than halide and the organic cation is other than a quaternary ammonium cation. The ionic solid is usually other than a primary ammonium halide salt. The ionic solid is often other than a secondary ammonium halide salt. The ionic solid is usually other than a tertiary ammonium halide salt. Thus, typically, the ionic solid is other than a primary, secondary, tertiary or quaternary ammonium halide salt, i.e. the counter-anion is other than halide and the organic cation is other than a primary, secondary, tertiary or quaternary ammonium cation. The ionic solid is typically other than a formamidinium halide salt, and usually other than a guanidinium halide salt. Thus, typically, the ionic solid is other than a primary, secondary, tertiary or quaternary ammonium halide salt and other than a formamidinium or guanidinium halide salt. The ionic solid is typically other than a halide salt of a cation of formula (X) as defined hereinbelow. Often, the ionic solid is other than a primary, secondary, tertiary or quaternary ammonium halide salt and other than a halide salt of a cation of formula (X) as defined hereinbelow. Typically, when the counter-anion of the ionic solid is halide, the organic cation of the ionic solid is other than each of the one or more A cations of the crystalline A/M/X material. Often, the organic cation of the ionic solid is other than each of the one or more A cations of the crystalline A/M/X material.

The invention also provides a process for producing an ionic solid-modified film of a crystalline A/M/X material, which crystalline A/M/X material comprises a compound of formula: [A]ₐ[M]_{b}[X]_{c}, wherein: [A] comprises one or more A cations; [M] comprises one or more M cations which are metal or metalloid cations; [X] comprises one or more X anions; wherein a is a number from 1 to 6; b is a number from 1 to 6; and c is a number from 1 to 18, the process comprising:

a) disposing a first solution on a substrate wherein the first solution comprises a solvent and one or more M cations, and optionally removing the solvent, to produce a treated substrate;

b) contacting the treated substrate with a second solution comprising a solvent and one or more A cations or with vapour comprising one or more A cations,

wherein: one or more X anions are present in one or both of: (i) the first solution employed in step (a), and (ii) the second solution or vapour employed in step (b); and the first solution employed in step (a) further comprises an organic cation and a counter-anion of an ionic solid, or step (b) further comprises contacting the treated substrate with an ionic solid, wherein the ionic solid is a salt which comprises an organic cation and a counter-anion. Typically, the ionic solid is other than a quaternary ammonium halide salt. The ionic solid is usually other than a primary ammonium halide salt. The ionic solid is often other than a secondary ammonium halide salt. The ionic solid is usually other than a tertiary ammonium halide salt. Thus, typically, the ionic solid is other than a primary, secondary, tertiary or quaternary ammonium halide salt. The ionic solid is typically other than a formamidinium halide salt, and usually other than a guanidinium halide salt. Thus, typically, the ionic solid is other than a primary, secondary, tertiary or quaternary ammonium halide salt and other than a formamidinium or guanidinium halide salt. The ionic solid is typically other than a halide salt of a cation of formula (X) as defined hereinbelow. Often, the ionic solid is other than a primary, secondary, tertiary or quaternary ammonium halide salt and other than a halide salt of a cation of formula (X) as defined hereinbelow. Typically, when the counter-anion of the ionic solid is halide, the organic cation of the ionic solid is other than each of the one or more A cations of the crystalline A/M/X material. Often, the organic cation of the ionic solid is other than each of the one or more A cations of the crystalline A/M/X material.

Typically, the first solution employed in step (a) further comprises the organic cation and the counter-anion of the ionic solid. Alternatively, step (b) may further comprise contacting the treated substrate with the ionic solid, optionally wherein step (b) comprises:
(i) contacting the treated substrate with said second solution wherein the second solution further comprises the organic cation and the counter-anion of the ionic solid; or
(ii) contacting the treated substrate with said vapour comprising one or more A cations and with vapour comprising the organic cation and the counter-anion of the ionic solid, optionally wherein step (b) comprises:
   b1) vapourising a composition, or compositions, which comprise the one or more A cations and the ionic solid, and
   b2) depositing the resulting vapour on the treated substrate.

The invention also provides a process for producing an ionic solid-modified film of a crystalline A/M/X material, wherein the crystalline A/M/X material comprises a compound of formula: [A]ₐ[M]_{b}[X]_{c}, wherein: [A] comprises one or more A cations; [M] comprises one or more M cations which are metal or metalloid cations; [X] comprises one or more X anions; a is a number from 1 to 6; b is a number from 1 to 6; and c is a number from 1 to 18; and wherein the ionic solid is a salt which comprises an organic cation and a counter-anion; which process comprises treating a film of the crystalline A/M/X material with the organic cation and the counter-anion of the ionic solid. Typically, the ionic solid is other than a quaternary ammonium halide salt. The ionic solid is usually other than a primary ammonium halide salt. The ionic solid is often other than a secondary ammonium halide salt. The ionic solid is usually other than a tertiary ammonium halide salt. Thus, typically, the ionic solid is other than a primary, secondary, tertiary or quaternary ammonium halide salt. The ionic solid is typically other than a formamidinium halide salt, and usually other than a guanidinium halide salt. Thus, typically, the ionic solid is other than a primary, secondary, tertiary or quaternary ammonium halide salt and other than a formamidinium or guanidinium halide salt. The ionic solid is typically other than a halide salt of a cation of formula (X) as defined hereinbelow. Often, the ionic solid is other than a primary, secondary, tertiary or quaternary ammonium halide salt and other than a halide salt of a cation of formula (X) as defined hereinbelow. Typically, when the counter-anion of the ionic solid is halide, the organic cation of the ionic solid is other than each of the one or more A cations of the crystalline A/M/X material. Often, the organic cation of the ionic solid is other than each of the one or more A cations of the crystalline A/M/X material.

The step of treating the film of the crystalline A/M/X material may comprise treating the film with a solution comprising the organic cation and the counter anion.

Alternatively, the step of treating the film of the crystalline A/M/X material may comprise exposing the film to vapour comprising the organic cation and vapour comprising the counter anion. The vapour comprising the organic cation and the vapour comprising the counter anion are typically one and the same vapour, but they may alternatively be different vapours.

Indeed, ionic solids may be vaporised by sublimation, meaning that employing an ionic solid facilitates the use of a vapour deposition process for producing an ionic solid modified film of a crystalline A/M/X material. Both the A/M/X material and the ionic solid may be deposited by vapour deposition, to produce an ionic solid modified film of a crystalline A/M/X material.

Accordingly, the invention also provides a process for producing an ionic solid-modified film of a crystalline A/M/X material, wherein the crystalline A/M/X material comprises a compound of formula: [A]ₐ[M]_{b}[X]_{c}, wherein: [A] comprises one or more A cations; [M] comprises one or more M cations which are metal or metalloid cations; [X] comprises one or more X anions; a is a number from 1 to 6; b is a number from 1 to 6; and c is a number from 1 to 18, and wherein the ionic solid is a salt comprising an organic cation and a counter anion; which process comprises:

exposing a substrate to vapour comprising the one or more A cations, vapour comprising the one or more M cations, vapour comprising the one or more X anions, vapour comprising the organic cation, and vapour comprising the counter anion. Typically, the ionic solid is other than a quaternary ammonium halide salt, i.e. the organic cation is other than a quaternary ammonium cation and the counter anion is other than a halide. The ionic solid is usually other than a primary ammonium halide salt. The ionic solid is often other than a secondary ammonium halide salt. The ionic solid is usually other than a tertiary ammonium halide salt. Thus, typically, the ionic solid is other than a primary, secondary, tertiary or quaternary ammonium halide salt, i.e. the counter-anion is other than halide and the organic cation is other than a primary, secondary, tertiary or quaternary ammonium cation. The ionic solid is typically other than a formamidinium halide salt, and usually other than a guanidinium halide salt. Thus, typically, the ionic solid is other than a primary, secondary, tertiary or quaternary ammonium halide salt and other than a formamidinium or guanidinium halide salt. The ionic solid is typically other than a halide salt of a cation of formula (X) as defined hereinbelow. Often, the ionic solid is other than a primary, secondary, tertiary or quaternary ammonium halide salt and other than a halide salt of a cation of formula (X) as defined hereinbelow. Typically, when the counter-anion of the ionic solid is halide, the organic cation of the ionic solid is other than each of the one or more A cations of the crystalline A/M/X material. Often, the organic cation of the ionic solid is other than each of the one or more A cations of the crystalline A/M/X material.

The vapour comprising the one or more A cations, vapour comprising the one or more M cations, vapour comprising the one or more X anions, vapour comprising the organic cation, and vapour comprising the counter anion, may be one and the same vapour. Thus, the process of this aspect of the invention may comprise exposing the substrate to vapour which comprises the one or more A cations, the one or more M cations, the one or more X anions, the organic cation, and the counter anion. The substrate may therefore be exposed to the one or more A cations, one or more M cations, one or more X anions, organic cation and counter anion at the same time.

Alternatively, the one or more A cations, the one or more M cations, the one or more X anions, the organic cation, and the counter anion, may be part of two or more different vapour phases, to which the substrate is exposed. The substrate may be exposed to the two or more different vapour phases at the same time or at different times, e.g. separately and/or sequentially. Accordingly, the process of this aspect of the invention may comprise exposing the substrate to two or more different vapour phases, wherein the two or more different vapour phases together comprise the one or more A cations, the one or more M cations, the one or more X anions, the organic cation, and the counter anion. The substrate may be exposed to the two or more different vapour phases simultaneously (at the same time), separately (at different times), for instance sequentially (in any order).

For instance, the substrate may be exposed to: (i) vapour comprising the one or more A cations, the one or more M cations, the one or more X anions; and (ii) vapour comprising the organic cation and the counter anion of the ionic solid. Alternatively, the substrate may be exposed to (i) vapour comprising the one or more M cations, (ii) vapour comprising the one or more A cations (wherein the one or more X anions may be present in the vapour comprising the one or more M cations, the vapour comprising the one or more A cations, or in both the vapour comprising the one or more M cations and the vapour comprising the one or more A cations), and (iii) vapour comprising the organic cation and the counter anion of the ionic solid. The substrate may be exposed to these vapour phases at the same time or at different times, e.g. sequentially in any order.

The invention also provides a process for producing an optoelectronic device, which process comprises producing, on a substrate, an ionic solid-modified film of a crystalline A/M/X material, by a process as described herein.

The invention also provides an ionic solid-modified film of a crystalline A/M/X material which is obtainable by a process as described herein.

The invention also provides an optoelectronic device which

(a) comprises an ionic solid-modified film of a crystalline A/M/X material obtainable by a process as described herein; or
(b) is obtainable by a process as described herein.

### BRIEF DESCRIPTION OF THE FIGURES

FIGS. 1a-f show device architecture, solar cell performance parameters and statistical results for adding ionic solid (1), 6,7-Dihydro-2-pentafluorophenyl-5H-pyrrolo[2,1-c]-1,2,4-triazolium tetrafluoroborate ([PF-PTAM][BF₄]), into the Cs_{0.17}FA_{0.83}Pb(I_{0.9}Br_{0.1})₃ perovskite precursor. The solar cells were made on 3 cm by 3 cm substrates with 0.2 cm² cell size. FIG. 1a shows the chemical structure of [PF-PTAM][BF₄] and a schematic device architecture of the planar heterojunction p-i-n perovskite solar cell. FIG. 1b shows the current density and voltage (J-V) characteristics of the forward bias (FB) to short-circuit (SC) scans for the perovskite solar cells, with a perovskite absorber layer of the Cs_{0.17}FA_{0.83}Pb(I_{0.9}Br_{0.1})₃ composition with 0.3 mol % (with respect to Pb atom) [PF-PTAM][BF₄] (0.3 mol %, circle) and without ionic solid (Ref., square), under simulated AM1.5 sunlight with the intensity of 100 mW/cm². FIG. 1c-f show statistical results of PCE (FIG. 1c), V_{OC} (FIG. 1d), J_{SC} (FIG. 1e) and FF (FIG. 1f) for perovskite solar cells fabricated from Cs_{0.17}FA_{0.83}Pb(I_{0.9}Br_{0.1})₃ perovskite precursors with [PF-PTAM][BF₄] using different concentrations in the range from 0 (i.e., Ref.) to 0.4 mol %. All device parameters are determined from the FB to SC J-V scan curves.

FIGS. 2a-f show device architecture, solar cell performance parameters and statistical results for ionic solid (1), [PF-PTAM][BF₄], deposited onto the Cs_{0.17}FA_{0.83}Pb(I_{0.9}Br_{0.1})₃ perovskite absorber layer. The solar cells were made on 3 cm by 3 cm substrates with 0.2 cm² cell size. FIG. 2a schematically shows the chemical structure of [PF-PTAM][BF₄] and the relative position of this ionic solid in the planar heterojunction p-i-n perovskite solar cell. FIG. 2b shows the J-V characteristics of the FB to SC scans for the perovskite solar cells with a perovskite absorber layer of the Cs_{0.17}FA_{0.83}Pb(I_{0.9}Br_{0.1})₃ composition. The J-V curves include the perovskite solar cells with 0.6 mol % (with respect to Pb atom) [PF-PTAM][BF₄] (top treatment, filled circle) and without ionic solid (Ref., filled square) under simulated AM1.5 sunlight with the intensity of 100 mW/cm² as well as 0.6 mol % [PF-PTAM][BF₄] (open circle) and without ionic solid (Ref., open square) in the dark. FIG. 2c-f show statistical results for perovskite solar cells fabricated from Cs_{0.17}FA_{0.83}Pb(I_{0.9}Br_{0.1})₃ perovskite precursors with an additional layer of 0.6 mol % [PF-PTAM][BF₄] and without ionic solid addition (Ref.): PCE (FIG. 2c), V_{OC} (FIG. 2d), J_{SC} (FIG. 2e), and FF (FIG. 2f). All device parameters are determined from the FB to SC J-V scan curves.

FIGS. 3a-f show device architecture, solar cell performance parameters and statistical results for adding ionic solid (2), 1,3-Diisopropylimidazolium tetrafluoroborate ([IPIM][BF₄]), into the Cs_{0.17}FA_{0.83}Pb(I_{0.9}Br_{0.1})₃ perovskite precursor. The solar cells were made on 2.8 cm by 2.8 cm substrates with 0.0919 cm² cell size. FIG. 3a shows the chemical structure of [IPIM][BF₄] and a schematic device architecture of the planar heterojunction p-i-n perovskite solar cell. FIG. 3b shows the J-V characteristics of the FB to SC scans for the perovskite solar cells, with a perovskite absorber layer of the Cs_{0.17}FA_{0.83}Pb(I_{0.9}Br_{0.1})₃ composition with [IPIM][BF₄] at the concentrations of 0.1 mol % (triangle) (with respect to Pb atom), 0.2 mol % (circle), 0.3 mol % (inverted triangle) and without ionic solid (i.e. Ref., square), under simulated AM1.5 sunlight with the intensity of 100 mW/cm². FIG. 3c-f show statistical results of PCE (FIG. 3c), V_{OC} (FIG. 3d), J_{SC} (FIG. 3e) and FF (FIG. 3f) for perovskite solar cells fabricated from Cs_{0.17}FA_{0.83}Pb(I_{0.9}Br_{0.1})₃ perovskite precursors with [IPIM][BF₄] using different concentrations in the range from 0 (i.e., Ref.) to 0.3 mol %. All device parameters are determined from the FB to SC J-V scan curves.

FIGS. 4a-g show device architecture, solar cell performance parameters and statistical results for ionic solid (1), [PF-PTAM][BF₄], deposited before the ionic solid (2), [IPIM][BF₄], containing Cs_{0.17}FA_{0.83}Pb(I_{0.9}Br_{0.1})₃ perovskite absorber layer. For the perovskite solar cells shown in FIG. 4, the bottom surface treatment using [PF-PTAM][BF₄] was prepared from an 0.6 mol % (with respect to Pb atom) [PF-PTAM][BF₄] ionic solid precursor, unless stated otherwise. The solar cells were made on 2.8 cm by 2.8 cm substrates with 0.0919 cm² cell size. FIG. 4a schematically shows the chemical structures of [PF-PTAM][BF₄] and [IPIM][BF₄] as well as the relative positions of the ionic solids in the planar heterojunction p-i-n perovskite solar cell. FIG. 4b shows the J-V characteristics of the FB to SC scans for the perovskite solar cells with a perovskite absorber layer of the Cs_{0.17}FA_{0.83}Pb(I_{0.9}Br_{0.1})₃ composition. The J-V curves include the perovskite solar cells with the ionic solid additions (0.1 mol %, filled circle), including 0.1 mol % [IPIM][BF₄] in the perovskite precursor and the perovskite bottom surface treated with [PF-PTAM][BF₄], and without ionic solid (Ref., filled square) under simulated AM1.5 sunlight with the intensity of 100 mW/cm² as well as in the dark (open circle for the cell with the ionic solid additions; open square for the cell without ionic solid). FIG. 4c shows the static state power output for the cells with (circle) and without (square) the ionic solid additions. FIG. 4d-g show statistical results for perovskite solar cells fabricated from Cs_{0.17}FA_{0.83}Pb(I_{0.9}Br_{0.1})₃ perovskite precursors with the ionic solid additions at different concentrations of [IPIM][BF₄] from 0.1 to 0.3 mol % and without the ionic solid additions (Ref.): PCE (FIG. 4d), V_{OC} (FIG. 4e), J_{SC} (FIG. 4f), and FF (FIG. 4g). All device parameters are determined from the FB to SC J-V scan curves.

FIGS. 5a-g show device architecture, solar cell performance parameters and statistical results for adding ionic solid (3), 1,3-Di-tert-butylimidazolium tetrafluoroborate ([Di-tBIM][BF₄], or ionic solid (4), N-((Diisopropylamino)methylene)-N-diisopropylaminium tetrafluoroborate ([Di-IPAM][BF₄]), to the Cs_{0.17}FA_{0.83}Pb(I_{0.9}Br_{0.1})₃ perovskite absorber layer. The solar cells were made on 2.8 cm by 2.8 cm substrates with 0.0919 cm² cell size. FIG. 5a schematically shows the chemical structures of [Di-tBIM][BF₄] and ([Di-IPAM][BF₄] as well as the planar heterojunction p-i-n perovskite solar cell. FIG. 5b shows the J-V characteristics of the FB to SC scans for the perovskite solar cells with a perovskite absorber layer of the Cs_{0.17}FA_{0.83}Pb(I_{0.9}Br_{0.1})₃ composition. The J-V curves include the perovskite solar cells with 0.2 mol % (with respect to Pb atom) [Di-tBIM][BF₄] (filled circle), 0.2 mol % [Di-IPAM][BF₄] (filled triangle) and without ionic solid (Ref., filled square) under simulated AM1.5 sunlight with the intensity of 100 mW/cm² as well as in the dark (open circle for the cell with [Di-tBIM][BF₄]; open triangle for the cell with [Di-IPAM][BF₄]; open square for the cell without ionic solid). FIG. 5c shows the static state power output for the cells with [Di-tBIM][BF₄], with [Di-IPAM][BF₄] (triangle) and without ionic solid (square). FIG. 5d-g show statistical results for perovskite solar cells fabricated from Cs_{0.17}FA_{0.83}Pb(I_{0.9}Br_{0.1})₃ perovskite precursors with ionic solids of [Di-tBIM][BF₄] and [Di-IPAM][BF₄] as well as without ionic solid (Ref.): PCE (FIG. 5d), V_{OC} (FIG. 5e), J_{SC} (FIG. 5f), and FF (FIG. 5g). All device parameters are determined from the FB to SC J-V scan curves.

FIGS. 6a-k show solar cell performance parameters and statistical results for adding ionic solid (3), 1,3-Di-tert-butylimidazolium tetrafluoroborate ([Di-tBIM][BF₄], or ionic solid (4), N-((Diisopropylamino)methylene)-N-di isopropylaminium tetrafluoroborate ([Di-IPAM][BF₄]), to the Cs_{0.17}FA_{0.83}Pb(I_{0.9}Br_{0.1})₃ perovskite absorber layer for a 72-hour ageing period under full spectrum sunlight and heat (85° C.). The solar cells were made on 2.8 cm by 2.8 cm substrates with 0.0919 cm² cell size. FIG. 6a-c show the J-V characteristics of the FB to SC scans for the perovskite solar cells with a perovskite absorber layer of the Cs_{0.17}FA_{0.83}Pb(I_{0.9}Br_{0.1})₃ composition without ionic solid (Ref., FIG. 6a), with 0.2 mol % (with respect to Pb atom) [Di-tBIM][BF₄] (FIG. 6b), and 0.2 mol % [Di-IPAM][BF₄] (FIG. 6c), before ageing (circle), after 24-hour ageing (square), and 72-hour ageing (triangle). FIG. 6d-g show statistical results for perovskite solar cells fabricated from Cs_{0.17}FA_{0.83}Pb(I_{0.9}Br_{0.1})₃ perovskite precursors with ionic solids of [Di-tBIM][BF₄] and [Di-IPAM][BF₄] as well as without ionic solid (Ref.) after 24-hour ageing: PCE (FIG. 6d), V_{OC} (FIG. 6e), J_{SC} (FIG. 6f), and FF (FIG. 6g). FIG. 6h-k show statistical results for perovskite solar cells fabricated from Cs_{0.17}FA_{0.83}Pb(I_{0.9}Br_{0.1})₃ perovskite precursors with ionic solids of [Di-tBIM][BF₄] and [Di-IPAM][BF₄] as well as without ionic solid (Ref) after 72-hour ageing: PCE (FIG. 6h), V_{OC} (FIG. 6i), J_{SC} (FIG. 6j), and FF (FIG. 6k). All device parameters are determined from the FB to SC J-V scan curves.

FIG. 7 shows perovskite solar cell characterization, in particular: (A) Schematic of the p-i-n perovskite solar cell and the chemical structure of [BMP]⁺[BF₄]⁻. (B) Scanning electron microscopic image of the full device stack made from Cs_{0.17}FA_{0.83}Pb(I_{0.9}Br_{0.1})₃ with 0.25 mol % [BMP]⁺[BF₄]⁻ (the scale bar is 500 nm). (C) J-V characteristics of the representative 0.25 mol % [BMP]⁺[BF₄]⁻ modified Cs_{0.17}FA_{0.83}Pb(I_{0.9}Br_{0.1})₃ and control devices measured from the forward-bias (FB) to short-circuit (SC) scans under simulated AM1.5 sunlight and corresponding SPO. (D) Statistical results of device parameters for Cs_{0.17}FA_{0.83}Pb(I_{0.90}Br_{0.10})₃ based devices. (E) J-V characteristics for the champion cell with 0.25 mol % [BMP]⁺[BF₄]⁻ modified Cs_{0.17}FA_{0.83}Pb(I_{0.90}Br_{0.10})₃. Inset: corresponding SPO and current density measured under SPO (J_{SPO}) (F) Modeling of the thickness-dependent subcell J_{sc} for perovskite-on-silicon tandem cells with perovskites of different band gaps. The evolution of perovskite subcell J_{sc} is shown in blue, while the corresponding Si subcell J_{sc} is shown in red. (G) J-V characteristics of the representative 0.25 mol % [BMP]⁺[BF₄]⁻ modified and control devices using Cs_{0.17}FA_{0.83}Pb(I_{0.77}Br_{0.23})₃. (H) Corresponding SPO, EQE and integrated J_{SC} for the devices shown in (G). The integrated J_{SC} values for the modified and control devices are 18.8 and 19.0 mA·cm⁻², respectively. (I) Statistical results of device parameters for Cs_{0.17}FA_{0.83}Pb(I_{0.77}Br_{0.23})₃ based devices.

FIG. 8 shows J-V scans of (A) an optimized 0.25 mol % [BMP]⁺[BF₄]⁻ p-i-n perovskite solar cell and (B) a control device with a perovskite composition of Cs_{0.17}FA_{0.83}Pb(I_{0.90}Br_{0.10})₃. Reverse and forward scans represent the measurements following the voltage sweeps from flat-band to short-circuit (FB-SC) and from short-circuit to flat-band (SC-FB), respectively. For the FB-SC scan, the obtained device performance parameters are as follows: (A) V_{OC}=1.12 V, J_{SC}=22.7 mA·cm⁻², FF=0.80, PCE=20.3%; (B) V_{OC}=1.07 V, J_{SC}=22.4 mA·cm⁻², FF=0.74, PCE=17.6%. For the SC-FB scan, the obtained device performance parameters are as follows: (A) V_{OC}=1.11 V, J_{SC}=22.5 mA·cm⁻², FF 0.73, PCE=18.1%; (B) V_{OC}=1.07 V, J_{SC}=22.4 mA·cm⁻², FF=0.72, PCE=17.2%.

FIG. 9 shows external quantum efficiency (EQE) spectra (line) and integrated photocurrent (scatters), integrated over the AM1.5 (100 mW·cm-2) solar spectrum, for the champion [BMP]⁺[BF_{4]}⁻ perovskite Cs_{0.17}FA_{0.83}Pb(I_{0.90}Br_{0.10})₃ solar cell shown in FIG. 7E. The integrated JSC values over the measured EQE was 22.1 mA·cm⁻².

FIG. 10 shows simulated subcell current densities (J_{SC}) for 500-nm thick perovskite top cells and Si bottom cells for different perovskite band gap (E_{g}). Parameters employed for this simulation are adopted from the previous publication (Mazzarella et al., Adv. Energy Mater. 9, 1803241 (2019)). Current-matching takes place at a perovskite band gap of ˜1.66 eV.

FIG. 11 shows external quantum efficiency (EQE, solid line) for the [BMP]⁺[BF₄]− modified perovskite solar cell shown in FIG. 7G. A band gap value of 1.66 eV is extracted from the first derivative of the EQE (grey dashed line) as a function of photon energy.

FIG. 12 shows statistics of the device performance parameters for solar cells fabricated from perovskite precursors with [BMP]⁺[BF₄]⁻ concentrations ranging from 0 mol % (i.e. the control device, Ctrl) to 0.3 mol % (with respect to the Pb concentration). (A) PCE, (B) V_{OC}, (C) J_{sc} and (D) FF were determined from the reverse J-V scans (i.e. from flat-band to short-circuit, FB-SC) of 12 cells for each condition.

FIG. 13 shows J-V scans of (A) an optimized 0.25 mol % [BMP]⁺[BF₄]⁻ p-i-n perovskite solar cell and (B) a control device with a perovskite composition of Cs_{0.17}FA_{0.83}Pb(I_{0.77}Br_{0.23})₃. Reverse and forward scans represent the measurements following the voltage sweeps from flat-band to short-circuit (FB-SC) and from short-circuit to flat-band (SC-FB), respectively. For the FB-SC scan, the obtained device performance parameters are as follows: (A) V_{OC}=1.16 V, J_{SC}=19.5 mA·cm⁻², FF=0.77, PCE=17.3%; (B) V_{OC}=1.11 V, J_{SC}=19.9 mA·cm⁻², FF=0.75, PCE=16.6%. For the SC-FB scan, the obtained device performance parameters are as follows: (A) V_{OC}=1.13 V, J_{SC}=19.4 mA·cm⁻², FF=0.70, PCE=15.1%; (B) V_{OC}=1.10 V, J_{SC}=19.8 mA·cm⁻², FF=0.69, PCE=14.9%.

FIG. 14 shows (A) Light intensity-dependent device V_{OC}. For the control and [BMP]⁺[BF₄]⁻ modified perovskite films, the ideality factors (n_{id}) of 2 and 1.55 are determined, respectively. (B) Total extracted charge as a function of light-induced V_{OC}, using a range of background illumination intensities. The [BMP][BF₄]⁻ content is 0.25 mol % with respect to the Pb atom in the perovskite films while the control sample (Ctrl) is Cs_{0.17}FA_{0.83}Pb(I_{0.77}Br_{0.23})₃ without any [BMP]⁺[BF₄]⁻ additive.

FIG. 15 shows (A) Time-resolved photoluminescence (TRPL) and (B) steady state photoluminescence (SSPL) results obtained from Cs_{0.17}FA_{0.83}Pb(I_{0.77}Br_{0.23})₃ with and without [BMP]⁺[BF₄]⁻ prepared on polyTPD:F4-TCNQ/FTO glass substrates. From the TRPL results, the initial decay of the control device (Ctrl) is faster than the [BMP]⁺[BF₄]⁻, suggesting stronger trapping process and faster recombination. Meanwhile, the SSPL intensity increases when adding more concentrated [BMP]⁺[BF₄]⁻ into the perovskite films (concentrations of [BMP]⁺[BF₄]⁻ with respect to the Pb content).

FIG. 16 shows (A) Charge carrier lifetime (measured at V_{OC}) as a function of total extracted charge. (B) Effective diffusion mobilities measured at short circuit conditions. The [BMP]⁺[BF₄]⁻ content is 0.25 mol % with respect to the Pb atom in the perovskite films while the control sample (Ctrl) is Cs_{0.17}FA_{0.83}Pb(I_{0.77}Br_{0.23})₃ without any [BMP]⁺[BF₄]⁻ additive.

FIG. 17 shows TRMC transients: photo-conductance (ΔG) as a function of time (t) for (A) control sample in a composition of Cs_{0.17}FA_{0.83}Pb(I_{0.77}Br_{0.23})₃, and (B) the same perovskite composition with 0.25 mol % [BMP]⁺[BF₄]⁻, for various incident optical fluences. All measurements were carried out in air at room temperature.

FIG. 18 shows time-resolved microwave conductivity (TRMC) figure of merit: ϕΣμ_{TRMC} as a function of laser fluence for the 0.25 mol % [BMP]⁻[BF₄]⁻ modified and control Cs_{0.17}FA_{0.83}Pb(I_{0.90}Br_{0.10})₃ perovskite samples. The points are experimental values and the lines are straight line fits to a numerical model that accounts for bimolecular and Auger recombination during the laser pulse. All measurements were carried out in air at room temperature.

FIG. 19 shows (A) sum of charge carrier mobilities ϕΣμ_{ip-TPC} of electrons and holes for the 0.25 mol % [BMP]⁺[BF₄]⁻ modified (light grey circle) and control (dark grey square) Cs_{0.17}FA_{0.83}Pb(I_{0.77}Br_{0.23})₃ perovskite samples obtained from in-plane transient photoconductivity under different excitation densities. The lines are guide for the eye while the error bars represent standard derivation. (B) Decay profile of photoconductivity measured as a function of time after pulse laser excitation. The data is fitted with a mono-exponential decay function (solid lines).

FIG. 20 shows (A) a typical depth profile acquired from a region (1.2 μm×1.2 μm) covering one ¹⁹F⁻ hotspot through the perovskite film thickness (see FIGS. 21A and 21D for corresponding information). (B) A line profile acquired from a region covering a ¹⁹F⁻ hotspot and nearby perovskite.

FIG. 21 shows high-resolution secondary ion mass spectrometry and X-ray diffraction analysis. (A and B) ¹⁹F⁻ and ¹¹B¹⁶O₂⁻ ion maps for the F and B distributions towards the top surface of a ˜500 nm Cs_{0.17}FA_{0.83}Pb(I_{0.77}Br_{0.23})₃ with 0.25 mol % [BMP]⁺[BF₄]⁻ perovskite film. (C) Secondary electron map for the sputtered surface morphology ˜60 nm below the sample surface. The squares denoted in (A-C) are to indicate the corresponding regions of highly localized F and B concentrations. (D) A reconstructed 3D map (stretched in the Z direction for clarity) showing the distribution of the ¹⁹F⁻ signals through the perovskite layer. (E) and (F) show XRD series for the gaining of the unencapsulated control and 0.25 mol % [BMP]⁺[BF₄]⁻ modified Cs_{0.17}FA_{0.83}Pb(I_{0.77}Br_{0.23})₃ perovskite films, respectively, prepared on FTO glass substrates. The XRD peaks corresponding to PbI₂ (+), FTO (*) and the secondary cubic perovskite phase (‡) are marked.

FIG. 22 shows solid-state nuclear magnetic resonance characterization of the control and [BMP]⁺[BF₄]⁻ modified Cs_{0.17}FA_{0.83}Pb(I_{0.77}Br_{0.23})₃ (CsFA) films: (A) 1D ¹H NMR for [BMP]⁺[BF_{4]}⁻ only (green line); control Cs/FA (blue line); [BMP]^{+[BF}₄]⁻ Cs/FA (red line) while (B) and (C) show ¹H—¹H 2D correlation for control Cs/FA and [BMP]⁺[BF₄]⁻ modified Cs/FA, respectively.

FIG. 23 provides a comparison of X-ray photoelectron spectroscopy C 1s and N 1s spectra of Cs_{0.17}FA_{0.83}Pb(I_{0.77}Br_{0.23})₃ with 0.25 mol % [BMP]⁺[BF₄]⁻. Perovskite films measured after aging under full spectrum sunlight at 60° C. in ambient air: (A) C 1s and (B) N 1s regions with 0.25 mol % [BMP]⁺[BF₄]⁻ additive and without (control).

FIG. 24 shows evolution of ultraviolet-visible absorbance spectra of Cs_{0.17}FA_{0.83}Pb(I_{0.77}Br_{0.23})₃ perovskite films measured after aged under full spectrum sunlight at 60° C. in ambient air: (A) with 0.25 mol % [BMF]+[BF₄]⁻ additive; (B) control.

FIG. 25 shows (A) Reverse J-V characteristics and (B) Normalized EQEs of the p-i-n perovskite solar cell using Cs_{0.17}FA_{0.83}Pb(I_{0.77}Br_{0.23})₃ (without any ionic additive) measured before and after aged for 192 h under full spectrum sunlight at 60° C. in ambient air.

FIG. 26 shows the full width at half maximum (FWHM) were obtained by fitting peaks using the CMPR software. The peaks at around 20.2 and 26.5 degrees were used as representative peaks for the perovskite and FTO glass, respectively. The FTO glass peaks were used as an internal standard.

FIG. 27 shows Pawley fittings of the unencapsulated (A) control and (B) 0.25 mol % [BMP]⁺[BF₄]⁻ modified Cs_{0.17}FA_{0.83}Pb(I_{0.77}Br_{0.23})₃ perovskite films before aging. The XRD peaks are marked by + for PbI₂ and * for FTO. Highlighted by the box, and shown in (C) for the control film, and (D) for the modified film, is the peak corresponding to the (110)_{c} peak in the cubic case, or (020)ₒ,(112)ₒ,(200)ₒ peak in the orthorhombic case. This is the peak which mostly clearly did not fit when fitting to the cubic unit cell, and required an orthorhombic cell to fit well. Using the orthorhombic cell increased the overall goodness of fit (G.O.F) from 1.17 to 1.12 for the control film, and from 1.18 to 1.11 for the treated film.

FIG. 28 shows: (A) the orthorhombic strain of the main orthorhombic perovskite phase for the unencapsulated control and 0.25 mol % [BMP]⁺[BF₄]⁻ modified Cs_{0.17}FA_{0.83}Pb(I_{0.77}Br_{0.23})₃ mixed-cation mixed-anion perovskite films, using the refined lattice parameters to calculate: S (%)=\sqrt{(\sqrt{2}a−\sqrt{2}b)²+(\sqrt{2}b−c)²+(\sqrt{2}a−c)²}. (B) The refined volume of the main orthorhombic perovskite phase over the aging time, showing a rapid increase and then decrease in the control sample, and a more gradual increase for the modified sample. (C) The refined volume of the second cubic perovskite phase, first seen at 168 h in the control and 360 h in the modified sample. When the cubic phase is first seen in the treated sample, it has lattice parameters of FAPbBr₃ (J. Phys. Chem. C 122, 13758-13766 (2018)).

FIG. 29 shows (A) and (B), respectively show the XRD peaks corresponding to the secondary phase for the control and 0.25 mol % [BMP]⁺[BF₄]⁻ modified Cs_{0.17}FA_{0.83}Pb(I_{0.77}Br_{0.23})₃ perovskite films before and after aging.

FIG. 30 shows (A) and (B) present the high-angle XRD data corresponding to the control and 0.25 mol % [BMP]⁺[BF₄]⁻ modified Cs_{0.17}FA_{0.83}Pb(I_{0.77}Br_{0.23})₃ perovskite films, respectively.

FIG. 31 shows optical microscopy (OM) measurements on (A and B) fresh Cs_{0.17}FA_{0.83}Pb(I_{0.77}Br_{0.23})₃ perovskite films and (C-F) after aged under full spectrum sunlight at 60° C. in ambient air for 500 hours. (A, C and E) are the OM images taken only under back illumination using a halogen lamp while (B, D and F) are taken under both the backlit halogen lamp and a frontlit 375-nm UV light source. Scale bar: 100 μm.

FIG. 32 shows optical microscopy (OM) measurements on (A and B) fresh 0.25 mol % [BMP]⁺[BF₄]⁻ modified Cs_{0.17}FA_{0.83}Pb(I_{0.77}Br_{0.23})₃ perovskite films and (C and D) after aged under full spectrum sunlight at 60° C. in ambient air for more than 500 hours. (A and C) are the OM images taken only under back illumination using a halogen lamp while (B and D) are taken under both the backlit halogen lamp and a frontlit 375-nm UV light source. Scale bar: 100 μm.

FIG. 33 shows corresponding top surface SEM images of the fresh and aged control and [BMP]⁺[BF₄]⁻ modified films shown in FIGS. 31 and 32: (A) fresh control; (B) aged control; (C) fresh modified; (D) aged modified. Scale bar: 2 μm.

FIG. 34 shows long-term operational stability. (A) Evolution of SPOs of unencapsulated 0.25 mol % [BMP]⁺[BF₄]⁻ modified and control (Ctrl) Cs_{0.17}FA_{0.83}Pb(I_{0.77}Br_{0.23})₃ perovskite solar cells (8 cells for each condition), aged under full-spectrum sunlight at 60° C. in ambient air. The 95% confidence interval for the SPOs of the modified devices is shown as the green band. The champion cell with the [BMP]⁺[BF₄]⁻ additive is denoted as stars, and the black dotted line is a guide to the eye. The intersections between the data points and the black and green dashed-dotted lines show T_{80,champ} for the champion cell and T_{80,ave} for 8 individual cells, respectively. (B) Corresponding PCEs for (A). (C) Evolution of SPOs of encapsulated 0.25 mol % [BMP]⁺[BF₄]⁻ modified and control Cs_{0.17}FA_{0.83}Pb(I_{0.77}Br_{0.23})₃ cells aged under full-spectrum sunlight at 85° C. in ambient air (6 cells for each condition). The early burn-in region (˜264 h) is determined using a linear model (coefficient of determination R²=96.8%). The intersection between the linear extrapolation for the data (red dashed line) and black dotted line estimated the lifetime for 95% of the post-burn-in SPO (Est. T_{95,ave}) from 6 individual cells. (D) Corresponding PCEs for (C). In all figures, the error bars denote standard deviations.

FIG. 35 shows evolution of device parameters for unencapsulated Cs_{0.17}FA_{0.83}Pb(I_{0.77}Br_{0.23})₃ perovskite solar cells under full-spectrum sunlight at 60° C.: (A) V_{OC}; (B) J_{SC}; (C) FF.

FIG. 36 shows evolution of current density-voltage (J-V) characteristics and static-state power output (SPO) curves for the most stable 0.25 mol % [BMP]⁺[BF₄]⁻ modified Cs_{0.17}FA_{0.83}Pb(I_{0.77}Br_{0.23})₃ perovskite solar cell (unencapsulated) under full-spectrum sunlight at 60° C.: (A) before aging; (B) 48 h; (C) 120 h; (D) 360 h; (E) 792 h; (F) 1008 h.

FIG. 37 shows evolution of device parameters for encapsulated Cs_{0.17}FA_{0.83}Pb(I_{0.77}Br_{0.23})₃ perovskite solar cells under full-spectrum sunlight at 85° C.: (A) V_{OC}; (B) J_{SC}; (C) FF.

FIG. 38 shows evolution of current density-voltage (J-V) characteristics and static-state power output (SPO) curves for the most stable 0.25 mol % [BMP]⁺[BF₄]⁻ modified Cs_{0.17}FA_{0.83}Pb(I_{0.77}Br_{0.23})₃ perovskite solar cell (encapsulated) under full-spectrum sunlight at 85° C.: (A) before aging; (B) 120 h; (C) 360 h; (D) 744 h.

FIG. 39 shows X-ray photoemission spectra of the Pb 4f and I 3d core levels for the fresh and aged control devices (control) and those treated with [BMP]⁺[BF₄]⁻.

FIG. 40 shows a direct comparison of Pb 4f core level spectra. The peaks corresponding to the aged control devices show a clear broadening in comparison to the fresh devices. This broadening is due to the emergence of PbOₓ in the perovskite layer as a result of aging. Conversely no such broadening is seen in the devices with [BMP]⁺[BF₄]⁻.

FIG. 41 shows full peak fittings for X-ray photoemission spectra of Br 3d, C 1s and N 1s core levels.

FIG. 42 shows (A) spectral irradiance of LED (blue) and AM1.5 (orange), and PbI₂ absorptance (green) for equivalent solar intensity calculation. (B) Evolution of appearance of PbI₂ films measured after aged under ˜0.32 suns white LED illumination at 85° C. in a nitrogen filled glove box.

FIG. 43 shows the evolution of corresponding UV-vis absorbance spectra of PbI₂ films shown in FIG. 42 measured before and after aging.

FIG. 44 shows corresponding XRD data of PbI₂ films shown in FIG. 42 measured before and after aging. The XRD peaks corresponding to PbI₂ (+), FTO (*) and Pb (⋄) are marked (Energy. Environ. Sci. 12, 3074-3088, 2019).

FIG. 45 shows Iodine-loss analysis of PbI₂ and perovskite films. (A and B) Top surface SEM images of PbI₂ films: (A) fresh and (B) aged under ˜0.32 suns white LED illumination at 85° C. in a nitrogen filled glove box for 6 hours (scale bar: 1 μm). (C) Schematic of the iodine-loss experimental setup: Vials filled and sealed in nitrogen containing perovskite films fully submerged within toluene were exposed to full spectrum sunlight at 60° C. in ambient air. (D) Photo of the sealed vials with the control and 0.25 mol % [BMP]⁺[BF₄]⁻ additive modified Cs_{0.17}FA_{0.83}Pb(I_{0.77}Br_{0.23})₃ perovskite samples, taken after 4-h light and heat exposure. (E and F) 10 UV-vis absorbance spectra recorded for the toluene solution taken from the (E) control and (F) [BMP]⁺[BF₄]⁻ vials at different aging times. (G) Evolution of absorbance recorded at 500 nm.

FIGS. 46A and 46B provide tables comparing Pb-based solar cell operational stability with the representative literature works. For unencapsulated cells, T_{80,SPO/MPP} (or T_{80,PCE} when T_{80,SPO/MPP} is not available) is listed, while T_{95,SPO/MPP} (or T_{95,PCE} when T_{95,SPO/MPP} is not available) is listed for encapsulated cells (Nat. Energy 5, 35-49, 2020). When performance is given in absolute values, the value of the SPO, MPP or PCE at the T₈₀ or T₉₅ is given.

### DETAILED DESCRIPTION OF THE INVENTION

### Definitions

The term “crystalline” as used herein indicates a crystalline compound, which is a compound having an extended 3D crystal structure. A crystalline compound is typically in the form of crystals or, in the case of a polycrystalline compound, crystallites (i.e. a plurality of crystals having particle sizes of less than or equal to 1 μm). The crystals together often form a layer. The crystals of a crystalline material may be of any size. Where the crystals have one or more dimensions in the range of from 1 nm up to 1000 nm, they may be described as nanocrystals.

The terms “organic compound” and “organic solvent” as used herein have their typical meaning in the art and would readily be understood by the skilled person. The term “organic cation” refers to a cation comprising carbon. The cation may comprise further elements, for example, the cation may comprise hydrogen, nitrogen or oxygen.

The term “crystalline A/M/X material”, as used herein, refers to a material with a crystal structure which comprises one or more A ions, one or more M ions, and one or more X ions. A ions and M ions are cations. X ions are anions. A/M/X materials typically do not comprise any further types of ions.

The term “perovskite”, as used herein, refers to a material with a three-dimensional crystal structure related to that of CaTiO₃ or a material comprising a layer of material, which layer has a structure related to that of CaTiO₃. The structure of CaTiO₃ can be represented by the formula ABX₃, wherein A and B are cations of different sizes and X is an anion. In the unit cell, the A cations are at (0,0,0), the B cations are at (1/2, 1/2, 1/2) and the X anions are at (1/2, 1/2, 0). The A cation is usually larger than the B cation. The skilled person will appreciate that when A, B and X are varied, the different ion sizes may cause the structure of the perovskite material to distort away from the structure adopted by CaTiO₃ to a lower-symmetry distorted structure. The symmetry will also be lower if the material comprises a layer that has a structure related to that of CaTiO₃. Materials comprising a layer of perovskite material are well known. For instance, the structure of materials adopting the K₂NiF₄-type structure comprises a layer of perovskite material. The skilled person will appreciate that a perovskite material can be represented by the formula [A][B][X]₃, wherein [A] is at least one cation, [B] is at least one cation and [X] is at least one anion. When the perovskite comprises more than one A cation, the different A cations may distribute over the A sites in an ordered or disordered way. When the perovskite comprises more than one B cation, the different B cations may distribute over the B sites in an ordered or disordered way. When the perovskite comprises more than one X anion, the different X anions may distribute over the X sites in an ordered or disordered way. The symmetry of a perovskite comprising more than one A cation, more than one B cation or more than one X cation, will be lower than that of CaTiO₃. For layered perovskites the stoichiometry can change between the A, B and X ions. As an example, the [A]₂[B][X]₄ structure can be adopted if the A cation has too large an ionic radius to fit within the 3D perovskite structure. The term “perovskite” also includes A/M/X materials adopting a Ruddleson-Popper phase. Ruddleson-Popper phase refers to a perovskite with a mixture of layered and 3D components. Such perovskites can adopt the crystal structure, Aₙ₋₁A′₂MₙX₃ₙ₊₁, where A and A′ are different cations and n is an integer from 1 to 8, or from 2 to 6. The term “mixed 2D and 3D” perovskite is used to refer to a perovskite film within which there exists both regions, or domains, of AMX₃ and Aₙ₋₁A′₂MₙX₃ₙ₊₁ perovskite phases.

The term “metal halide perovskite”, as used herein, refers to a perovskite, the formula of which contains at least one metal cation and at least one halide anion.

The term “mixed halide perovskite” as used herein refers to a perovskite or mixed perovskite which contains at least two types of halide anion.

The term “mixed cation perovskite” as used herein refers to a perovskite of mixed perovskite which contains at least two types of A cation.

The term “organic-inorganic metal halide perovskite”, as used herein, refers to a metal halide perovskite, the formula of which contains at least one organic cation.

The term “monocation”, as used herein, refers to any cation with a single positive charge, i.e. a cation of formula A⁺ where A is any moiety, for instance a metal atom or an organic moiety. The term “dication”, as used herein, refers to any cation with a double positive charge, i.e. a cation of formula A²⁺ where A is any moiety, for instance a metal atom or an organic moiety. The term “trication”, as used herein, refers to any cation with a triple positive charge, i.e. a cation of formula A³⁺ where A is any moiety, for instance a metal atom or an organic moiety. The term “tetracation”, as used herein, refers to any cation with a quadruple positive charge, i.e. a cation of formula A⁴⁺ where A is any moiety, for instance a metal atom.

The term “alkyl”, as used herein, refers to a linear or branched chain saturated hydrocarbon radical. An alkyl group may be a C₁₋₂₀ alkyl group, a C₁₋₁₄ alkyl group, a C₁₋₁₀ alkyl group, a C₁₋₆ alkyl group or a C₁₋₄ alkyl group. Examples of a C₁₋₁₀ alkyl group are methyl, ethyl, propyl, butyl, pentyl, hexyl, heptyl, octyl, nonyl or decyl. Examples of C₁₋₆ alkyl groups are methyl, ethyl, propyl, butyl, pentyl or hexyl. Examples of C₁₋₄ alkyl groups are methyl, ethyl, i-propyl, n-propyl, t-butyl, s-butyl or n-butyl. If the term “alkyl” is used without a prefix specifying the number of carbons anywhere herein, it has from 1 to 6 carbons (and this also applies to any other organic group referred to herein).

The term “cycloalkyl”, as used herein, refers to a saturated or partially unsaturated cyclic hydrocarbon radical. A cycloalkyl group may be a C₃₋₁₀ cycloalkyl group, a C₃₋₈ cycloalkyl group or a C₃₋₆ cycloalkyl group. Examples of a C₃₋₈ cycloalkyl group include cyclopropyl, cyclobutyl, cyclopentyl, cyclohexyl, cyclohexenyl, cyclohex-1,3-dienyl, cycloheptyl and cyclooctyl. Examples of a C₃₋₆ cycloalkyl group include cyclopropyl, cyclobutyl, cyclopentyl, and cyclohexyl.

The term “alkenyl”, as used herein, refers to a linear or branched chain hydrocarbon radical comprising one or more double bonds. An alkenyl group may be a C₂₋₂₀ alkenyl group, a C₂₋₁₄ alkenyl group, a C₂₋₁₀ alkenyl group, a C₂₋₆ alkenyl group or a C₂₋₄ alkenyl group. Examples of a C₂₋₁₀ alkenyl group are ethenyl (vinyl), propenyl, butenyl, pentenyl, hexenyl, heptenyl, octenyl, nonenyl or decenyl. Examples of C₂₋₆ alkenyl groups are ethenyl, propenyl, butenyl, pentenyl or hexenyl. Examples of C₂₋₄ alkenyl groups are ethenyl, i-propenyl, n-propenyl, s-butenyl or n-butenyl. Alkenyl groups typically comprise one or two double bonds.

The term “alkynyl”, as used herein, refers to a linear or branched chain hydrocarbon radical comprising one or more triple bonds. An alkynyl group may be a C₂₋₂₀ alkynyl group, a C₂₋₄ alkynyl group, a C₂₋₁₀ alkynyl group, a C₂₋₆ alkynyl group or a C₂₋₄ alkynyl group. Examples of a C₂₋₁₀ alkynyl group are ethynyl, propynyl, butynyl, pentynyl, hexynyl, heptynyl, octynyl, nonynyl or decynyl. Examples of C₁₋₆ alkynyl groups are ethynyl, propynyl, butynyl, pentynyl or hexynyl. Alkynyl groups typically comprise one or two triple bonds.

An alkylene group is an unsubstituted or substituted bidentate moiety obtained by removing two hydrogen atoms, either both from the same carbon atom, or one from each of two different carbon atoms, of a hydrocarbon compound typically having from 1 to 20 carbon atoms (C₁₋₂₀ alkylene), which may be aliphatic or alicyclic, and which may be saturated, partially unsaturated, or fully unsaturated. Thus, the term “alkylene” includes the sub-classes alkenylene (C₂₋₂₀ alkenylene), alkynylene (C₂₋₂₀ alkynylene), cycloalkylene, etc. Typically it is C₁₋₁₀ alkylene, or C₁₋₆ alkylene. Typically it is C₁₋₄ alkylene, for example methylene, ethylene, i-propylene, n-propylene, t-butylene, s-butylene or n-butylene. It may also be pentylene, hexylene, heptylene, octylene and the various branched chain isomers thereof. An alkylene group may be unsubstituted or substituted.

The terms “heterocyclyl” and “heterocyclic ring”, as used herein, refer to a monocyclic, bicyclic or polycyclic heterocyclic ring, which ring is saturated or unsaturated, is unsubstituted or substituted, and which typically contains from 5 to 14, more typically from 5 to 10, covalently linked atoms in the ring portion, wherein at least one of the ring atoms is a heteroatom, for example, nitrogen, phosphorus, silicon, oxygen, selenium or sulfur (though more commonly nitrogen, oxygen, or sulfur). A heterocyclic ring may or may not be an aromatic ring. The subset of heterocyclic rings which are aromatic rings are referred to herein as heteroaryl rings or heteroaromatic rings. The terms “heterocyclyl” and “heterocyclic ring” as used herein therefore embrace heteroaryl rings as well as non-aromatic rings. A heterocyclic ring which contains from 5 to 10 covalently linked atoms in the ring portion may be referred to as a C₅₋₁₀ heterocyclic ring, or as a C₅₋₁₀ heterocyclyl. Typically, the heterocyclic ring has from 1 to 4 heteroatoms, and the remainder of the ring atoms are carbon. Typically, the heterocyclic ring is a C₅₋₆ heterocyclic ring in which from 1 to 4 of the ring atoms are ring heteroatoms, and the remainder of the ring atoms are carbon atoms. In this context, the prefixes C₅₋₁₀ and C₅₋₆ denote the number of ring atoms, or range of number of ring atoms. A heterocyclyl, or heterocyclic ring, may be unsubstituted or substituted by, typically, one to four substituents (e.g. one, two, three or four). Where two or more substituents are present, these may be the same or different, and any two of the substituents may be bonded to one another.

The terms “aryl” and “aryl ring”, as used herein, refer to a monocyclic, bicyclic or polycyclic aromatic ring which contains, typically from 6 to 14, more typically from 6 to 10, carbon atoms in the ring portion. Examples include phenyl, naphthyl, indenyl, indanyl, anthrecenyl and pyrenyl groups. The term “aryl group”, as used herein, includes heteroaryl groups. An aryl ring may be unsubstituted or substituted by, typically, one to five substituents (e.g. one, two, three, four or five). Where two or more substituents are present, these may be the same or different, and any two of the substituents may be bonded to one another. An example of a substituted aryl group is pentafluorophenyl.

The terms “heteroaryl” and “heteroaryl ring” as used herein, refers to monocyclic or bicyclic heteroaromatic rings which typically contains from six to fourteen, more typically from six to ten, atoms in the ring portion including one or more heteroatoms. A heteroaryl group is generally a 5- or 6-membered ring, containing at least one heteroatom selected from O, S, N, P, Se and Si. It may contain, for example, one, two, three or four heteroatoms. Examples of heteroaryl groups include pyridyl, pyrazinyl, pyrimidinyl, pyridazinyl, furanyl, thienyl, pyrazolidinyl, pyrrolyl, oxazolyl, oxadiazolyl, isoxazolyl, thiadiazolyl, thiazolyl, isothiazolyl, imidazolyl, pyrazolyl, quinolyl and isoquinolyl. A heteroaryl ring may be unsubstituted or substituted by, typically, one to four substituents (e.g. one, two, three or four). Where two or more substituents are present, these may be the same or different, and any two of the substituents may be bonded to one another.

The term “substituted”, as used herein in the context of substituted organic groups, refers to an organic group which bears one or more substituents selected from C₁₋₁₀ alkyl, aryl (as defined herein), cyano, amino, nitro, C₁₋₁₀ alkylamino, di(C₁₋₁₀)alkylamino, arylamino, diarylamino, aryl(C₁₋₁₀)alkylamino, amido, acylamido, hydroxy, oxo, halo, carboxy, ester, acyl, acyloxy, C₁₋₁₀ alkoxy, aryloxy, halo(C₁₋₁₀)alkyl, sulfonic acid, thiol, C₁₋₁₀ alkylthio, arylthio, sulfonyl, phosphoric acid, phosphate ester, phosphonic acid and phosphonate ester. Examples of substituted alkyl groups include haloalkyl, perhaloalkyl, hydroxyalkyl, aminoalkyl, alkoxyalkyl and alkaryl groups. When a group is substituted, it may bear 1, 2 or 3 substituents. For instance, a substituted group may have 1 or 2 substitutents.

The term “halide” as used herein indicates the singly charged anion of an element in group VIII of the periodic table. “Halide” includes fluoride, chloride, bromide and iodide.

The term “halo” as used herein indicates a halogen atom. Exemplary halo species include fluoro, chloro, bromo and iodo species.

As used herein, an amino group is a radical of formula —NR₂, wherein each R is the same or different and is a substituent. R is usually selected from hydrogen, alkyl, alkenyl, cycloalkyl, or aryl, wherein each of alkyl, alkenyl, cycloalkyl and aryl are as defined herein and may be substituted or unsubstituted, provided that the two R groups may together form an alkylene group. Typically, each R is selected from hydrogen, C₁₋₁₀ alkyl, C₂₋₁₀ alkenyl, and C₃₋₁₀ cycloalkyl. Preferably, each R is selected from hydrogen, C₁₋₆ alkyl, C₂₋₆ alkenyl, and C₃₋₆ cycloalkyl. More preferably, each R is selected from hydrogen and C₁₋₆ alkyl.

A typical amino group is an alkylamino group, which is a radical of formula —NR₂ wherein at least one R is an alkyl group as defined herein. Often, one R is an alkyl group as defined herein, and the other R is as defined above for an amino group, i.e. the other R is selected from hydrogen, alkyl, alkenyl, cycloalkyl, or aryl. Typically, the other R is hydrogen. A C₁₋₁₀ alkylamino group is an alkylamino group wherein at least one R is an C₁₋₁₀ alkyl group. A C₁₋₆ alkylamino group is an alkylamino group wherein at least one R is an C₁₋₆ alkyl group.

Another typical amino group is an dialkylamino group, which is a radical of formula —NR₂ wherein each R is the same or different and is an alkyl group as defined herein, provided that the two alkyl groups R may be joined together to form an alkylene group. A di(C₁₋₁₀)alkylamino group is a dialkylamino group wherein each R is the same or different C₁₋₁₀ alkyl group. A di(C₁₋₆)alkylamino group is a dialkylamino group wherein each R is the same or different C₁₋₆ alkyl group.

As used herein, an imino group is a radical of formula R₂C═N— or —C(R)═NR, wherein each R is a substituent. That is, an imino group is a radical comprising a C═N moiety, having the radical moiety either at the N atom or attached to the C atom of said C═N bond. R is as defined herein: that is, R is usually selected from hydrogen, alkyl, alkenyl, cycloalkyl, or aryl, wherein each of alkyl, alkenyl, cycloalkyl and aryl are as defined herein. Typically, each R is selected from hydrogen, C₁₋₆ alkyl, C₂₋₆ alkenyl, and C₃₋₁₀ cycloalkyl. Preferably, each R is selected from hydrogen, C₁₋₆ alkyl, C₂₋₆ alkenyl, and C₃₋₆ cycloalkyl. More preferably, each R is selected from hydrogen and C₁₋₆ alkyl.

A typical imino group is an alkylimino group, which is a radical of formula R₂C═N— or —C(R)═NR wherein at least one R is an alkyl group as defined herein. A C₁₋₆ alkylimino group is an alkylimino group wherein the R substituents comprise from 1 to 6 carbon atoms.

The term “ester” as used herein indicates an organic compound of the formula alkyl-C(═O)—O-alkyl, wherein the alkyl radicals are the same or different and are as defined herein. The alkyl radicals may be optionally substituted.

The term “ether” as used herein indicates an oxygen atom substituted with two alkyl radicals as defined herein. The alkyl radicals may be optionally substituted, and may be the same or different.

As used herein, the term “ammonium” indicates an organic cation of formula R¹R²R³R⁴N⁺. R¹, R², R³, and R⁴ are substituents. Each of R¹, R², R³, and R⁴ is bonded to the nitrogen atom, N, via a single bond. Each of R¹, R², R³, and R⁴ is typically independently selected from hydrogen, or from optionally substituted alkyl, alkenyl, aryl, cycloalkyl and cycloalkenyl; the optional substituent is preferably a hydroxyl or an amino or imino substituent. Usually, each of R¹, R², R³, and R⁴ is independently selected from hydrogen, and optionally substituted C₁₋₁₀ alkyl, C₂₋₁₀ alkenyl, C₃₋₁₀ cycloalkyl, C₃₋₁₀ cycloalkenyl and C₆₋₁₂ aryl; where present, the optional substituent is preferably a hydroxyl or an amino group; for instance C₁₋₆ amino. Preferably, each of R¹, R², R³, and R⁴ is independently selected from hydrogen, and unsubstituted C₁₋₁₀ alkyl, C₂₋₁₀ alkenyl, C₃₋₁₀ cycloalkyl, C₃₋₁₀ cycloalkenyl, and C₆₋₁₂ aryl. In a particularly preferred embodiment, R¹, R², R³, and R⁴ are independently selected from hydrogen, C₁₋₁₀ alkyl, and C₂₋₁₀ alkenyl. Further preferably, R¹, R², R³, and R⁴ are independently selected from hydrogen, C₁₋₆ alkyl and C₂₋₆ alkenyl.

As used herein, the term “iminium” indicates an organic cation of formula (R¹R²C═NR³R⁴)⁺, wherein R¹, R², R³, and R⁴ are as defined in relation to the ammonium cation. Thus, in a particularly preferred embodiment, of the iminium cation, R¹, R², R³, and R⁴ are independently selected from hydrogen, C₁₋₁₀ alkyl, C₂₋₁₀ alkenyl and C₁₋₆ amino. In a further preferable embodiment of the iminium cation, R¹, R², R³, and R⁴ are independently selected from hydrogen, C₁₋₆ alkyl, C₂₋₆ alkenyl and C₁₋₆ amino. Often, the iminium cation is formamidinium, i.e. R^{c} is NH₂ and R², R³ and R⁴ are all H.

As used herein, the term “primary ammonium” indicates an organic cation of formula R^{a}H₃N⁺. R^{a} is a substituent other than hydrogen. R^{a} is bonded to the nitrogen atom, N, via a single bond. Thus, a moiety of formula (I) hereinbelow in which the positively-charged nitrogen atom is bonded to a carbon atom via a double bond is not a primary ammonium cation. R^{a} is usually a hydrocarbyl group. A hydrocarbyl group is a group which is formed by removing a hydrogen atom from a hydrocarbon. Hydrocarbyl group R^{a} may be unsubstituted or substituted, for example substituted with a hydroxyl group. R^{a} is typically selected from unsubstituted or substituted alkyl, unsubstituted or substituted alkenyl, unsubstituted or substituted aryl, unsubstituted or substituted cycloalkyl and unsubstituted or substituted cycloalkenyl; the optional substituent is preferably a hydroxyl, amino or imino substituent. Usually, R^{a} is selected from unsubstituted or substituted C₁₋₁₀ alkyl, unsubstituted or substituted C₂₋₁₀ alkenyl, unsubstituted or substituted C₃₋₁₀ cycloalkyl, unsubstituted or substituted C₃₋₁₀ cycloalkenyl, and unsubstituted or substituted C₆₋₁₂ aryl; where present, the optional substituent is preferably a hydroxyl or an amino group (for instance C₁₋₆ amino). For instance, R^{a} may be selected from unsubstituted C₁₋₁₀ alkyl, C₂₋₁₀ alkenyl, C₃₋₁₀ cycloalkyl, C₃₋₁₀ cycloalkenyl and C₆₋₁₂ aryl. R^{a} may for instance be selected from unsubstituted or substituted C₁₋₁₀ alkyl groups, or, for instance, from unsubstituted or substituted C₁₋₆ alkyl groups or, for example, from methyl, ethyl and propyl groups. The optional substituent may for instance be a hydroxyl group. An example of a primary ammonium cation is methylammonium (“MA”).

The term “primary ammonium halide”, as used herein, therefore refers to a salt having a primary ammonium cation and a halide anion. A primary ammonium halide is often a primary ammonium chloride, a primary ammonium bromide or a primary ammonium iodide. It may for instance be methylammonium chloride, bromide or iodide.

As used herein, the term “secondary ammonium” indicates an organic cation of formula R^{a}R^{b}H₂N⁺. R^{a} and R^{b} are substituents other than hydrogen. Each of R^{a} and R^{b} is bonded to the nitrogen atom, N, via a single bond. Thus, a moiety of formula (I) hereinbelow in which the positively-charged nitrogen atom is bonded to a carbon atom via a double bond is not a secondary ammonium cation. R^{a} and R^{b} are usually both hydrocarbyl groups. A hydrocarbyl group is a group which is formed by removing a hydrogen atom from a hydrocarbon. Hydrocarbyl groups R^{a} and R^{b} in the secondary ammonium cation may be the same or different. R^{a} and R^{b} may optionally be joined (bonded) to each other (i.e. other than via the nitrogen) to form a bidentate group (which together with the nitrogen atom, N, will form a heterocylic ring). Each hydrocarbyl group may be unsubstituted or substituted, for example one or more both the hydrocarbyl groups in the secondary ammonium cation may be substituted with a hydroxyl group. Each of R^{a} and R^{b} is typically independently selected from unsubstituted or substituted alkyl, unsubstituted or substituted alkenyl, unsubstituted or substituted aryl, unsubstituted or substituted cycloalkyl and unsubstituted or substituted cycloalkenyl; the optional substituent is preferably a hydroxyl, amino or imino substituent. Usually, each of R^{a} and R^{b} is independently selected from unsubstituted or substituted C₁₋₁₀alkyl, unsubstituted or substituted C₂₋₁₀ alkenyl, unsubstituted or substituted C₃₋₁₀ cycloalkyl, unsubstituted or substituted C₃₋₁₀ cycloalkenyl, and unsubstituted or substituted C₆₋₁₂ aryl; where present, the optional substituent is preferably a hydroxyl or an amino group (for instance C₁₋₆ amino). For instance, each of R^{a} and R^{b} may be independently selected from unsubstituted C₁₋₁₀ alkyl, C₂₋₁₀ alkenyl, C₃₋₁₀ cycloalkyl, C₃₋₁₀ cycloalkenyl and C₆₋₁₂ aryl. R^{a} and R^{b} may for instance be independently selected from unsubstituted or substituted C₁₋₁₀ alkyl groups, or, for instance, from unsubstituted or substituted C₁₋₆ alkyl groups or, for example, from methyl, ethyl and propyl groups. The optional substituent may for instance be a hydroxyl group. An example of a secondary ammonium cation is phenylethylammonium.

The term “secondary ammonium halide”, as used herein, therefore refers to a salt having a secondary ammonium cation and a halide anion. A secondary ammonium halide is often a secondary ammonium chloride, a secondary ammonium bromide or a secondary ammonium iodide. It may for instance be phenylethylammonium chloride, bromide or iodide.

As used herein, the term “tertiary ammonium” indicates an organic cation of formula R^{a}R^{b}R^{c}HN⁺. R^{a}, R^{b} and R^{c} are substituents other than hydrogen. Each of R^{a}, R^{b} and R^{c} is bonded to the nitrogen atom, N, via a single bond. Thus, a moiety of formula (I) hereinbelow in which the positively-charged nitrogen atom is bonded to a carbon atom via a double bond is not a tertiary ammonium cation. R^{a}, R^{b} and R^{c} are usually all hydrocarbyl groups. A hydrocarbyl group is a group which is formed by removing a hydrogen atom from a hydrocarbon. Hydrocarbyl groups R^{a}, R^{b} and R^{c} in the tertiary ammonium cation may be the same or different. One of R^{a}, R^{b} and R^{c} may optionally be joined (bonded) to another one of R^{a}, R^{b} and R^{c} (i.e. other than via the nitrogen) to form a bidentate group (which together with the nitrogen atom, N, will form a heterocylic ring). Each hydrocarbyl group may be unsubstituted or substituted, for example one or more of the hydrocarbyl groups in the tertiary ammonium cation may be substituted with a hydroxyl group. Each of R^{a}, R^{b} and R^{c} is typically independently selected from unsubstituted or substituted alkyl, unsubstituted or substituted alkenyl, unsubstituted or substituted aryl, unsubstituted or substituted cycloalkyl and unsubstituted or substituted cycloalkenyl; the optional substituent is preferably a hydroxyl, amino or imino substituent. Usually, each of R^{a}, R^{b} and R^{c} is independently selected from unsubstituted or substituted C₁₋₁₀ alkyl, unsubstituted or substituted C₂₋₁₀ alkenyl, unsubstituted or substituted C₃₋₁₀ cycloalkyl, unsubstituted or substituted C₃₋₁₀ cycloalkenyl, and unsubstituted or substituted C₆₋₁₂ aryl; where present, the optional substituent is preferably a hydroxyl or an amino group (for instance C₁₋₆ amino). For instance, each of R^{a}, R^{b} and R^{c} may be independently selected from unsubstituted C₁₋₁₀ alkyl, C₂₋₁₀ alkenyl, C₃₋₁₀ cycloalkyl, C₃₋₁₀ cycloalkenyl and C₆₋₁₂ aryl. R^{a}, R^{b} and R^{c} may for instance be independently selected from unsubstituted or substituted C₁₋₁₀ alkyl groups, or, for instance, from unsubstituted or substituted C₁₋₆ alkyl groups or, for example, from methyl, ethyl and propyl groups. The optional substituent may for instance be a hydroxyl group. An example of a tertiary ammonium cation is trimethylammonium.

The term “tertiary ammonium halide”, as used herein, therefore refers to a salt having a tertiary ammonium cation and a halide anion. A tertiary ammonium halide is often a tertiary ammonium chloride, a tertiary ammonium bromide or a tertiary ammonium iodide. It may for instance be trimethylammonium chloride, bromide or iodide.

As used herein, the term “quaternary ammonium” indicates an organic cation of formula R^{a}R^{b}R^{c}R^{d}N⁺. R^{a}, R^{b}, R^{c}, and R^{d} are substituents other than hydrogen. Each of R^{a}, R^{b}, R^{c} and R^{d} is bonded to the nitrogen atom, N, via a single bond. Thus, a moiety of formula (I) hereinbelow in which the positively-charged nitrogen atom is bonded to a carbon atom via a double bond is not a quaternary ammonium cation. Unlike the ammonium ion (NH₄⁺, which is an inorganic cation) and the primary, secondary, or tertiary ammonium cations, a quaternary ammonium cation is permanently charged, independent of the pH of a solution in which is may be present. R^{a}, R^{b}, R^{c}, and R^{d} are usually all hydrocarbyl groups. A hydrocarbyl group is a group which is formed by removing a hydrogen atom from a hydrocarbon. Hydrocarbyl groups R^{a}, R^{b}, R^{c} and R^{d} in the quaternary ammonium cation may be the same or different. One of R^{a}, R^{b}, R^{c} and R^{d} may optionally be joined (bonded) to another one of R^{a}, R^{b}, R^{c} and R^{d} (i.e. other than via the nitrogen) to form a bidentate group (which together with the nitrogen atom, N, will form a heterocylic ring). Each hydrocarbyl group may be unsubstituted or substituted, for example one or more of the hydrocarbyl groups in the quaternary ammonium cation may be substituted with a hydroxyl group. Each of R^{a}, R^{b}, R^{c}, and R^{d} is typically independently selected from unsubstituted or substituted alkyl, unsubstituted or substituted alkenyl, unsubstituted or substituted aryl, unsubstituted or substituted cycloalkyl and unsubstituted or substituted cycloalkenyl; the optional substituent is preferably a hydroxyl, amino or imino substituent. Usually, each of R^{a}, R^{b}, R^{c}, and R^{d} is independently selected from unsubstituted or substituted C₁₋₁₀ alkyl, unsubstituted or substituted C₂₋₁₀ alkenyl, unsubstituted or substituted C₃₋₁₀ cycloalkyl, unsubstituted or substituted C₃ cycloalkenyl, and unsubstituted or substituted C₆₋₁₂ aryl; where present, the optional substituent is preferably a hydroxyl or an amino group (for instance C₁₋₆ amino). For instance, each of R^{a}, R^{b}, R^{c}, and R^{d} may be independently selected from unsubstituted C₁₋₁₀ alkyl, C₂₋₁₀ alkenyl, C₃₋₁₀ cycloalkyl, C₃₋₁₀ cycloalkenyl and C₆₋₁₂ aryl. R^{a}, R^{b}, R^{c}, and R^{d} may for instance be independently selected from unsubstituted or substituted C₁₋₁₀ alkyl groups, or, for instance, from unsubstituted or substituted C₁₋₆ alkyl groups. The optional substituent may for instance be a hydroxyl group. Examples of quaternary ammonium cations include tetramethylammonium, choline and n-hexyl trimethyl ammonium.

The term “quaternary ammonium halide”, as used herein, therefore refers to a salt having a quaternary ammonium cation and a halide anion. A quaternary ammonium halide is often a quaternary ammonium chloride, a quaternary ammonium bromide or a quaternary ammonium iodide. Examples of a quaternary ammonium halides include tetramethylammonium chloride, choline halides, and n-hexyl trimethyl ammonium bromide.

The term “optoelectronic device”, as used herein, refers to devices which source, control or detect light. Light is understood to include any electromagnetic radiation. Examples of optoelectronic devices include photovoltaic devices (including solar cells), photodiodes, phototransistors, photomultipliers, photoresistors, and light emitting diodes.

The term “consisting essentially of” refers to a composition comprising the components of which it consists essentially as well as other components, provided that the other components do not materially affect the essential characteristics of the composition. Typically, a composition consisting essentially of certain components will comprise greater than or equal to 95 wt % of those components or greater than or equal to 99 wt % of those components.

The terms “disposing on” or “disposed on”, as used herein, refer to the making available or placing of one component on another component. The first component may be made available or placed directly on the second component, or there may be a third component which intervenes between the first and second component. For instance, if a first layer is disposed on a second layer, this includes the case where there is an intervening third layer between the first and second layers. Typically, “disposing on” refers to the direct placement of one component on another.

Similarly, the terms “disposing between” or “disposed between”, as used herein, refer to the making available or placing of one (first) component between two other (second and third) components. The first component may be made available or placed directly between the second and third components, or there may be a further component which intervenes between the first and second components and/or between the first and third components. For instance, if a first layer is disposed between a second layer and a third layer, this includes the case where there is an intervening fourth layer between the first and second layers and an intervening fifth layer between the first and third layers. Often, however, “disposing between” refers to the direct placement or making available of one (first) component between two other (second and third) components.

The term “layer”, as used herein, refers to any structure which is substantially laminar in form (for instance extending substantially in two perpendicular directions, but limited in its extension in the third perpendicular direction). A layer may have a thickness which varies over the extent of the layer. Typically, a layer has approximately constant thickness. The “thickness” of a layer, as used herein, refers to the average thickness of a layer. The thickness of layers may easily be measured, for instance by using microscopy, such as electron microscopy of a cross section of a film, or by surface profilometry for instance using a stylus profilometer.

The term “band gap”, as used herein, refers to the energy difference between the top of the valence band and the bottom of the conduction band in a material. The skilled person of course is readily able to measure the band gap of a semiconductor (including that of a perovskite) by using well-known procedures which do not require undue experimentation. For instance, the band gap of a semiconductor can be estimated by constructing a photovoltaic diode or solar cell from the semiconductor and determining the photovoltaic action spectrum. Alternatively the band gap can be estimated by measuring the light absorption spectra either via transmission spectrophotometry or by photo thermal deflection spectroscopy. The band gap can be determined by making a Tauc plot, as described in Tauc, J., Grigorovici, R. & Vancu, a. Optical Properties and Electronic Structure of Amorphous Germanium. Phys. Status Solidi 15, 627-637 (1966) where the square of the product of absorption coefficient times photon energy is plotted on the Y-axis against photon energy on the x-axis with the straight line intercept of the absorption edge with the x-axis giving the optical band gap of the semiconductor. Alternatively, the optical band gap may be estimated by taking the onset of the incident photon-to-electron conversion efficiency, as described in [Barkhouse DAR, Gunawan O, Gokmen T, Todorov TK, Mitzi DB. Device characteristics of a 10.1% hydrazineprocessed Cu2ZnSn(Se,S)4 solar cell. Progress in Photovoltaics: Research and Applications 2012; published online DOI: 10.1002/pip.1160.] The term “semiconductor” or “semiconducting material”, as used herein, refers to a material with electrical conductivity intermediate in magnitude between that of a conductor and a dielectric. A semiconductor may be a negative (n)-type semiconductor, a positive (p)-type semiconductor or an intrinsic (i) semiconductor. A semiconductor may have a band gap of from 0.5 to 3.5 eV, for instance from 0.5 to 2.5 eV or from 1.0 to 2.0 eV (when measured at 300 K).

The term “n-type region”, as used herein, refers to a region of one or more electron-transporting (i.e. n-type) materials. Similarly, the terms “n-type layer” refers to a layer of an electron-transporting (i.e. an n-type) material. An electron-transporting (i.e. an n-type) material could be a single electron-transporting compound or elemental material, or a mixture of two or more electron-transporting compounds or elemental materials. An electron-transporting compound or elemental material may be undoped or doped with one or more dopant elements.

The term “p-type region”, as used herein, refers to a region of one or more hole-transporting (i.e. p-type) materials. Similarly, the term “p-type layer” refers to a layer of a hole-transporting (i.e. a p-type) material. A hole-transporting (i.e. a p-type) material could be a single hole-transporting compound or elemental material, or a mixture of two or more hole-transporting compounds or elemental materials. A hole-transporting compound or elemental material may be undoped or doped with one or more dopant elements.

The term “electrode material”, as used herein, refers to any material suitable for use in an electrode. An electrode material will have a high electrical conductivity. The term “electrode” as used herein indicates a region or layer consisting of, or consisting essentially of, an electrode material.

The term “ionic solid”, as used herein, refers to a salt which is in the solid state at room temperature. Typically, the ionic solid is a salt which is in the solid state at 50° C. and at temperatures of less than 50° C. In other words, the ionic solid is typically a salt whose melting point is greater than 50° C. Preferably, the ionic solid is a salt which is in the solid state at 100° C. and at temperatures of less than 100° C. In other words, the ionic solid is preferably a salt whose melting point is greater than 100° C. Often, the ionic solid is a salt which is in the solid state at 120° C. and at temperatures of less than 120° C. In other words, the ionic solid is preferably a salt whose melting point is greater than 120° C.

### Optoelectronic Device

The present invention provides an optoelectronic device comprising:
(a) a layer comprising a crystalline A/M/X material, wherein the crystalline A/M/X material comprises a compound of formula:

[A]ₐ[M]_{b}[X]_{c}

wherein: [A] comprises one or more A cations; [M] comprises one or more M cations which are metal or metalloid cations; [X] comprises one or more X anions; a is a number from 1 to 6; b is a number from 1 to 6; and c is a number from 1 to 18; and
(b) an ionic solid which is a salt comprising an organic cation and a counter anion. Optionally, the ionic solid is other than a quaternary ammonium halide salt. The ionic solid is usually other than a primary ammonium halide salt. The ionic solid is often other than a secondary ammonium halide salt. The ionic solid is usually other than a tertiary ammonium halide salt. Thus, typically, the ionic solid is other than a primary, secondary, tertiary or quaternary ammonium halide salt. The ionic solid is typically other than a formamidinium halide salt, and usually other than a guanidinium halide salt. Thus, typically, the ionic solid is other than a primary, secondary, tertiary or quaternary ammonium halide salt and other than a formamidinium or guanidinium halide salt. The ionic solid is typically other than a halide salt of a cation of formula (X) as defined herein. Often, the ionic solid is other than a primary, secondary, tertiary or quaternary ammonium halide salt and other than a halide salt of a cation of formula (X) as defined herein. Typically, when the counter-anion of the ionic solid is halide, the organic cation of the ionic solid is other than each of the one or more A cations of the crystalline A/M/X material. Often, the organic cation of the ionic solid is other than each of the one or more A cations of the crystalline A/M/X material.

The organic cation is typically present on or within the layer comprising the crystalline A/M/X material.

The organic cation is typically in contact with the crystalline A/M/X material. It may be present within the crystalline A/M/X material, on an outer surface of the crystalline A/M/X material, or both.

Often, the organic cation is present within the layer comprising the crystalline A/M/X material. The organic cation being present within the layer comprising the crystalline A/M/X material typically means that the organic cation is present not just at the outer edges of the layer comprising the crystalline A/M/X material but also exists throughout the bulk of the layer comprising the crystalline A/M/X material. Thus, the organic cation may be present at a surface of the layer comprising the crystalline A/M/X material and throughout the bulk of the layer comprising the crystalline A/M/X material. The surface may be the top or bottom surface of the layer comprising the crystalline A/M/X material, i.e. either of its two surfaces. The organic cation may be present at both of the surfaces of the layer comprising the crystalline A/M/X material (i.e. at both the top and bottom surfaces of the layer) and throughout the bulk of the layer comprising the crystalline A/M/X material. Thus, the organic cation may be present at the surfaces of the layer comprising the crystalline A/M/X material and throughout the bulk of the layer comprising the crystalline A/M/X material.

The organic cation may be present at a surface of the layer comprising the crystalline A/M/X material. The surface may be the top or bottom surface of the layer comprising the crystalline A/M/X material, i.e. either of its two surfaces. The organic cation may be present at both of the surfaces of the layer comprising the crystalline A/M/X material (i.e. at both the top and bottom surfaces of the layer). Thus, the organic cation may be present at the surfaces of the layer comprising the crystalline A/M/X material.

The organic cation may be present on a surface of the crystalline A/M/X material. The organic cation may be present on an outer surface of the crystalline A/M/X material. The surface may be a top or a bottom surface of the crystalline A/M/X material. The organic cation may be present on both a top and a bottom surface of the crystalline A/M/X material. Thus, the organic cation may be present on the surfaces of the crystalline A/M/X material.

Often, the organic cation is present within the crystalline A/M/X material.

Typically, the crystalline A/M/X material is a polycrystalline A/M/X material comprising crystallites of the A/M/X material and grain boundaries between the crystallites. Thus, the layer comprising a crystalline A/M/X material may comprise multiple crystallites of the A/M/X material with grain boundaries between the crystallites.

When the crystalline A/M/X material is a polycrystalline A/M/X material comprising crystallites of the A/M/X material and grain boundaries between the crystallites, the organic cation is typically present at grain boundaries between the crystallites. For instance, the organic cation may be present throughout the bulk of the layer comprising the crystalline A/M/X material, at grain boundaries between the crystallites.

Thus, the organic cation may be present within the crystalline A/M/X material, at grain boundaries within the crystalline A/M/X material.

The organic cation may be present on the surface of the crystalline A/M/X material and within the crystalline A/M/X material. Thus, the organic cation may be present on an outer surface of the crystalline A/M/X material and within the crystalline A/M/X material. The A/M/X material may be polycrystalline and the organic cation may be present (i) on an outer surface of the polycrystalline A/M/X material, and (ii) within the polycrystalline A/M/X material, at grain boundaries between crystallites of the polycrystalline A/M/X material. Thus, the organic cation may be present on the surface of the crystalline A/M/X material, for instance on an outer surface of the material, and also within the crystalline A/M/X material, at grain boundaries within the crystalline A/M/X material.

### Ionic Solid

Typically, the counter anion is other than a halide anion, or the organic cation is other than a quaternary ammonium cation. Thus, in one embodiment the counter anion is other than a halide anion. In another embodiment the organic cation is other than a quaternary ammonium cation. Thus, in one embodiment the ionic solid comprises an organic cation other than a quaternary ammonium cation and counter-anion which is a halide anion. In another embodiment, the ionic solid comprises an organic cation which is a quaternary ammonium cation and a counter-anion that is other than a halide anion. In another embodiment, the organic cation is other than a quaternary ammonium cation and the counter anion is other than a halide anion.

Typically, the counter anion is other than a halide anion, or the organic cation is other than a tertiary ammonium cation. Thus, in one embodiment the counter anion is other than a halide anion. In another embodiment the organic cation is other than a tertiary ammonium cation.

Thus, in one embodiment the ionic solid comprises an organic cation other than a tertiary ammonium cation and counter-anion which is a halide anion. In another embodiment, the ionic solid comprises an organic cation which is a tertiary ammonium cation and a counter-anion that is other than a halide anion. In another embodiment, the organic cation is other than a tertiary ammonium cation and the counter anion is other than a halide anion.

Typically, the counter anion is other than a halide anion, or the organic cation is other than a secondary ammonium cation. Thus, in one embodiment the counter anion is other than a halide anion. In another embodiment the organic cation is other than a secondary ammonium cation.

Thus, in one embodiment the ionic solid comprises an organic cation other than a secondary ammonium cation and counter-anion which is a halide anion. In another embodiment, the ionic solid comprises an organic cation which is a secondary ammonium cation and a counter-anion that is other than a halide anion. In another embodiment, the organic cation is other than a secondary ammonium cation and the counter anion is other than a halide anion.

Typically, the counter anion is other than a halide anion, or the organic cation is other than a primary ammonium cation. Thus, in one embodiment the counter anion is other than a halide anion. In another embodiment the organic cation is other than a primary ammonium cation.

Thus, in one embodiment the ionic solid comprises an organic cation other than a primary ammonium cation and counter-anion which is a halide anion. In another embodiment, the ionic solid comprises an organic cation which is a primary ammonium cation and a counter-anion that is other than a halide anion. In another embodiment, the organic cation is other than a primary ammonium cation and the counter anion is other than a halide anion.

Typically, the counter anion is other than a halide anion, or the organic cation is other than a primary, secondary, tertiary or quaternary ammonium cation. Thus, in one embodiment the counter anion is other than a halide anion. In another embodiment the organic cation is other than a primary, secondary, tertiary or quaternary ammonium cation.

Thus, in one embodiment the ionic solid comprises an organic cation other than a primary, secondary, tertiary or quaternary ammonium cation and counter-anion which is a halide anion. In another embodiment, the ionic solid comprises an organic cation which is a primary, secondary, tertiary or quaternary ammonium cation and a counter-anion that is other than a halide anion. In another embodiment, the organic cation is other than a primary, secondary, tertiary or quaternary ammonium cation and the counter anion is other than a halide anion.

Typically, the counter anion is other than a halide anion, or the organic cation is other than a formamidinium or guanidinium cation. A formamidinium (FA) cation has the formula [H₂N—C(H)═NH₂]⁺ and a guanidinium cation has the formula [H₂N—C(NH₂)═NH₂]⁺. Thus, in one embodiment the counter anion is other than a halide anion. In another embodiment the organic cation is other than a formamidinium or guanidinium cation.

Thus, in one embodiment the ionic solid comprises an organic cation other than a formamidinium or guanidinium cation and counter-anion which is a halide anion. In another embodiment, the ionic solid comprises an organic cation which is a formamidinium or guanidinium cation and a counter-anion that is other than a halide anion. In another embodiment, the organic cation is other than a formamidinium or guanidinium cation and the counter anion is other than a halide anion.

Typically, the counter anion is other than a halide anion, or the organic cation is other than a cation of formula (X)

[R^{P}R^{Q}N—C(R^{S})═NH₂]⁺ (X)

wherein

R^{S} is R^{T} or NR^{U}R^{V}, wherein R^{T}, R^{U} and R^{V} are independently selected from H, methyl, ethyl and phenyl;

R^{P} and R^{Q} are independently selected from hydrogen, unsubstituted or substituted C₁₋₁₀ alkyl, unsubstituted or substituted aryl, unsubstituted or substituted C₃₋₁₀ cycloalkyl, unsubstituted or substituted heterocyclyl, unsubstituted or substituted amino, unsubstituted or substituted (C₁₋₁₀ alkyl)amino, or unsubstituted or substituted di(C₁₋₁₀ alkyl)amino, provided that R^{x} may together with R^{y} form a C₁₋₆ alkylene group. Usually, R^{P} and R^{Q} are independently selected from H, methyl, ethyl and phenyl. Typically, R^{P} and R^{Q} are both H. Often, R^{P}, R^{Q}, R^{T}, R^{U} and R^{V} are all H.

Thus, in one embodiment the counter anion is other than a halide anion. In another embodiment the organic cation is other than a cation of formula (X).

Thus, in one embodiment the ionic solid comprises an organic cation other than a cation of formula (X) and counter-anion which is a halide anion. In another embodiment, the ionic solid comprises an organic cation which is a cation of formula (X) and a counter-anion that is other than a halide anion. In another embodiment, the organic cation is a cation of formula (X) and the counter anion is other than a halide anion, i.e. the ionic solid is not a halide salt of a formula (X) cation.

Typically, the counter anion is other than a halide anion, or the organic cation is other than a primary, secondary, tertiary or quaternary ammonium cation and other than a formamidinium or guanidinium cation. Thus, in one embodiment the counter anion is other than a halide anion. In another embodiment the organic cation is other than a primary, secondary, tertiary or quaternary ammonium cation and other than a formamidinium or guanidinium cation.

Thus, in one embodiment the ionic solid comprises (i) an organic cation other than a primary, secondary, tertiary or quaternary ammonium cation and other than a formamidinium or guanidinium cation, and (ii) a counter-anion which is a halide anion. In another embodiment, the ionic solid comprises (i) an organic cation which is a primary, secondary, tertiary or quaternary ammonium cation or a formamidinium or guanidinium cation, and (ii) a counter-anion that is other than a halide anion. In another embodiment, the organic cation is other than a primary, secondary, tertiary or quaternary ammonium cation and other than a formamidinium or guanidinium cation, and the counter anion is other than a halide anion.

Typically, the counter anion is other than a halide anion, or the organic cation is other than a primary, secondary, tertiary or quaternary ammonium cation and other than a cation of formula (X). Thus, in one embodiment the counter anion is other than a halide anion. In another embodiment the organic cation is other than a primary, secondary, tertiary or quaternary ammonium cation and other than a cation of formula (X).

Thus, in one embodiment the ionic solid comprises (i) an organic cation other than a primary, secondary, tertiary or quaternary ammonium cation and other than a cation of formula (X), and (ii) a counter-anion which is a halide anion. In another embodiment, the ionic solid comprises (i) an organic cation which is a primary, secondary, tertiary or quaternary ammonium cation or a cation of formula (X), and (ii) a counter-anion that is other than a halide anion. In another embodiment, the organic cation is other than a primary, secondary, tertiary or quaternary ammonium cation and other than a cation of formula (X), and the counter anion is other than a halide anion.

Especially when the counter anion of the ionic solid is halide, the organic cation is typically other than each of the one or more A cations of the crystalline A/M/X material.

Counter anions other than halide anions are well known to the skilled person. For instance the counter anion may be a hydroxide, a cyanide, a chalcogenide, a borate, a phosphate, a nitrate, a nitrite, a carborane anion, a carbonate, a sulphate, a polyatomic anion comprising a halogen, a thiocyanate anion, a triflate, an oxyanion of a transition metal, a negatively charged metal complex or an organic anion.

The counter anion may, for instance, be a monoanion, a dianion or a trianion. It is typically a monoanion or a dianion. Often, however, the counter anion is a monoanion, i.e. it has a single negative charge.

Examples of chalcogenides include sulphide, selenide, and telluride. Examples of polyatomic anions comprising a halogen include hexahalophosphates (including hexafluorophosphate), tetrahaloborates (including tetrafluoroborate), hypofluorite, hypochorite, chlorite, chlorate, perchlorate, hypobromite, bromite, bromate, perbromate, hypoiodite, hypoioidite, iodate and periodiate. Oxyanions of a transition metal include manganite ([MnO₄]⁻), chromate ([CrO₄]²⁻) and dichromate ([Cr₂O₇]²⁻). Examples of negatively charged metal complexes include [Al(OC(CF₃)₃)₄)]⁻.

Typically, the counter-anion is a polyatomic anion. In other words, the counter-anion may be a molecule comprising two or more atoms that carries a negative charge. Preferably the polyatomic anion is a non-coordinating anion. Examples of non-coordinating anions include borates (including tetrahaloborates), chlorates, triflates, carborane anions (e.g. CB₁₁H₁₂⁻), phosphates (including hexahalophosphates) and [Al(OC(CF₃)₃)₄)]⁻. Often, the non-coordinating polyatomic anion employed in the ionic solid is a hexahalophosphate or a tetrahaloborate; it is often hexafluorophosphate ([PF₆]⁻) or tetrafluoroborate (BF₄⁻).

Examples of phosphates include hexahalophosphates such as hexafluorophosphate ([PF₆]⁻). Thus, in one embodiment, the counter-anion is hexafluorophosphate ([PF₆]⁻).

Typically, the counter-anion is a borate anion. Typically, the borate anion is an anion of the formula [BX₄]⁻, wherein each X is independently selected from hydrogen, halo, unsubstituted or substituted alkyl, unsubstituted or substituted alkeynyl, unsubstituted or substituted alkynyl, unsubstituted or substituted aryl, or unsubstituted or substituted heteroaryl. For instance, each X may be independently selected from halo or unsubstituted or substituted aryl, typically pentafluorophenyl or 3,5-bis(trifluoromethyl)phenyl.

Typically, all four X atoms are halo. Thus, preferably the counter-anion is a tetrahaloborate. Often, the counter-anion is tetrafluoroborate (BF₄⁻).

Alternatively, all four X groups may be substituted aryl. For instance all four X groups may be pentafluorophenyl or 3,5-bis(trifluoromethyl)phenyl. Thus, the counter-anion may be tetrakis(pentafluorophenyl)borate ([B(C₆F₅)₄]⁻) or tetrakis[3,5-bis(trifluoromethyl)phenyl]borate ([B(3,5-(CF₃)₂C₆H₃)₄]⁻).

The organic cation may, for instance, be a monocation, a dication or a trication. It is typically a monocation or a dication. Usually, however, the organic cation is a monocation, i.e. it has a single positive charge.

The organic cation is typically other than each of the one or more A cations of the crystalline A/M/X material.

The organic cation is often other than a primary ammonium cation. The organic cation may be other than a secondary ammonium cation. The organic cation is often other than a tertiary ammonium cation. The organic cation may be other than a quaternary ammonium cation. The organic cation may be other than a cation of formula (X) as defined hereinbefore. For instance, the organic cation is often other than a formamidinium cation and other than a guanidinium cation. The organic cation may be other than a primary, secondary, tertiary or quaternary ammonium cation and other than a cation of formula (X). The organic cation may be other than a primary, secondary, tertiary or quaternary ammonium cation, other than a formamidinium cation and other than a guanidinium cation.

It is understood that the organic cation of the ionic solid is not NH₄⁺, because NH₄⁺ is an inorganic cation, not an organic cation.

The organic cation typically comprises at least one heteroatom, for instance at least one heteroatom selected from O, S, N, P, Se and Si. Typically, the organic cation comprises at least one heteroatom selected from O, S, N, P and Si. The organic cation may for instance comprise at least one heteroatom selected from O, S and N.

Often, the organic cation comprises at least one nitrogen atom. For instance, the organic cation may be an unsubstituted or substituted heterocyclyl cation which comprises a nitrogen atom (or, more specifically, whose heterocyclic ring contains a nitrogen ring atom). Alternatively, the organic cation may comprise, or be, a non-cyclic moiety which comprises a nitrogen atom. In the organic cation, said nitrogen atom may be positively charged.

Thus, the organic cation typically comprises a positively-charged nitrogen atom. The organic cation typically comprises a moiety of formula (I):

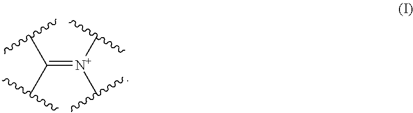 [SMILES: *C(*)=[N+](*)*]

The moiety of formula (I) may be within an unsubstituted or substituted heterocyclyl cation. In particular, the carbon atom and the positively-charged nitrogen atom may be adjacent ring atoms in a heterocyclic ring of such a cation. Alternatively, the moiety of formula (I) may be part of a non-cyclic moiety. It may for instance be part of an iminium cation.

The organic cation may be an unsubstituted or substituted iminium cation, for instance an iminium cation of formula II:

[R^{x}R^{y}C═NR^{z}R^{w}]⁺ (II)

wherein

R^{x} is hydrogen, unsubstituted or substituted C₁₋₂₀ alkyl, unsubstituted or substituted C₂₋₂₀ alkenyl, unsubstituted or substituted C₂₋₂₀ alkynyl, unsubstituted or substituted aryl, unsubstituted or substituted C₃₋₁₀ cycloalkyl, unsubstituted or substituted heterocyclyl, unsubstituted or substituted amino, unsubstituted or substituted (C₁₋₁₀ alkyl)amino, or unsubstituted or substituted di(C₁₋₁₀ alkyl)amino, provided that R^{x} may together with R^{y} form a C₁₋₂₀ alkylene group (typically a C₁₋₆ alkylene group); R^{z} is hydrogen, unsubstituted or substituted C₁₋₂₀ alkyl, unsubstituted or substituted C₂₋₂₀ alkenyl, unsubstituted or substituted C₂₋₂₀ alkynyl, unsubstituted or substituted aryl, unsubstituted or substituted C₃₋₁₀ cycloalkyl, unsubstituted or substituted heterocyclyl, unsubstituted or substituted amino, unsubstituted or substituted (C₁₋₁₀ alkyl)amino, or unsubstituted or substituted di(C₁₋₁₀ alkyl)amino, provided that R^{y} may together with R^{x} form a C₁₋₂₀ alkylene group (typically a C₁₋₆ alkylene group);

R^{z} is unsubstituted or substituted C₁₋₂₀ alkyl, unsubstituted or substituted C₂₋₂₀ alkenyl, unsubstituted or substituted C₂₋₂₀ alkynyl, unsubstituted or substituted aryl, unsubstituted or substituted C₃₋₁₀ cycloalkyl, unsubstituted or substituted heterocyclyl, unsubstituted or substituted amino, unsubstituted or substituted (C₁₋₁₀ alkyl)amino, or unsubstituted or substituted di(C₁₋₁₀ alkyl)amino, provided that R^{z} may together with R^{w} form a C₁₋₂₀ alkylene group (typically a C₁₋₆ alkylene group); and

R^{w} is unsubstituted or substituted C₁₋₂₀ alkyl, unsubstituted or substituted C₂₋₂₀ alkenyl, unsubstituted or substituted C₂₋₂₀ alkynyl, unsubstituted or substituted aryl, unsubstituted or substituted C₃₋₁₀ cycloalkyl, unsubstituted or substituted heterocyclyl, unsubstituted or substituted amino, unsubstituted or substituted (C₁₋₁₀ alkyl)amino, or unsubstituted or substituted di(C₁₋₁₀ alkyl)amino, provided that R^{w} may together with R^{z} form a C₁₋₂₀ alkylene group (typically a C₁₋₆ alkylene group).

Usually, in the iminium cation of formula II, R^{x} is unsubstituted or substituted di(C₁₋₁₀ alkyl)amino, for instance unsubstituted di(C₁₋₁₀alkyl)amino. R^{x} may for instance be unsubstituted or substituted di(C₁₋₆ alkyl)amino, for instance unsubstituted di(C₁₋₆ alkyl)amino. R^{x} is typically unsubstituted di(C₁₋₄ alkyl)amino. For instance R^{x} is often di(isopropyl)amino.

Often, in the iminium cation of formula II, R^{y} is hydrogen. Typically, therefore, R^{y} is H.

Typically, in the iminium cation of formula II, R^{z} and R^{w} are the same or different and are independently selected from unsubstituted or substituted C₁₋₂₀ alkyl groups. Thus, R² and R^{w} are typically unsubstituted or substituted C₁₋₁₀ alkyl groups, and more typically unsubstituted or substituted C₁₋₆ alkyl groups. R^{z} and R^{w} are often the same or different unsubstituted di(C₁₋₄ alkyl)amino groups. For instance, R^{z} and R^{w} may both be isopropyl groups.

Thus, often, in the iminium cation of formula II, R^{x} is unsubstituted or substituted di(C₁₋₁₀ alkyl)amino, R^{y} is hydrogen, and R^{z} and R^{w} are the same or different and are independently selected from unsubstituted or substituted C₁₋₂₀ alkyl groups.

An example of an iminium cation of formula II is N-((diisopropylamino)methylene)-N-diisopropylaminium (Di-IPAM).

Thus, the ionic solid may comprise an organic cation that is an iminium cation of formula II and a counter-anion that is a polyatomic anion. For instance, the ionic solid may comprise an organic cation that is an iminium cation of formula II and a counter-anion that is a non-coordinating polyatomic anion, for instance a borate, chlorate, triflate, carborane (e.g. CB₁₁H₁₂⁻), phosphate or [Al(OC(CF₃)₃)₄)]⁻ anion. Typically, the ionic solid comprises an organic cation that is iminium cation of formula II, and a counter-anion that is a borate anion, typically BF₄⁻, or a phosphate anion, typically PF₆⁻. Often, the counter-anion is a borate anion, typically BF₄⁻. Thus, the organic cation may be N-((diisopropylamino)methylene)-N-diisopropylaminium and the counter-anion may be BF₄⁻.

The ionic solid may for instance be N-((diisopropylamino)methylene)-N-diisopropylaminium tetrafluoroborate.

Alternatively, the ionic solid may comprise an organic cation that is an iminium cation of formula (II) and a counter-anion that is a halide. Thus, the organic cation may be N-((diisopropylamino)methylene)-N-diisopropylaminium and the counter-anion may be a halide anion.

The organic cation is often however a cation of an unsubstituted or substituted heterocyclic ring. Thus, the organic cation may be referred to as an unsubstituted or substituted heterocyclyl cation. The unsubstituted or substituted heterocyclyl cation, may for instance be an unsubstituted or substituted imidazolium cation, an unsubstituted or substituted pyrazolium cation, an unsubstituted or substituted triazolium cation, an unsubstituted or substituted tetrazolium cation, an unsubstituted or substituted pyridinium cation, an unsubstituted or substituted piperidinium cation or an unsubstituted or substituted pyrrolidinium cation.

Typically, when the organic cation is an unsubstituted or substituted heterocyclyl cation, the heterocyclic ring comprises at least one nitrogen atom, for instance from 1 to 4 nitrogen atoms. The heterocyclic ring may for instance comprise 2 or 3 nitrogen atoms, for instance the organic cation may be an unsubstituted or substituted imidazolium cation, an unsubstituted or substituted pyrazolium cation, or an unsubstituted or substituted triazolium cation.

Usually, the organic cation is a cation of an unsubstituted or substituted heterocyclic ring, wherein the cation comprises a positively-charged ring nitrogen atom.

The heterocyclic ring may or may not be an aromatic ring. When the heterocyclic ring is an aromatic ring (i.e. a heteroaromatic ring) the organic cation is a cation of an unsubstituted or substituted heteroaryl ring. In this case, the organic cation may be referred to as an unsubstituted or substituted heteroaryl cation. The unsubstituted or substituted heteroaryl cation may for instance be an unsubstituted or substituted pyridinium cation.

The organic cation is often an unsubstituted or substituted imidazolium cation or an unsubstituted or substituted triazolium cation.

The organic cation may for instance be an imidazolium cation of formula III:

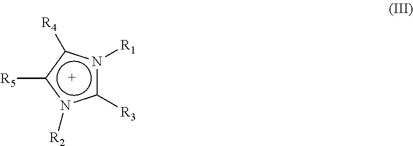

wherein each of R₁, R₂, R₃, R₄ and R₅ is independently selected from hydrogen, unsubstituted or substituted C₁₋₂₀ alkyl, unsubstituted or substituted C₂₋₂₀ alkenyl, unsubstituted or substituted C₂₋₂₀ alkynyl, unsubstituted or substituted aryl, unsubstituted or substituted C₃₋₁₀ cycloalkyl, unsubstituted or substituted heterocyclyl, unsubstituted or substituted amino, unsubstituted or substituted (C₁₋₁₀ alkyl)amino, and unsubstituted or substituted di(C₁₋₁₀ alkyl)amino, provided that R¹ and R⁴, or R⁴ and R⁵, or R⁵ and R², or R² and R³, or R³ and R¹, may together form a C₁₋₁₀ alkylene group (typically a C₁₋₄ alkylene group).

Typically, in formula III, each of R₁, R₂, R₃, R₄ and R₅ is independently selected from hydrogen, unsubstituted C₁₋₂₀ alkyl, unsubstituted C₂₋₂₀ alkenyl, unsubstituted C₂₋₂₀ alkynyl, unsubstituted aryl, unsubstituted C₃₋₁₀ cycloalkyl, and unsubstituted heterocyclyl, provided that R¹ and R⁴, or R⁴ and R⁵, or R⁵ and R², or R² and R³, or R³ and R¹, may together form an unsubstituted C₁₋₁₀ alkylene group (typically an unsubstituted C₁₋₃ alkylene group).

Often, in formula III, R₁ and R₂ are the same or different and are both unsubstituted or substituted C₁₋₂₀ alkyl or C₃₋₁₀ cycloalkyl groups. R₁ and R₂ are typically for instance both unsubstituted or substituted C₃₋₂₀ alkyl or C₃₋₁₀ cycloalkyl groups, more typically unsubstituted C₃₋₁₀ alkyl or C₃₋₁₀ cycloalkyl groups. R₁, and R₂ may for instance both be the same or different unsubstituted C₁₋₂₀ alkyl or C₃₋₁₀ cycloalkyl groups. R₁ and R₂ are typically for instance both the same or different unsubstituted C₃₋₂₀ alkyl or C₃₋₁₀ cycloalkyl groups for instance unsubstituted C₃₋₁₀ alkyl or C₃₋₁₀ cycloalkyl groups. For instance, R₁ and R₂ may both be isopropyl groups or R₁ and R₂ may both be cyclohexyl groups.

Often, in formula III, R₁ and R₂ are the same or different and are both unsubstituted or substituted C₁₋₂₀ alkyl groups. R₁, and R₂ are typically for instance both unsubstituted or substituted C₃₋₂₀ alkyl groups, more typically unsubstituted C₃₋₁₀ alkyl groups. R₁ and R₂ may for instance both be the same or different unsubstituted C₁₋₂₀ alkyl groups. R₁, and R₂ are typically for instance both the same or different unsubstituted C₃₋₂₀ alkyl groups, for instance unsubstituted C₃₋₁₀ alkyl groups. For instance, R₁ and R₂ may both be isopropyl groups, or may both be tert-butyl groups. R₁ and R₂ may for instance both be unsubstituted or substituted C₁₋₃ alkyl groups; they may both for instance be unsubstituted C₁₋₃ alkyl groups, e.g. they may both be isopropyl groups. Alternatively, R₁, and R₂ may both be unsubstituted or substituted C₄₋₂₀ alkyl groups, e.g. they may both be tert-butyl groups.

Alternatively, in formula III, R₁ and R₂ are the same or different and are both unsubstituted or substituted C₃₋₁₀ cycloalkyl groups. R₁ and R₂ are typically for instance both unsubstituted C₃₋₁₀ cycloalkyl groups. For instance, R₁ and R₂ may both be cyclohexyl groups.

Usually, in formula III, R₃, R₄ and R₅ are each hydrogen. Thus, typically, R₃ is H, R₄ is H and R₅ is H.

Thus, in one embodiment, in formula III, R₃, R₄ and R₅ are hydrogen, and R₁ and R₂ are both unsubstituted C₃₋₂₀ alkyl groups or unsubstituted C₃₋₁₀ cycloalkyl groups, more typically unsubstituted C₃₋₁₀ alkyl groups or unsubstituted C₃₋₁₀ cycloalkyl groups. R₁ and R₂ may both be isopropyl groups. R₁, and R₂ may both be tert-butyl groups. R₁ and R₂ may both be cyclohexyl groups. Thus, the organic cation may be 1,3-diisopropylimidazolium or 1,3-di-tert-butylimidazolium or 1,3-dicyclohexylimidazolium.

The organic cation may for instance be a triazolium cation of formula IV:

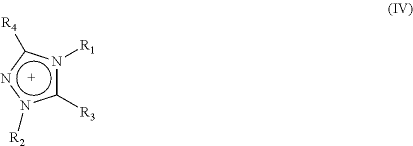

wherein each of R₁, R₂, R₃ and R₄ is independently selected from hydrogen, unsubstituted or substituted C₁₋₂₀ alkyl, unsubstituted or substituted C₂₋₂₀ alkenyl, unsubstituted or substituted C₂₋₂₀ alkynyl, unsubstituted or substituted aryl, unsubstituted or substituted C₃₋₁₀ cycloalkyl, unsubstituted or substituted heterocyclyl, unsubstituted or substituted amino, unsubstituted or substituted (C₁₋₁₀ alkyl)amino, and unsubstituted or substituted di(C₁₋₁₀ alkyl)amino, provided that R¹ and R⁴, or R² and R³, or R¹ and R³, may together form a C₁₋₁₀ alkylene group (typically a C₁₋₄ alkylene group, for instance a C₁₋₃ alkylene group).

Typically, in formula IV, each of R₁, R₂, R₃ and R₄ is independently selected from hydrogen, unsubstituted C₁₋₂₀ alkyl, unsubstituted C₂₋₂₀ alkenyl, unsubstituted C₂₋₂₀ alkynyl, unsubstituted or substituted aryl, unsubstituted C₃₋₁₀ cycloalkyl, and unsubstituted heterocyclyl, provided that R¹ and R⁴, or R² and R³, or R¹ and R³, may together form an unsubstituted C₁₋₄ alkylene group (typically an unsubstituted C₁₋₃ alkylene group).

Often, in formula IV, R₁ and R₄ together form an unsubstituted or substituted C₁₋₄ alkylene group. R₁ and R₄ may for instance together form an unsubstituted C₁₋₃ alkylene group. Thus, R₁ and R₄ may together form a propylene group.

Alternatively, in formula IV, R₁ and R₄ may both be unsubstituted or substituted C₁₋₂₀ alkyl groups, more typically unsubstituted C₁₋₁₀ alkyl groups; or R₄ may be hydrogen and R₁ may be an unsubstituted or substituted C₁₋₂₀ alkyl group, more typically an unsubstituted or substituted C₁₋₁₀ alkyl group, for instance an unsubstituted or substituted C₃₋₁₀ alkyl group, e.g. unsubstituted C₃₋₁₀ alkyl.

R₂ in formula IV is typically unsubstituted or substituted aryl, unsubstituted or substituted C₃₋₁₀ cycloalkyl, or unsubstituted or substituted heterocyclyl. Usually, for instance, R₂ is unsubstituted or substituted aryl. R₂ is often substituted aryl, such as, for example, pentafluorophenyl.

Usually, R₃ in formula IV is hydrogen. Thus, typically, R₃ is H.

Thus, in one embodiment in formula IV, R₃ is hydrogen; R₂ is unsubstituted or substituted aryl, unsubstituted or substituted C₃₋₁₀ cycloalkyl, or unsubstituted or substituted heterocyclyl; and R₁, and R₄ together form an unsubstituted or substituted C₁₋₄ alkylene group. Often, R₂ is substituted aryl, for instance pentafluorophenyl, and R₁ and R₄ together form an unsubstituted C₁₋₄ alkylene group, for instance a propylene group.

Thus, the organic cation may be 6,7-dihydro-2-pentafluorophenyl-5H-pyrrolo[2,1-c]-1,2,4-triazolium.

Thus, the ionic solid may comprise an organic cation that is an unsubstituted or substituted imidazolium or triazolium cation, and a counter-anion that is a polyatomic anion. For instance, the ionic solid may comprise an organic cation that is an imidazolium or triazolium cation of formula III or IV respectively and a counter-anion that is a non-coordinating polyatomic anion, for instance a borate, chlorate, triflate, carborane (e.g. CB₁₁H₁₂⁻), phosphate or [Al(OC(CF₃)₃)₄)]⁻ anion. Typically, the ionic solid comprises an organic cation that is an imidazolium cation of formula III or a triazolium cation of formula IV, and a counter-anion that is a borate anion, typically BF₄⁻, or a phosphate anion, typically PF₆⁻. Often, the counter-anion is a borate anion, typically BF₄⁻. Thus, preferably, the organic cation is 1,3-diisopropylimidazolium or 1,3-di-tert-butylimidazolium or 6,7-dihydro-2-pentafluorophenyl-5H-pyrrolo[2,1-c]-1,2,4-triazolium, and the counter-anion is BF₄⁻.

Hence, in one embodiment the organic cation is 1,3-diisopropylimidazolium and the counter anion is BF₄⁻. In another embodiment the organic cation is 1,3-di-tert-butylimidazolium and the counter anion is BF₄⁻. In another embodiment the organic cation is 1,3-dicyclohexylimidazolium and the counter anion is BF₄⁻. In another embodiment the organic cation is 6,7-dihydro-2-pentafluorophenyl-5H-pyrrolo[2,1-c]-1,2,4-triazolium and the counter anion is BF₄⁻.

The ionic solid may for instance be 1,3-diisopropylimidazolium tetrafluoroborate (m.p. 62-79° C.), 1,3-dicyclohexylimidazolium tetrafluoroborate (m.p. 171-175° C.), 1,3-di-tert-butylimidazolium tetrafluoroborate (m.p. 157-198° C.) or 6,7-dihydro-2-pentafluorophenyl-5H-pyrrolo[2,1-c]-1,2,4-triazolium tetrafluoroborate (m.p. 245° C.).

The ionic solid may however comprise an organic cation that is an unsubstituted or substituted imidazolium or triazolium cation, and a counter-anion which is a halide anion. For instance, the ionic solid may comprise an organic cation that is an imidazolium or triazolium cation of formula III or IV respectively and a counter-anion that is a halide anion. Thus, the organic cation may be 1,3-diisopropylimidazolium or 1,3-di-tert-butylimidazolium or 1,3-dicyclohexylimidazolium or 6,7-dihydro-2-pentafluorophenyl-5H-pyrrolo[2,1-c]-1,2,4-triazolium, and the counter-anion may be a halide anion.

The ionic solid may for instance comprise an organic cation that is an unsubstituted or substituted imidazolium cation, and a counter-anion which is a halide anion. For instance, the ionic solid may comprise an organic cation that is an imidazolium cation of formula III and a counter-anion that is a halide anion. Thus, the organic cation may be 1,3-diisopropylimidazolium or 1,3-di-tert-butylimidazolium or 1,3-dicyclohexylimidazolium and the counter-anion may be a halide anion, for instance chloride. The ionic solid may for instance be 1,3-diisopropylimidazolium chloride (m.p. 182-186° C.).

The ionic solid may comprise an organic cation that is an unsubstituted or substituted triazolium cation, and a counter-anion which is a halide anion. For instance, the ionic solid may comprise an organic cation that is a triazolium cation of formula IV and a counter-anion that is a halide anion. Thus, the organic cation may be 6,7-dihydro-2-pentafluorophenyl-5H-pyrrolo[2,1-c]-1,2,4-triazolium, and the counter-anion may be a halide anion, for instance chloride.

The organic cation may however be other than an unsubstituted or substituted imidazolium cation and the counter-anion may be a halide. The organic cation may be other than an imidazolium cation of formula III and the counter-anion may be a halide.

The organic cation may be other than an unsubstituted or substituted imidazolium cation and the counter-anion may be any of the anions other than halide described herein. The organic cation may be other than an imidazolium cation of formula III and the counter-anion may be any of the anions other than halide described herein.

The organic cation may alternatively be an unsubstituted or substituted pyridinium cation, an unsubstituted or substituted piperidinium cation or an unsubstituted or substituted pyrrolidinium cation.

The organic cation may in particular be an unsubstituted or substituted pyridinium cation. The unsubstituted or substituted pyridinium cation may be a pyridinium cation of formula V:

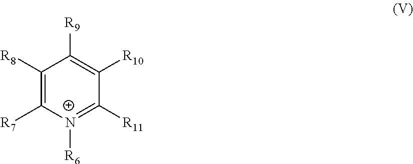

wherein each of R₆, R₇, R₈, R₉, R₁₀ and R₁₁, is independently selected from hydrogen, unsubstituted or substituted C₁₋₁₀ alkyl, unsubstituted or substituted C₂₋₁₀ alkenyl, unsubstituted or substituted C₂₋₁₀ alkynyl, unsubstituted or substituted C₆₋₁₂ aryl, unsubstituted or substituted C₃₋₁₀ cycloalkyl, unsubstituted or substituted C₃₋₁₀ cycloalkenyl, amino, unsubstituted or substituted (C₁₋₆ alkyl)amino and unsubstituted or substituted di(C₁₋₆ alkyl)amino.

Typically, each of R₆, R₇, R₈, R₉, R₁₀ and R₁₁ is independently selected from hydrogen, unsubstituted or substituted C₁₋₁₀ alkyl, unsubstituted C₂₋₁₀ alkenyl, unsubstituted C₂₋₁₀ alkynyl, unsubstituted C₆₋₁₂ aryl, unsubstituted C₃₋₁₀ cycloalkyl, unsubstituted C₃₋₁₀ cycloalkenyl, amino, unsubstituted (C₁₋₆ alkyl)amino and unsubstituted di(C₁₋₆ alkyl)amino.

Often R₇, R₈, R₁₀ and R₁₁ are hydrogen and each of R₆ and R₉ is independently selected from unsubstituted C₁₋₁₀ alkyl and C₁₋₁₀ alkyl substituted with a phenyl group. For instance, R₇, R₈, R₁₀ and R₁₁ may be hydrogen and R₆ and R₉ are unsubstituted C₁₋₁₀ alkyl, preferably C₁₋₆ alkyl. Thus, R₇, R₈, R₁₀ and R₁₁ may be hydrogen, R₉ may be methyl and R₆ may be selected from methyl, ethyl, propyl, butyl, pentyl and hexyl.

The organic cation may alternatively be an unsubstituted or substituted piperidinium cation. In this case the counter anion is often other than a halide. The unsubstituted or substituted piperidinium cation may be a piperidinium cation of formula VI:

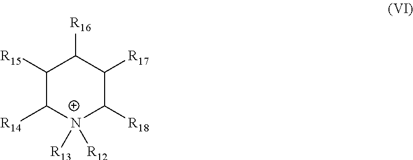

wherein each of R₁₂, R₁₃, R₁₄, R₁₅, R₁₆, R₁₇ and R₁₈ is independently selected from hydrogen, unsubstituted or substituted C₁₋₁₀ alkyl, unsubstituted or substituted C₂₋₁₀ alkenyl, unsubstituted or substituted C₂₋₁₀ alkynyl, unsubstituted or substituted C₆₋₁₂ aryl, unsubstituted or substituted C₃₋₁₀ cycloalkyl, unsubstituted or substituted C₃₋₁₀ cycloalkenyl, amino, unsubstituted or substituted (C₁₋₆ alkyl)amino and unsubstituted or substituted di(C₁₋₆ alkyl)amino. Often, in this embodiment, when one of R₁₂ and R₁₃ is methyl, the other of R₁₂ and R₁₃ is not butyl. Often, for instance, when each of R₁₄, R₁₅, R₁₆, R₁₇ and R₁₈ is hydrogen, and one of R₁₂ and R₁₃ is methyl, the other of R₁₂ and R₁₃ is not butyl. Similarly, it is often the case that when the counter-anion is BF₄⁻ and one of R₁₂ and R₁₃ is methyl, the other of R₁₂ and R₁₃ is not butyl. Thus, typically, when each of R₁₄, R₁₅, R₁₆, R₁₇ and R₁₈ is hydrogen, and the counter-anion is BF₄ ^{- and one of R}₁₂ and R₁₃ is methyl, the other of R₁₂ and R₁₃ is not butyl.

Thus, in the present invention, the organic cation may be other than a piperidinium cation of formula VI wherein each of R₁₄, R₁₅, R₁₆, R₁₇ and R₁₈ is hydrogen, one of R₁₂ and R₁₃ is methyl, and the other of R₁₂ and R₁₃ is butyl. In particular, in the present invention, the ionic solid may be other than the tetrafluoroborate salt of a piperidinium cation of formula VI wherein each of R₁₄, R₁₅, R₁₆, R₁₇ and R₁₈ is hydrogen, one of R₁₂ and R₁₃ is methyl, and the other of R₁₂ and R₁₃ is butyl.

Typically, in the piperidinium cation of formula VI, each of R₁₂, R₁₃, R₁₄, R₁₅, R₁₆, R₁₇ and R₁₈ is independently selected from hydrogen, unsubstituted or substituted C₁₋₁₀ alkyl, unsubstituted C₂₋₁₀ alkenyl, unsubstituted C₂₋₁₀ alkynyl, unsubstituted C₆₋₁₂ aryl, unsubstituted C₃₋₁₀ cycloalkyl, unsubstituted C₃₋₁₀ cycloalkenyl, amino, unsubstituted (C₁₋₆ alkyl)amino and unsubstituted di(C₁₋₆ alkyl)amino. However, often, in this embodiment, when one of R₁₂ and R₁₃ is methyl, the other of R₁₂ and R₁₃ is not butyl. Typically, for instance, when the counter-anion is BF₄ ⁻ and one of R₁₂ and R₁₃ is methyl, the other of R₁₂ and R₁₃ is not butyl.

Often, in the piperidinium cation of formula VI, R₁₄, R₁₅, R₁₆, R₁₇ and R₁₈ are hydrogen and each of R₁₂ and R₁₃ is independently selected from unsubstituted C₁₋₁₀ alkyl and C₁₋₁₀ alkyl substituted with a phenyl group. However, often, in this embodiment, when one of R₁₂ and R₁₃ is methyl, the other of R₁₂ and R₁₃ is not butyl. Typically, for instance, when the counter-anion is BF₄⁻ and one of R₁₂ and R₁₃ is methyl, the other of R₁₂ and R₁₃ is not butyl.

Typically, in the piperidinium cation of formula VI, R₁₄, R₁₅, R₁₆, R₁₇ and R₁₈ are hydrogen and each of R₁₂ and R₁₃ is independently selected from unsubstituted C₁₋₁₀ alkyl, preferably unsubstituted C₁₋₆ alkyl. For instance, R₁₄, R₁₅, R₁₆, R₁₇ and R₁₈ may be hydrogen, R₁₃ may be methyl and R₁₂ may be selected from methyl, ethyl, propyl, butyl, pentyl or hexyl. Often, for instance, each of R₁₄, R₁₅, R₁₆, R₁₇ and R₁₈ is hydrogen, R₁₃ is methyl and R₁₂ is butyl. In a preferred embodiment, each of R₁₄, R₁₅, R₁₆, R₁₇ and R₁₈ is hydrogen, R₁₃ is methyl and R₁₂ is n-butyl.

Alternatively, however, each of R₁₄, R₁₅, R₁₆, R₁₇ and R₁₈ is hydrogen and each of R₁₂ and R₁₃ is independently selected from unsubstituted C₁₋₁₀ alkyl, preferably unsubstituted C₁₋₆ alkyl, provided that if one of R₁₂ and R₁₃ is methyl, the other of R₁₂ and R₁₃ is not butyl. For instance, it may be that each of R₁₄, R₁₅, R₁₆, R₁₇ and R₁₈ is hydrogen, R₁₃ is methyl and R₁₂ is selected from methyl, ethyl, propyl, pentyl or hexyl.

Often, the organic cation is a piperidinium cation of formula VI as defined herein provided that the counter-anion is other than tetrafluoroborate. The organic cation may be a piperidinium cation of formula VI as defined herein provided that the counter-anion is other than a halide and other than tetrafluoroborate. For instance, the organic cation may be a piperidinium cation of formula VI as defined herein and the counter-anion may be a polyatomic anion, as described herein, other than tetrafluoroborate.

The organic cation may for instance be a piperidinium cation of formula VI as defined herein provided that, when the counter-anion is BF₄⁻ and one of R₁₂ and R₁₃ is methyl, the other of R₁₂ and R₁₃ is not butyl. The organic cation may for instance be a piperidinium cation of formula VI as defined herein wherein the counter-anion is not a halide, and provided that, when the counter-anion is BF₄ ⁻ and one of R₁₂ and R₁₃ is methyl, the other of R₁₂ and R₁₃ is not butyl.

In a preferred embodiment, the organic cation is an unsubstituted or substituted piperidinium cation as defined herein, for instance a piperidinium cation of formula VI, and the compound of [A]ₐ[M]_{b}[X]_{c} either does not comprise the methylammonium cation (i.e. it is free of methylammonium) or it only contains a small amount of methylammonium. Here, a “small amount” of methylammonium typically means that [A] of the compound of formula [A]ₐ[M]_{b}[X]_{c} consists of methylammonium and at least one A cation other than methylammonium, provided that the molar fraction of the methylammonium in [A] is less than 15% of [A]. Preferably the molar fraction of the methylammonium in [A] is less than 10% of [A], and more preferably less than 5% of [A], for instance less than 2% of [A], more preferably less than 1% of [A]. Thus, a compound of formula [A]ₐ[M]_{b}[X]_{c} which contains only a small amount of methylammonium is a compound of formula [(CH₃NH₃)ₓ(A′)₁₋ₓ]ₐ[M]_{b}[X]_{c} wherein (A′) represents at least one A cation other than methylammonium, and x is less than 0.15, preferably less than 0.10, for instance less than 0.05, and more preferably less than 0.02, for instance less than 0.01. In a preferred embodiment, the organic cation is an unsubstituted or substituted piperidinium cation as defined herein, for instance a piperidinium cation of formula VI, and the compound of [A]ₐ[M]_{b}[X]_{c} does not comprise the methylammonium cation, i.e. it is free of methylammonium.

The organic cation may alternatively be an unsubstituted or substituted pyrrolidinium cation. In this case the counter anion is often other than a halide. The unsubstituted or substituted pyrrolidinium cation may be a pyrrolidinium cation of formula VII:

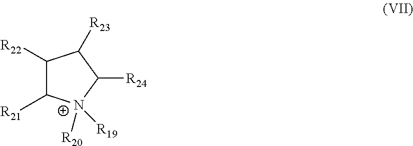

wherein each of R₁₉, R₂₀, R₂₁, R₂₂, R₂₃ and R₂₄ is independently selected from hydrogen, unsubstituted or substituted C₁₋₁₀ alkyl, unsubstituted or substituted C₂₋₁₀ alkenyl, unsubstituted or substituted C₂₋₁₀ alkynyl, unsubstituted or substituted C₆₋₁₂ aryl, unsubstituted or substituted C₃₋₁₀ cycloalkyl, unsubstituted or substituted C₃₋₁₀ cycloalkenyl, amino, unsubstituted or substituted (C₁₋₆ alkyl)amino and unsubstituted or substituted di(C₁₋₆ alkyl)amino.

Typically each of R₁₉, R₂₀, R₂₁, R₂₂, R₂₃ and R₂₄ is independently selected from hydrogen, unsubstituted or substituted C₁₋₁₀ alkyl, unsubstituted C₂₋₁₀ alkenyl, unsubstituted C₂₋₁₀ alkynyl, unsubstituted C₆₋₁₂ aryl, unsubstituted C₃₋₁₀ cycloalkyl, unsubstituted C₃₋₁₀ cycloalkenyl, amino, unsubstituted (C₁₋₆ alkyl)amino and unsubstituted di(C₁₋₆ alkyl)amino. Often, R₂₁, R₂₂, R₂₃ and R₂₄ are hydrogen and each of R₁₉ and R₂₀ is independently selected from unsubstituted C₁₋₁₀ alkyl and C₁₋₁₀ alkyl substituted with a phenyl group.

Typically, R₂₁, R₂₂, R₂₃ and R₂₄ are hydrogen and each of R₁₉ and R₂₀ is independently selected from unsubstituted C₁₋₁₀ alkyl, preferably unsubstituted C₁₋₆ alkyl. For instance, R₂₁, R₂₂, R₂₃ and R₂₄ may be hydrogen, R₁₉ may be methyl and R₂₀ may be selected from methyl, ethyl, propyl, butyl, pentyl or hexyl.

In one embodiment, the organic cation is an unsubstituted or substituted pyridinium cation, an unsubstituted or substituted piperidinium cation or an unsubstituted or substituted pyrrolidinium cation as described above and the counter-anion is a halide anion.

In another embodiment, the organic cation is an unsubstituted or substituted pyridinium cation, an unsubstituted or substituted piperidinium cation or an unsubstituted or substituted pyrrolidinium cation as described above and the counter-anion is a polyatomic anion as described herein. For instance, the organic cation may be an unsubstituted or substituted piperidinium cation as described above and the counter-anion may be a polyatomic anion as described herein. Typically, the counter-anion is a borate anion, preferably BF₄⁻.

The ionic solid is often however selected from 1,3-diisopropylimidazolium tetrafluoroborate (m.p. 62-79° C.), 1,3-dicyclohexylimidazolium tetrafluoroborate (m.p. 171-175° C.), 1,3-di-tert-butylimidazolium tetrafluoroborate (m.p. 157-198° C.), 6,7-dihydro-2-pentafluorophenyl-5H-pyrrolo[2,1-c]-1,2,4-triazolium tetrafluoroborate (m.p. 245° C.), N-((diisopropylamino)methylene)-N-diisopropylaminium tetrafluoroborate (m.p. 280-284° C.), and 1,3-diisopropylimidazolium chloride (m.p. 182-186° C.). The ionic solid may for instance be selected from 1,3-diisopropylimidazolium tetrafluoroborate (m.p. 62-79° C.), 1,3-dicyclohexylimidazolium tetrafluoroborate (m.p. 171-175° C.), 1,3-di-tert-butylimidazolium tetrafluoroborate (m.p. 157-198° C.), 6,7-dihydro-2-pentafluorophenyl-5H-pyrrolo[2,1-c]-1,2,4-triazolium tetrafluoroborate (m.p. 245° C.), N-((diisopropylamino)methylene)-N-diisopropylaminium tetrafluoroborate (m.p. 280-284° C.), 1,3-diisopropylimidazolium chloride (m.p. 182-186° C.) and 1-n-butyl-1-methylpiperidinium tetrafluoroborate (m.p. 149° C.). The ionic solid may for instance be selected from 1,3-diisopropylimidazolium tetrafluoroborate (m.p. 62-79° C.), 1,3-di-tert-butylimidazolium tetrafluoroborate (m.p. 157-198° C.), 6,7-dihydro-2-pentafluorophenyl-5H-pyrrolo[2,1-c]-1,2,4-triazolium tetrafluoroborate (m.p. 245° C.), N-((diisopropylamino)methylene)-N-diisopropylaminium tetrafluoroborate (m.p. 280-284° C.) and 1-n-butyl-1-methylpiperidinium tetrafluoroborate (m.p. 149° C.).

The ionic solid may be: (a) a salt which is in the solid state at 100° C. and at temperatures of less than 100° C.; or (b) a salt other than (a) which comprises an imidazolium cation of formula III as defined anywhere herein and a tetrafluoroborate anion. In other words, the ionic solid may be: (a) a salt whose melting point is greater than 100° C.; or (b) a salt other than (a) which comprises an imidazolium cation of formula III as defined anywhere herein and a tetrafluoroborate anion. In the cation of formula III, R₁ and R₂ may both for instance be unsubstituted or substituted C₁₋₃ alkyl groups, e.g. they may both be unsubstituted C₁₋₃ alkyl groups, for instance isopropyl groups. Thus, the ionic solid may be: (a) a salt which is in the solid state at 100° C. and at temperatures of less than 100° C.; or (b) 1,3-diisopropylimidazolium tetrafluoroborate. In other words, the ionic solid may be: (a) a salt whose melting point is greater than 100° C.; or (b) 1,3-diisopropylimidazolium tetrafluoroborate.

Typically, the ionic solid is present in an amount of less than 50 mol %, for instance less than 10 mol % or less than or equal to 2.5 mol %, particularly less than 1.0 mol % with respect to the number of moles of the one or more metal or metalloid cations M in the crystalline A/M/X material. The ionic solid may be present in an amount of from 0.01 to 5 mol %, or from 0.02 to 2.5 mol %, more preferably in an amount of from 0.05 to 2.0 mol %, or from 0.05 to 1.0 mol %, and even more preferably in an amount of from 0.1 to 1.5 mol %, or from 0.1 to 1.0 mol %, with respect to the number of moles of the one or more metal or metalloid cations M in the crystalline A/M/X material. For instance, the ionic solid may be present in an amount of from 0.1 mol % to 0.9 mol % with respect to the number of moles of the one or more metal or metalloid cations M in the crystalline A/M/X material, for instance from 0.1 mol % to 0.8 mol %, from 0.2 mol % to 0.8 mol %, from 0.2 mol % to 0.7 mol % or less than 0.5 mol %, or from 0.1 mol % to 0.5 mol %, from 0.2 mol % to 0.5 mol %, or from 0.3 mol % to 0.5 mol %.

Further Layers, n-Type Materials, p-Type Materials and Electrode Materials

Often, the optoelectronic device further comprises a layer comprising a charge-transporting material. Typically, the layer comprising the crystalline A/M/X material is disposed on the layer comprising the charge-transporting material. Preferably, the layer comprising the crystalline A/M/X material is disposed directly on the layer comprising the charge-transporting material, such that the layer comprising the crystalline A/M/X material and the layer comprising the charge-transporting material are in physical contact.

The layer comprising the charge-transporting material is typically disposed on a first electrode. Thus, the layer comprising the charge-transporting material is typically disposed between the layer comprising the crystalline A/M/X material and the first electrode. The first electrode may be as further defined herein. It is typically a transparent electrode. The first electrode is typically an anode. The first electrode typically comprises a transparent conducting oxide, for instance fluorine doped tin oxide (FTO), aluminium doped zine oxide (AZO) or indium doped tin oxide (ITO).

In one embodiment, the layer comprising a charge transporting material is a layer of an electron transporting (n-type) material (an n-type layer). Thus, the charge-transporting material may be a hole-transporting (p-type) material. In another embodiment, the layer comprising a charge transporting material is a layer of a hole transporting (p-type) material (a p-type layer). Typically, the layer comprising a charge transporting material is a layer of a hole transporting (p-type) material. Thus, typically the charge-transporting material is a hole-transporting (p-type) material.

Typically, the layer comprising a charge transporting material has a thickness of less than 1000 nm, or less than 500 nm, or less than 250 nm, preferably less than 100 nm. For instance, the layer comprising a charge transporting material may have a thickness of from 1 to 500 nm, for instance from 5 to 250 nm, or from 10 to 75 nm. In some embodiments, the layer of a charge transporting material may have a thickness of from 20 to 50 nm or from 30 to 40 nm.

Examples of electron transporting (n-type) materials are known to the skilled person. A suitable n-type material may be an organic or inorganic material. A suitable inorganic n-type material may be selected from a metal oxide, a metal sulphide, a metal selenide, a metal telluride, a perovskite, amorphous Si, an n-type group IV semiconductor, an n-type group III-V semiconductor, an n-type group II-VI semiconductor, an n-type group I-VII semiconductor, an n-type group IV-VI semiconductor, an n-type group V-VI semiconductor, and an n-type group II-V semiconductor, any of which may be doped or undoped. More typically, the n-type material is selected from a metal oxide, a metal sulphide, a metal selenide, and a metal telluride.

Thus, the n-type layer may comprise an inorganic material selected from oxide of titanium, tin, zinc, niobium, tantalum, tungsten, indium, gallium, neodymium, palladium, or cadmium, or an oxide of a mixture of two or more of said metals. For instance, the n-type layer may comprise TiO₂, SnO₂, ZnO, Nb₂O₅, Ta₂O₅, WO₃, W₂O₅, In₂O₃, Ga₂O₃, Nd₂O₃, PbO, or CdO.

Other suitable n-type materials that may be employed include sulphides of cadmium, tin, copper, or zinc, including sulphides of a mixture of two or more of said metals. For instance, the sulphide may be FeS₂, CdS, ZnS, SnS, BiS, SbS, or Cu₂ZnSnS₄.

The n-type layer may for instance comprise a selenide of cadmium, zinc, indium, or gallium or a selenide of a mixture of two or more of said metals; or a telluride of cadmium, zinc, cadmium or tin, or a telluride of a mixture of two or more of said metals. For instance, the selenide may be Cu(In,Ga)Se₂. Typically, the telluride is a telluride of cadmium, zinc, cadmium or tin. For instance, the telluride may be CdTe.

The n-type layer may for instance comprise an inorganic material selected from oxide of titanium (e.g. TiO₂), tin (e.g. SnO₂), zinc (e.g. ZnO), niobium, tantalum, tungsten, indium, gallium, neodymium, palladium, cadmium, or an oxide of a mixture of two or more of said metals; a sulphide of cadmium, tin, copper, zinc or a sulphide of a mixture of two or more of said metals; a selenide of cadmium, zinc, indium, gallium or a selenide of a mixture of two or more of said metals; or a telluride of cadmium, zinc, cadmium or tin, or a telluride of a mixture of two or more of said metals.

Examples of other semiconductors that may be suitable n-type materials, for instance if they are n-doped, include group IV elemental or compound semiconductors; amorphous Si; group III-V semiconductors (e.g. gallium arsenide); group II-VI semiconductors (e.g. cadmium selenide); group I-VII semiconductors (e.g. cuprous chloride); group IV-VI semiconductors (e.g. lead selenide); group V-VI semiconductors (e.g. bismuth telluride); and group II-V semiconductors (e.g. cadmium arsenide).

Other n-type materials may also be employed, including organic and polymeric electron-transporting materials, and electrolytes. Suitable examples include, but are not limited to a fullerene or a fullerene derivative (for instance C₆₀, C₇₀, phenyl-C₆₁-butyric acid methyl ester (PCBM), PC₇₁BM (i.e. phenyl C₇₁ butyric acid methyl ester), bis[C₆₀] BM (i.e. bis-C₆₀ butyric acid methyl ester), and 1′,1″,4′4″-Tetrahydro-di[1,4]methanonaphthaleno[1,2:2′,3′,56,60:2″,3″][5,6] fullerene-C₆₀ (ICBA)), an organic electron transporting material comprising perylene or a derivative thereof, poly{[N,N0-bis(2-octyldodecyl)-naphthalene-1,4,5,8-bis(dicarboximide)-2,6-diyl]-alt-5,50-(2,20-bithiophene)}(P(NDI2OD-T2)) or bathocuproine (BCP).

Typically, the electron-transporting n-type material is phenyl-C₆₁-butyric acid methyl ester (PCBM).

Examples of hole transporting (p-type) materials are known to the skilled person. The p-type material may be a single p-type compound or elemental material, or a mixture of two or more p-type compounds or elemental materials, which may be undoped or doped with one or more dopant elements.

The p-type material may comprise an inorganic or an organic p-type material. For instance, the p-type material may be an organic p-type material.

Suitable p-type materials may be selected from polymeric or molecular hole transporters. The p-type material may for instance comprise spiro-OMeTAD (2,2′,7,7′-tetrakis-(N,N-di-p-methoxyphenylamine)9,9′-spirobifluorene)), P3HT (poly(3-hexylthiophene)), PCPDTBT (Poly[2,1,3-benzothiadiazole-4,7-diyl[4,4-bis(2-ethylhexyl)-4H-cyclopenta[2,1-b:3,4-b′]dithiophene-2,6-diyl]]), PVK (poly(N-vinylcarbazole)), HTM-TFSI (1-hexyl-3-methylimidazolium bis(trifluoromethylsulfonyl)imide), Li-TFSI (lithium bis(trifluoromethanesulfonyl)imide), spiro-OMETAD⁺-bis(trifluoromethanesulfonyl)imide⁻ (spiro(TFSI)₂), tBP (tert-butylpyridine), m-MTDATA (4,4′,4″-tris(methylphenylphenylamino)triphenylamine), MeOTPD (N,N,N′,N′-tetrakis(4-methoxyphenyl)-benzidine), BP2T (5,5′-di(biphenyl-4-yl)-2,2′-bithiophene), Di-NPB (N,N′-Di-[(1-naphthyl)-N,N′-diphenyl]-1,1′-biphenyl)-4,4′-diamine), α-NPB (N,N′-di(naphthalen-1-yl)-N,N′-diphenyl-benzidine), TNATA (4,4′,4″-tris-(N-(naphthylen-2-yl)-N-phenylamine)triphenylamine), BPAPF (9,9-bis[4-(N,N-bis-biphenyl-4-yl-amino)phenyl]-9H-fluorene), spiro-NPB (N2,N7-Di-1-naphthalenyl-N2,N7-diphenyl-9,9′-spirobi[9H-fluorene]-2,7-diamine), 4P-TPD (4,4-bis-(N,N-diphenylamino)-tetraphenyl), polyTPD (i.e. Poly[N,N′-bis(4-butylphenyl)-N,N′-bisphenylbenzidine], also known as poly(4-butylphenyl-diphenyl-amine)), PTAA (i.e. poly(triaryl amine), also known as poly[bis(4-phenyl)(2,4,6-trimethylphenyl)amine]) or PEDOT:PSS. The p-type material may comprise carbon nanotubes. Usually, the p-type material is selected from spiro-OMeTAD, P3HT, PCPDTBT, polyTPD, spiro(TFSI)₂ and PVK.

Typically, the hole-transporting (p-type) material comprises polyTPD. Often, the hole-transporting (p-type) material comprises N,N′-bis(1-naphthyl)-N,N′-diphenyl-1,1′-biphenyl-4,4′-diamine (NPD). The hole-transporting (p-type) material may for instance comprise polyTPD and NPD. For instance, the layer comprising the charge transporting material may be a p-type layer which comprises a first sub-layer which comprises polyTPD and a second sub-layer which comprises NPD. The second sub-layer which comprises NPD may be adjacent the layer comprising the crystalline A/M/X material. The second sub-layer which comprises NPD may form a planar heterojunction with the layer comprising the crystalline A/M/X material.

Suitable p-type materials also include molecular hole transporters, polymeric hole transporters and copolymer hole transporters. The p-type material may for instance be a molecular hole transporting material, a polymer or copolymer comprising one or more of the following moieties: thiophenyl, phenelenyl, dithiazolyl, benzothiazolyl, diketopyrrolopyrrolyl, ethoxydithiophenyl, amino, triphenyl amino, carbozolyl, ethylene dioxythiophenyl, dioxythiophenyl, or fluorenyl.

The p-type material may be doped, for instance with tertbutyl pyridine and LiTFSI. The p-type material may be doped to increase the hole-density. The p-type material may for instance be doped with NOBF₄ (Nitrosonium tetrafluoroborate), to increase the hole-density. The p-type material may for instance be doped with 2,3,5,6-Tetrafluoro-7,7,8,8-tetracyanoquinodimethane (F4-TCNQ). Often, when the hole-transporting (p-type) material comprises poly[N,N′-bis(4-butylphenyl)-N,N′-bisphenylbenzidine] (polyTPD), the polyTPD is doped with F4-TCNQ. PolyTPD doped with F4-TCNQ is commonly referred to as polyTPD:F4-TCNQ.

Thus the hole-transporting (p-type) material typically comprises polyTPD:F4-TCNQ. The hole-transporting (p-type) material may for instance comprise polyTPD:F4-TCNQ and NPD. For instance, the layer comprising the charge transporting material may be a p-type layer which comprises a first sub-layer which comprises polyTPD:F4-TCNQ and a second sub-layer which comprises NPD. The second sub-layer which comprises NPD may be adjacent the layer comprising the crystalline A/M/X material. The second sub-layer which comprises NPD may form a planar heterojunction with the layer comprising the crystalline A/M/X material.

The hole-transporting material (p-type material) may alternatively comprise a solid state inorganic hole transporting material. For instance, the p-type layer may comprise an inorganic hole transporter comprising an oxide of nickel (e.g. NiO), vanadium, copper, gallium, chromium or molybdenum, or any combination thereof; CuI, CuBr, CuSCN, Cu₂O, CuO or CIS; a perovskite; amorphous Si; a p-type group IV semiconductor, a p-type group III-V semiconductor, a p-type group II-VI semiconductor, a p-type group I-VII semiconductor, a p-type group IV-VI semiconductor, a p-type group V-VI semiconductor, and a p-type group II-V semiconductor, which inorganic material may be doped or undoped. The p-type layer may be a compact layer of said inorganic hole transporter.

The p-type material may be an inorganic p-type material, for instance a material comprising an oxide of nickel, vanadium, copper or molybdenum; CuI, CuBr, CuSCN, Cu₂O, CuO or CIS; amorphous Si; a p-type group IV semiconductor, a p-type group III-V semiconductor, a p-type group II-VI semiconductor, a p-type group I-VII semiconductor, a p-type group IV-VI semiconductor, a p-type group V-VI semiconductor, and a p-type group II-V semiconductor, which inorganic material may be doped or undoped. The p-type material may for instance comprise an inorganic hole transporter selected from CuI, CuBr, CuSCN, Cu₂O, CuO and CIS.

The layer of a hole transporting (p-type) material may be a solid state inorganic hole transporting material comprising an oxide of nickel, vanadium, copper or molybdenum. The solid state inorganic hole transporting material is typically present as a compact layer. Often, the solid state inorganic hole transporting material comprises nickel oxide. For instance, the optoelectronic device may comprise a compact layer of nickel oxide. Thus, the layer comprising the charge-transporting material on which the crystalline A/M/X material is disposed may be a solid state inorganic hole transporting material comprising an oxide of nickel, vanadium, copper or molybdenum, as discussed above, for instance nickel oxide.

Typically, however, the layer comprising the charge-transporting material on which the crystalline A/M/X material is disposed is a layer of a hole transporting (p-type) material and this is typically an organic hole transporting material. The organic hole transporting material typically comprises polyTPD, or NPD. The organic hole transporting material typically comprises polyTPD, but it may comprise polyTPD and NPD (for instance in the form of a first layer comprising the polyTPD and a second layer comprising the NPD). Often the polyTPD is p-doped. The dopant may be F4-TCNQ. Thus, the organic hole transporting material typically comprises polyTPD:F4-TCNQ. It may comprise polyTPD:F4-TCNQ and NPD (for instance in the form of a first layer comprising the polyTPD:F4-TCNQ and a second layer comprising the NPD). Typically, the layer comprising a crystalline A/M/X material is disposed directly on the layer of a hole transporting (p-type) material, for instance directly on the layer comprising the organic hole transporting material comprising polyTPD, preferably comprising polyTPD:F4-TCNQ, or directly on the layer comprising the organic hole transporting material comprising NPD.

Hence, the optoelectronic device of the present invention may comprise the following layers in the following order:
Layer comprising a charge transporting material (typically a p-type material as described herein, for instance comprising polyTPD, NPD, polyTPD and NPD, or nickel oxide; but this may alternatively be a n-type material);
Layer of a crystalline A/M/X material, modified with an ionic solid as described herein.

The optoelectronic device of the present invention may further comprise a first electrode. Hence, the optoelectronic device of the present invention may comprise the following layers in the following order:
First electrode, which is typically an anode. It is typically a transparent electrode. The first electrode typically comprises a transparent conducting oxide.
Layer comprising a charge transporting material (typically a p-type material as described herein, for instance comprising polyTPD, NPD, polyTPD and NPD, or nickel oxide; but this may alternatively be a n-type material);
Layer of a crystalline A/M/X material, modified with an ionic solid as described herein.

In one embodiment, the optoelectronic device comprises two layers comprising a charge transporting material as described herein. The two layers are typically disposed above and below the layer of the crystalline A/M/X material respectively. Generally, one of the two layers is an n-type layer and the other is a p-type layer. Thus, typically, one of the layers comprising a charge transporting material comprises a hole-transporting (p-type) material, which may be any of the p-type materials described herein (for instance it may comprise polyTPD, NPD, polyTPD and NPD (e.g. in two sub-layers comprising the polyTPD and the NPD respectively), or nickel oxide), and the other of the layers comprising a charge transporting material comprises an electron-transporting (n-type) material, which may be any of the n-type materials described herein (for instance it may comprise PCBM or titanium oxide). Hence, the optoelectronic device of the present invention may comprise the following layers in the following order:
Layer comprising a charge transporting material (typically a p-type material as described herein, but this may alternatively be an n-type material);
Layer of a crystalline A/M/X material, modified with an ionic solid as described herein;
Layer comprising a charge transporting material (typically an n-type material as described herein, but this may alternatively be a p-type material)

The optoelectronic device of the present invention typically further comprises a first electrode and a second electrode.

The first electrode may comprise a metal (for instance silver, gold, aluminium or tungsten), an organic conducting material such as PEDOT:PSS, or a transparent conducting oxide (for instance fluorine doped tin oxide (FTO), aluminium doped zinc oxide (AZO) or indium doped tin oxide (ITO)). Typically the first electrode is a transparent electrode, and typically this is the anode. Thus, the first electrode is typically the anode (and the second electrode is typically the cathode). Thus, the first electrode typically comprises a transparent conducting oxide, preferably FTO, ITO or AZO. The thickness of the layer of a first electrode is typically from 10 nm to 1000 nm, more typically from 40 to 400 nm.

The second electrode may be as defined above for the first electrode, for instance, the second electrode may comprise a metal (for instance silver, gold, aluminium or tungsten), an organic conducting material such as PEDOT:PSS, or a transparent conducting oxide (for instance fluorine doped tin oxide (FTO), aluminium doped zinc oxide (AZO) or indium doped tin oxide (ITO)). However, typically, the second electrode comprises, or consists essentially of, a metal for instance an elemental metal. Thus, the second electrode is typically the cathode (and the first electrode is typically the anode). Examples of metals which the second electrode material may comprise, or consist essentially of, include silver, gold, copper, aluminium, platinum, palladium, or tungsten. The second electrode may be disposed by vacuum evaporation. The thickness of the layer of a second electrode material is typically from 10 to 1000 nm, preferably from 50 nm to 150 nm.

The second electrode (usually the cathode) may optionally include a further layer comprising a metal/metal oxide, typically a layer comprising mixture of chromium and chromium (III) oxide (Cr/Cr₂O₃). The thickness of the Cr/Cr₂O₃ layer is typically between 1 to 10 nm.

Typically, the optoelectronic device comprises one or more layers comprising a charge transporting material as described herein. Typically, it comprises two layers (i.e. at least two layers) comprising a charge transporting material as described herein. The two layers are typically disposed above and below the layer of the crystalline A/M/X material respectively. As discussed above, typically one of the layers comprising a charge transporting material comprises a hole-transporting (p-type) material, which may be any of the p-type materials described herein (for instance it may comprise polyTPD, NPD, polyTPD and NPD (e.g. in two sub-layers comprising the polyTPD and the NPD respectively), or nickel oxide), and the other of the layers comprising a charge transporting material comprises an electron-transporting (n-type) material, which may be any of the n-type materials described herein (for instance it may comprise PCBM or titanium oxide). As described herein, typically the layer comprising the crystalline A/M/X material is disposed on the layer comprising the hole-transporting material, and a layer comprising an electron-transporting (n-type) material is disposed on the layer comprising the crystalline A/M/X material. Thus, the optoelectronic device of the present invention may comprise the following layers in the following order:
First electrode, which is typically an anode. The first electrode typically comprises a transparent conducting oxide, e.g. FTO;
Layer comprising a charge transporting material (this is typically a p-type material as defined herein, for instance comprising polyTPD:F4-TCNQ, or NPD, or both polyTPD:F4-TCNQ and NPD (e.g. in two sub-layers comprising the polyTPD:F4-TCNQ and the NPD respectively), or nickel oxide, but it may alternatively be a n-type material);
Layer of a crystalline A/M/X material as described herein, modified with an ionic solid as described herein;
Layer comprising a charge transporting material (this is typically an n-type material as described herein, for instance comprising PCBM or comprising both PCBM and BCP (e.g. in two sub-layers comprising the PCBM and the BCP respectively), but this may alternatively be a p-type material if the other layer comprising a charge transporting material comprises an n-type material);
Second electrode, which is typically a cathode. The first electrode typically comprises an elemental metal.

The optoelectronic device of the present invention may have a positive-intrinsic-negative (p-i-n) structure or an negative-intrinsic-positive (n-i-p) structure. In a positive-intrinsic-negative (p-i-n) structure, the layer of a crystalline A/M/X material is deposited upon the p-type layer, and the n-type layer is deposited on top of the layer of a crystalline A/M/X material. Typically, light enters the device from the side where the p-type layer is, i.e. the p-type layer is disposed on the transparent electrode (generally the anode).

In a negative-intrinsic-positive (n-i-p) structure, the layer of a crystalline A/M/X material is deposited upon the n-type layer, with the p-type layer deposited on top of the layer of a crystalline A/M/X material. Typically, light enters the n-i-p device from the side where the n-type layer is, i.e. the n-type layer is disposed on the first, transparent electrode (generally the anode). However, in the instance where the second electrode is semi-transparent, then light can be incident through the p-type layer in an n-i-p cell structure.

Typically, the optoelectronic device of the present invention has a positive-intrinsic-negative (p-i-n) structure. Hence, the optoelectronic device may comprise a layer comprising the hole-transporting (p-type) material (which may be as further defined anywhere herein), wherein the layer comprising the crystalline A/M/X material is disposed on the layer comprising the hole-transporting material, and may further comprise:

a first electrode comprising a transparent conducting oxide, wherein the layer comprising the hole-transporting material is disposed between the layer comprising the crystalline A/M/X material and the first electrode;

a layer comprising an electron-transporting (n-type) material (which may be as further defined anywhere herein); and

a second electrode which comprises a metal in elemental form, wherein the layer comprising the electron-transporting material is disposed between the layer comprising the crystalline A/M/X material and the second electrode.

It should be appreciated that the optoelectronic devices described above may comprise one or more additional layers disposed between the layers described above. For instance, the optoelectronic device may comprise one or more additional layers disposed between the first electrode and the layer of a charge transporting material. The optoelectronic device may comprise one or more additional layers disposed between the either of the layers of a charge transporting material and the layer of the crystalline A/M/X material. The optoelectronic device may comprise one or more additional layers disposed between the layer of a charge transporting material and the second electrode.

For instance, the optoelectronic device may comprise one or more additional layers that comprise an electron transporting (n-type) material, or one or more buffer layers. Typically such layers are disposed between the layer comprising a charge transporting material (typically an electron transporting material) and the second electrode. In this instance, the additional layers comprising an electron transport material may comprise an electron transporting material as described herein. Typically, the electron transporting material is an organic electron transporting material, for instance fullerene or a fullerene derivative (for instance C₆₀, C₇₀, phenyl-C₆₁-butyric acid methyl ester (PCBM), PC₇₁BM (i.e. phenyl C₇₁ butyric acid methyl ester), bis[C₆₀] BM (i.e. bis-C₆₀ butyric acid methyl ester), and 1′,1″,4′,4″-Tetrahydro-di[1,4]methanonaphthaleno[1,2:2′,3′,56,60:2″,3″ ][5,6] fullerene-C₆₀ (ICBA)), an organic electron transporting material comprising perylene or a derivative thereof, poly{[N,N0-bis(2-octyldodecyl)-naphthalene-1,4,5,8-bis(dicarboximide)-2,6-diyl]-alt-5,50-(2,20-bithiophene)} (P(NDI2OD-T2)) or bathocuproine (BCP), preferably bathocuproine (BCP). Preferably, the optoelectronic device comprises a buffer layer disposed between the layer comprising a charge transporting material (typically an electron transporting material) and the second electrode. Preferably, the buffer layer comprises bathocuproine (BCP).

Thus, the present invention typically employs two layers in between the second electrode and the layer of the crystalline A/M/X material; an n-type layer and a buffer layer, or two n-type layers. Hence, in one embodiment, the optoelectronic device of the present invention comprises the following layers in the following order, where the preferences reflect a p-i-n device:
First electrode, which is typically an anode. It is typically a transparent electrode. The first electrode typically comprises a transparent conducting oxide as described herein, e.g. FTO, ITO or AZO. Often, the transparent conducting oxide is disposed on glass;
Layer of a hole transporting (p-type) material, typically comprising polyTPD (preferably polyTPD:F4-TCNQ), NPD, polyTPD and NPD (preferably polyTPD:F4-TCNQ and NPD), or nickel oxide;
Layer of a crystalline A/M/X material as described herein, typically comprising a compound of Formula I, IA or ID as described below, modified with an ionic solid as described herein;
Layer of an electron transporting (n-type) material, preferably comprising PCBM;
Optional buffer layer or further layer of an electron transporting (n-type) material; this is preferably a layer comprising bathocuproine (BCP);
Second electrode, which is typically a cathode. The second electrode preferably comprises a metal, for instance Au, or Cr, or Au and Cr. Typically, it comprises an elemental metal. The second electrode may comprise a mixture of chromium and chromium (III) oxide (Cr/Cr₂O₃). The second electrode may comprise Au and a mixture of chromium and chromium (III) oxide (Cr/Cr₂O₃).

Typically, the layer of the hole transporting (p-type) material forms a planar heterojunction with the layer of the crystalline A/M/X material which is modified with the ionic solid. Typically, the layer of the electron transporting (n-type) material forms a planar heterojunction with the layer of the crystalline A/M/X material which is modified with the ionic solid. Thus, typically, the layer of the crystalline A/M/X material which is modified with the ionic solid forms a first planar heterojunction with the electron transporting (n-type) material and a second planar heterojunction with the hole transporting (p-type) material.

In one embodiment the optoelectronic device of the present invention comprises the following layers in the following order, in a p-i-n device:
First electrode, which is a transparent anode typically comprising a transparent conducting oxide, e.g. FTO, ITO or AZO. Preferably, the transparent conducting oxide is disposed on glass;
Layer of a hole transporting (p-type) material, typically comprising polyTPD (preferably polyTPD:F4-TCNQ), polyTPD and NPD (preferably polyTPD:F4-TCNQ and NPD), or nickel oxide;
Layer of a crystalline A/M/X material as described herein, typically comprising a compound of Formula I, IA or ID as described below, modified with an ionic solid as described herein;
Layer of an electron transporting (n-type) material, preferably comprising PCBM;
Optional buffer layer or further layer of an electron transporting (n-type) material; this is preferably a layer comprising bathocuproine;
Second electrode, which is a cathode typically comprising an elemental metal, for instance Au. Typically, it further comprises Cr. For instance, the second electrode may further comprise chromium and chromium (III) oxide (Cr/Cr₂O₃).

Typically, the layer of the hole transporting (p-type) material forms a planar heterojunction with the layer of the crystalline A/M/X material which is modified with the ionic solid. Typically, the layer of the electron transporting (n-type) material forms a planar heterojunction with the layer of the crystalline A/M/X material which is modified with the ionic solid. Thus, typically, the layer of the crystalline A/M/X material which is modified with the ionic solid forms a first planar heterojunction with the electron transporting (n-type) material and a second planar heterojunction with the hole transporting (p-type) material.

The optoelectronic device of the present invention may however have a negative-intrinsic-positive (n-i-p) structure. Hence, the optoelectronic device may comprise a layer comprising an electron-transporting (n-type) material (which may be as further defined anywhere herein), wherein the layer comprising the crystalline A/M/X material is disposed on the layer comprising the electron-transporting material, and may further comprise:

a first electrode comprising a transparent conducting oxide, wherein the layer comprising the electron-transporting material is disposed between the layer comprising the crystalline A/M/X material and the first electrode;

a layer comprising a hole-transporting (p-type) material (which may be as further defined anywhere herein); and

a second electrode which comprises a metal in elemental form, wherein the layer comprising the hole-transporting material is disposed between the layer comprising the crystalline A/M/X material and the second electrode.

In another embodiment, the optoelectronic device of the present invention comprises the following layers in the following order, in a n-i-p device:
First electrode, which is a transparent anode typically comprising a transparent conducting oxide, e.g. FTO, ITO or AZO. Preferably, the transparent conducting oxide is disposed on glass;
Layer of an electron transporting (n-type) material, which may be any of the electron transporting (n-type) materials defined herein, and in particular any of the preferred inorganic n-type materials or the preferred organic n-type materials;
Layer of a crystalline A/M/X material as described herein, typically comprising a compound of Formula I, IA or ID as described below, modified with an ionic solid as described herein,;
Layer of a hole transporting (p-type) material, which may be any of the hole transporting (p-type) materials defined herein, and in particular any of the preferred inorganic p-type materials or the preferred organic p-type materials;
Second electrode, which is a cathode typically comprising an elemental metal, for instance Au. Typically, it further comprises Cr. For instance, the second electrode may further comprise chromium and chromium (III) oxide (Cr/Cr₂O₃).

Typically, the layer of the hole transporting (p-type) material forms a planar heterojunction with the layer of the crystalline A/M/X material which is modified with the ionic solid. Typically, the layer of the electron transporting (n-type) material forms a planar heterojunction with the layer of the crystalline A/M/X material which is modified with the ionic solid. Thus, typically, the layer of the crystalline A/M/X material which is modified with the ionic solid forms a first planar heterojunction with the electron transporting (n-type) material and a second planar heterojunction with the hole transporting (p-type) material.

The optoelectronic device of the present invention may be a photovoltaic device (for instance a solar cell), a photodiode, a phototransistor, a photomultiplier, a photoresistor, or a light emitting device.

Typically, the optoelectronic device of the present invention is a photovoltaic device or a light-emitting device. It is often a photovoltaic device. Preferably, the photovoltaic device is a positive-intrinsic-negative (p-i-n) planar heterojunction photovoltaic device. Alternatively, it may be a negative-intrinsic-positive (n-i-p) planar heterojunction photovoltaic device. The photovoltaic device of the invention may be a solar cell.

The photovoltaic device of the invention may be a single-junction photovoltaic device. Alternatively, it may be a tandem junction or multi-junction photovoltaic device, for instance a tandem junction or multi-junction solar cell. In a tandem junction or multi-junction photovoltaic device of the invention, the herein disclosed A/M/X technology may be combined with known technologies to deliver optimised performance.

Typically, when the photovoltaic device of the invention is a tandem junction photovoltaic device, the device additionally comprises a further photoactive region, i.e. a further region which absorbs light and which may then generate free charge carriers. The further photoactive region is other than the region which comprises the layer comprising the crystalline A/M/X material and the adjacent layers comprising charge-transporting materials (electron- and hole-transporting materials, respectively). The further photoactive region is generally outside of the region which comprises the layer comprising the crystalline A/M/X material and the adjacent layers comprising charge (electron- and hole-) transporting materials. Thus, the further photoactive region may be disposed between the first electrode and the layer comprising a charge (electron or hole) transporting material, or between the second electrode and the layer comprising a charge (hole or electron) transporting material, in the device of the invention as defined herein. Typically, when the photovoltaic device of the invention is a multi-junction photovoltaic device, the device additionally comprises a plurality of further photoactive regions. Each one of the further photoactive regions may be disposed between the first electrode and the layer comprising a charge (electron or hole) transporting material, or between the second electrode and the layer comprising a charge (hole or electron) transporting material, in the device of the invention as defined herein.

Typically, the or each further photoactive region comprises at least one layer of a semiconductor material. The semiconductor material may for instance comprise silicon. It may for instance comprise crystalline silicon. Alternatively, for example, the semiconductor material may comprise copper zinc tin sulphide, copper zinc tin selenide, copper zinc tin selenide sulphide, copper indium gallium selenide, copper indium gallium diselenide or copper indium selenide.

Thus, for instance, when the photovoltaic device of the invention is a tandem junction photovoltaic device, the further photoactive region may be a conventional silicon solar cell. The further photoactive region may be a conventional thin film solar cell which may, for instance, comprise crystalline silicon or another thin film technology such as copper zinc tin sulphide, copper zinc tin selenide, copper zinc tin selenide sulphide, copper indium gallium selenide, copper indium gallium diselenide or copper indium selenide. The further photoactive region is preferably a silicon sub-cell.

When the photovoltaic device of the invention is a multi-junction photovoltaic device, at least one of the further photoactive regions may be a conventional silicon solar cell. At least one of the further photoactive regions may be a conventional thin film solar cell which may, for instance, comprise crystalline silicon or another thin film technology such as copper zinc tin sulphide, copper zinc tin selenide, copper zinc tin selenide sulphide, copper indium gallium selenide, copper indium gallium diselenide or copper indium selenide. Preferably. at least one of the further photoactive regions is a silicon sub-cell.

In one preferred embodiment, however, the optoelectronic device of the present invention is a light-emitting device. It may for instance be a light emitting diode.

Location of Counter Anion

The counter-anion may be present (a) within the layer comprising the crystalline A/M/X material, (b) between the layer comprising the crystalline A/M/X material and the layer comprising the charge-transporting material, and/or (c) within the layer comprising the charge-transporting material.

In one embodiment, the counter-anion is present within the layer comprising the crystalline A/M/X material. In another embodiment the counter-anion is present between the layer comprising the crystalline A/M/X material and the layer comprising the charge-transporting material. In another embodiment the counter-anion is present within the layer comprising the charge-transporting material.

For instance, the counter-anion may be present: (a) within the layer comprising the crystalline A/M/X material and (b) between the layer comprising the crystalline A/M/X material and the layer comprising the charge-transporting material. The counter-anion may be present: (a) within the layer comprising the crystalline A/M/X material and (c) within the layer comprising the charge-transporting material. The counter-anion may be present: (b) between the layer comprising the crystalline A/M/X material and (c) within the layer comprising the charge-transporting material. The counter-anion may be present (a) within the layer comprising the crystalline A/M/X material, (b) between the layer comprising the crystalline A/M/X material and the layer comprising the charge-transporting material and (c) within the layer comprising the charge-transporting material.

Typically, some or all of the counter-anion is present: (b) between the layer comprising the crystalline A/M/X material and the layer comprising the charge-transporting material and (c) within the layer comprising the charge-transporting material. Some of the counter-anion may be present: (b) between the layer comprising the crystalline A/M/X material and the layer comprising the charge-transporting material and (c) within the layer comprising the charge-transporting material. All of the counter-anion may be present: (b) between the layer comprising the crystalline A/M/X material and the layer comprising the charge-transporting material and (c) within the layer comprising the charge-transporting material.

Typically, some or all of the counter-anion is present within the layer comprising the charge-transporting material. For instance, some of the counter-anion may be present within the layer comprising the charge-transporting material. For instance, all of the counter-anion may be present within the layer comprising the charge-transporting material.

Typically, the organic cation and the counter anion are present within the layer comprising the crystalline A/M/X material. Typically, the crystalline A/M/X material is a polycrystalline A/M/X material comprising crystallites of the A/M/X material and grain boundaries between the crystallites, wherein the organic cation and the counter anion are present at grain boundaries between the crystallites and on an outer surface of the crystalline A/M/X material.

Typically, some or all of the counter-anion is not present within the crystalline A/M/X material. For instance, at least some of the counter-anion may be present on an outer surface of the crystalline A/M/X material. For instance, all of the counter-anion may be present on an outer surface of the crystalline A/M/X material. Therefore, the counter-anion may not be present in the bulk material of the layer comprising the crystalline A/M/X material and may, for instance, be present at the interface with the charge transporting material.

As discussed above, the layer comprising the charge-transporting material may be a layer of an electron transporting (n-type) material, as described herein, or a layer of a hole transporting (p-type) material, as described herein. Typically, the layer comprising a charge transporting material is a layer of a hole transporting (p-type) material. Thus, typically the charge-transporting material is a hole-transporting (p-type) material. Typically, the layer comprising the charge-transporting material comprises polyTPD, or nickel oxide, for instance it may be polyTPD:F4-TCNQ or a compact layer of nickel oxide. Typically, the layer comprising the crystalline A/M/X material is directly disposed on the layer comprising the charge transporting material, such that the layer comprising the crystalline A/M/X material and the layer comprising the charge-transporting material are in physical contact. Thus, the layer comprising the crystalline A/M/X material may be directly disposed on a layer comprising polyTPD (preferably polyTPD:F4-TCNQ).

Hence, the counter-anion may be present (a) within the layer comprising the crystalline A/MI/X material, (b) between the layer comprising the crystalline A/M/X material and the layer of hole transporting (p-type) material, which preferably comprises polyTPD or nickel oxide, and/or (c) within the layer of hole transporting (p-type) material, which preferably comprises polyTPD or nickel oxide. It is thought that the ionic solid provides improved interaction at the interface between the layer of the crystalline A/M/X material and the layer of hole transporting (p-type) material, thereby enhancing V_{OC}, fill factor (FF) and efficiency (PCE).

Further Treatment with a Second Ionic Compound

The optoelectronic device of the invention may further comprise a second ionic compound, wherein the second ionic compound is a salt comprising an organic cation and a counter anion which is different from the ionic solid. The second ionic compound is typically in the solid state at room temperature. It is usually in the solid state at 50° C. and at temperatures of less than 50° C. In other words, the second ionic compound typically has a melting point of greater than 50° C. Often, the second ionic compound is in the solid state at 100° C. and at temperatures of less than 100° C. In other words, the second ionic compound usually has a melting point of greater than 100° C. The second ionic compound may be as further defined anywhere herein for the ionic solid provided that, when the second ionic compound is present in the optoelectronic device of the invention it is different from the ionic solid that is present in that optoelectronic device of the invention. Usually, the organic cation, the counter-anion, or both the organic cation and the counter anion, of the second ionic compound are different from those of the ionic solid.

Often, the organic cation of the second ionic compound is different from that of the ionic solid.

For instance, the optoelectronic device of the invention may further comprise a second ionic compound which is a salt comprising an organic cation and a counter anion wherein the organic cation of the second ionic compound is an iminium cation of formula II (for instance N-((diisopropylamino)methylene)-N-diisopropylaminium) and the organic cation of the ionic solid is an imidazolium cation of formula III or a triazolium cation of formula IV.

Alternatively, for instance, the optoelectronic device of the invention may further comprise a second ionic compound which is a salt comprising an organic cation and a counter anion wherein the organic cation of the second ionic compound is a triazolium cation of formula IV (for instance 6,7-dihydro-2-pentafluorophenyl-5H-pyrrolo[2,1-c]-1,2,4-triazolium) and the organic cation of the ionic solid is an imidazolium cation of formula III.

Often, the counter-anion of the second ionic compound is the same as that of the ionic solid. It is often the same polyatomic counter-anion, for instance the same borate counter anion. It is often tetrafluoroborate.

The second ionic compound is typically disposed between the layer comprising a charge-transporting material and the layer comprising the crystalline A/M/X material. Thus, the second ionic compound may be disposed at an interface between the layer comprising the crystalline A/M/X material and a layer comprising a charge-transporting material. The layer comprising the charge-transporting material may be as further defined anywhere herein; it typically comprises a hole transporting (p-type) material as described herein, for instance polyTPD, NPD, polyTPD and NPD, or nickel oxide; but it may alternatively comprise an electron transporting (n-type) material, for instance PCBM.

The second ionic compound may be disposed on either side of the layer comprising the crystalline A/M/X material, or on both sides of (i.e. both “above” and “below”) the layer comprising the crystalline A/M/X material. The second ionic compound may be disposed at both interfaces between the layer comprising the crystalline A/M/X material and the two layers either side of the A/M/X material which comprise charge-transporting materials. Thus, the second ionic compound may be disposed between the layer comprising the crystalline A/M/X material and the layer comprising the hole transporting (p-type) material and between the layer comprising the crystalline A/M/X material and the layer comprising the electron transporting (n-type) material. Thus, the second ionic compound may be disposed at an interface between the layer comprising the crystalline A/M/X material and the hole transporting (p-type) material and at an interface between the layer comprising the crystalline A/M/X material and the electron transporting (n-type) material. The second ionic compound disposed between the layer comprising the crystalline A/M/X material and the layer comprising the hole transporting (p-type) material may be the same as or different from the second ionic compound disposed between the layer comprising the crystalline A/M/X material and the layer comprising the electron transporting (n-type) material.

Thus, the optoelectronic device may comprise a first second ionic compound disposed between the layer comprising the crystalline A/M/X material and the layer comprising the hole transporting (p-type) material and a second second ionic compound disposed between the layer comprising the crystalline A/M/X material and the layer comprising the electron transporting (n-type) material, wherein the first and second second ionic compounds are the same or different.

A/M/X Material

The optoelectronic device of the present invention comprises a layer comprising a crystalline A/M/X material, the crystalline A/M/X material comprising a compound of formula: [A]ₐ[M]_{b}[X]_{c}, wherein: [A] comprises one or more A cations; [M] comprises one or more M cations which are metal or metalloid cations; [X] comprises one or more X anions; a is a number from 1 to 6; b is a number from 1 to 6; and c is a number from 1 to 18. a is often a number from 1 to 4, b is often a number from 1 to 3, and c is often a number from 1 to 8.

Each of a, b and c may or may not be an integer. For instance, a, b or c may not be an integer where the compound adopts a structure having vacancies such that the crystal lattice is not completely filled. The method of the invention provides very good control over stoichiometry of the product and so is well-suited for forming structures where a, b or c is not an integer (for instance a structure having vacancies in one or more of the A, M or X sites). Accordingly, in some embodiments, one or more of a, b and c is a non-integer value. For example, one of a, b and c may be a non-integer value. In one embodiment, a is a non-integer value. In another embodiment, b is a non-integer value. In yet another embodiment, c is a non-integer value.

In other embodiments, each of a, b and c are integer values. Thus, in some embodiments, a is an integer from 1 to 6; b is an integer from 1 to 6; and c is an integer from 1 to 18. a is often an integer from 1 to 4, b is often an integer from 1 to 3, and c is often an integer from 1 to 8.

In the compound of formula [A]ₐ[M]_{b}[X]_{c}, generally: [A] comprises one or more A cations, which A cations may for instance be selected from alkali metal cations or organic monocations; [M] comprises one or more M cations which are metal or metalloid cations selected from Pd⁴⁺, W⁴⁺, Re⁴⁺, Os⁴⁺, Ir⁴⁺, Pt⁴⁺, Sn⁴⁺, Pb⁴⁺, Ge⁴⁺, Te⁴⁺, Bi³⁺, Sb³⁺, Ca²⁺, Sr²⁺, Cd²⁺, Cu²⁺, Ni²⁺, Mn²⁺, Fe²⁺, Co²⁺, Pd²⁺, Ge²⁺, Sn²⁺, Pb²⁺, Yb²⁺ and Eu²⁺, preferably Sn²⁺, Pb²⁺, Cu²⁺, Ge²⁺, and Ni²⁺; particularly preferably Pb²⁺ and Sn²⁺; [X] comprises one or more X anions selected from halide anions (e.g. Cl⁻, Br⁻, and I⁻), O²⁻, S²⁻, Se²⁻, and Te²⁻; a is a number from 1 to 4; b is a number from 1 to 3; and c is a number from 1 to 8. Often, the compound of [A]ₐ[M]_{b}[X]_{c} either does not comprise the methylammonium cation (i.e. it is free of methylammonium) or it only contains a small amount of methylammonium. Here, a “small amount” of methylammonium typically means that [A] of the compound of formula [A]ₐ[M]_{b}[X]_{c} consists of methylammonium and at least one A cation other than methylammonium, provided that the molar fraction of the methylammonium in [A] is less than 15% of [A]. Preferably the molar fraction of the methylammonium in [A] is less than 10% of [A], and more preferably less than 5% of [A], for instance less than 2% of [A], more preferably less than 1% of [A]. Thus, a compound of formula [A]ₐ[M]_{b}[X]_{c} which contains only a small amount of methylammonium is a compound of formula [(CH₃NH₃)ₓ(A′)₁₋ₓ]ₐ[M]_{b}[X]_{c}, wherein (A′) represents at least one A cation other than methylammonium, and x is less than 0.15, preferably less than 0.10, for instance less than 0.05, and more preferably less than 0.02, for instance less than 0.01. Typically, the compound of [A]ₐ[M]_{b}[X]_{c} does not comprise the methylammonium cation, i.e. it is free of methylammonium cation.

Preferably the compound of formula [A]ₐ[M]_{b}[X]_{c} comprises a perovskite. The compound of formula [A]ₐ[M]_{b}[X]_{c} often comprises a metal halide perovskite. [M] comprises one or more M cations which are metal or metalloid cations. [M] may comprise two or more different M cations. [M] may comprise one or more monocations, one or more dications, one or more trications or one or more tetracations.

Typically, the one or more M cations are selected from Ca²⁺, Sr²⁺, Cd²⁺, Cu²⁺, Ni²⁺, Mn²⁺, Fe²⁺, Co²⁺, Pd²⁺, Ge²⁺, Sn²⁺, Pb²⁺, Yb²⁺, Eu²⁺, Bi³⁺, Sb³⁺+, Pd⁴⁺+, W⁴⁺, Re⁴⁺, Os⁴⁺, Ir⁴⁺, Pt⁴⁺, Sn⁴⁺, Pb⁴⁺, Ge⁴⁺ or Te⁴⁺. Preferably, the one or more M cations are selected from Cu²⁺, Pb²⁺, Ge²⁺ or Sn²⁺.

Typically, [M] comprises one or more metal or metalloid dications. For instance, each M cation may be selected from Ca²⁺, Sr²⁺, Cd²⁺, Cu²⁺, Ni²⁺, Mn²⁺, Fe²⁺, Co²⁺+, Pd²⁺, Ge²⁺, Sn²⁺, Pb²⁺, Yb²⁺ and Eu²⁺, preferably Sn²⁺, Pb²⁺, Cu²⁺, Ge²⁺, and Ni²⁺; preferably Sn²⁺ and Pb²⁺. In some embodiments, [M] comprises two different M cations, typically where said cations are Sn²⁺ and Pb²⁺, preferably Pb²⁺.

In general, said one or more A cations are monocations. [A] typically comprises one or more A cations which may be organic and/or inorganic monocations. Typically, [A] comprises two or more different A cations. For instance, [A] may comprise at least two A cations which may be organic and/or inorganic monocations, or at least three A cations which may be organic and/or inorganic monocations. Thus, the compound of formula [A]ₐ[M]_{b}[X]_{c} may be a mixed cation perovskite. [A] may comprise at least one A cation which is an organic cation and at least one A cation which is an inorganic cation. [A] may comprise at least two A cations which are both organic cations. [A] may comprise at least two A cations which are both inorganic cations. In one embodiment, [A] comprises two A cations which are both organic cations and an A cation which is an inorganic cation.

Where an A species is an inorganic monocation, A is typically an alkali metal monocation (that is, a monocation of a metal found in Group 1 of the periodic table), for instance Li⁺, Na⁺, K⁺, RB⁺, Cs⁺, for example Cs⁺ or RB⁺. Typically, [A] comprises at least one organic monocation. Where an A species is an organic monocation, A is typically an ammonium cation, for instance methylammonium, or an iminium cation, for instance formamidimium.

Thus, typically each A cation is selected from: an alkali metal cation, for instance Li+, Na+, K+, Rb+, Cs+; a cation of the formula [R₁R₂R₃R₄N]⁺, wherein each of R₁, R₂, R₃, R₄ is independently selected from hydrogen, unsubstituted or substituted C₁₋₂₀ alkyl, and unsubstituted or substituted C₆₋₁₂ aryl, and at least one of R₁, R₂, R₃ and R₄ is not hydrogen; a cation of the formula [R₅R₆N═CH—NR₇R₈]⁺, wherein each of R₅, R₆, R₇ and R₈ is independently selected from hydrogen, unsubstituted or substituted C₁₋₂₀ alkyl, and unsubstituted or substituted C₆₋₁₂ aryl; and C₁₋₁₀ alkylamammonium, C₂₋₁₀ alkenylammonium, C₁₋₁₀ alkyliminium, C₃₋₁₀ cycloalkylammonium and C₃₋₁₀ cycloalkyliminium, each of which is unsubstituted or substituted with one or more substituents selected from amino, C₁₋₆ alkylamino, imino, C₁₋₆ alkylimino, C₁₋₆ alkyl, C₂₋₆ alkenyl, C₃₋₆ cycloalkyl and C₆₋₁₂ aryl.

For instance, each A cation is selected from Cs⁺, Rb⁺, methylammonium [(CH₃NH₃)⁺], ethylammonium [(CH₃CH₂NH₃)⁺], propylammonium [(CH₃CH₂CH₂NH₃)⁺]. Butylammonium [(CH₃CH₂CH₂CH₂NH₃)⁺], pentylammoium [(CH₃CH₂CH₂CH₂CH₂NH₃)⁺], hexylammonium [(CH₃CH₂CH₂CH₂CH₂CH₂NH₃)⁺], heptylammonium [(CH₃CH₂CH₂CH₂CH₂CH₂CH₂NH₃)⁺], octylammonium [(CH₃CH₂CH₂CH₂CH₂CH₂CH₂CH₂NH₃)⁺], tetramethylammonium [(N(CH₃)₄)⁺], formamidinium [(H₂N—C(H)═NH₂)⁺], 1-aminoethan-1-iminium [(H₂N—C(CH₃)═NH₂)⁺] and guanidinium [(H₂N—C(NH₂)═NH₂)⁺]. Preferably each A cation is selected from Cs⁺, Rb⁺, methylammonium, ethylammonium, propylammonium. butylammonium, pentylammoium, hexylammonium, heptylammonium, octylammonium, formamidinium and guanidinium. Often, however, [A] does not comprise methylammonium.

[A] usually comprises one, two or three A monocations. [A] may comprises a single cation selected from methylammonium [(CH₃NH₃)⁺], ethylammonium [(CH₃CH₂NH₃)⁺], propylammonium [(CH₃CH₂CH₂NH₃)+], dimethylammonium [(CH₃)₂NH⁺], tetramethylammonium [(N(CH₃)₄)⁺], formamidinium [(H₂N—C(H)═NH2)+], 1-aminoethan-1-iminium [(H₂N—C(CH₃)═NH₂)⁺], guanidinium [(H₂N—C(NH₂)═NH₂)⁺], Cs⁺ and Rb⁺. For instance [A] may comprise a single cation that is methylammonium [(CH₃NH₃)⁺].

Alternatively, [A] may comprise two cations selected from this group, for instance Cs⁺ and formamidinium [(H₂N—C(H)═NH₂)⁺], or for instance Cs⁺ and Rb⁺, or for instance methylammonium [(CH₃NH₃)⁺] and formamidinium [(H₂N—C(H)═NH₂)⁺], preferably Cs⁺ and formamidinium [(H₂N—C(H)═NH₂)⁺]. Often, however, [A] does not comprise methylammonium.

Alternatively, [A] may comprise three cations selected from this group, for instance methylammonium [(CH₃NH₃)⁺], formamidinium [(H₂N—C(H)═NH₂)⁺] and Cs⁺. Often, however, [A] does not comprise methylammonium. Thus, [A] may for instance comprise formamidinium and Cs⁺ but not methylammonium. [A] may for instance consist only of formamidinium and Cs⁺.

Often, [A] comprises Cs⁺ and formamidinium and:
(i) the compound of formula [A]ₐ[M]_{b}[X]_{c} does not comprise methylammonium, or
(ii) [A] of the compound of formula [A]ₐ[M]_{b}[X]_{c} comprises methylammonium, Cs⁺ and formamidinium, provided that the molar fraction of methylammonium in [A] is less than 15% of [A]. Preferably the molar fraction of the methylammonium in [A] is less than 10% of [A], and more preferably less than 5% of [A], for instance less than 2% of [A], more preferably less than 1% of [A].

[X] comprises one or more X anions. Typically, [X] comprises one or more halide anions, i.e. an anion selected from F⁻, Br⁻, Cl⁻ and I⁻. Typically, each X anion is a halide. [X] typically comprises one, two or three X anions and these are generally selected from Br⁻, Cl⁻ and I⁻.

X may comprise two more different X anions. Typically, [X] comprises two or more different halide anions. [X] may for instance consist of two X anions, such as Cl and Br, or Br and I, or Cl and I. Therefore, the compound of formula [A]ₐ[M]_{b}[X]_{c} often comprises a mixed halide perovskite. When [A] comprises one or more organic cations, the compound of formula [A]ₐ[M]_{b}[X]_{c} may be an organic-inorganic metal halide perovskite.

Typically, said one or more A cations are monocations, said one or more M cations are dications, and said one or more X anions are one or more halide anions.

Often, [A] comprises at least two different A cations as described herein and [X] comprises at least two different X anions as described herein. In some embodiments, [A] comprises at least three different A cations as described herein and [X] comprises at least two different X anions as described herein.

Often, [X] comprises I and Br. In some embodiments, [X] comprises I and Br, wherein the molar ratio of I to Br in the compound of formula [A]ₐ[M]_{b}[X]_{c} is less than 9:1, preferably less than 7:1, more preferably equal to or less than 4:1. [X] may consist only of I and Br. Thus, [X] may consist only of I and Br and the molar ratio of I to Br in the compound of formula [A]ₐ[M]_{b}[X]_{c} may be less than 9:1, and is preferably less than 7:1, more preferably equal to or less than 4:1.

Often, [X] comprises I and Br, wherein the molar ratio of I to Br in the compound of formula [A]ₐ[M]_{b}[X]_{c} is less than 9:1, preferably less than 7:1, more preferably equal to or less than 4:1, and [A] comprises Cs⁺ and formamidinium wherein:
(i) the compound of formula [A]ₐ[M]_{b}[X]_{c} does not comprise methylammonium, or
(ii) [A] of the compound of formula [A]ₐ[M]_{b}[X]_{c} comprises methylammonium, Cs⁺ and formamidinium, provided that the molar fraction of methylammonium in [A] is less than 15% of [A]. Preferably the molar fraction of the methylammonium in [A] is less than 10% of [A], and more preferably less than 5% of [A], for instance less than 2% of [A], more preferably less than 1% of [A].

Often, in the compound of formula [A]ₐ[M]_{b}[X]_{c}, [X] is [Br_{y}I_{1-y}] wherein y is greater than 0 and less than 1, thus the compound of formula [A]ₐ[M]_{b}[X]_{c} is a compound of formula [A]ₐ[M]_{b}[Br_{y}I_{1-y}]_{c}, wherein [A], a, [M] and b are as defined herein and y is greater than 0 and less than 1. In such embodiments, it may be preferred that y is greater than 0.10 and less than 1. For instance, y may be greater than 0.15 and less than 1. Typically, for instance, y is at least 0.20 and less than 1, for instance y may be at least 0.22 and less than 1, or at least 0.23 and less than 1. y may, for instance be from 0.15 to 0.50, for instance from 0.20 to 0.40 or from 0.20 to 0.30.

Often, in the compound of formula [A]ₐ[M]_{b}[X]_{c}, [X] is [Br_{y}I_{1-y}] wherein y is greater than 0.10 and less than 1, preferably greater than 0.15 and less than 1, and more preferably at least 0.20 and less than 1, and [A] comprises Cs⁺ and formamidinium wherein:
(i) the compound of formula [A]ₐ[M]_{b}[X]_{c} does not comprise methylammonium, or
(ii) [A] of the compound of formula [A]ₐ[M]_{b}[X]_{c} comprises methylammonium, Cs⁺ and formamidinium, provided that the molar fraction of methylammonium in [A] is less than 15% of [A]. Preferably the molar fraction of the methylammonium in [A] is less than 10% of [A], and more preferably less than 5% of [A], for instance less than 2% of [A], more preferably less than 1% of [A].

The compound of formula [A]ₐ[M]_{b}[X]_{c} may be other than: [{(H₂N—C(H)═NH₂)_{0.83} (CH₃NH₃)_{0.17}}_{0.95}Cs_{0.05}]Pb[Br_{0.1}I_{0.9}]₃. In other words, the compound may be other than (FA_{0.83}MA_{0.17})_{0.95}Cs_{0.05}Pb(I_{0.9}Br_{0.1})₃, where FA is formamidinium and MA is methylammonium.

Typically, a=1, b=1 and c=3. Thus, the compound of formula [A]ₐ[M]_{b}[X]_{c} may be a compound of formula [A][M][X]₃, wherein [A], [M] and [X] are as described herein. Typically, the crystalline A/M/X material comprises: a perovskite of formula (I):

[A][M][X]₃ (I)

wherein: [A] comprises one or more A cations which are monocations; [M] comprises one or more M cations which are metal or metalloid dications; and [X] comprises one or more anions which are halide anions.

In some embodiments, the perovskite of formula (I) comprises a single A cation, a single M cation and a single X cation. i.e., the perovskite is a perovskite of the formula (IA):

AMX₃ (IA)

wherein A, M and X are as defined above. In a preferred embodiment, A is selected from (CH₃NH₃)⁺, (CH₃CH₂NH₃)⁺, (CH₃CH₂CH₂NH₃)⁺, (N(CH₃)₄), (H₂N—C(H)═NH₂)%, (H₂N—C(CH₃)═NH₂)⁺, (H₂N—C(NH₂)═NH₂)⁺, Cs⁺ and Rb⁺; M is Pb²⁺ or Sn²⁺ and X is selected from Br⁻, Cl⁻ and I⁻.

For instance, the crystalline A/M/X material may comprise, or consist essentially of, a perovskite compound of formula (IA) selected from APbI₃, APbBr₃, APbCl₃, ASnI₃, ASnBr₃ and ASnCl₃, wherein A is a cation as described herein.

For instance, the crystalline A/M/X material may comprise, or consist essentially of, a perovskite compound of formula (IA) selected from CH₃NH₃PbI₃, CH₃NH₃PbBr₃, CH₃NH₃PbCl₃, CH₃NH₃SnI₃, CH₃NH₃SnBr₃, CH₃NH₃SnCl₃, CsPbI₃, CsPbBr₃, CsPbCl₃, CsSnI₃, CsSnBr₃, CsSnCl₃, (H₂N—C(H)═NH₂)PbI₃, (H₂N—C(H)═NH₂)PbBr₃, (H₂N—C(H)═NH₂)PbCl₃, (H₂N—C(H)═NH₂)SnI₃, (H₂N—C(H)═NH₂)SnBr₃ and (H₂N—C(H)═NH₂)SnCl₃, in particular CH₃NH₃PbI₃ or CH₃NH₃PbBr₃, preferably CH₃NH₃PbI₃.

In one embodiment, the perovskite is a perovskite of the formula (IB):

[A^{I}ₓA^{II}₁₋ₓ]MX₃ (IB)

wherein A^{I} and A^{II} are as defined above with respect to A, wherein M and X are as defined above and wherein x is greater than 0 and less than 1. In a preferred embodiment, A^{I} and A^{II} are each selected from (CH₃NH₃)⁺, (CH₃CH₂NH₃)⁺, (CH₃CH₂CH₂NH₃)⁺, (N(CH₃)₄)⁺, (H₂N—C(H)═NH₂)⁺, (H₂N—C(CH₃)═NH₂)⁺, (H₂N—C(NH₂)═NH₂)⁺, Cs⁺ and Rb⁺; M is Pb²⁺ or Sn²⁺ and X is selected from Br⁻, Cl⁻ and I⁻. A^{I} and A^{II} may for instance be (H₂N—C(H)═NH₂)⁺ and Cs³⁰ respectively, or they may be (CH₃NH₃)⁺ and (H₂N—C(H)═NH₂)⁺ respectively. Alternatively, they may be Cs⁺ and Rb⁺ respectively. Preferably, A^{I} and A^{II} are (H₂N—C(H)═NH₂)⁺ and Cs⁺ respectively

For instance, the crystalline A/M/X material may comprise, or consist essentially of, a perovskite compound of formula (IB) selected from (CsₓRb₁₋ₓ)PbBr₃, (CsₓRb₁₋ₓ)PbCl₃, (CsₓRb₁₋ₓ)PbI₃, [(CH₃NH₃)ₓ(H₂N—C(H)═NH₂)₁₋ₓ]PbCl₃, [(CH₃NH₃)ₓ(H₂N—C(H)═NH₂)₁₋ₓ]PbBr₃, [(CH₃NH₃)ₓ(H₂N—C(H)═NH₂)₁₋ₓ]PbI₃, [(CH₃NH₃)ₓCs₁₋ₓ]PbCl₃, [(CH₃NH₃)ₓCs₁₋ₓ,]PbBr₃, [(CH₃NH₃)ₓCs₁₋ₓ,]PbI₃, [(H₂N—C(H)═NH₂)ₓCs₁₋ₓ]PbCl₃, [(H₂N—C(H)═NH₂)ₓCs₁₋ₓ]PbBr₃, [(H₂N—C(H)═NH₂)ₓCs₁₋ₓ]PbI₃, [(CH₃NH₃)ₓ(H₂N—C(H)═NH₂)₁₋ₓ]SnCl₃, [(CH₃NH₃)ₓ(H₂N—C(H)═NH₂)₁₋ₓ]SnBr₃, [(CH₃NH₃)ₓ(H₂N—C(H)═NH₂)₁₋ₓ]SnI₃, [(CH₃NH₃)ₓCs₁₋ₓ]SnCl₃, [(CH₃NH₃)ₓCs₁₋ₓ]SnBr₃, [(CH₃NH₃)ₓCs₁₋ₓ]SnI₃, [(H₂N—C(H)═NHz)ₓCs₁₋ₓ]SnCl₃, [(H₂N—C(H)═NH₂)ₓCs₁₋ₓ]SnBr₃, and [(H₂N—C(H)═NH₂)ₓCs₁₋ₓ]SnI₃, where x is greater than 0 and less than 1, for instance x may be from 0.01 to 0.99 or from 0.05 to 0.95 or 0.1 to 0.9.

In one embodiment, the perovskite is a perovskite compound of the formula (IC):

AM[X^{I}_{y}X^{II}_{1-y}]₃ (IC)

wherein A and M are as defined above, wherein X^{I} and X^{II} are as defined above in relation to X and wherein y is greater than 0 and less than 1. In a preferred embodiment, A is selected from (CH₃NH₃)⁺, (CH₃CH₂NH₃)⁺, (CH₃CH₂CH₂NH₃)⁺, (N(CH₃)₄)⁺, (H₂N—C(H)—NH2)⁺, (H₂N—C(CH₃)═NH₂)⁺, (H₂N—C(NH₂)═NH₂)⁺, Cs⁺ and Rb⁺; M is Pb²⁺ or Sn²⁺; and X^{I} and X^{II} are each selected from Br⁻, Cl⁻ and I⁻.

For instance, the crystalline A/M/X material may comprise, or consist essentially of, a perovskite compound of formula (IC) selected from APb[Br_{y}I_{1-y}]₃, APb[Br_{y}Cl_{1-y}]₃, APb[I_{y}Cl_{1-y}]₃, ASn[Br_{y}I_{1-y}]₃, ASn[Br_{y}Cl_{1-y}]₃, ASn[I_{y}Cl_{1-y}]₃, where y is greater than 0 and less than 1, and wherein A is a cation as described herein. y may be from 0.01 to 0.99. For instance, y may be from 0.05 to 0.95 or 0.1 to 0.9.

For instance, the crystalline A/M/X material may comprise, or consist essentially of, a perovskite compound of formula (IC) selected from CH₃NH₃Pb[Br_{y}I_{1-y}]₃, CH₃NH₃Pb[Br_{y}Cl_{1-y}]₃, CH₃NH₃Pb[I_{y}Cl_{1-y}]₃, CH₃NH₃Sn[Br_{y}I_{1-y}]₃, CH₃NH₃Sn[Br_{y}Cl_{1-y}]₃, CH₃NH₃Sn[I_{y}Cl_{1-y}]₃, CsPb[Br_{y}I_{1-y}]₃, CsPb[Br_{y}Cl_{1-y}]₃, CsPb[I_{y}Cl_{1-y}]₃, CsSn[Br_{y}I_{1-y}]₃, CsSn[Br_{y}Cl_{1-y}]₃, CsSn[I_{y}Cl_{1-y}]₃, (H₂N—C(H)═NH₂)Pb[Br_{y}I_{1-y}]₃, (H₂N—C(H)═NH₂)Pb[Br_{y}Cl_{1-y}]₃, (H₂N—C(H)═NH₂)Pb[I_{y}Cl_{1-y}]₃, (H₂N—C(H)═NH₂)Sn[Br_{y}I_{1-y}]₃, (H₂N—C(H)═NH₂)Sn[Br_{y}Cl_{1-y}]₃, and (H₂N—C(H)═NH₂)Sn[I_{y}Cl_{1-y}]₃, where y is greater than 0 and less than 1, for instance y may be from 0.01 to 0.99 or from 0.05 to 0.95 or 0.1 to 0.9.

In a preferred embodiment, the perovskite is a perovskite of the formula (ID):

[A^{I}ₓA^{II}₁₋ₓ]M[X^{I}_{y}X^{II}_{1-y}]₃ (ID)

wherein A^{I} and A^{II} are as defined above with respect to A, M is as defined above, X^{I} and X^{II} are as defined above in relation to X and wherein x and y are both greater than 0 and less than 1. In a preferred embodiment, A^{I} and A^{II} are each selected from ((CH₃NH₃)⁺, (CH₃CH₂NH₃)⁺, (CH₃CH₂CH₂NH₃)⁺, (N(CH₃)₄)⁺, (H₂N—C(H)═NH₂)⁺, (H₂N—C(CH₃)═NH₂)⁺, (H₂N—C(NH₂)═NH₂)⁺, Cs⁺ and Rb⁺, preferably A^{I} and A^{II} are (H₂N—C(H)═NH₂)⁺ and Cs⁺ respectively; M is Pb²⁺ or Sn²⁺;and X^{I} and X^{II} are each selected from Br⁻, Cl⁻ and I⁻.

For instance, the crystalline A/M/X material may comprise, or consist essentially of, a perovskite compound of formula (ID) selected from (CsₓRb₁₋ₓ)Pb(Br_{y}Cl_{1-y})₃, (CsₓRb₁₋ₓ)Pb(Br_{y}I_{1-y})₃, and (CsₓRb₁₋ₓ)Pb(Cl_{y}I_{1-y})₃, [(CH₃NH₃)ₓ(H₂N—C(H)═NH₂)_{1-X}]Pb[Br_{y}I_{1-y}]₃, [(CH₃NH₃)ₓ(H₂N—C(H)═NH2)₁-x]Pb[Br_{y}Cl_{1-y}]₃, [(CH₃NH₃)ₓ(H₂N—C(H)═NH₂)₁₋ₓ]Pb[I_{y}Cl_{1-y}]₃, [(CH₃NH₃)ₓCs₁₋ₓ]Pb[Br_{y}I_{1-y}]₃, [(CH₃NH₃)ₓCs₁₋ₓ]Pb[Br_{y}Cl_{1-y}]₃, [(CH₃NH₃)ₓCs₁₋ₓ]Pb[I_{y}Cl_{1-y}]₃, [(H₂N—C(H)═NH₂)ₓCs_{1- x}]Pb[Br_{y}I_{1-y}]₃, [(H₂N—C(H)═NH₂)ₓCs₁₋ₓ]Pb[Br_{y}Cl_{1-y}]₃, [(H₂N—C(H)═NH₂)ₓCs₁₋ₓ]Pb[I_{y}Cl_{1-y}]₃, [(CH₃NH₃)ₓ(H₂N—C(H)═NH₂)₁₋ₓ]Sn[Br_{y}I_{1-y}]₃, [(CH₃NH₃)ₓ(H₂N—C(H)═NH₂)₁₋ₓ]Sn[Br_{y}Cl_{1-y}]₃, [(CH₃NH₃)ₓ(H₂N—C(H)═NH₂)₁₋ₓ]Sn[I_{y}Cl_{1-y}]₃, [(CH₃NH₃)ₓCs₁₋ₓ]Sn[Br_{y}I_{1-y}]₃, [(CH₃NH₃)ₓCs₁₋ₓ]Sn[Br_{y}Cl_{1- y}]₃, [(CH₃NH₃)ₓCs₁₋ₓ]Sn[I_{y}Cl_{1-y}]₃, [(H₂N—C(H)═NH₂)ₓCs₁₋ₓ]Sn[Br_{y}I_{1-y}]₃, [(H₂N—C(H)═NH₂)ₓCs_{1-X}]Sn[Br_{y}Cl_{1-y}]₃, and [(H₂N—C(H)═NH₂)ₓCS₁₋ₓ]Sn[I_{y}Cl_{1-y}]₃, where x and y are both greater than 0 and less than 1, for instance x and y may both be from 0.01 to 0.99 or from 0.05 to 0.95 or 0.1 to 0.9.

[(H₂N—C(H)═NH₂)ₓCs₁₋ₓ]Pb[Br_{y}I_{1-y}]₃ is particularly preferred. Thus, the crystalline A/M/X material preferably comprises, or consists essentially of, a perovskite compound of formula (ID) which is [(H₂N—C(H)═NH₂)ₓCs₁₋ₓ]Pb[Br_{y}I_{1-y}]₃, where x and y are both greater than 0 and less than 1, for instance x and y may both be from 0.01 to 0.99 or from 0.05 to 0.95 or 0.1 to 0.9. Often, x is greater than 0 and less than 1, and y is greater than 0.10 and less than 1. For instance, x may be greater than 0 and less than 1, and y may be greater than 0.15 and less than 1. Typically, x is greater than 0 and less than 1, and y is greater than 0.2 and less than 1. Often, x is greater than 0 and less than 1, and y is at least 0.22 and less than 1, for instance y may be at least 0.23 and less than 1. y may, for instance be from 0.15 to 0.40, for instance from 0.20 to 0.30.

[(H₂N—C(H)═NH₂)_{0.83}Cs_{0.17}]Pb[Br_{0.23}I_{0.77}]₃ is a particularly preferred perovskite compound of formula (ID). Thus, typically, [A]ₐ[M]_{b}[X]_{c} is [(H₂N—C(H)═NH₂)_{0.83}Cs_{0.17}]Pb[Br_{0.23}I_{0.77}]₃. In other words, the crystalline A/M/X material preferably comprises, or consists essentially of, or consists of, a perovskite compound of formula [(H₂N—C(H)═NH₂)_{0.83}Cs_{0.17}]Pb[Br_{0.23}I_{0.77}]₃.

In one embodiment, the perovskite is a perovskite of the formula (IE):

A[M^{I}_{z}M^{II}_{1-z}]X₃ (IE)

wherein M^{I} and M^{II} are as defined above with respect to M, A and X are as defined above, and wherein z is greater than 0 and less than 1. In a preferred embodiment, A is selected from (CH₃NH₃)⁺, (CH₃CH₂NH₃)⁺, (CH₃CH₂CH₂NH₃)⁺, (N(CH₃)₄)⁺, (H₂N—C(H)═NH₂)⁺, (H₂N—C(CH₃)═NH₂)⁺, (H₂N—C(NH₂)═NH₂)⁺, Cs⁺ and Rb⁺; M^{I} is Pb²⁺ and M^{II} is Sn²⁺; and X is selected from Br⁻, Cl⁻ and I⁻.

For instance, the crystalline A/M/X material may comprise, or consist essentially of, a perovskite compound of formula (IE) selected from CH₃NH₃[Pb_{z}Sn_{1-z}]Cl₃, CH₃NH₃[Pb_{z}Sn_{1-z}]Br₃, CH₃NH₃[Pb_{z}Sn_{1-z}]I₃, Cs[Pb_{z}Sn_{1-z}]Cl₃, Cs[Pb_{z}Sn_{1-z}]Br₃, Cs[Pb_{z}Sn_{1-z}]₃, (H₂N—C(H)═NH₂)[Pb_{z}Sn_{1-z}]Cl₃, (H₂N—C(H)═NH₂)[Pb_{z}Sn_{1-z}]Br₃, and (H₂N—C(H)═NH₂)[Pb_{z}Sn_{1-z}]I₃, where z is greater than 0 and less than 1, for instance z may be from 0.01 to 0.99 or from 0.05 to 0.95 or 0.1 to 0.9.

In one embodiment, the perovskite is a perovskite of the formula (IF):

[A^{I}ₓA^{II}₁₋ₓ][M^{I}_{z}M^{II}_{1-z}]X₃ (IF)

wherein A^{I} and A^{II} are as defined above with respect to A, M^{I} and M^{II} are as defined above with respect to M, and X is as defined above and wherein x and z are both greater than 0 and less than 1. In a preferred embodiment, A^{I} and A^{II} are each selected from (CH₃NH₃)⁺, (CH₃CH₂NH₃), (CH₃CH₂CH₂NH₃)⁺, (N(CH₃)₄)⁺, (H₂N—C(H)═NH₂)⁺, (H₂N—C(CH₃)═NH₂)⁺, (H₂N—C(NH₂)═NH₂)⁺, Cs⁺ and Rb⁺; M^{I} is Pb²⁺ and M^{II} is Sn²⁺; and X is selected from Br⁻, Cl⁻ and I⁻. A^{I} and A^{II} may for instance be (H₂N—C(H)═NH₂)⁺ and Cs⁺ respectively, or they may be (CH₃NH₃)⁺ and (H₂N—C(H)═NH₂)⁺ respectively. Alternatively, they may be Cs⁺ and Rb⁺ respectively.

For instance, the crystalline A/M/X material may comprise, or consist essentially of, a perovskite compound of formula (IF) selected from [(CH₃NH₃)ₓ(H₂N—C(H)═NH₂)₁₋ₓ][Pb_{z}Sn_{1-z}]Cl₃, [(CH₃NH₃)ₓ(H₂N—C(H)═NH₂)₁₋ₓ][Pb_{z}Sn_{1-z}]Br₃, [(CH₃NH₃)ₓ(H₂N—C(H)═NH₂)₁₋ₓ][Pb_{z}Sn_{1-z}]I₃, [(CH₃NH₃)ₓCs₁₋ₓ][Pb_{z}Sn_{1-z}]Cl₃, [(CH₃NH₃)ₓCs₁₋ₓ][Pb_{z}Sn_{1-z}]Br₃, [(CH₃NH₃)ₓCs₁₋ₓ][Pb_{z}Sn_{1-z}]I₃, [(H₂N—C(H)═NH₂)ₓCs₁₋ₓ][Pb_{z}Sn_{1-z}]Cl₃, [(H₂N—C(H)═NH₂)ₓCs₁₋ₓ][Pb_{z}Sn_{1-z}]Br₃, [(H₂N—C(H)═NH₂)ₓCs₁₋ₓ][Pb_{z}Sn_{1-z}]I₃, where x and z are both greater than 0 and less than 1, for instance x and z may each be from 0.01 to 0.99 or from 0.05 to 0.95 or 0.1 to 0.9.

In one embodiment, the perovskite is a perovskite compound of the formula (IG):

A[M^{I}_{z}M^{II}_{1-z}][X^{I}_{y}X^{II}_{1-y}]₃ (IG)

wherein A is as defined above, M^{I} and M^{II} are as defined above with respect to M, and wherein X^{I} and X^{II} are as defined above in relation to X and wherein y and z are both greater than 0 and less than 1. In a preferred embodiment, A is selected from (CH₃NH₃)⁺, (CH₃CH₂NH₃)⁺, (CH₃CH₂CH₂NH₃), (N(CH₃)₄)⁺, (H₂N—C(H)═NH₂)⁺, (H₂N—C(CH₃)═NH₂)⁺, (H₂N—C(NH₂)═NH₂)⁺, Cs⁺ and Rb⁺; M^{I} is Pb²⁺ and M^{II} is Sn²⁺; and X^{I} and X^{II} are each selected from Br⁻, Cl⁻ and I⁻.

For instance, the crystalline A/M/X material may comprise, or consist essentially of, a perovskite compound of formula (IG) selected from A[Pb_{z}Sn_{1-z}][Br_{y}I_{1-y}]₃, A[Pb_{z}Sn_{1-z}][Br_{y}Cl_{1-y}]₃, A[Pb_{z}Sn_{1-z}][I_{y}Cl_{1-y}]₃, where y and z are both greater than 0 and less than 1, and wherein A is a cation as described herein. y and z may each be from 0.01 to 0.99. For instance, y and z may each be from 0.05 to 0.95 or 0.1 to 0.9.

For instance, the crystalline A/M/X material may comprise, or consist essentially of, a perovskite compound of formula (IG) selected from CH₃NH₃[Pb_{z}Sn_{1-z}][Br_{y}I_{1-y}]₃, CH₃NH₃[Pb_{z}Sn_{1-z}][Br_{y}Cl_{1-y}]₃, CH₃NH₃[Pb_{z}Sn_{1-z}][I_{y}Cl_{1-y}]₃, Cs[Pb_{z}Sn_{1-z}][Br_{y}I_{1-y}]₃, Cs[Pb_{z}Sn_{1-z}][Br_{y}Cl_{1-y}]₃, Cs[Pb_{z}Sn_{1-z}][I_{y}Cl_{1-y}]₃, (H₂N—C(H)═NH₂)[Pb_{z}Sn_{1-z}][Br_{y}I_{1-y}]₃, (H₂N—C(H)═NH₂)[Pb_{z}Sn_{1-z}][Br_{y}Cl_{1-y}]₃, and (H₂N—C(H)═NH₂)[Pb_{z}Sn_{1-z}][I_{y}Cl_{1-y}]₃, where y and z are both greater than 0 and less than 1, for instance y and z may each be from 0.01 to 0.99 or from 0.05 to 0.95 or 0.1 to 0.9.

In a preferred embodiment, the perovskite is a perovskite of the formula (IH):

[A^{I}ₓA^{II}₁₋ₓ][M^{I}_{z}M^{II}_{1-z}][X^{I}_{y}X^{II}_{1-y}]₃ (IH)

wherein A^{I} and A^{II} are as defined above with respect to A, M^{I} and M^{II} are as defined above with respect to M, X^{I} and X^{II} are as defined above in relation to X and wherein x, y and z are each greater than 0 and less than 1. In a preferred embodiment, A^{I} and A^{II} are each selected from ((CH₃NH₃)⁺, (CH₃CH₂NH₃)⁺, (CH₃CH₂CH₂NH₃)⁺, (N(CH₃)₄)⁺, (H₂N—C(H)═NH₂)⁺, (H₂N—C(CH₃)═NH₂)⁺, (H₂N—C(NH₂)═NH₂)⁺, Cs⁺ and Rb⁺; M^{I} is Pb²⁺ and M^{II} is Sn²⁺; and X^{I} and X^{II} are each selected from Br⁻, Cl⁻ and I⁻.

For instance, the crystalline A/M/X material may comprise, or consist essentially of, a perovskite compound of formula (IH) selected from [(CH₃NH₃)ₓ(H₂N—C(H)═NH₂)₁₋ₓ][Pb_{z}Sn_{1-z}][Br_{y}I_{1-y}]₃, [(CH₃NH₃)ₓ(H₂N—C(H)═NH₂)₁₋ₓ][Pb_{z}Sn_{1-z}][Br_{y}Cl_{1-y}]₃, (CH₃NH₃)ₓ(H₂N—C(H)═NH₂)₁₋ₓ][Pb_{z}Sn_{1-z}][I_{y}Cl_{1-y}]₃, [(CH₃NH₃)ₓCS₁₋ₓ][Pb_{z}Sn_{1-z}][Br_{y}I_{1-y}]₃, [(CH₃NH₃)ₓCS₁₋ₓ][Pb_{z}Sn_{1-z}][Br_{y}Cl_{1-y}]₃, [(CH₃NH₃)ₓCS₁₋ₓ][Pb_{z}Sn_{1-z}][I_{y}Cl_{1-y}]₃, [(H₂N—C(H)═NH₂)ₓCs₁₋ₓ][Pb_{z}Sn_{1-z}][Br_{y}I_{1-y}]₃, [(H₂N—C(H)═NH₂)ₓCs₁₋ₓ][Pb_{z}Sn_{1-z}][Br_{y}Cl_{1-y}]₃, and [(H₂N—C(H)═NH₂)ₓCs₁₋ₓ][Pb_{z}Sn_{1-z}][I_{y}Cl_{1-y}]₃, where x, y and z are each greater than 0 and less than 1, for instance x, y and z may each be from 0.01 to 0.99 or from 0.05 to 0.95 or 0.1 to 0.9.

In one embodiment, a=2, b=1 and c=4. In that embodiment, the crystalline A/M/X material comprises a compound (a “2D layered perovskite”) of formula (II):

[A]₂[M][X]₄ (II)

wherein: [A] comprises one or more A cations which are monocations; [M] comprises one or more M cations which are metal or metalloid dications; and [X] comprises one or more X anions which are halide anions. In this embodiment, the A and M cations, and the X anions, are as defined above.

In another embodiment, a=2, b=1 and c=6. In that embodiment, the crystalline A/M/X material may in that case comprise a hexahalometallate of formula (III):

[A]₂[M][X]₆ (III)

wherein: [A] comprises one or more A cations which are monocations; [M] comprises one or more M cations which are metal or metalloid tetracations; and [X] comprises one or more X anions which are halide anions.

The hexahalometallate of formula (III) may in a preferred embodiment be a mixed monocation hexahalometallate. In a mixed monocation hexahalometallate, [A] comprises at least two A cations which are monocations; [M] comprises at least one M cation which is a metal or metalloid tetracation (and typically [M] comprises a single M cation which is a metal or metalloid tetracation); and [X] comprises at least one X anion which is a halide anion (and typically [X] comprises a single halide anion or two types of halide anion). In a mixed metal hexahalometallate, [A] comprises at least one monocation (and typically [A] is a single monocation or two types of monocation); [M] comprises at least two metal or metalloid tetracations (for instance Ge⁴⁺ and Sn⁴⁺); and [X] comprises at least one halide anion (and typically [X] is a single halide anion or two types of halide anion). In a mixed halide hexahalometallate, [A] comprises at least one monocation (and typically [A] is a single monocation or two types of monocation); [M] comprises at least one metal or metalloid tetracation (and typically [M] is a single metal tetra cation); and [X] comprises at least two halide anions, for instance Br⁻ and Cl⁻ or Br⁻ and I⁻.

[A] may comprise at least one A monocation selected from any suitable monocations, such as those described above for a perovskite. In the case of a hexahalometallate, each A cation is typically selected from Li⁺, Na⁺, K⁺, Rb⁺, Cs⁺, NH₄⁺ and monovalent organic cations. Monovalent organic cations are singly positively charged organic cations, which may, for instance, have a molecular weight of no greater than 500 g/mol. For instance, [A] may be a single A cation which is selected from Li⁺, Na⁺, K⁺, Rb⁺, Cs⁺, NH₄⁺ and monovalent organic cations. [A] preferably comprises at least one A cation which is a monocation selected from Rb⁺, Cs⁺, NH₄⁺ and monovalent organic cations. For instance, [A] may be a single inorganic A monocation selected from Li⁺, Na⁺, K⁺, Rb⁺, Cs⁺, NH₄⁺ and NH₄⁺. In another embodiment, [A] may be at least one monovalent organic A cation. For instance, [A] may be a single monovalent organic A cation. In one embodiment, [A] is (CH₃NH₃)⁺. In another embodiment, [A] is (H₂N—C(H)═NH₂)⁺.

Preferably, [A] comprises two or more types of A cation. [A] may be a single A monocation, or indeed two A monocations, each of which is independently selected from K⁺, Rb⁺, Cs⁺, NH₄⁺, (CH₃NH₃)⁺, (CH₃CH₂NH₃)⁺, (CH₃CH₂CH₂NH₃)⁺, (N(CH₃)₄)⁺, (N(CH₂CH₃)₄), (N(CH₂CH₂CH₃)₄), (H₂N—C(H)═NH₂)⁺ and (H₂N—C(CH₃)═NH₂)⁺.

[M] may comprise one or more M cations which are selected from suitable metal or metalloid tetracations. Metals include elements of groups 3 to 12 of the Periodic Table of the Elements and Ga, In, Tl, Sn, Pb, Bi and Po. Metalloids include Si, Ge, As, Sb, and Te. For instance, [M] may comprise at least one M cation which is a metal or metalloid tetracation selected from Ti⁴⁺, V⁴⁺, Mn⁴⁺, Fe⁴⁺, Co⁴⁺, Zr⁴⁺, Nb⁴⁺, Mo⁴⁺, Ru⁴⁺, Rh⁴⁺, Pd⁴⁺, Hf⁴⁺, Ta⁴⁺, W⁴⁺, Re⁴⁺, OS⁴⁺, Ir⁴⁺, Pt⁴⁺, Sn⁴⁺, Pb⁴⁺, Po⁴⁺, Si⁴⁺, Ge⁴⁺, and Te⁴⁺. Typically, [M] comprises at least one metal or metalloid tetracation selected from Pd⁴⁺, W⁴⁺, Re⁴⁺, Os⁴⁺, Ir⁴⁺, Pt⁴⁺, Sn⁴⁺, Pb⁴⁺, Ge⁴⁺, and Te⁴⁺. For instance, [M] may be a single metal or metalloid tetracation selected from Pd⁴⁺, W⁴⁺, Re⁴⁺, Os⁴⁺, Ir⁴⁺, Pt⁴⁺, Sn⁴⁺, Pb⁴⁺, Ge⁴⁺, and Te⁴⁺.

Typically, [M] comprises at least one M cation which is a metal or metalloid tetracation selected from Sn⁴⁺, Te⁴⁺, Ge⁴⁺ and Re⁴⁺. In one embodiment [M] comprises at least one M cation which is a metal or metalloid tetracation selected from Pb⁴⁺, Sn⁴⁺, Te⁴⁺, Ge⁴⁺ and Re⁴⁺. For instance, [M] may comprise an M cation which is at least one metal or metalloid tetracation selected from Pb⁴⁺, Sn⁴⁺, Te⁴⁺ and Ge⁴⁺. Preferably, [M] comprises at least one metal or metalloid tetracation selected from Sn⁴⁺, Te⁴⁺, and Ge⁴⁺. As discussed above, the hexahalometallate compound may be a mixed-metal or a single-metal hexahalometallate. Preferably, the hexahalometallate compound is a single-metal hexahalometallate compound. More preferably, [M] is a single metal or metalloid tetracation selected from Sn⁴⁺, Te⁴⁺, and Ge⁴⁺. For instance, [M] may be a single metal or metalloid tetracation which is Te⁴⁺. For instance, [M] may be a single metal or metalloid tetracation which is Ge⁴⁺. Most preferably, [M] is a single metal or metalloid tetracation which is Sn⁴⁺.

[X] may comprise at least one X anion which is a halide anion. [X] therefore comprises at least one halide anion selected from F⁻, Cl⁻, Br⁻ and I⁻. Typically, [X] comprises at least one halide anion selected from Cl⁻, Br⁻ and I⁻. The hexahalometallate compound may be a mixed-halide hexahalometallate or a single-halide hexahalometallate. If the hexahalometallate is mixed, [X] comprises two, three or four halide anions selected from F⁻, Cl⁻, Br⁻ and I⁻. Typically, in a mixed-halide compound, [X] comprises two halide anions selected from F⁻, Cl⁻, Br⁻ and I⁻.

In some embodiments, [A] is a single monocation and [M] is a single metal or metalloid tetracation. Thus, the crystalline A/M/X material may, for instance, comprise a hexahalometallate compound of formula (IIIA)

A₂M[X]₆ (IIIA)

wherein: A is a monocation; M is a metal or metalloid tetracation; and [X] is at least one halide anion. [X] may be one, two or three halide anions selected from F⁻, Cl⁻, Br⁻ and I⁻, and preferably selected from Cl⁻, Br⁻ and I⁻. In formula (IIIA), [X] is preferably one or two halide anions selected from Cl⁻, Br⁻ and I⁻.

The crystalline A/M/X material may, for instance, comprise, or consist essentially of, a hexahalometallate compound of formula (IIIB)

A₂MX_{6-y}X′_{y} (IIIB)

wherein: A is a monocation (i.e. the second cation); M is a metal or metalloid tetracation (i.e. the first cation); X and X′ are each independently a (different) halide anion (i.e. two second anions); and y is from 0 to 6. When y is 0 or 6, the hexahalometallate compound is a single-halide compound. When y is from 0.01 to 5.99 the compound is a mixed-halide hexahalometallate compound. When the compound is a mixed-halide compound, y may be from 0.05 to 5.95. For instance, y may be from 1.00 to 5.00.

The hexahalometallate compound may, for instance, be A₂SnF_{6-y}Cl_{y}, A₂SnF_{6-y}Br_{y}, A₂SnF_{6-y}, A₂SnCl_{6-y}Br_{y}, A₂SnCl_{6-y}I_{y}, A₂SnBr_{6-y}I_{y}, A₂TeF_{6-y}Cl_{y}, A₂TeF_{6-y}Br_{y}, A₂TeF_{6-y}I_{y}, A₂TeCl_{6-y}Br_{y}, A₂TeCl_{6-y}I_{y}, A₂TeBr_{6-y}I_{y}, A₂GeF_{6-y}Cl_{y}, A₂GeF_{6-y}Br_{y}, A₂GeF_{6-y}I_{y}, A₂GeCl_{6-y}Br_{y}, A₂GeCl_{6-y}I_{y}, A₂GeBr_{6-y}I_{y}, A₂ReF_{6-y}Cl_{y}, A₂ReF_{6-y}Br_{y}, A₂ReF_{6-y}I_{y}, A₂ReCl_{6-y}Br_{y}, A₂ReCl_{6-y}I_{y} or A₂ReBr_{6-y}I_{y}, wherein: A is K⁺, Rb⁺, Cs⁺, (R¹NH₃)⁺, (NR²₄)⁺, or (H₂N—C(R¹)═NH₂)⁺, wherein R¹ is H, a substituted or unsubstituted C₁₋₂₀ alkyl group or a substituted or unsubstituted aryl group, and R² is a substituted or unsubstituted C₁₋₁₀ alkyl group; and y is from 0 to 6. Optionally, y is from 0.01 to 5.99. If the hexahalometallate compound is a mixed-halide compound, y is typically from 1.00 to 5.00. A may be as defined above. For instance, A may be Cs⁺, NH₄⁺, (CH₃NH₃)⁺, (CH₃CH₂NH₃)⁺, (N(CH₃)₄)⁺, (N(CH₂CH₃)₄)⁺, (H₂N—C(H)═NH₂)⁺ or (H₂N—C(CH₃)═NH₂)⁺, for instance Cs⁺, NH₄⁺, or (CH₃NH₃)⁺.

The hexahalometallate compound may typically be A₂SnF_{6-y}Cl_{y}, A₂SnF_{6-y}Br_{y}, A₂SnF_{6-y}I_{y}, A₂SnCl_{6-y}Br_{y}, A₂SnCl_{6-y}I_{y}, or A₂SnBr_{6-y}I_{y}, wherein: A is K⁺, Rb⁺, Cs⁺, (R¹NH₃)⁺, (NR²₄)⁺, or (H₂N—C(R¹)═NH₂)⁺, or A is as defined herein, wherein R¹ is H, a substituted or unsubstituted C₁₋₂₀ alkyl group or a substituted or unsubstituted aryl group, or R² is a substituted or unsubstituted C₁₋₁₀ alkyl group; and y is from 0 to 6.

In another embodiment, the hexahalometallate compound is A₂GeF_{6-y}Cl_{y}, A₂GeF_{6-y}Br_{y}, A₂GeF_{6-y}I_{y}, A₂GeCl_{6-y}Br_{y}, A₂GeCl_{6-y}I_{y}, or A₂GeBr_{6-y}I_{y}, wherein: A is K⁺, Rb⁺, Cs⁺, (R¹NH₃)⁺, (NR²₄)⁺, or (H₂N—C(R¹)═NH₂)⁺, or A is as defined herein, wherein R¹ is H, a substituted or unsubstituted C₁₋₂₀ alkyl group or a substituted or unsubstituted aryl group, and R² is a substituted or unsubstituted C₁₋₁₀ alkyl group; and y is from 0 to 6.

The hexahalometallate compound may, for instance, be A₂TeF_{6-y}Cl_{y}, A₂TeF_{6-y}Br_{y}, A₂TeF_{6-y}I_{y}, A₂TeCl_{6-y}Br_{y}, A₂TeCl_{6-y}I_{y}, or A₂TeBr_{6-y}I_{y}, wherein: A is K⁺, Rb⁺, Cs⁺, (R¹NH₃)⁺, (NR²₄)⁺, or (H₂N—C(R¹)═NH₂)⁺, or A is as defined herein, wherein R¹ is H, a substituted or unsubstituted C₁₋₂₀ alkyl group or a substituted or unsubstituted aryl group, and R² is a substituted or unsubstituted C₁₋₁₀ alkyl group; and y is from 0 to 6 or y is as defined herein.

Often, y will be from 1.50 to 2.50. For instance, y may be from 1.80 to 2.20. This may occur if the compound is produced using two equivalents of AX′ and one equivalent of MX₄, as discussed below. In some embodiments, all of the ions are single anions or cations. Thus, the crystalline A/M/X material may comprise, or consist essentially of, a hexahalometallate compound of formula (IIIC)

A₂MX₆ (IIIC)

wherein: A is a monocation; M is a metal or metalloid tetracation; and X is a halide anion. A, M and X may be as defined herein.

The hexahalometallate compound may be A₂SnF₆, A₂SnCl₆, A₂SnBr₆, A₂SnI₆, A₂TeF₆, A₂TeCl₆, A₂TeBr₆, A₂TeI₆, A₂GeF₆, A₂GeCl₆, A₂GeBr₆, A₂GeI₆, A₂ReF₆, A₂ReCl₆, A₂ReBr₆ or A₂ReI₆, wherein: A is K⁺, Rb⁺, Cs⁺, (R¹NH₃)⁺, (NR²₄)⁺, or (H₂N—C(R′)═NH₂)⁺, wherein R¹ is H, a substituted or unsubstituted C₁₋₂₀ alkyl group or a substituted or unsubstituted aryl group, and R² is a substituted or unsubstituted C₁₋₁₀ alkyl group. A may be as defined herein.

Preferably, the hexahalometallate compound is Cs₂SnI₆, Cs₂SnBr₆, Cs₂SnBr_{6-y}I_{y}, Cs₂SnCl_{6-y}I_{y}, Cs₂SnCl_{6-y}Br_{y}, (CH₃NH₃)₂SnI₆, (CH₃NH₃)₂SnBr₆, (CH₃NH₃)₂SnBr_{6-y}I_{y}, (CH₃NH₃)₂SnCl_{6-y}I_{y}, (CH₃NH₃)₂SnCl_{6-y}Br_{y}, (H₂N—C(H)═NH₂)₂SnI₆, (H₂N—C(H)═NH₂)₂SnBr₆, (H₂N—C(H)═NH₂)₂SnBr_{6-y}I_{y}, (H₂N—C(H)═NH₂)₂SnCl_{6-y}I_{y} or (H₂N—C(H)═NH₂)₂SnCl_{6-y}Br_{y} wherein y is from 0.01 to 5.99. For example, the hexahalometallate compound may be (CH₃NH₃)₂SnI₆, (CH₃NH₃)₂SnBr₆, (CH₃NH₃)₂SnCl₆, (H₂N—C(H)═NH₂)₂SnI₆, (H₂N—C(H)═NH₂)₂SnBr₆ or (H₂N—C(H)═NH₂)₂SnCl₆. The hexahalometallate compound may be Cs₂SnI₆, Cs₂SnBr₆, Cs₂SnCl_{6-y}Br_{y}, (CH₃NH₃)₂SnI₆, (CH₃NH₃)₂SnBr₆, or (H₂N C(H)═NH₂)₂SnI₆.

The crystalline A/M/X material may comprise a bismuth or antimony halogenometallate. For instance, the crystalline A/M/X material may comprise a halogenometallate compound comprising: (i) one or more monocations ([A]) or one or more dications ([B]); (ii) one or more metal or metalloid trications ([M]); and (iii) one or more halide anions ([X]). The compound may be a compound of formula BBiX₅, B₂BiX₇ or B₃BiX₉ where B is (H₃NCH₂NH₃)²⁺, (H₃N(CH₂)₂NH₃)²⁺, (H₃N(CH₂)₃NH₃)²⁺, (H₃N(CH₂)₄NH₃)²⁺, (H₃N(CH₂)₅NH₃)²⁺, (H₃N(CH₂)₆NH₃)²⁺, (H₃N(CH₂)₇NH₃)²⁺, (H₃N(CH₂)₈NH₃)²⁺ or (H₃N—C₆H₄—NH₃)²⁺ and X is I⁻, Br⁻ or Cl⁻, preferably I⁻.

In yet further embodiments, the crystalline A/M/X materials may be double perovskites. Such compounds are defined in WO 2017/037448, the entire contents of which is incorporated herein by reference. Typically, the compound is a double perovskite compound of formula (IV):

[A]₂[B⁺][B³⁺][X]₆ (IV);

wherein: [A] comprises one or more A cations which are monocations, as defined herein; [B⁺] and [B³⁺] are equivalent to [M] where M comprises one or more M cations which are monocations and one or more M cations which are trications; and [X] comprises one or more X anions which are halide anions.

The one or more M cations which are monocations comprised in [B⁺] are typically selected from metal and metalloid monocations. Preferably, the one or more M cations which are monocations are selected from Li⁺, Na⁺, K⁺, Rb⁺, Cs⁺, Cu⁺, Ag⁺, Au⁺ and Hg⁺. More preferably, the one or more M cations which are monocations are selected from Cu⁺, Ag⁺ and Au⁺. Most preferably, the one or more M cations which are monocations are selected from Ag⁺ and Au⁺. For instance, [B⁺] may be one monocation which is Ag⁺ or [B⁺] may be one monocation which is Au⁺.

The one or more M cations which are trications comprised in [B³⁺] are typically selected from metal and metalloid trications. Preferably, the one or more M cations which are trications are selected from Bi³⁺, Sb³⁺, Cr³⁺, Fe³⁺, Co³⁺, Ga³⁺, As³⁺, Ru³⁺, Rh³⁺, In³⁺, Ir³⁺ and Au³⁺. More preferably, the one or more M cations which are trications are selected from Bi³⁺ and Sb³⁺. For instance, [B³⁺] may be one trication which is Bi³⁺ or [B³⁺] may be one trication which is Sb³⁺+. Bismuth has relatively low toxicity compared with heavy metals such as lead.

In some embodiments, the one or more M cations which are monocations (in [B⁺]) are selected from Cu⁺, Ag⁺ and Au⁺ and the one or more M cations which are trications (in [B³⁺]) are selected from Bi³⁺ and Sb³⁺. An exemplary double perovskite is Cs₂BiAgBr₆.

Typically, where the compound is a double perovskite it is a compound of formula (IVa):

A₂B⁺B³⁺[X]₆ (IVa);

wherein: the A cation is as defined herein; B⁺ is an M cation which is a monocation as defined herein; B³⁺ is an M cation which is a trication as defined herein; and [X] comprises one or more X anions which are halide anions, for instance two or more halide anions, preferably a single halide anion.

In yet another embodiment, the compound may be a layered double perovskite compound of formula (V):

[A]₄[B⁺][B³⁺][X]₈ (V);

wherein: [A], [B⁺], [B³⁺] and [X] are as defined above. In some embodiments, the layered double perovskite compound is a double perovskite compound of formula (Va):

A₄B+B³⁺[X]₈ (Va);

wherein: the A cation is as defined herein; B⁺ is an M cation which is a monocation as defined herein; B³⁺ is an M cation which is a trication as defined herein; and [X] comprises one or more X anions which are halide anions, for instance two or more halide anions, preferably a single halide anion or two kinds of halide anion.

In yet another embodiment, the compound may be a compound of formula (VI):

[A]₄[M][X]₆ (VI);

wherein: [A], [M] and [X] are as defined above (in relation to, for instance, compounds of formula (I) or (II)). However, preferably the compound is not a compound of formula (VI). Where the compound is a compound of formula (VI), the compound may preferably be a compound of formula (VIA)

[A^{I}A^{II}]₄[M][X]₆ (VIA);

that is, a compound wherein [A] comprises two types of A monoacation. In other preferred embodiments, the compound of formula (VI) may be a compound of formula (VIB):

[A]₄[M][X^{I}X^{II}]₆ (VIB);

that is, a compound of formula (VI) wherein [X] comprises two types of X anion. In yet other preferred embodiments, the compound of formula (VI) may be a compound of formula (VIC):

[A^{I}A^{II}]₄[M][X^{I}X^{II}]₆ (VIC);

that is, a compound of formula (VI) wherein [A] comprises two types of A monoacation and [X] comprises two types of X anion. In formulae (VIa), (VIb) and (VIc), each of: [A], [M] and [X] are as defined above (in relation to, for instance, compounds of formula (I) or (II)).

In another embodiment, a=1, b=1 and c=4. In that embodiment, the crystalline A/M/X material may in that case comprise a compound of formula (VII):

[A][M][X]₄ (VII)

wherein: [A] comprises one or more A cations which are monocations; [M] comprises one or more M cations which are metal or metalloid trications; and [X] comprises one or more X anions which are halide anions. The A monocations and M trications are as defined herein. An exemplary compound of formula (VII) is AgBiI₄.

It should be understood that the invention also encompasses processes for producing variants of the above-described structures (I), (II), (III), (IV), (V), (VI) and (VII) where one or more of the relevant a, b and c values are non-integer values.

Preferably, the compound of formula [A]ₐ[M]_{b}[X]_{c} is a compound of formula [A][M][X]₃, a compound of formula [A]₄[M][X]₆ or a compound of formula [A]₂[M][X]₆. For example, in preferred embodiments the compound of formula [A]ₐ[M]_{b}[X]_{c} is a compound of formula (I), for instance a compound of formula (IA), (IB), (IC), (ID), (IE), (IF), (IG), (IH), (IIIA), or a compound of formula (IIIB), (IIIC), (VIA), (VIB), or (VIC). Generally, the compound of formula [A]ₐ[M]_{b}[X]_{c} is a compound of formula (I), for instance a compound of formula (IA), (IB), (IC), (ID), (IE), (IF), (IG) or (IH).

In some embodiments, the compound of formula [A]ₐ[M]_{b}[X]_{c} is a compound wherein [A] comprises two or more different A cations. For examples, [A] may contain two types of cation or three types of A cation. In some embodiments, the compound of formula [A]ₐ[M]_{b}[X]_{c} is a compound wherein [X] comprises two or more different X anions. For example, [X] may contain two types of anion, e.g. halide anions. In some embodiments, the compound of formula [A]ₐ[M]_{b}[X]_{c} is a compound wherein [M] comprises two or more different M cations. For example, [X] may contain two types of anion, e.g. Sn²⁺ and Pb²⁺.

In one aspect of each of these embodiments, the compound of formula [A]ₐ[M]_{b}[X]_{c} is a compound wherein [A] comprises two or more different A cations and wherein [X] comprises two or more different X anions. For example, [A] may contain two types of A cation and [X] may contain two types of X anion (e.g. two types of halide anion). [A] may contain three types of A cation and [X] may contain two types of X anion (e.g. two types of halide anion).

In one aspect of each of these embodiments, the compound of formula [A]ₐ[M]_{b}[X]_{c} is a compound wherein [A] comprises two or more different A cations and wherein [M] comprises two or more different M cations. For example, [A] may contain two types of A cation and [M] may contain two types of M cation (e.g. Sn²⁺ and Pb²⁺).

In one aspect of each of these embodiments, the compound of formula [A]ₐ[M]_{b}[X]_{c} is a compound wherein [X] comprises two or more different X anions and wherein [M] comprises two or more different M cations. For example, [X] may contain two types of X anion (e.g. two types of halide anion) and [M] may contain two types of M cation (e.g. Sn²⁺ and Pb²⁺).

In one aspect of each of these embodiments, the compound of formula [A]ₐ[M]_{b}[X]_{c} is a compound wherein [A] comprises two or more different A cations and wherein [X] comprises two or more different X anions and wherein [M] comprises two or more different M cations. For example, [A] may contain two types of A cation, [X] may contain two types of X anion (e.g. two types of halide anion) and [M] may contain two types of M cation (e.g. Sn²⁺ and Pb²⁺).

Process 1—Deposition of Solution with AMX and Ionic Solid

The present invention also relates to a first process for producing an ionic solid-modified film of a crystalline A/M/X material, wherein the crystalline A/M/X material comprises a compound of formula: [A]ₐ[M]_{b}[X]_{c}, wherein: [A] comprises one or more A cations; [M] comprises one or more M cations which are metal or metalloid cations; [X] comprises one or more X anions; wherein a is a number from 1 to 6; b is a number from 1 to 6; and c is a number from 1 to 18, and wherein the ionic solid is a salt which comprises an organic cation and a counter-anion,

the process comprising:
disposing a film-forming solution on a substrate, wherein the film-forming solution comprises a solvent, the one or more A cations, the one or more M cations, the one or more X anions, the organic cation and the counter-anion.

The ionic solid is optionally other than a quaternary ammonium halide salt. The ionic solid is usually other than a primary ammonium halide salt. The ionic solid is often other than a secondary ammonium halide salt. The ionic solid is usually other than a tertiary ammonium halide salt. Thus, typically, the ionic solid is other than a primary, secondary, tertiary or quaternary ammonium halide salt, i.e. the counter-anion is other than halide and the organic cation is other than a primary, secondary, tertiary or quaternary ammonium cation. The ionic solid is typically other than a formamidinium halide salt, and usually other than a guanidinium halide salt, i.e. the counter-anion is other than halide and the organic cation is other than formamidinium or guanidinium. Thus, typically, the ionic solid is other than a primary, secondary, tertiary or quaternary ammonium halide salt and other than a formamidinium or guanidinium halide salt. The ionic solid is typically other than a halide salt of a cation of formula (X) as defined herein. Often, the ionic solid is other than a primary, secondary, tertiary or quaternary ammonium halide salt and other than a halide salt of a cation of formula (X) as defined herein. Typically, when the counter-anion of the ionic solid is halide, the organic cation of the ionic solid is other than each of the one or more A cations of the crystalline A/M/X material. Often, the organic cation of the ionic solid is other than each of the one or more A cations of the crystalline A/M/X material.

In the first process of the invention, the crystalline A/M/X material, the one or more A cations, the one or more M cations, the one or more X anions, the ionic solid, the organic cation and the counter-anion may be as further defined anywhere herein; for instance, they may be as further defined anywhere hereinbefore for the optoelectronic device of the invention.

Typically, the ionic solid is present in the film-forming solution in an amount of less than or equal to 50 mol %, less than or equal to 10 mol %, or less than or equal to 2.5 mol % with respect to the number of moles of the one or more M cations in the solution, preferably in an amount of from 0.01 to 5 mol %, or from 0.02 to 2.5 mol %, more preferably in an amount of from 0.05 to 2.0 mol %, or from 0.05 to 1.0 mol %, and even more preferably in an amount of from 0.1 to 1.5 mol %, or from 0.1 to 1.0 mol %, with respect to the number of moles of the one or more M cations in the solution. For instance, the ionic solid may be present in the film-forming solution in an amount of less than 1.0 mol % with respect to the number of moles of the one or more M cations in the solution, preferably wherein the ionic solid is present in an amount of from 0.1 mol % to 0.9 mol % with respect to the number of moles of the one or more M cations in the solution, more preferably from 0.1 mol % to 0.8 mol %, from 0.2 mol % to 0.8 mol %, from 0.2 mol % to 0.7 mol % or less than 0.5 mol %, or for instance from 0.1 mol % to 0.5 mol %, or from 0.2 mol % to 0.5 mol %, or from 0.3 mol % to 0.5 mol %.

Suitable solvents are known to the skilled person. For instance, the solvent may comprise one or more organic solvents, for instance one or more organic polar solvents, for instance one or more organic polar aprotic solvents. For instance, the solvent may comprise dimethyl sulfoxide (DMSO), dimehtylformamide (DMF), N-methyl-2-pyrrolidinone (NMP), γ-butyrolactone (GBL), N,N-dimethylacetamide (DMAC), 2-methoxyethanol (2ME), acetonitrile (ACN) or mixtures thereof. Often, the solvent comprises DMF and DMSO, typically in a volume ratio of from 2:1 to 6:1, e.g. 4:1, DMF:DMSO.

The process of the present invention may comprise a step of forming the film-forming solution by dissolving the ionic solid, at least one M precursor, at least one A precursor and optionally at least one X precursor in the solvent. Usually, the relative concentrations of the at least one M precursor and the at least one A precursor in the solvent correspond to the stoichiometries of the A and M ions in the desired crystalline A/M/X material. Often, the relative concentrations of the at least one M precursor, at least one A precursor and the at least one X precursor in the solvent correspond to the stoichiometries of the A, M and X ions in the desired crystalline A/M/X material. Often, the perovskite precursor concentration used is from 0.5 M to 2.5 M, for instance from 1 M to 2 M.

The ionic solid may be any ionic solid comprising an organic cation and counter-anion as described herein.

As is discussed in more detail below, an M precursor is a compound comprising one or more M cations present in [M] as described herein. Where [M] (that is, [M] in the compound of formula [A]ₐ[M]_{b}[X]_{c}) comprises only one type of M cation, only one M precursor is necessary in the process of the invention.

As is discussed in more detail below, an A precursor is a compound comprising one or more A cations present in [A]. Where [A] (that is, [A] in the compound of formula [A]ₐ[M]_{b}[X]_{c}) comprises only one type of A cation, only one A precursor is necessary in the process of the invention.

As regards the source of X anions in the process of the invention, it may not be necessary to provide a separate X precursor in the process of the invention. This is because in some embodiments, the A precursor (or where the process involves a plurality of A precursors, at least one of them) and/or the M precursor (or where the process involves a plurality of M precursors, at least one of them) is a salt comprising one or more X anions, for instance a halide salt. In a preferred embodiment, the A precursor (or where present the plurality of A precursors) and the M precursor (or where present the plurality of M precursors) together comprise each of the X cations present in [X]. For example, the precursors CsI, FAI, PbI₂, and PbBr₂ may be employed to produce the A/M/X material Cs_{0.17}FA_{0.83}Pb(I_{0.9}Br_{0.1})₃, where FA is formamidinium. Thus, the film-forming solution may comprise CsI, FAI, PbI₂, and PbBr₂, and the organic cation and counter-anion of the ionic solid.

The M precursor typically comprises one or more counter-anions. Thus, typically, the film-forming solution comprises one or more M precursor counter-anions. Many such counter-anions are known to the skilled person. The one or more M cations and the one or more M precursor counter anions may both be from a first precursor compound, which is dissolved in the solvent as described herein to form the film-forming solution.

The M precursor counter-anion in the film-forming solution may be a halide anion, a thiocyanate anion (SCN⁻), a tetrafluoroborate anion (BF₄⁻) or an organic anion. Preferably, the M precursor counter-anion as described herein is a halide anion or an organic anion. The film-forming solution may comprise two or more counter-anions, e.g. two or more halide anions.

Typically, the M precursor counter-anion is an anion of formula RCOO⁻, ROCOO⁻, RSO₃⁻, ROP(O)(OH)O⁻ or RO⁻, wherein R is H, substituted or unsubstituted C₁₋₁₀ alkyl, substituted or unsubstituted C₂₋₁₀ alkenyl, substituted or unsubstituted C₂₋₁₀ alkynyl, substituted or unsubstituted C₃₋₁₀ cycloalkyl, substituted or unsubstituted C₃₋₁₀ heterocyclyl or substituted or unsubstituted aryl. For instance R may be H, substituted or unsubstituted C₁₋₁₀ alkyl, substituted or unsubstituted C₃₋₁₀ cycloalkyl or substituted or unsubstituted aryl. Typically R is H substituted or unsubstituted C₁₋₆ alkyl or substituted or unsubstituted aryl. For instance, R may be H, unsubstituted C₁₋₆ alkyl or unsubstituted aryl. Thus, R may be selected from H, methyl, ethyl, propyl, butyl, pentyl, hexyl, cyclopentyl, cyclohexyl and phenyl.

Often, (one or more) counter-anions are selected from halide anions (e.g. F⁻, Cl⁻, Br⁻ and I⁻) and anions of formula RCOO⁻, wherein R is H or methyl.

Typically, the M precursor counter-anion is F⁻, Cl⁻, Br⁻, I⁻, formate or acetate. Preferably, the M precursor counter-anion is Cl⁻, Br⁻, I⁻ or F⁻. More preferably, the M precursor counter-anion is Cl⁻, Br⁻ or I⁻.

Typically, the M precursor is a compound of formula MY₂, MY₃, or MY₄, wherein M is a metal or metalloid cation as described herein, and Y is said counter-anion.

Thus, the M precursor may be a compound of formula MY₂, wherein M is Ca²⁺, Sr²⁺, Cd²⁺, Cu²⁺, Ni²⁺, Mn²⁺, Fe²⁺, Co²⁺, Pd²⁺, Ge²⁺, Sn²⁺, Pb²⁺, Yb²⁺ or Eu²⁺ and Y is F⁻, Cl⁻, Br⁻, I⁻, formate or acetate. Preferably M is Cu²⁺, Pb²⁺, Ge²⁺ or Sn²⁺ and Y is Cl⁻, Br⁻, I⁻, formate or acetate, preferably Cl⁻, Br⁻ or I⁻.

Typically, the M precursor is lead (II) acetate, lead (II) formate, lead (II) fluoride, lead (II) chloride, lead (II) bromide, lead (II) iodide, tin (II) acetate, tin (II) formate, tin (II) fluoride, tin (II) chloride, tin (II) bromide, tin (II) iodide, germanium (II) acetate, germanium (II) formate, germanium (II) fluoride, germanium (II) chloride, germanium (II) bromide or germanium (II) iodide. In some cases, the M precursor comprises lead (II) acetate. In some cases, the M precursor comprises lead (II) iodide.

The M precursor is typically a compound of formula MY₂. Preferably, the M precursor is a compound of formula SnI₂, SnBr₂, SnCl₂, PbI₂, PbBr₂ or PbCl₂.

The M precursor may be a compound of formula MY₃, wherein M is Bi³⁺ or Sb³⁺ and Y is F⁻, Cl⁻, Br⁻, I⁻, SCN⁻, BF₄⁻, formate or acetate. Preferably M is Bi³⁺ and Y is Cl⁻, Br⁻ or I⁻. In that case, the A/M/X material typically comprises a bismuth or antimony halogenometallate.

The M precursor may be a compound of formula MY₄, wherein M is Pd⁴⁺, W⁴⁺, Re⁴⁺, Os⁴⁺, Ir⁴⁺, Pt⁴⁺, Sn⁴⁺, Pb⁴⁺, Ge⁴⁺ or Te⁴⁺ and Y is F⁻, Cl⁻, Br⁻, I⁻, SCN⁻, BF₄⁻, formate or acetate. Preferably M is Sn⁴⁺, Pb⁴⁺ or Ge⁴⁺ and Cl⁻, Br⁻ or I⁻. In that case, the A/M/X material typically comprises a hexahalometallate.

Typically, the total concentration of [M] cations in the film-forming solution is between 0.01 and 5 M, for instance between 0.1 and 2.5 M, 0.25 and 2.0 M, preferably between 0.5 and 2.5 M, or for instance from 1.0 M to 2.0 M. The total concentration of [M] cations in the film-forming solution may for instance be from 0.5 M to 2.5 M. It may for instance be from 1 M to 2 M.

The A cations and X anions may both be from the same precursor compound or compounds, which are dissolved in the solvent as described herein to form the film-forming solution. Preferably, the A/X precursor compound is a compound of formula [A][X] wherein: [A] comprises the one or more A cations as described herein; and [X] comprises the one or more X anions as described herein. The A/X precursor compound is typically a compound of formula AX, wherein X is a halide anion and the A cation is as defined herein. When more than one A cation or more than one X anion is present in the compound of formula [A]ₐ[M]_{b}[X]_{c}, more than one compound of formula AX may be dissolved in the film-forming solution.

The A/X precursor compound (or compounds) may, for example, be selected from CH₃NH₃Cl, CH₃NH₃Br, CH₃NH₃I, CH₃CH₂NH₃Cl, CH₃CH₂NH₃Br, CH₃CH₂NH₃I, CH₃CH₂CH₂NH₃Cl, CH₃CH₂CH₂NH₃Br, CH₃CH₂CH₂NH₃I, N(CH₃)₄Cl, N(CH₃)₄Br, N(CH₃)₄I, (H₂N—C(H)═NH₂)Cl, (H₂N—C(H)═NH₂)Br, (H₂N—C(H)═NH₂)I, (H₂N—C(CH₃)═NH₂)Cl, (H₂N—C(CH₃)═NH₂)Br, (H₂N—C(CH₃)═NH₂)I, (H₂N—C(NH₂)═NH₂)Cl, (H₂N—C(NH₂)═NH₂)Br, (H₂N—C(NH₂)═NH₂)I, CsCl, CsBr, CsI, RbCl, RbBr and RbI.

Typically, the total concentration of [A] cations in the film-forming solution is between 0.01 and 5 M, for instance between 0.1 and 2.5 M, 0.25 and 2.0 M, preferably between 0.5 and 2.5 M, or for instance from 1.0 M to 2.0 M. The total concentration of [A] cations in the film-forming solution may for instance be from 0.5 M to 2.5 M. It may for instance be from 1 M to 2 M.

Typically, the total concentration of X anions depends on the total concentration of A and/or M cations. For instance, when an A/X precursor compound and/or an M precursor compound comprising one or more X anions are used, the total concentration of X anions will depend on the total amount of A/X precursor compound and/or an M precursor compound present, as described above.

Typically, the film-forming solution is disposed on the substrate by solution phase deposition, for instance gravure coating, slot dye coating, screen printing, ink jet printing, doctor blade coating, spray coating, roll-to-roll (R2R) processing, or spin-coating. Typically, disposing the film-forming composition on the substrate comprises a step of spin-coating the film-forming solution on the substrate.

Typically, the spin coating is performed at a speed of at least 1000 RPM, for instance at least 2000 RPM, at least 3000 RPM or at least 4000 RPM, for example between 1000 and 10000 RPM, between 2000 and 8000 RPM, between 2500 and 7500 RPM, preferably about 5000 RPM. Typically, the spin coating is performed for a time of at least one second, at least 5 seconds or at least 10 seconds, for example from 1 second to 1 minute, from 10 seconds to 50 seconds, preferably about 20 to 40 seconds.

The process may further comprise using an anti-solvent to facilitate precipitation of the crystalline A/M/X material. Typically, the antisolvent is dropped onto the film-forming solution either during disposing the film-forming solution on the substrate or after the film-forming solution has been disposed on the substrate. For instance, the antisolvent may be dropped onto the film-forming solution during the spin-coating. Typically, the antisolvent is selected from toluene, chlorobenzene, chloroform, dichlorobenzene, isopropyl alcohol, tetrahydrofuran, benzene, xylene, anisole and mixtures thereof.

Typically, the process further comprises removing the solvent, and optionally the anti-solvent, to form the layer comprising the crystalline A/M/X material. Removing the solvent (and optionally the anti-solvent) may comprise heating the solvent, or allowing the solvent to evaporate.

The solvent (and optionally the anti-solvent) is usually removed by heating (annealing) the film-forming solution treated substrate. For instance, the film-forming solution treated substrate may be heated to a temperature of from 30° C. to 400° C., for instance from 50° C. to 200° C. Preferably, the film-forming solution treated is heated to a temperature of from 50° C. to 200° C. for a time of from 5 to 200 minutes, preferably from 10 to 100 minutes.

Process 2—Two-Step AMX Deposition, with Ionic Solid

The present invention also provides a second process for producing an ionic solid-modified film of a crystalline A/M/X material, which crystalline A/M/X material comprises a compound of formula: [A]ₐ[M]_{b}[X]_{c}, wherein: [A] comprises one or more A cations; [M] comprises one or more M cations which are metal or metalloid cations; [X] comprises one or more X anions; wherein a is a number from 1 to 6; b is a number from 1 to 6; and c is a number from 1 to 18,

the process comprising:
a) disposing a first solution on a substrate wherein the first solution comprises a solvent and one or more M cations, and optionally removing the solvent, to produce a treated substrate;
b) contacting the treated substrate with a second solution comprising a solvent and one or more A cations or with vapour comprising one or more A cations,

wherein: one or more X anions are present in one or both of: (i) the first solution employed in step (a), and (ii) the second solution or vapour employed in step (b); and the first solution employed in step (a) further comprises an organic cation and a counter-anion of an ionic solid or step (b) further comprises contacting the treated substrate with an ionic solid, wherein the ionic solid is a salt which comprises an organic cation and a counter-anion. Optionally, the ionic solid is other than a quaternary ammonium halide salt. The ionic solid is usually other than a primary ammonium halide salt. The ionic solid is often other than a secondary ammonium halide salt. The ionic solid is usually other than a tertiary ammonium halide salt. Thus, typically, the ionic solid is other than a primary, secondary, tertiary or quaternary ammonium halide salt, i.e. the counter-anion is other than halide and the organic cation is other than a primary, secondary, tertiary or quaternary ammonium cation. The ionic solid is typically other than a formamidinium halide salt, and usually other than a guanidinium halide salt, i.e. the counter-anion is other than halide and the organic cation is other than formamidinium or guanidinium. Thus, typically, the ionic solid is other than a primary, secondary, tertiary or quaternary ammonium halide salt and other than a formamidinium or guanidinium halide salt. The ionic solid is typically other than a halide salt of a cation of formula (X) as defined herein. Often, the ionic solid is other than a primary, secondary, tertiary or quaternary ammonium halide salt and other than a halide salt of a cation of formula (X) as defined herein. Typically, when the counter-anion of the ionic solid is halide, the organic cation of the ionic solid is other than each of the one or more A cations of the crystalline A/M/X material. Often, the organic cation of the ionic solid is other than each of the one or more A cations of the crystalline A/M/X material.

In the second process of the invention, the crystalline A/M/X material, the one or more A cations, the one or more M cations, the one or more X anions, the ionic solid, the organic cation and the counter-anion may be as further defined anywhere herein; for instance, they may be as further defined anywhere hereinbefore for the optoelectronic device of the invention.

The first solution employed in step (a) may further comprise the organic cation and the counter-anion of the ionic solid. For instance, the first solution may comprise a solvent, one or more M cations, optionally one or more X anions and the organic cation and the counter-anion of the ionic solid.

Alternatively, step (b) may further comprise contacting the treated substrate with the ionic solid. For instance, step (b) may comprise contacting the treated substrate with a second solution wherein the second solution further comprises the organic cation and the counter-anion of the ionic solid. The second solution may therefore comprise a solvent, one or more A cations, optionally one or more X anions and the organic cation and the counter-anion of the ionic solid.

In one embodiment, the process comprises:
a) disposing a first solution on a substrate wherein the first solution comprises a solvent, one or more M cations, one or more X anions and the organic cation and the counter-anion of the ionic solid, and optionally removing the solvent, to produce a treated substrate;
b) contacting the treated substrate with a second solution comprising a solvent, one or more A cations and one or more X anions.

In another embodiment, the process comprises:
a) disposing a first solution on a substrate wherein the first solution comprises a solvent, one or more M cations and one or more X anions, and optionally removing the solvent, to produce a treated substrate;
b) contacting the treated substrate with a second solution comprising a solvent, one or more A cations, one or more X anions and the organic cation and the counter-anion of the ionic solid.

The solvent in steps (a) and (b) may be any solvent as described above for the first process of the invention.

The process may further comprise a step of forming the first solution by dissolving at least one M precursor as described herein, optionally one or more X precursors as described herein and optionally the ionic solid in a solvent.

When step (b) comprises contacting the treating substrate with a second solution comprising a solvent and one or more A cations, the process may further comprise a step of forming the second solution by dissolving at least one A precursor as described herein, optionally one or more X precursors as described herein and optionally the ionic solid in a solvent.

As regards the source of X anions in the process of the invention, it may not be necessary to provide a separate X precursor in the process of the invention. This is because in some embodiments, the A precursor (or where the process involves a plurality of A precursors, at least one of them) and/or the M precursor (or where the process involves a plurality of M precursors, at least one of them) is a salt comprising one or more X anions, for instance a halide salt. In a preferred embodiment, the A precursor (or where present the plurality of A precursors) and the M precursor (or where present the plurality of M precursors) together comprise each of the X cations present in [X].

The first and second solutions may be disposed on the substrate by any of the methods described herein. Typically, the first and second solutions are disposed on the substrate by solution phase deposition, for instance gravure coating, slot dye coating, screen printing, ink jet printing, doctor blade coating, spray coating, roll-to-roll (R2R) processing, or spin-coating. Typically, the process comprises a step of disposing the first solution on the substrate by spin-coating and disposing the second solution on the substrate by spin-coating. An anti-solvent may be used as described above when disposing either or both of the first and second solutions on the substrate.

Typically, the spin coating is performed at a speed of at least 1000 RPM, for instance at least 2000 RPM, at least 3000 RPM or at least 4000 RPM, for example between 1000 and 10000 RPM, between 2000 and 8000 RPM, between 2500 and 7500 RPM, preferably about 5000 RPM. Typically, the spin coating is performed for a time of at least one second, at least 5 seconds or at least 10 seconds, for example from 1 second to 1 minute, from 10 seconds to 50 seconds, preferably about 20 to 40 seconds.

Step (b) may comprise contacting the treated substrate with vapour comprising one or more A cations. For instance, step (b) may comprise contacting the treated substrate with said vapour comprising one or more A cations and with vapour comprising the organic cation and the counter-anion of the ionic solid.

Thus, the process may comprise:
a) disposing a first solution on a substrate wherein the first solution comprises a solvent, one or more M cations, one or more X anions and the organic cation and the counter-anion of the ionic solid, and optionally removing the solvent, to produce a treated substrate;
b) contacting the treated substrate with vapour comprising one or more A cations and one or more X anions.

Alternatively, the process may comprise:
a) disposing a first solution on a substrate wherein the first solution comprises a solvent, one or more M cations and one or more X anions, and optionally removing the solvent, to produce a treated substrate;
b) contacting the treated substrate with vapour comprising one or more A cations, one or more X anions and the organic cation and the counter-anion of the ionic solid.

Typically, step (b) comprises:
b1) vapourising a composition, or compositions, which comprise the one or more A cations and the ionic solid, and
b2) depositing the resulting vapour on the treated substrate.

For instance, step (b1) may comprise vapourising a composition, or compositions, which comprise the one or more A cations, one or more X anions and the ionic solid. Said composition or compositions may comprise, consist essentially of or consist of the A cation precursor, optionally one or more X anion precursors and the ionic solid. Hence, the process may comprise a step of preparing a composition or compositions by mixing one or more A cation precursors, the ionic solid and optionally one or more X anion precursors.

Removing the solvent may comprise heating the solvent, or allowing the solvent to evaporate. Thus, typically, the process comprises annealing the substrate.

The solvent is usually removed by heating (annealing) the first solution-treated substrate. For instance, the film-forming solution treated substrate may be heated to a temperature of from 30° C. to 400° C., for instance from 50° C. to 200° C. Preferably, the film-forming solution treated is heated to a temperature of from 50° C. to 200° C. for a time of from 5 to 200 minutes, preferably from 10 to 100 minutes.

An additional step of removing the solvent may also be performed after step b) as described above when the treated substrate is contacted with a second solution comprising a solvent, one or more A cations, optionally one or more X anions and optionally the organic cation and the counter-anion of the ionic solid.

Process 3—all Vapour Deposition: AMX and Ionic Solid

Ionic solids may be vaporised by sublimation, meaning that employing an ionic solid facilitates the use of a vapour deposition process for producing an ionic solid modified film of a crystalline A/M/X material. Both the A/M/X material and the ionic solid may be deposited by vapour deposition, to produce an ionic solid modified film of a crystalline A/M/X material.

Accordingly, the present invention also provides a third process for producing an ionic solid-modified film of a crystalline A/M/X material, wherein the crystalline A/M/X material comprises a compound of formula: [A]ₐ[M]_{b}[X]_{c}, wherein: [A] comprises one or more A cations; [M] comprises one or more M cations which are metal or metalloid cations; [X] comprises one or more X anions; a is a number from 1 to 6; b is a number from 1 to 6; and c is a number from 1 to 18, and wherein the ionic solid is a salt comprising an organic cation and a counter anion; which process comprises:

exposing a substrate to vapour comprising the one or more A cations, vapour comprising the one or more M cations, vapour comprising the one or more X anions, vapour comprising the organic cation, and vapour comprising the counter anion. Typically, the ionic solid is other than a quaternary ammonium halide salt, i.e. the organic cation is other than a quaternary ammonium cation and the counter anion is other than a halide. The ionic solid is usually other than a primary ammonium halide salt. The ionic solid is often other than a secondary ammonium halide salt. The ionic solid is usually other than a tertiary ammonium halide salt. Thus, typically, the ionic solid is other than a primary, secondary, tertiary or quaternary ammonium halide salt, i.e. the counter-anion is other than halide and the organic cation is other than a primary, secondary, tertiary or quaternary ammonium cation. The ionic solid is typically other than a formamidinium halide salt, and usually other than a guanidinium halide salt, i.e. the counter-anion is other than halide and the organic cation is other than formamidinium or guanidinium. Thus, typically, the ionic solid is other than a primary, secondary, tertiary or quaternary ammonium halide salt and other than a formamidinium or guanidinium halide salt. The ionic solid is typically other than a halide salt of a cation of formula (X) as defined herein. Often, the ionic solid is other than a primary, secondary, tertiary or quaternary ammonium halide salt and other than a halide salt of a cation of formula (X) as defined herein. Typically, when the counter-anion of the ionic solid is halide, the organic cation of the ionic solid is other than each of the one or more A cations of the crystalline A/M/X material. Often, the organic cation of the ionic solid is other than each of the one or more A cations of the crystalline A/M/X material.

In the third process of the invention, the crystalline A/M/X material, the one or more A cations, the one or more M cations, the one or more X anions, the ionic solid, the organic cation and the counter-anion may be as further defined anywhere herein; for instance, they may be as further defined anywhere hereinbefore for the optoelectronic device of the invention.

The vapour comprising the one or more A cations, vapour comprising the one or more M cations, vapour comprising the one or more X anions, vapour comprising the organic cation, and vapour comprising the counter anion, may be one and the same vapour. Thus, the process of this aspect of the invention may comprise exposing the substrate to vapour which comprises the one or more A cations, the one or more M cations, the one or more X anions, the organic cation, and the counter anion.

Alternatively, the one or more A cations, the one or more M cations, the one or more X anions, the organic cation, and the counter anion, may be part of two or more different vapour phases, to which the substrate is exposed. The substrate may be exposed to the two or more different vapour phases at the same time or at different times, e.g. separately and/or sequentially. Accordingly, the process of this aspect of the invention may comprise exposing the substrate to two or more different vapour phases, wherein the two or more different vapour phases together comprise the one or more A cations, the one or more M cations, the one or more X anions, the organic cation, and the counter anion. The substrate may be exposed to the two or more different vapour phases simultaneously (at the same time), separately (at different times), for instance sequentially (in any order).

For instance, the substrate may be exposed to: (i) vapour comprising the one or more A cations, the one or more M cations, the one or more X anions; and (ii) vapour comprising the organic cation and the counter anion of the ionic solid. Alternatively, the substrate may be exposed to (i) vapour comprising the one or more M cations, (ii) vapour comprising the one or more A cations (wherein the one or more X anions may be present in the vapour comprising the one or more M cations, the vapour comprising the one or more A cations, or in both the vapour comprising the one or more M cations and the vapour comprising the one or more A cations), and (iii) vapour comprising the organic cation and the counter anion of the ionic solid. The substrate may be exposed to these vapour phases at the same time or at different times, e.g. sequentially in any order.

In the first, second and third processes described above, the substrate may comprise a first charge-transporting material, which may be a charge-transporting material as defined anywhere herein, particularly as defined hereinbefore for the optoelectronic device of the invention. Typically, the first charge-transporting material is disposed on a first electrode. The first electrode may be as defined anywhere hereinbefore for the optoelectronic device of the invention.

Thus, the substrate may comprise the following layers in the following order:
First electrode (typically an anode; typically a transparent electrode, which typically comprises a transparent conducting oxide, optionally itself disposed on glass);
Layer of a charge transporting material (typically a p-type material as described hereinbefore, e.g. in relation to the p-i-n optoelectronic devices, but it may alternatively be an n-type material as described hereinbefore, e.g. in relation to the n-i-p optoelectronic devices).

Often, the first charge-transporting material comprises a hole-transporting (p-type) material as described herein. Typically, the first electrode is a transparent electrode, for instance an electrode comprising a transparent conducing oxide as described herein.

Process 4—Treatment of AMX Film with Ionic Solid

The invention further relates to a process for producing an ionic solid-modified film of a crystalline A/M/X material, wherein the crystalline A/M/X material comprises a compound of formula: [A]ₐ[M]_{b}[X]_{c} wherein: [A] comprises one or more A cations; [M] comprises one or more M cations which are metal or metalloid cations; [X] comprises one or more X anions; a is a number from 1 to 6; b is a number from 1 to 6; and e is a number from 1 to 18; and wherein the ionic solid is a salt which comprises an organic cation and a counter-anion; which process comprises treating a film of the crystalline A/M/X material with the organic cation and the counter-anion of the ionic solid. Typically, the ionic solid is other than a quaternary ammonium halide salt. The ionic solid is usually other than a primary ammonium halide salt. The ionic solid is often other than a secondary ammonium halide salt. The ionic solid is usually other than a tertiary ammonium halide salt. Thus, typically, the ionic solid is other than a primary, secondary, tertiary or quaternary ammonium halide salt, i.e. the counter-anion is other than halide and the organic cation is other than a primary, secondary, tertiary or quaternary ammonium cation. The ionic solid is typically other than a formamidinium halide salt, and usually other than a guanidinium halide salt, i.e. the counter-anion is other than halide and the organic cation is other than formamidinium or guanidinium. Thus, typically, the ionic solid is other than a primary, secondary, tertiary or quaternary ammonium halide salt and other than a formamidinium or guanidinium halide salt. The ionic solid is typically other than a halide salt of a cation of formula (X) as defined herein. Often, the ionic solid is other than a primary, secondary, tertiary or quaternary ammonium halide salt and other than a halide salt of a cation of formula (X) as defined herein. Typically, when the counter-anion of the ionic solid is halide, the organic cation of the ionic solid is other than each of the one or more A cations of the crystalline A/M/X material. Often, the organic cation of the ionic solid is other than each of the one or more A cations of the crystalline A/M/X material.

In the fourth process of the invention, the crystalline A/M/X material, the one or more A cations, the one or more M cations, the one or more X anions, the ionic solid, the organic cation and the counter-anion may be as further defined anywhere herein; for instance, they may be as further defined anywhere hereinbefore for the optoelectronic device of the invention.

The step of treating the film of the crystalline A/M/X material with the ionic solid may comprise disposing the ionic solid on the film of the crystalline A/M/X material using any technique known to the skilled person or any technique as described herein. For instance, the ionic solid may be disposed on the film of the crystalline A/M/X material by vapour deposition or solution deposition, for instance by gravure coating, slot dye coating, screen printing, ink jet printing, doctor blade coating, spray coating, roll-to-roll (R2R) processing, or spin-coating. Typically the ionic solid is disposed on the film of the crystalline A/M/X material by spin coating.

Accordingly, the step of treating the film of the crystalline A/M/X material may comprise treating the film with a solution comprising the organic cation and the counter anion. The step of treating the film with the solution may comprise gravure coating, slot dye coating, screen printing, ink jet printing, doctor blade coating, spray coating, roll-to-roll (R2R) processing, or spin-coating.

Alternatively, the step of treating the film of the crystalline A/M/X material may comprise exposing the film to vapour comprising the organic cation of the ionic solid and vapour comprising the counter anion of the ionic solid. The vapour comprising the organic cation and the vapour comprising the counter anion are typically one and the same vapour, but they may alternatively be different vapours.

The ionic solid may be any ionic solid as described herein, i.e. may be an ionic solid comprising any organic cation and counter-anion as described herein. The crystalline A/M/X material may be any crystalline A/M/X material as described herein.

Typically, the film of the crystalline A/M/X material is disposed on a substrate. The substrate may be any substrate as described herein. For example it may comprise a layer comprising a charge-transporting material as defined herein and a first electrode as defined herein, wherein the crystalline A/M/X material is disposed on the layer comprising the charge-transporting material and the layer comprising the charge-transporting material is disposed on the first electrode.

The fourth process may further comprise a step of depositing the crystalline A/M/X material on a substrate. For instance, the crystalline A/M/X material may be deposited by vapour deposition, or by any of the solution-based techniques as described herein. In one embodiment, the process comprises depositing the crystalline A/M/X material by vapour deposition, then depositing the ionic solid on the film of the crystalline A/M/X material by vapour deposition or solution deposition, as described herein. In another embodiment, the process comprises depositing the crystalline A/M/X material by any of the solution-based techniques as described herein, then depositing the ionic solid on the film of the crystalline A/M/X material by vapour deposition or solution deposition, as described herein.

Process for Producing an Optoelectronic Device

The present invention also relates process for producing an optoelectronic device, which process comprises producing, on a substrate, an ionic solid-modified film of a crystalline A/M/X material, by any process as described herein, for instance by any of the first, second, third and fourth processes defined hereinbefore. The ionic solid may be any ionic solid as described herein, i.e. may be an ionic solid comprising any organic cation and counter-anion as described herein. The crystalline A/M/X material may be any crystalline A/M/X material as described herein. The substrate may be any substrate as described herein.

Typically, the substrate comprises a first charge-transporting material disposed on a first electrode which is a transparent electrode. Typically, the first electrode (which is typically an anode) comprises a transparent conducting oxide, for instance fluorine doped tin oxide (FTO), aluminium doped zinc oxide (AZO) or indium doped tin oxide (ITO). Typically, the first charge-transporting material is a hole transporting (p-type) material as described herein, although it may alternatively be an electron-transporting (n-type) material as described herein. Preferably, the first charge-transporting material comprises polyTPD or nickel oxide, for instance the first charge-transporting material comprise polyTPD or may be a compact layer of nickel oxide.

Typically, the first charge-transporting material comprises polyTPD; it is typically p-doped polyTPD; thus, the first charge-transporting material typically comprises polyTPD:F4-TCNQ. To improve wettability, NPD was typically deposited after polyTPD. Thus, the first charge-transporting material often comprises polyTPD and NPD.

The process may comprise a step of forming the substrate by disposing the first charge-transporting material on the first electrode. Typically, the first charge-transporting material is disposed on the first electrode by spin coating a solution comprising a solvent and a first charge-transporting material or first-charge transporting material precursor onto the first electrode. The process of forming the substrate may comprise a step of removing the solvent using any method as described herein, to produce a treated substrate. Typically, the solvent is removed by heating the solution-treated first electrode. For instance, the solution treated first electrode may be heated to a temperature of from 30° C. to 400° C., for instance from 50° C. to 200° C. Preferably, the solution treated first electrode is heated to a temperature of from 50° C. to 200° C. for a time of from 2 to 200 minutes, preferably from 5 to 30 minutes.

Typically, the first charge transporting material comprises polyTPD, therefore the first charge-transporting material is disposed on the first electrode by disposing (e.g. by spin-coating) a solution comprising polyTPD onto the first electrode. This is typically followed by annealing by heating to a temperature of from 50° C. to 200° C. for a time of from 2 to 200 minutes, preferably from 5 to 30 minutes.

The first charge transporting material often comprises both polyTPD and NPD. The first charge-transporting material may be disposed on the first electrode by firstly disposing (e.g. spin-coating) a solution comprising polyTPD onto the first electrode to form a first sub-layer comprising polyTPD, and secondly disposing (e.g. spin-coating) a solution comprising NPD onto the first sub-layer to form a second sub-layer comprising NPD. The polyTPD may be p-doped, for instance it may be polyTPD:F4-TCNQ. Each one of the first and the second disposing (e.g. spin-coating) steps is typically followed by an annealing step, comprising heating to a temperature of from 50° C. to 200° C. for a time of from 2 to 200 minutes, preferably from 5 to 30 minutes.

If, on the other hand, the first charge transporting material comprises nickel oxide, the first charge-transporting material may be disposed on the first electrode by disposing (e.g. spin-coating) a solution comprising nickel oxide onto the first electrode. The substrate may optionally be sintered. Sintering typically involves a step of heating the substrate to an elevated temperature, for instance a temperature of at least 100° C., at least 200° C., at least 300° C. or at least 400° C. for a period of from 10 to 100 minutes, typically from 20 to 60 minutes. Alternatively a nickel oxide layer can be deposited via vacuum deposition techniques such as sputter coating.

The step of forming the substrate may further comprise disposing a second ionic compound as defined herein on the first charge transporting material. This may comprise treating the first charge transporting material with the organic cation and the counter-anion of the second ionic compound. The step of treating the first charge transporting material with the organic cation and the counter-anion of the second ionic compound may comprise disposing the second ionic compound on the first charge transporting material using any technique known to the skilled person or any technique as described herein. For instance, the second ionic compound may be disposed on the first charge transporting material by vapour deposition or solution deposition, for instance by gravure coating, slot dye coating, screen printing, ink jet printing, doctor blade coating, spray coating, roll-to-roll (R2R) processing, or spin-coating. This may be followed by annealing, for instance at 50° C. to 150° C., typically 100° C., typically for under 10 minutes, for instance 5 minutes. Typically the second ionic compound is disposed on the first charge transporting material by spin coating. This is typically followed by annealing as mentioned above.

Typically, the layer comprising a crystalline A/M/X material is disposed directly on the layer of the first charge-transporting material (or, if present, on the second ionic compound which is itself disposed on the layer of the first charge-transporting material). The layer of the first charge-transporting material preferably comprises polyTPD, or polyTPD and NPD (e.g. in two sub-layers comprising the polyTPD and the NPD respectively, preferably wherein the sub-layer comprising polyTPD is adjacent the first electrode and preferably wherein the sub-layer comprising NPD is adjacent the layer comprising a crystalline A/M/X material), or is a compact layer of nickel oxide. The polyTPD may be p-doped, for instance it may be polyTPD:F4-TCNQ.

The process may further comprise: disposing a second charge-transporting material on the ionic solid-modified film of a crystalline A/M/X material, and disposing a second electrode on the second charge-transporting material. Disposing a second charge-transporting material may be as described above for disposing the first charge-transporting material. Disposing the second charge-transporting material typically comprises disposing a solution comprising a solvent and the second charge-transporting material on the ionic solid-modified film of a crystalline A/M/X material. Solution disposition may comprise gravure coating, slot dye coating, screen printing, ink jet printing, doctor blade coating, spray coating, roll-to-roll (R2R) processing, or spin-coating. This is typically followed by a step of removing the solvent using any method as described herein. Typically, the solvent is removed by heating, for instance, to a temperature of from 30° C. to 400° C., for instance from 50° C. to 200° C. Typically, the heating comprises heating to a temperature of from 50° C. to 200° C. for a time of from 1 to 200 minutes, preferably from 1 to 30 minutes, for instance from 2 to 8 minutes. Where there are two layers (or two sub-layers) comprising different second charge-transporting materials, each layer (or sub-layer) may be disposed by disposing a solution comprising a solvent and the particular charge-transporting material, as described above. Optionally, each solution disposition step is followed by a heating step as described above. For instance, a layer (or sub-layer) of PCBM and a layer (or sub-layer) of BCP may be disposed consecutively in this way.

Before a second charge-transporting material is disposed on the ionic solid-modified film of a crystalline A/M/X material, the process may further comprise disposing a second ionic compound as defined herein on the ionic solid-modified film of a crystalline A/M/X material. This may comprise treating the ionic solid-modified film of a crystalline A/M/X material with the organic cation and the counter-anion of the second ionic compound. The step of treating the ionic solid-modified film of a crystalline A/M/X material with the organic cation and the counter-anion of the second ionic compound may comprise disposing the second ionic compound on the ionic solid-modified film of a crystalline A/M/X material using any technique known to the skilled person or any technique as described herein. For instance, the second ionic compound may be disposed on the ionic solid-modified film of a crystalline A/M/X material by vapour deposition or solution deposition, for instance by gravure coating, slot dye coating, screen printing, ink jet printing, doctor blade coating, spray coating, roll-to-roll (R2R) processing, or spin-coating. This may be followed by annealing, for instance at 50° C. to 150° C., typically 100° C., typically for under 10 minutes, for instance 5 minutes. Typically the second ionic compound is disposed on the ionic solid-modified film of a crystalline A/M/X material by spin coating. This is typically followed by annealing as mentioned above. The process may then further comprise: disposing a second charge-transporting material on the second ionic compound, and disposing a second electrode on the second charge-transporting material.

Typically the first charge transporting material is a hole-transporting (p-type) material as described herein and the second charge transporting material is an electron-transporting (n-type) material as described herein. Alternatively, the first charge transporting material may be an electron-transporting (n-type) material as described herein and the second charge transporting material is a hole-transporting (p-type) material as described herein. For instance the first charge-transporting material may comprise polyTPD, or polyTPD and NPD (e.g. in two sub-layers comprising the polyTPD and the NPD respectively), or nickel oxide, and the second charge transporting material may be an organic electron-transporting (n-type) material, preferably PCBM.

Typically, the first electrode comprises a transparent conducting oxide and the second electrode comprises an elemental metal.

Typically, the second electrode comprises, or consists essentially of, a metal for instance an elemental metal. Examples of metals which the second electrode material may comprise, or consist essentially of, include chromium, silver, gold, copper, aluminium, platinum, palladium, or tungsten. The second electrode may be disposed by vacuum evaporation. The thickness of the layer of a second electrode material is typically from 1 to 250 nm, preferably from 5 nm to 100 nm.

The optoelectronic device produced by the process may comprise any additional layers, as described herein, for instance additional electron-transporting (n-type) layers or interface modifying layers or buffer layers, e.g. a layer of BCP. Similarly, the process may further comprise adding any such additional layers.

The present invention also relates to an ionic solid-modified film of a crystalline A/M/X material which is obtainable by any process as described herein. The present invention also relates to an ionic solid-modified film of a crystalline A/M/X material which is obtained by any process as described herein.

The present invention also relates to optoelectronic device which
(a) comprises an ionic solid-modified film of a crystalline A/M/X material as described herein; or
(b) is obtainable by a process as described herein.

The advantages of the invention will hereafter be described with reference to some specific examples.

### Example 1

By introducing ionic solids into the perovskite absorber layer, ion migration may be cohesively suppressed and film stability improved under combined light and heat in ambient air. An ionic-solid-containing perovskite preferably in a p-i-n or n-i-p planar device structure, may deliver not just an improvement in efficiency, but also almost “non-degrading” solar cells when stressed under full spectrum sunlight at elevated temperature. This represents a key step towards the commercial upscale and deployment of the perovskite PV technology.

Methods

Materials. Lead iodide (PbI₂, 99.999%, metals basis) was purchased from Alfa-Aesar and lead bromide (PbBr₂, ≥98%) from Alfa-Aesar. Cesium iodide (CsI, 99.99%) was purchased from Alfa-Aesar. Formamidinium iodide (FAI) was purchased from GreatCell Solar. [6,6]-phenyl-C₆₁-butyric acid methyl ester (PCBM, >99.5%) was purchased from Solenne BV. Poly(4-butylphenyl-diphenyl-amine) (polyTPD) was purchased from 1-Material. 2,3,5,6-Tetrafluoro-7,7,8,8-tetracyanoquinodimethane (F4-TCNQ) was purchased from Lumtec. N,N′-Di(1-naphthyl)-N,N′-diphenyl-(1,1′-biphenyl)-4,4′-diamine (NPD) was purchased from Sigma-Aldrich. Bathocuproine (BCP, 98%) was purchased from Alfa Aesar. Unless stated otherwise, all other materials and solvents were purchased from Sigma-Aldrich. All the materials were used as received without further purification.

Preparation of metal-halide perovskites and organic transporting semiconductors. To form the mixed-cation lead mixed-anion perovskite precursor solutions, CsI, FAI, PbI₂, and PbBr₂ were prepared in the way corresponding to the exact stoichiometry for the desired metal-halide perovskite composition [e.g. Cs_{0.17}FA_{0.83}Pb(I_{0.9}Br_{0.1})₃] in a mixed organic solvent system comprising anhydrous N,N-dimethylformamide (DMF) and anhydrous dimethyl sulfoxide (DMSO) at the ratio of DMF:DMSO=4:1. The perovskite precursor concentration used was 1.45 M. In parallel, the ionic solids containing perovskite precursor solutions were prepared by dissolving the same perovskite components as the non-ionic-solid containing perovskite precursor solutions in the DMF/DMSO mixed solvent system with different ionic solids in the desired molar ratios with respect to the Pb content. The perovskite precursor solutions were stirred overnight in a nitrogen-filled glovebox and used without any further treatment. For the hole transporting material, polyTPD was dissolved in toluene in a concentration of 1 mg/mL along with 20 wt % of F4-TCNQ. NPD, a type of hole transporting material, was dissolved in m-Xylene at a concentration of 3 mg/mL. For the electron transporting (PCBM) and hole blocking (BCP) materials, PCBM and BCP were dissolved in a mixed organic solvent system of chlorobenzene (CB) and 1,2-dichlorobenzene (DCB) (CB:DCB=3:1 in volume) and isopropanol (IPA) at a concentration of 20 and 0.5 mg/mL, respectively. For perovskite bottom surface and top surface treatment, ionic solids were dissolved in the mixed solvent containing DMF and DMSO in the ratio of DMF:DMSO=4:1 and IPA, respectively.

Substrates preparation and device fabrication. Fluorine-doped tin oxide (FTO) coated glass (Pilkington TEC 7, 7Ω/□ sheet resistivity) was etched with zinc powder and 2 M HCl to obtain desired transparent electrode patterns. Other than FTO-coated glass substrates, pre-patterned tin-doped indium oxide (ITO) glass substrates were also used for the fabrication of perovskite solar cells. The substrates were cleaned in a series of ultrasonic cleaning baths using various solutions and solvents in the following sequence: 1) deionized water with 2% v/v solution of Decon 90 cleaning detergent; 2) deionized water; 3) acetone and 4) IPA (each step for 5˜8 mins). After ultrasonic cleaning, substrates were dried with dry nitrogen and then treated with UV-Ozone for 15˜20 mins before use. After the substrate cleaning procedure, F4-TCNQ doped polyTPD was deposited by dispensing the as-prepared organic solution onto a spinning substrate at 2000 rpm for 20 sec, followed by thermal annealing at 130° C. for 10 min in ambient air. To improve the wettability when processing perovskites on ITO substrates, NPD was deposited after polyTPD using the same processing protocol as polyTPD. The deposition of the perovskite layers was carried out using a spin coater in a nitrogen-filled glove box with the following processing parameters: starting at 1000 rpm for 5 sec (ramping time of 5 sec from stationary status) and then 5000 rpm (ramping time of 5 sec from 1000 rpm) for 30 sec. Before the end of the spinning process, a solvent-quenching method [Jeon, N. J. et al. Solvent engineering for high-performance inorganic-organic hybrid perovskite solar cells. Nat. Mater. 13, 897, (2014)] was used by dropping 300-μL anisole onto the spinning substrates at 40 sec after the start of the spin-coating process. The thermal annealing process (100° C. for 50˜60 min) was then carried out for the formation of the perovskite layer. The as-prepared PCBM solution was dynamically spun onto the perovskite layers at a speed of 2000 rpm for 20 sec. The samples were then annealed at 100° C. for 3˜5 min. After cooling down to room temperature, the as-prepared BCP solution was dynamically spun onto the PCBM layer at a speed of 4000 rpm for 20 see, followed by a brief thermal annealing process at 100° C. for ˜1 min. Both PCBM and BCP were processed inside the nitrogen-filled glovebox. For perovskite bottom surface and top surface treatment, ionic solid solutions were dynamically spun at 6000 rpm onto the hole transporting layer (i.e. p-type semiconductors) and the perovskite layer, respectively, followed by a thermal-annealing process at 100° C. for 5 min. The ionic-solid surface treatments were carried out in the glovebox. The perovskite solar cells were completed by thermal evaporation of Cr₂O₃/Cr (3.5 nm) and Au electrodes (100 nm) through shadow masks under high vacuum (6×10⁻⁶ torr) using a thermal evaporator (Nano 36, Kurt J. Lesker) placed in ambient environment.

Solar cell characterisation. The current density and voltage (J-V) characteristics for perovskite solar cells were measured in air with a Keithley 2400 source meter under AM1.5 sunlight at 100 mW/cm² irradiance generated using an ABET Sun 2000 Class A simulator. The J-V curves were recorded at a scan rate of 200 mV/s (with a voltage step of 10 mV and delay time of 10 ms). The light intensity was calibrated using a National Renewable Energy Laboratories (NREL) calibrated KG 5 filtered silicon reference cell with the mismatch factor less than 1%. All devices were masked with metal aperture to define the active area and to eliminate edge effects.

Device stability test. The perovskite solar cells were encapsulated with a cover glass (LT-Cover, Lumtec) and UV adhesive (LT-U001, Lumtec) in a nitrogen-filled glovebox. All the devices were aged using an Atlas SUNTEST XLS+(1,700 W air-cooled Xenon lamp) light-soaking chamber under simulated full-spectrum AM1.5 sunlight with 76 mW/cm² irradiance. All devices were aged under open-circuit conditions. The J-V characteristics for the ageing cells were recorded at different time intervals under a separate solar simulator (AM1.5, 100 mW/cm²). No additional ultraviolet filter was used during the aging process. The chamber was air-cooled with the temperature controlled at 85° C. as measured by a black standard temperature control unit. The relative humidity in the laboratory was monitored in the range of 50-60% during the aging period.

Results

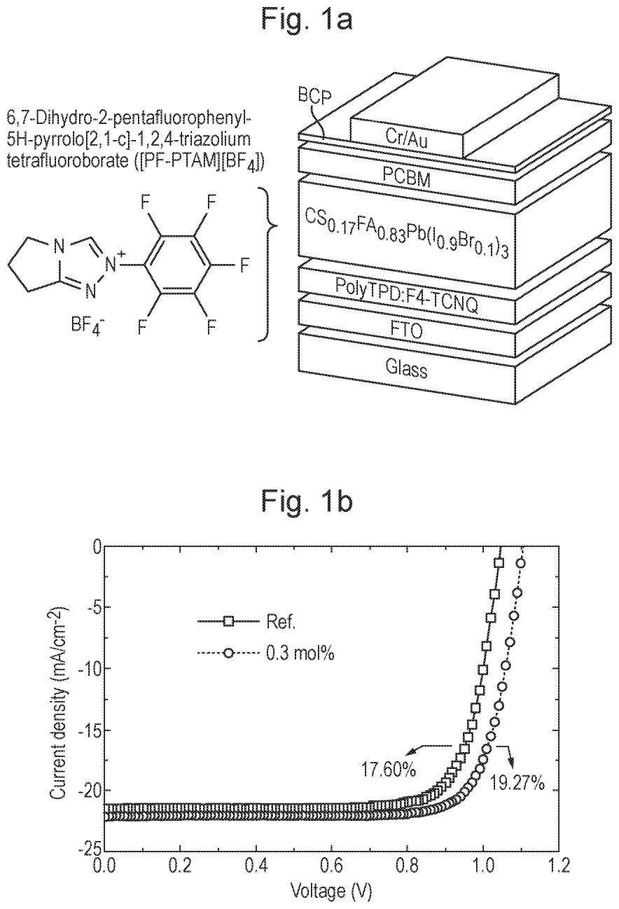

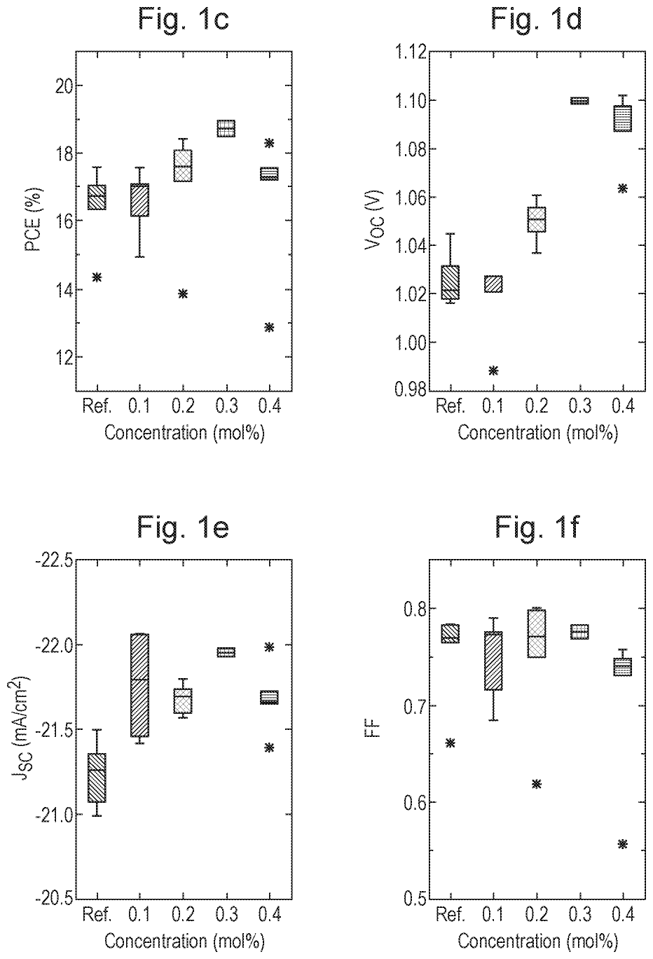

FIGS. 1a-f show device architecture, solar cell performance parameters and statistical results for adding ionic solid (1), 6,7-Dihydro-2-pentafluorophenyl-5H-pyrrolo[2,1-c]-1,2,4-triazolium tetrafluoroborate ([PF-PTAM][BF₄]), into the Cs_{0.17}FA_{0.83}Pb(I_{0.9}Br_{0.1})₃ perovskite precursor. The solar cells were made on 3 cm by 3 cm substrates with 0.2 cm² cell size. FIG. 1a shows the chemical structure of [PF-PTAM][BF₄] and a schematic device architecture of the planar heterojunction p-i-n perovskite solar cell. FIG. 1b shows the current density and voltage (J-V) characteristics of the forward bias (FB) to short-circuit (SC) scans for the perovskite solar cells, with a perovskite absorber layer of the Cs_{0.17}FA_{0.83}Pb(I_{0.9}Br_{0.1})₃ composition with 0.3 mol % (with respect to Pb atom) [PF-PTAM][BF₄] (0.3 mol %, circle) and without ionic solid (Ref., square), under simulated AM1.5 sunlight with the intensity of 100 mW/cm². The corresponding performance parameters, including power conversion efficiency (PCE), open-circuit voltage (V_{OC}), short-circuit current (J_{SC}), and fill factor (FF), are summarised in Table 1 for the cells measured in FIG. 1b. FIGS. 1c-f show statistical results of PCE (FIG. 1c), V_{OC} (FIG. 1d), J_{SC} (FIG. 1e) and FF (FIG. 1f for perovskite solar cells fabricated from Cs_{0.17}FA_{0.83}Pb(I_{0.9}Br_{0.1})₃ perovskite precursors with [PF-PTAM][BF₄] using different concentrations in the range from 0 (i.e., Ref.) to 0.4 mol %. All device parameters are determined from the FB to SC J-V scan curves.

TABLE 1
Corresponding performance parameters, including power
conversion efficiency (PCE), open-circuit voltage (V_{OC}), short-circuit
current (J_{SC}), and fill factor (FF), are summarised
for the cells measured in FIG. 1b.

| | PCE | V_{OC} | J_{SC} | |
|---|---|---|---|---|
| | (%) | (V) | (mA/cm²) | FF |
| Ref. | 17.60 | 1.05 | −21.50 | 0.78 |
| [PF-PTAM][BF₄] | 19.27 | 1.11 | −22.06 | 0.79 |

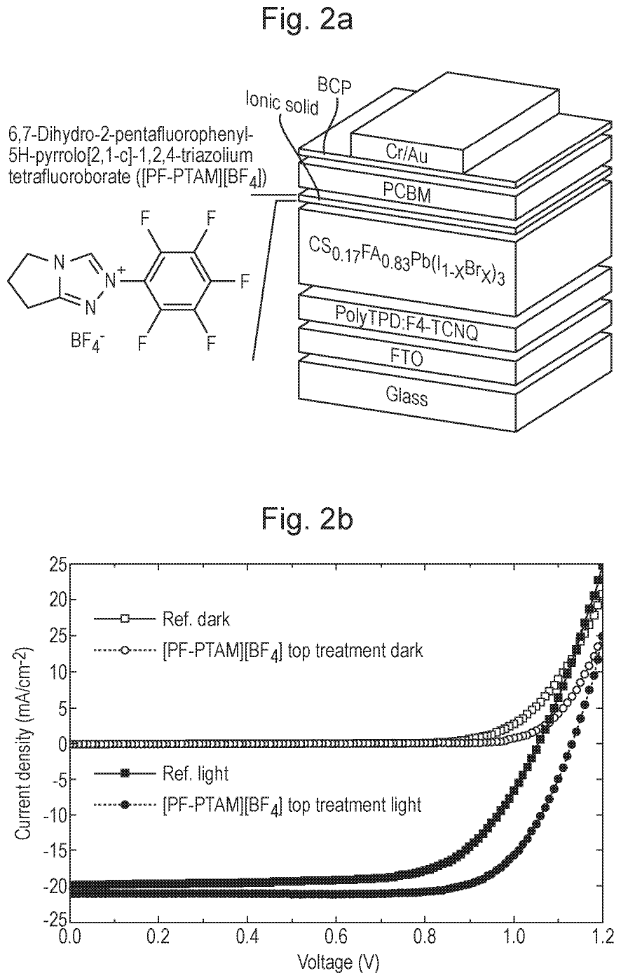

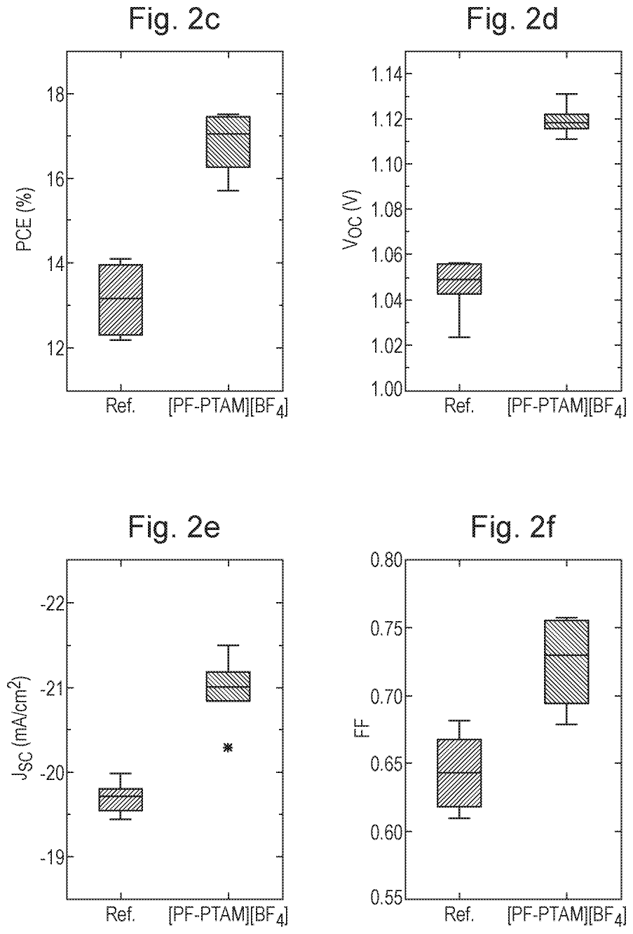

FIGS. 2a-f show device architecture, solar cell performance parameters and statistical results for ionic solid (1), [PF-PTAM][BF₄], deposited onto the Cs_{0.17}FA_{0.83}Pb(I_{0.9}Br_{0.1})₃ perovskite absorber layer. The solar cells were made on 3 cm by 3 cm substrates with 0.2 cm² cell size. FIG. 2a schematically shows the chemical structure of [PF-PTAM][BF₄] and the relative position of this ionic solid in the planar heterojunction p-i-n perovskite solar cell. FIG. 2b shows the J-V characteristics of the FB to SC scans for the perovskite solar cells with a perovskite absorber layer of the Cs_{0.17}FA_{0.83}Pb(I_{0.9}Br_{0.1})₃ composition. The J-V curves include the perovskite solar cells with 0.6 mol % (with respect to Pb atom) [PF-PTAM][BF₄] (top treatment, filled circle) and without ionic solid (Ref., filled square) under simulated AM1.5 sunlight with the intensity of 100 mW/cm² as well as 0.6 mol % [PF-PTAM][BF₄] (open circle) and without ionic solid (Ref., open square) in the dark. The corresponding performance parameters, including PCE, V_{OC}, J_{SC}, and FF, are summarised in Table 2 for the cells measured in FIG. 2b. FIGS. 2c-f show statistical results for perovskite solar cells fabricated from Cs_{0.17}FA_{0.83}Pb(I_{0.9}Br_{0.1})₃ perovskite precursors with an additional layer of 0.6 mol % [PF-PTAM][BF₄] and without ionic solid addition (Ref.): PCE (FIG. 2c), V_{OC} (FIG. 2d), J_{SC} (FIG. 2e), and FF (FIG. 2f). All device parameters are determined from the FB to SC J-V scan curves.

TABLE 2
Corresponding performance parameters, including PCE, V_{OC},
J_{SC}, and FF, are summarised for the cells measured in FIG. 2b.

| | PCE | V_{OC} | J_{SC} | |
|---|---|---|---|---|
| | (%) | (V) | (mA/cm²) | FF |
| Ref. | 14.11 | 1.06 | −19.79 | 0.68 |
| [PF-PTAM][BF₄] | 17.46 | 1.13 | −20.99 | 0.74 |
| top treatment | | | | |

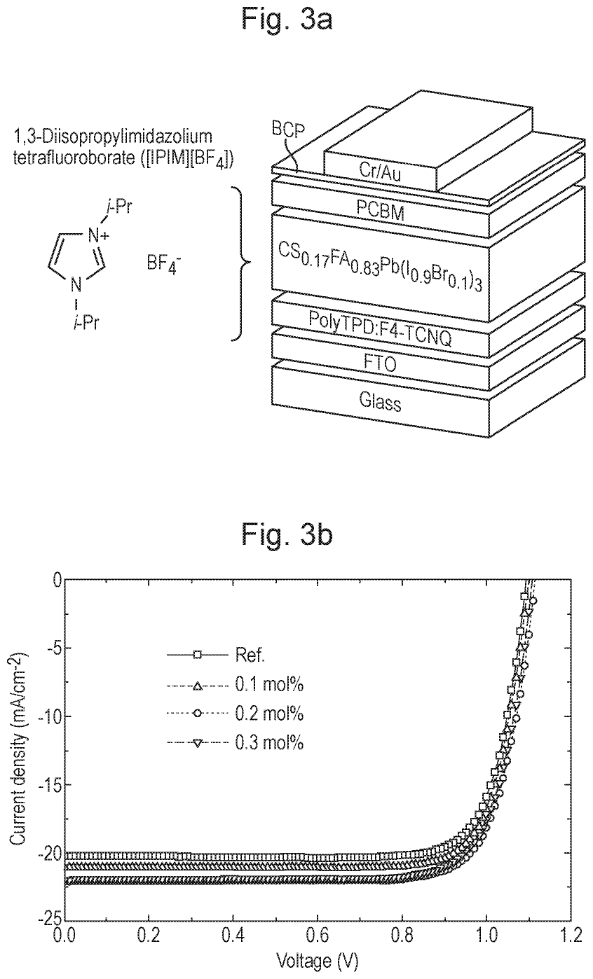

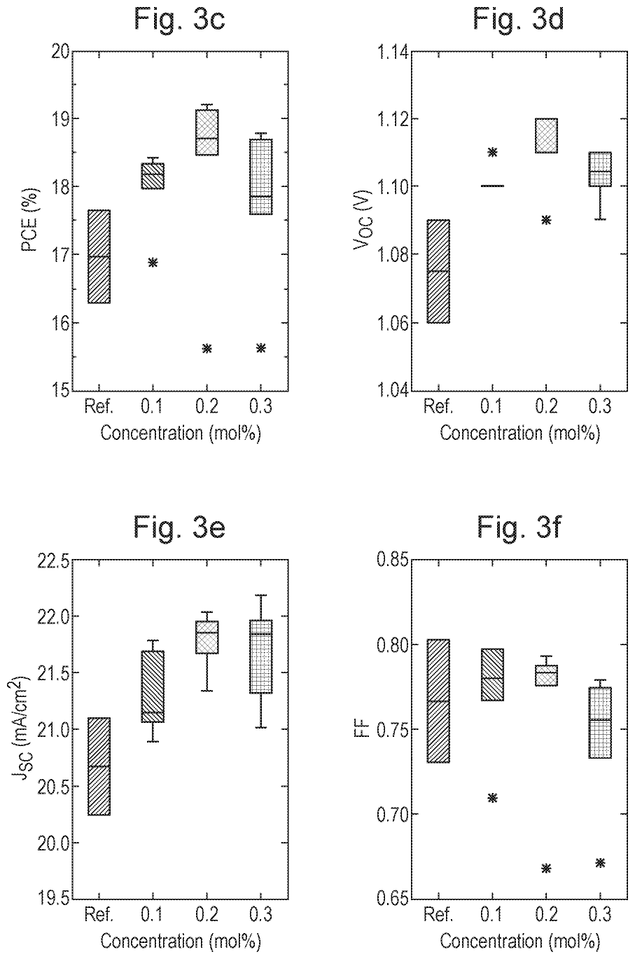

FIGS. 3a-f show device architecture, solar cell performance parameters and statistical results for adding ionic solid (2), 1,3-Diisopropylimidazolium tetrafluoroborate ([IPIM][BF₄]), into the Cs_{0.17}FA_{0.83}Pb(I_{0.9}Br_{0.1})₃ perovskite precursor. The solar cells were made on 2.8 cm by 2.8 cm substrates with 0.0919 cm² cell size. FIG. 3a shows the chemical structure of [IPIM][BF₄] and a schematic device architecture of the planar heterojunction p-i-n perovskite solar cell. FIG. 3b shows the J-V characteristics of the FB to SC scans for the perovskite solar cells, with a perovskite absorber layer of the Cs_{0.17}FA_{0.83}Pb(I_{0.9}Br_{0.1})₃ composition with [IPIM][BF₄] at the concentrations of 0.1 mol % (triangle) (with respect to Pb atom), 0.2 mol % (circle), 0.3 mol % (inverted triangle) and without ionic solid (i.e. Ref., square), under simulated AM1.5 sunlight with the intensity of 100 mW/cm². The corresponding performance parameters, including PCE, V_{OC}, J_{SC}, and FF, are summarised in Table 3 for the cells measured in FIG. 3b. FIG. 3c-f show statistical results of PCE (FIG. 3c), V_{OC} (FIG. 3d), J_{SC} (FIG. 3e) and FF (FIG. 3f) for perovskite solar cells fabricated from Cs_{0.17}FA_{0.83}Pb(I_{0.9}Br_{0.1})₃ perovskite precursors with [IPIM][BF₄] using different concentrations in the range from 0 (i.e., Ref.) to 0.3 mol %. All device parameters are determined from the FB to SC J-V scan curves.

TABLE 3
Corresponding performance parameters, including PCE, V_{OC},
J_{SC}, and FF, are summarised for the cells measured in FIG. 3b.

| | | PCE | V_{OC} | J_{SC} | |
|---|---|---|---|---|---|
| | Concentration | (%) | (V) | (mA/cm²) | FF |
| Ref. | N.A. | 17.65 | 1.09 | −20.24 | 0.80 |
| [IPIM][BF₄] | 0.1 mol % | 18.34 | 1.10 | −21.07 | 0.80 |
| | 0.2 mol % | 19.13 | 1.12 | −22.04 | 0.78 |
| | 0.3 mol % | 18.79 | 1.11 | −21.96 | 0.78 |

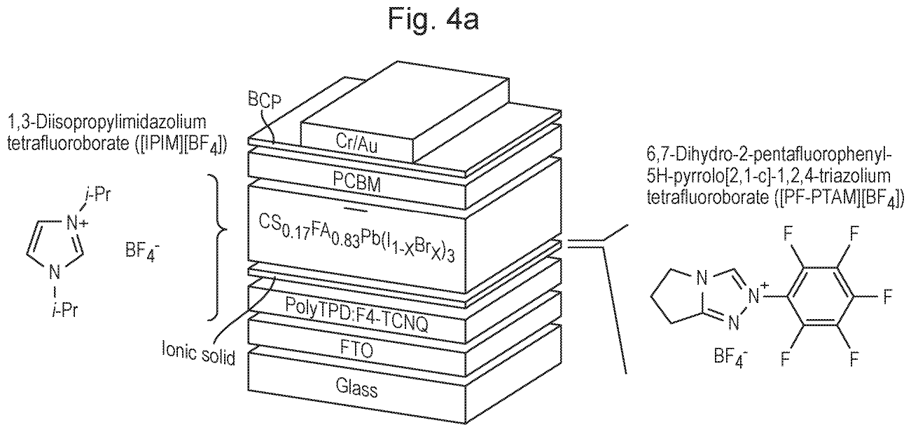

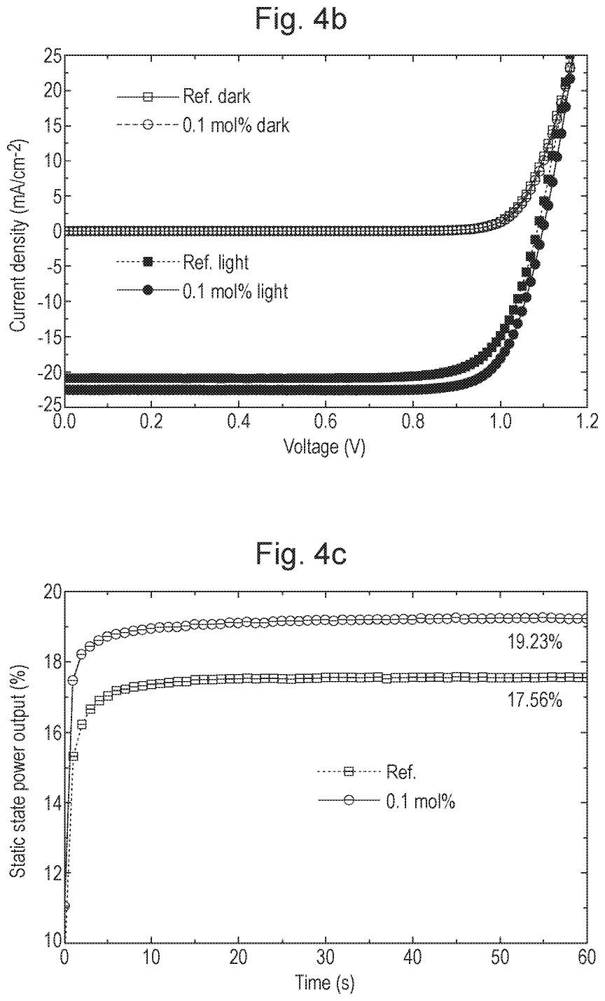

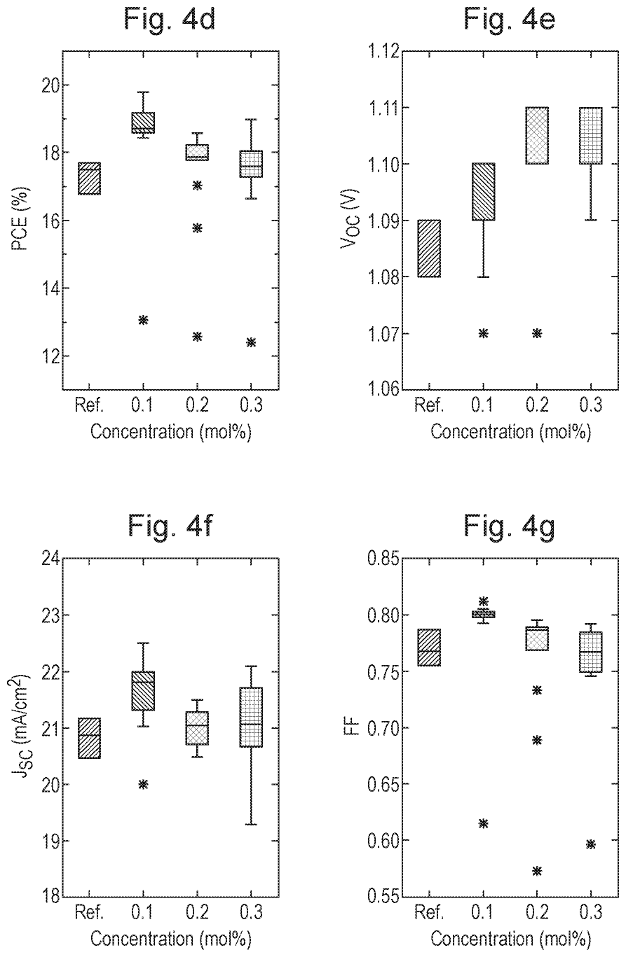

FIGS. 4a-g show device architecture, solar cell performance parameters and statistical results for ionic solid (1), [PF-PTAM][BF₄], deposited before the ionic solid (2), [IPIM][BF₄], containing Cs_{0.17}FA_{0.83}Pb(I_{0.9}Br_{0.1})₃ perovskite absorber layer. For the perovskite solar cells shown in FIG. 4, the bottom surface treatment using [PF-PTAM][BF₄] was prepared from an 0.6 mol % (with respect to Pb atom) [PF-PTAM][BF₄] ionic solid precursor, unless stated otherwise. The solar cells were made on 2.8 cm by 2.8 cm substrates with 0.0919 cm² cell size. FIG. 4a schematically shows the chemical structures of [PF-PTAM][BF₄] and [IPIM][BF₄] as well as the relative positions of the ionic solids in the planar heterojunction p-i-n perovskite solar cell. FIG. 4b shows the J-V characteristics of the FB to SC scans for the perovskite solar cells with a perovskite absorber layer of the Cs_{0.7}FA_{0.83}Pb(I_{0.9}Br_{0.1})₃ composition. The J-V curves include the perovskite solar cells with the ionic solid additions (0.1 mol %, filled circle), including 0.1 mol % [IPIM][BF₄] in the perovskite precursor and the perovskite bottom surface treated with [PF-PTAM][BF₄], and without ionic solid (Ref., filled square) under simulated AM1.5 sunlight with the intensity of 100 mW/cm² as well as in the dark (open circle for the cell with the ionic solid additions; open square for the cell without ionic solid). The corresponding performance parameters, including PCE, V_{OC}, J_{SC}, and FF, are summarised in Table 4 for the cells measured in FIG. 4b. FIG. 4c shows the static state power output for the cells with (circle) and without (square) the ionic solid additions. FIG. 4d-g show statistical results for perovskite solar cells fabricated from Cs_{0.17}FA_{0.83}Pb(I_{0.9}Br_{0.1})₃ perovskite precursors with the ionic solid additions at different concentrations of [IPIM][BF₄] from 0.1 to 0.3 mol % and without the ionic solid additions (Ref.): PCE (FIG. 4d), V_{OC} (FIG. 4e), J_{SC} (FIG. 4f), and FF (FIG. 4g). All device parameters are determined from the FB to SC J-V scan curves.

TABLE 4
Corresponding performance parameters, including PCE, V_{OC},
J_{SC}, and FF, are summarised for the cells measured in FIG. 4b.

| | PCE | V_{OC} | J_{SC} | |
|---|---|---|---|---|
| | (%) | (V) | (mA/cm²) | FF |
| Ref. | 17.68 | 1.08 | −20.88 | 0.79 |
| [IPIM][BF₄] | 19.80 | 1.10 | −22.51 | 0.81 |
| 0.1 mol % with | | | | |
| [PF-PTAM][BF₄] | | | | |
| bottom treatment | | | | |

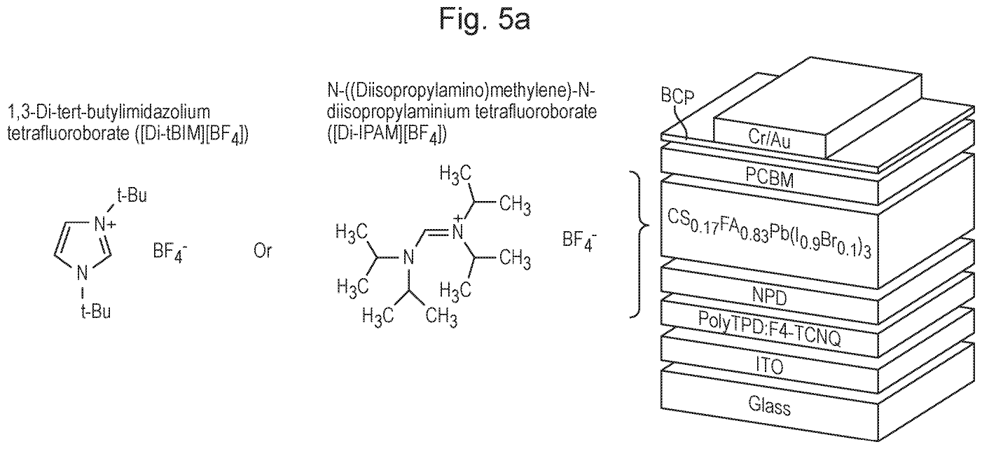

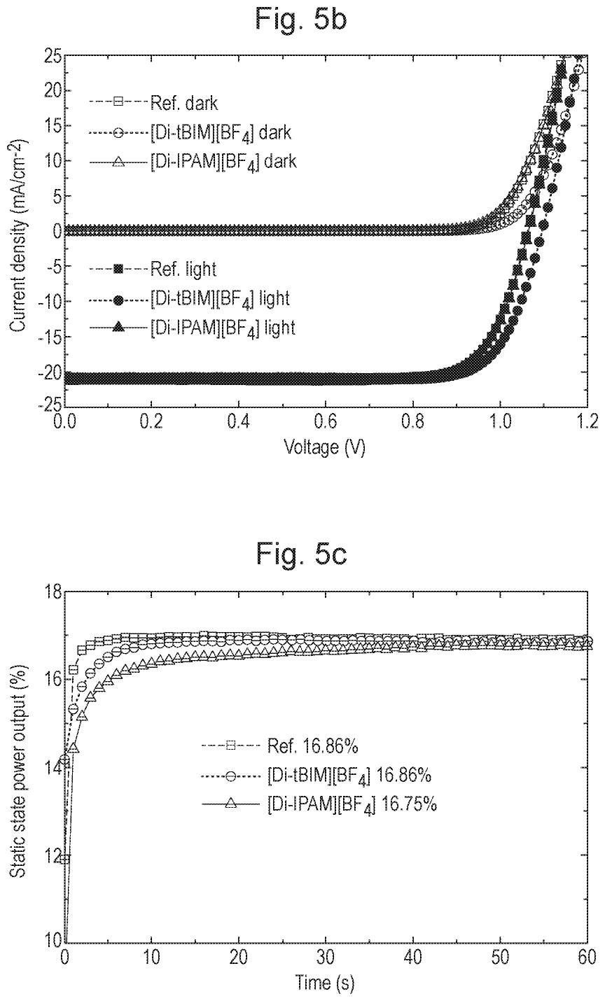

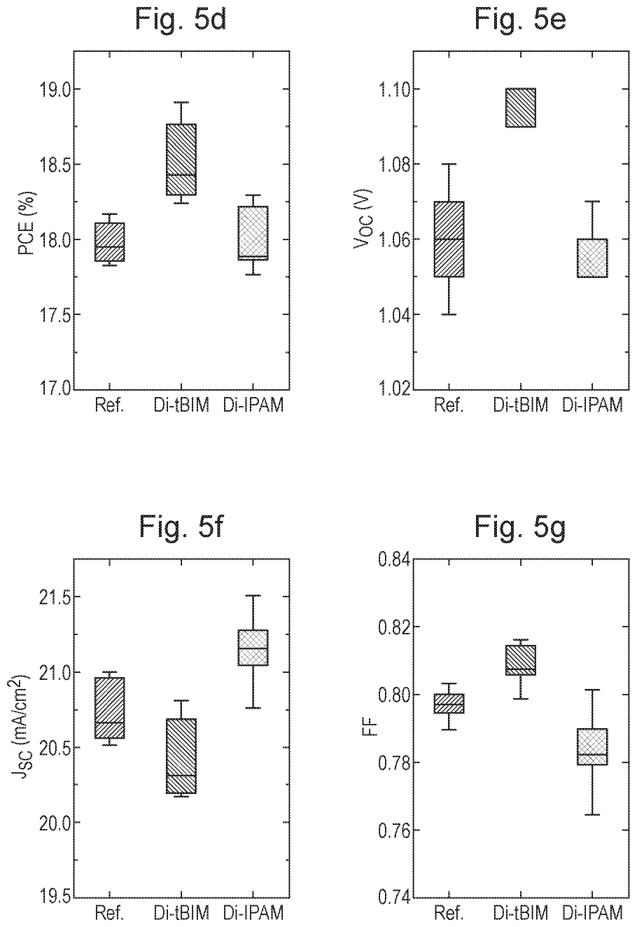

FIGS. 5a-g show device architecture, solar cell performance parameters and statistical results for adding ionic solid (3), 1,3-Di-tert-butylimidazolium tetrafluoroborate ([Di-tBIM][BF₄], or ionic solid (4), N-((Diisopropylamino)methylene)-N-diisopropylaminium tetrafluoroborate ([Di-IPAM][BF₄]), to the Cs_{0.17}FA_{0.83}Pb(I_{0.9}Br_{0.1})₃ perovskite absorber layer. The solar cells were made on 2.8 cm by 2.8 cm substrates with 0.0919 cm² cell size. FIG. 5a schematically shows the chemical structures of [Di-tBIM][BF₄] and ([Di-IPAM][BF₄] as well as the planar heterojunction p-i-n perovskite solar cell. FIG. 5b shows the J-V characteristics of the FB to SC scans for the perovskite solar cells with a perovskite absorber layer of the Cs_{0.1}FA_{0.83}Pb(I_{0.9}Br_{0.1})₃ composition. The J-V curves include the perovskite solar cells with 0.2 mol % (with respect to Pb atom) [Di-tBIM][BF₄] (filled circle), 0.2 mol % [Di-IPAM][BF₄] (filled triangle) and without ionic solid (Ref., filled square) under simulated AM1.5 sunlight with the intensity of 100 mW/cm² as well as in the dark (open circle for the cell with [Di-tBIM][BF₄]; open triangle for the cell with [Di-IPAM][BF₄]; open square for the cell without ionic solid). The corresponding performance parameters, including PCE, V_{OC}, J_{SC}, and FF, are summarised in Table 5 for the cells measured in FIG. 5b. FIG. 5c shows the static state power output for the cells with [Di-tBIM][BF₄], with [Di-IPAM][BF₄] (triangle) and without ionic solid (square). FIG. 5d-g show statistical results for perovskite solar cells fabricated from Cs_{0.17}FA_{0.83}Pb(I_{0.9}Br_{0.1})₃ perovskite precursors with ionic solids of [Di-tBIM][BF₄] and [Di-IPAM][BF₄] as well as without ionic solid (Ref.): PCE (FIG. 5d), V_{OC} (FIG. 5e), J_{SC} (FIG. 5f), and FF (FIG. 5g). All device parameters are determined from the FB to SC J-V scan curves.

TABLE 5
Corresponding performance parameters, including PCE, V_{OC}, J_{SC},
and FF, are summarised in Table 5 for the cells measured in FIG. 5b.

| | PCE | V_{OC} | J_{SC} | |
|---|---|---|---|---|
| | (%) | (V) | (mA/cm²) | FF |
| Ref. | 18.17 | 1.06 | −21.00 | 0.80 |
| [Di-tBIM][BF₄] | 18.91 | 1.10 | −20.81 | 0.81 |
| [Di-IPAM][BF₄] | 18.29 | 1.07 | −21.28 | 0.78 |

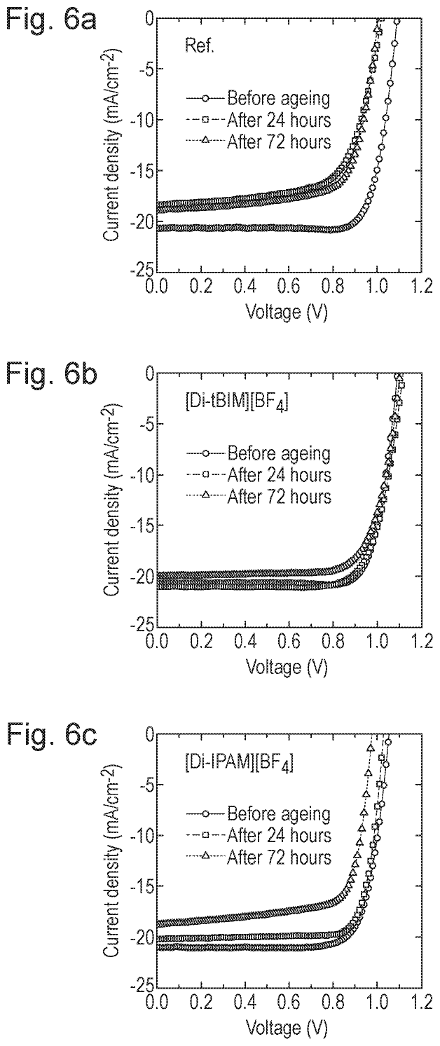

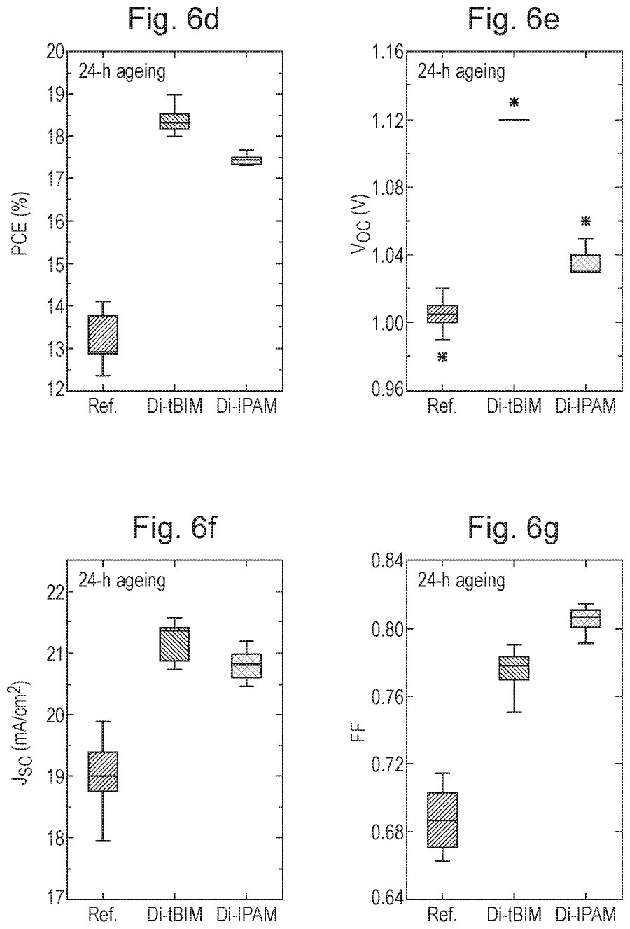

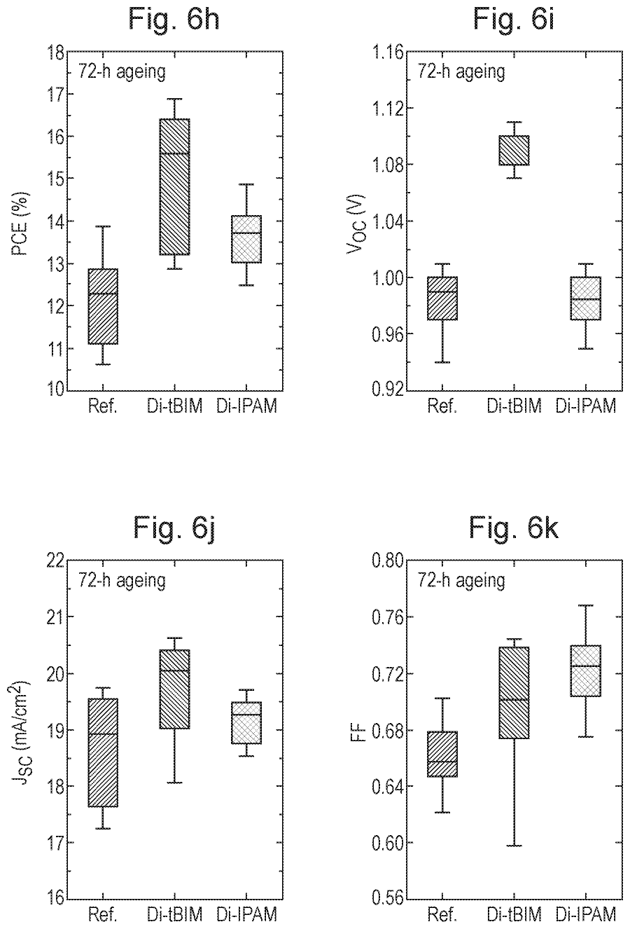

FIGS. 6a-k show solar cell performance parameters and statistical results for adding ionic solid (3), 1,3-Di-tert-butylimidazolium tetrafluoroborate ([Di-tBIM][BF₄], or ionic solid (4), N-((Diisopropylamino)methylene)-N-diisopropylaminium tetrafluoroborate ([Di-IPAM][BF₄]), to the Cs_{0.17}FA_{0.83}Pb(I_{0.9}Br_{0.1})₃ perovskite absorber layer for a 72-hour ageing period under full spectrum sunlight and heat (85° C.). The solar cells were made on 2.8 cm by 2.8 cm substrates with 0.0919 cm² cell size. FIG. 6a-c show the J-V characteristics of the FB to SC scans for the perovskite solar cells with a perovskite absorber layer of the Cs_{0.17}FA_{0.83}Pb(I_{0.9}Br_{0.1})₃ composition without ionic solid (Ref., FIG. 6a), with 0.2 mol % (with respect to Pb atom) [Di-tBIM][BF₄] (FIG. 6b), and 0.2 mol % [Di-IPAM][BF₄] (FIG. 6c), before ageing (circle), after 24-hour ageing (square), and 72-hour ageing (triangle). The corresponding performance parameters, including PCE, V_{OC}, J_{SC}, and FF, are summarised in Table 6 for the cells measured in FIG. 6a-c. FIG. 6d-g show statistical results for perovskite solar cells fabricated from Cs_{0.17}FA_{0.83}Pb(I_{0.9}Br_{0.1})₃ perovskite precursors with ionic solids of [Di-tBIM][BF₄] and [Di-IPAM][BF₄] as well as without ionic solid (Ref.) after 24-hour ageing: PCE (FIG. 6d), V_{OC} (FIG. 6e), J_{SC} (FIG. 6f), and FF (FIG. 6g). FIGS. 6h-k show statistical results for perovskite solar cells fabricated from Cs_{0.17}FA_{0.83}Pb(I_{0.9}Br_{0.1})₃ perovskite precursors with ionic solids of [Di-tBIM][BF₄] and [Di-IPAM][BF₄] as well as without ionic solid (Ref.) after 72-hour ageing: PCE (FIG. 6h), V_{OC} (FIG. 6i), J_{SC} (FIG. 6j), and FF (FIG. 6k). All device parameters are determined from the FB to SC J-V scan curves.

TABLE 6
Corresponding performance parameters, including PCE, V_{OC},
J_{SC}, and FF, are summarised for the cells measured in FIG. 6a-c.

| | Ageing | PCE | V_{OC} | J_{SC} | |
|---|---|---|---|---|---|
| | time | (%) | (V) | (mA/cm²) | FF |
| Ref. | Before | 17.83 | 1.06 | −20.57 | 0.80 |
| | 24 h | 12.88 | 1.02 | −18.37 | 0.70 |
| | 72 h | 13.87 | 1.01 | −18.96 | 0.70 |
| [Di-tBIM][BF₄] | Before | 18.51 | 1.09 | −20.69 | 0.80 |
| | 24 h | 18.20 | 1.12 | −21.03 | 0.75 |
| | 72 h | 16.88 | 1.10 | −20.00 | 0.74 |
| [Di-IPAM][BF₄] | Before | 17.64 | 1.05 | −20.97 | 0.78 |
| | 24 h | 17.07 | 1.03 | −20.19 | 0.80 |
| | 72 h | 13.83 | 0.98 | −18.80 | 0.73 |

### Example 2

Methods

Precursor Material Preparation

Lead iodide (PbI₂, 99.999%, metals basis), lead bromide (PbBr₂, ≥98%) and cesium iodide (CsI, 99.99%) were purchased from Alfa-Aesar. Formamidinium iodide (FAI) was purchased from GreatCell Solar. 1-n-butyl-1-methylpiperidinium tetrafluoroborate ([BMP]⁺[BF₄]⁻, 99%) was purchased from Sigma-Aldrich. [6,6]-phenyl-C₆₁-butyric acid methyl ester (PC₆₁BM, ≥99.5%) was purchased from Solenne BV. Bathocuproine (BCP, 98%) was purchased from Alfa Aesar. Poly(4-butylphenyl-diphenyl-amine) (polyTPD) was purchased from 1-Material. 2,3,5,6-Tetrafluoro-7,7,8,8-tetracyanoquinodimethane (F4-TCNQ) was purchased from Lumtec. Unless stated otherwise, all other materials and solvents were purchased from Sigma-Aldrich. In this work, all the materials were used as received without further purification.

To form the mixed-cation lead mixed-anion perovskite precursor solutions, CsI, FAI, PbI₂, and PbBr₂ were prepared in the way corresponding to the exact stoichiometry for the hybrid perovskite composition [e.g. Cs_{0.17}FA_{0.83}Pb(I_{0.9}Br_{0.1})₃ and Cs_{0.17}FA_{0.83}Pb(I_{0.90}Br_{0.10})₃] in a mixed organic solvent system comprising anhydrous N,N-dimethylformamide (DMF) and anhydrous dimethyl sulfoxide (DMSO) at the volume ratio of DMF:DMSO=4:1. The perovskite precursor concentration used was 1.45 M. In parallel, the piperidinium ionic solid [BMP]⁺[BF₄]⁻ containing perovskite precursor solutions were prepared by dissolving the same perovskite components as the non-ionic-solid containing perovskite precursor solutions in the DMF/DMSO mixed solvent system with [BMP]⁺[BF₄]⁻ in the desired molar ratios with respect to the Pb content. The perovskite precursor solutions were stirred overnight in a nitrogen-filled glovebox and used without any further treatment. For the hole-transporting material, polyTPD was dissolved in toluene in a concentration of 1 mg·mL⁻¹ along with 20 wt % of F4-TCNQ. For the electron-transporting (PC₆₁BM) and hole blocking (BCP) materials, PC₆₁BM and BCP were prepared separately by dissolving PC₆₁BM and BCP in a mixed organic solvent system of chlorobenzene (CB) and 1,2-dichlorobenzene (DCB) (CB:DCB=3:1 in volume) and pure isopropanol (IPA) at a concentration of 20 and 0.5 mg·mL⁻¹, respectively.

Perovskite Cell and Film Fabrication

Fluorine-doped tin oxide (FTO) coated glass (Pilkington TEC 7, 7Ω/ [symbol] sheet resistivity) was etched with zinc powder and 2 M HCl to obtain desired transparent electrode patterns. The substrates were cleaned in a series of ultrasonic cleaning baths using various solutions and solvents in the following sequence: 1) deionized water with 2% v/v solution of Decon 90 cleaning detergent; 2) deionized water; 3) acetone and 4) IPA (each step for 5˜8 mins). After ultrasonic cleaning, substrates were dried with dry nitrogen and then treated with UV-Ozone for 15˜20 mins before use. After the substrate cleaning procedure, F4-TCNQ doped polyTPD was deposited by dispensing the as-prepared organic solution onto a spinning substrate at 2000 rpm for 20 see, followed by thermal annealing at 130° C. for 5 min in ambient air. The deposition of the perovskite layers was carried out using a spin coater in a nitrogen-filled glove box with the following processing parameters: starting at 1000 rpm for 5 sec (ramping time of 5 see from stationary status) and then 5000 rpm (ramping time of 5 sec from 1000 rpm) for 30 sec. Before the end of the spinning process, a solvent-quenching method (Nat. Mater. 13, 897-903, 2014) was used by dropping anisole of 250˜300 μL onto the spinning substrates at 40 sec after the start of the spin-coating process. The thermal annealing process (100° C. for 50˜55 min) was then carried out for the formation of the perovskite layer. The as-prepared PC₆₁BM solution was dynamically spun onto the perovskite layers at a speed of 2000 rpm for 20 sec. The samples were then annealed at 100° C. for 3˜5 min. After cooling down to room temperature, the as-prepared BCP solution was dynamically spun onto the PCBM layer at a speed of 4000 rpm for 20 see, followed by a brief thermal annealing process at 100° C. for ˜1 min. Both PCBM and BCP were processed inside the nitrogen-filled glovebox. The hybrid perovskite single-junction solar cells were completed by thermal evaporation of Cr (3.5 nm) and Au electrodes (100 nm) through shadow masks under high vacuum (6×10⁻⁶ torr) using a thermal evaporator (Nano 36, Kurt J. Lesker) placed in ambient environment.

Perovskite Solar Cell Characterization

The current density and voltage (J-V) characteristics for perovskite solar cells were measured in air with a Keithley 2400 source meter under AM1.5 sunlight at 98˜102 mW·cm⁻² irradiance generated using an ABET Sun 2000 Class A simulator. The mismatch factor for the test cell, the light source and the National Renewable Energy Laboratories (NREL)-calibrated KG5 filtered silicon reference cell was estimated and applied in order to correctly estimate the equivalent AM1.5 irradiance level. Before the start of the measurement for each set of devices, the intensity of the solar simulator was automatically measured using a KG5 reference cell, and this recorded intensity was used to calculate the precise power conversion efficiency, where power conversion efficiency (PCE) is: (electrical power out/solar light power in)×100%. Unless otherwise stated, all perovskite single-junction devices were masked with a 0.0919 cm² metal aperture to define the active area and to eliminate edge effects. The J-V curves were taken at a scan rate of 100 mV·s⁻¹ (delay time of 100 ms) from 1.3 V to −0.1 V and then back again (from −0.1 V to 1.3 V). A stabilization time of 1 s at forward bias of 1.3 V under illumination was done before scanning. We note that the cells could use multiple, continuous measurements (typically up to five J-V scans) in order to reach a peak performance. External quantum efficiency (EQE) measurements were carried out using custom-built Fourier transform photocurrent spectroscopy based on a Bruker Vertex 80v Fourier transform spectrometer. A tungsten-halogen lamp was used as the light source and the intensity was calibrated against a Newport-calibrated reference silicon photodiode.

Perovskite Solar Cell Stability Test and Device Characterization

The perovskite solar cells were encapsulated with a cover glass (LT-Cover, Lumtec) and UV adhesive (LT-U001, Lumtec) in a nitrogen-filled glovebox. All the encapsulated devices were aged using an Atlas SUNTEST XLS+ (1,700 W air-cooled Xenon lamp) light-soaking chamber under simulated full-spectrum AM1.5 sunlight with 76 mW·cm^{- 2} irradiance. For the unencapsulated devices were aged using an Atlas SUNTEST CPS+ light-soaking chamber under simulated full-spectrum AM1.5 sunlight with 77 mW·cm^{- 2} irradiance. All aging tests were conducted in open-circuit conditions, and to perform J-V characterization the samples were taken out from the chamber and tested at different time intervals, following the measurement protocol as described above. No ultraviolet filter was applied during the aging process. The aging chamber for storing the encapsulated samples was air-cooled with the temperature controlled at 85° C. while the temperature for the chamber where the unencapsulated samples were aged was set to 60° C. The temperature for both the aging chambers were measured by a black standard temperature control unit. During the aging period the relative humidity in the laboratory was monitored in the range of ˜50±5%.

PbI₂ Film Preparation and Stability Test

PbI₂ films prepared from 1 M PbI₂ solution (DMF:DMSO=4:1) were spun onto FTO glass substrates at 4000 rpm for 30 see followed by thermal annealing at 100° C. for 30 min. The entire deposition was carried out in N₂. A light-emitting diode (LED) luminaire (Intelligent LED Solutions, ILF-GD72-WMWH-SD401-WIR200) positioned above a mirrored box (open at both the top and bottom) was used as the illumination source. The luminaire was supplied with power from a Voltcraft DPPS-60-10 set in constant current mode to provide 2 A (˜39.7 V) to the LED array. PbI₂ films were illuminated by placing them below the luminaire/mirror box assembly in the center of illuminated area on a hotplate (Fisher Scientific, 11-102-50H) set to 85° C. inside a nitrogen-filled glovebox with O₂ and H₂O<1 ppm. The estimation of the equivalent solar illumination intensity of the LED luminaire is provided in the Supplementary Text.

Photoluminescence Characterization

Steady-state photoluminescence spectra were recorded using an excitation wavelength of 510 nm and slit widths of 5 mm on a commercial spectrofluorometer (Horiba, Fluorolog). Time-resolved PL measurements were acquired using a time-correlated single photon counting (TCSPC) setup (FluoTime 300 PicoQuant GmbH). Film samples were photoexcited using a 507-nm laser head (LDH-P-C-510, PicoQuant GmbH) pulsed at 0.2 MHz. Perovskite films were prepared on polyTPD coated FTO glass substrates.

High-Resolution Secondary Ion Mass Spectrometry Characterization

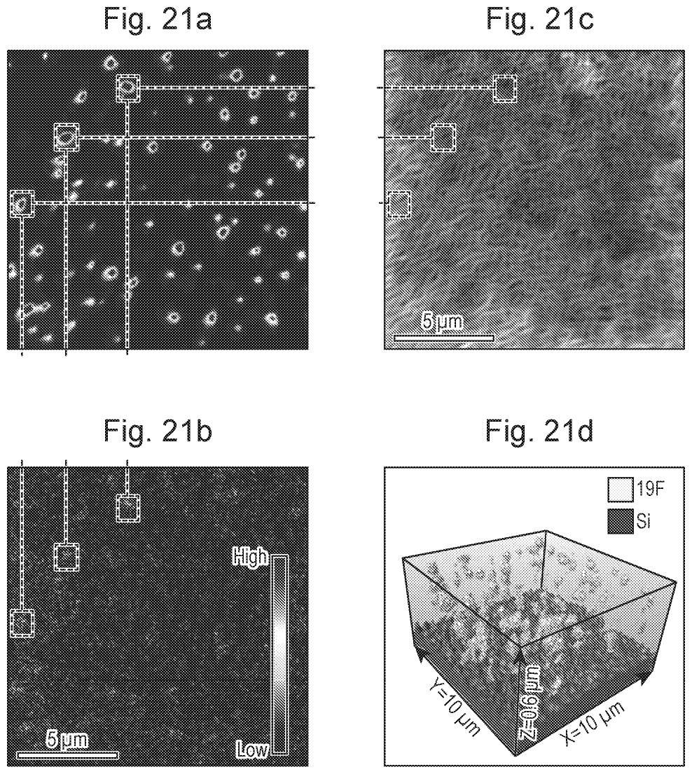

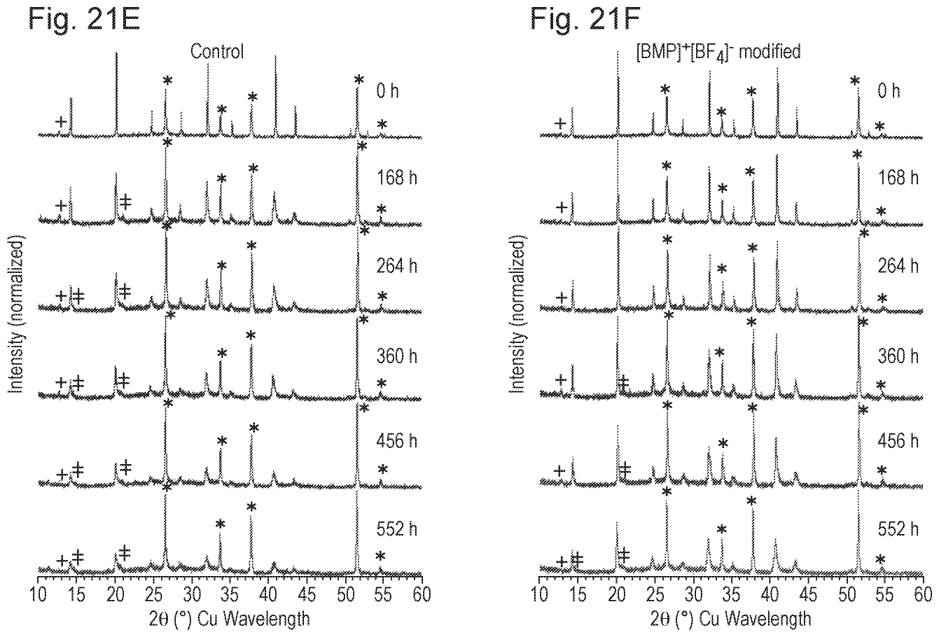

To investigate the localisation of B and F in these samples we deposited ˜500 nm perovskite films onto Si/SiO₂ substrates with 0.25 mol % [BMP]⁺[BF₄]⁻ (with to the Pb content of the perovskite used) and employed high-resolution secondary ion mass spectrometry (NanoSIMS) to perform elemental mapping in a CAMECA NanoSIMS 50 system using a focused 16 keV Cs⁺ primary beam. The D1 aperture was set to D1-3 (150 μm diameter), which provides a primary beam of ˜1 pA (beam diameter ˜150 nm) which was rastered over the selected measurement area. Entrance and aperture slits were chosen to be 40×220 μm (ES-2) and 200×200 μm (AS-2) respectively. The raster size was 15 μm×15 μm (256×256 pixels) and the dwell time was 2000 μs per pixel. Ion maps were collected simultaneously for ¹²C⁻, ¹⁹F⁻, ²⁸Si⁻ and ¹¹B¹⁶O₂⁻ ion signals, together with the secondary electron signal produced during the sputtering process, which can be used to show both sample morphology and surface topography. These scans were repeated 200 times from the same area, giving a set of stacked images of the distributions of each element, and sputtering to a total depth of ˜700 nm below the sample surface. The typical two-dimensional (2D) SIMS maps in FIG. 21A-C are summed from images #21 to #40 to give better signal-to-noise ratios (SNRs). The ‘auto-track’ feature in ImageJ was used to correct any image drift before the images were summed, and the FEI Avizo software package was then applied for the three-dimensional (3D) visualization shown in FIG. 21D following the methodologies described in Appl. Surf. Sci. 464, 311-320 (2019). We have plotted depth and line profiles using ImageJ with the OpenMIMS plugin (Harvard) from small regions covering one ¹⁹F hotspot and part of the perovskite matrix to compare the ¹⁹F⁻ intensities.

Solid-State Nuclear Magnetic Resonance

All the solid-state nuclear magnetic resonance (ssNMR) experiments were carried out using a triple-channel 1.3 mm ssNMR probe on a 700 MHz Bruker AV III spectrometer. All the ¹H pulses were at a nutation frequency of 67 kHz with the sample spun at a magic-angle spinning frequency of 55 kHz. The number of scans for 1D Bloch-decay experiments was 3200. Standard three-pulse scheme was used for the 2D spin-diffusion experiments. The 2D spectra were acquired with 96 scans for each of the 160 increments corresponding to final ti evolution time of 4 ms, and 100 ms of mixing time. States-TPPI was used for frequency discrimination in the indirect dimension. Data were processed using Topspin 3.5 with an exponential line broadening of 20 Hz which were edited using inkscape 0.92. The temperature during the experiment was maintained at 298 K.

Transient Photovoltage and Photocurrent Characterization

The illumination was provided by a ring of 12 white light-emitting diodes (LEDs) with a fast-switching metal oxide semiconductor field-effect transistor. The one sun equivalent illumination was calibrated by matching the value of J_{SC} and V_{OC} obtained under the AM1.5G solar simulator measurement. The light was switched on for approximately 2 ms to allow a steady state to be reached, and a much longer time with the light switched off was applied to avoid overheating. The potential bias was applied by a Keithley 2400 source-meter, and the current and the voltage across were measured by a Tektronix TDS3032B oscilloscope with a 1-MΩ input impedance. Charge extraction was used to determine the average charge carrier densities in devices under different illumination levels and different biases (open circuit and short circuit in this study).

The desired light intensity was provided by a ring of 12 white LEDs the same as above, which is capable of a power up to 5 sun equivalents. The device was held under the initial bias at certain background light and then switched to short circuit and turn off light, and the transient was acquired with a DAQ card connected to a Tektronix TDS3032B oscilloscope. The voltage transients were converted into current transients through Ohm's law, the current transients were then integrated to obtain total charge to calculate charge carrier density in the device. During TPV measurements, the device was held at open circuit condition under different background light intensities controlled by a ring of white LEDs as described before; then a small optical excitation was provided by a pulsed Continuum Minilite Nd:YAG laser at 532 nm with a pulse width of smaller than 10 nm. This small excitation produced a small voltage transient decay was then measured by the oscilloscope. The results were fitted with a mono-exponential decay function to obtain the small perturbation carrier lifetime and to estimate the total charge carrier lifetime within the device.

Time-Resolved Microwave Conductivity Characterization

The experimental setup for the time-resolved microwave conductivity (TRMC) measurement can be found in previous work (Chem. Mater. 31, 3359-3369, 2019). A microwave-frequency oscillatory electric signal is generated using a Sivers IMA VO4280X/00 voltage-controlled oscillator (VCO). The signal has an approximate power of 16 dBm and a tunable frequency between 8 GHz and 15 GHz. The VCO is powered with an NNS1512 TDK-Lambda constant 12V power supply, and the output frequency is controlled by a Stahl Electronics BSA-Series voltage source. The oscillatory signal is incident on an antenna inside a WR90 copper-alloy waveguide. The microwaves emitted from the antenna pass through an isolator and an attenuator before they are incident on a circulator (Microwave Communication Laboratory Inc. CSW-3). The circulator acts as a unidirectional device in which the incident microwaves pass through a fixed iris (6.35 mm diameter) into a sample cavity. The cavity supports a TE₁₀₃ mode standing wave and consists of an ITO-coated glass window that allows optical access to the sample. The sample is mounted inside the cavity at a maximum of the electric-field component of the standing microwaves, using a 3D-printed PLA sample holder.

Microwaves reflected from the cavity are then incident on the circulator, directed through an isolator, and onto a zero-bias Schottky diode detector (Fairview Microwave SMD0218). The detector outputs a voltage which is linearly proportional to the amplitude of the incident microwaves. The detected voltage signal is amplified by a Femto HAS-X-1-40 high-speed amplifier (gain=×100). The amplified detector voltage is measured as a function of time by a Textronix TDS 3032C digital oscilloscope. A Continuum Minilite II pulsed Nd:YaG laser is used to illuminate the sample. The laser pulse has a wavelength of 532 nm, a full width at half-maxima of approximately 5 ns and a maximum fluence incident on the sample of ˜-10¹⁵ photons per cm² per pulse. An external trigger link is employed to trigger the oscilloscope before the laser fires. The photoconductance was evaluated from changes in the detector voltage using standard analysis as described in previous works (Chem. Mater. 31, 3359-3369, 2019; J. Mater. Chem. C 5, 5930-5938, 2017; J. Phys. Chem. C 117, 24085-24103, 2013). All the measurements were conducted in air, without encapsulation, in the over-coupled regime. Perovskite films were prepared on quartz substrates.

In-Plane Transient Photoconductivity Characterization

The experimental setup for the in-plane transient photoconductivity (ip-TPC) measurement can be found in our previous work (Adv. Funct. Mater. 29, 1901371, 2019; Energy. Environ. Sci. 12, 169-176, 2019). A Nd:YAG pulsed laser excitation source pumped at 10 Hz with FWHM=3.74 ns was set to 470 nm with a range of fluences in order to vary the charge carrier densities of FACs. A bias of 2.96 V is applied on one of the in-plane electrodes. A variable resistor is connected in series with the samples. The voltage drop on this resistor was monitored through an oscilloscope with a high internal resistance (1 MΩ) connected in parallel to determine the change of the potential across the two in-plane Au electrodes. The ip-TPC was calculated using the following equation

$${σ}_{TPC} = \frac{{V}_{r}}{{R}_{r} \times ( {V}_{appl} - {V}_{r} )} \times \frac{l}{w \times t} \qquad ( S 1 )$$

where Rᵣ is resistance for the variable resistor, Vᵣ is the potential drop measured across the resistor, Vₐₚₚₗ is applied voltage, l is channel length, w is channel width, and t is film thickness. Perovskite films were prepared on glass substrates.

X-Ray Diffraction Characterization

The X-ray diffraction (XRD) patterns were measured from perovskite samples deposited onto FTO glass substrates using a Cu Kα X-ray source and a Panalytical X'PERT Pro X-ray diffractometer. Topas-6 software was used to implement Pawley fits in order to extract lattice parameters, using FTO as an internal standard.

X-Ray Photoemission Spectroscopy Characterization

X-ray photoemission spectroscopy measurements were carried out using a Thermo Scientific Kα photoelectron spectrometer using a monochromated Al Kα X-ray source at a take-off angle of 90°. The core level XPS spectra were recorded using a pass energy of 20 eV (resolution approximately 0.4 eV) from an analysis area of 400 μm×400 μm. The spectrometer work function and binding energy scale were calibrated using the Fermi edge and 3d peak recorded from a polycrystalline silver (Ag) sample prior to the commencement of the experiments. Fitting procedures to extract peak positions were carried out using CasaXPS, the background of the spectra was fitted using a Shirley lineshape and the peaks were fit using a mixture of Gaussian/Lorentzian (Lorentzian=20%) line shapes. For characterizing perovskite films, the samples were prepared on polyTPD coated FTO glass substrates.

Ultraviolet-Visible Spectroscopy Characterization

The ultraviolet-visible (UV-vis) transmission measurements were performed using an Agilent Cary 60 UV-vis spectrophotometer. The samples were prepared on FTO substrates using the same deposition parameters described above.

Optical microscopy characterization. The optical microscopy measurements were performed using a Nikon motorized microscope (Eclipse LV100ND). A UV-375 nm LED bulb (BSIL100LEDC) is used for carrying out the PL-mode characterization.

Scanning Electron Microscopy Characterization

A Hitachi S-4300 scanning electron microscope was used to acquire cross-sectional images of target samples.

Iodine Loss Characterization

Perovskite films were prepared on FTO glass substrates and deposited using the same protocols as detailed above. For aging, perovskite samples (with a total surface area of 3 cm×3 cm) were immersed in 10-mL toluene in clear vials and exposed to the same aging environment as for unencapsulated perovskite cells. The iodine loss from perovskites was studied by preparing the aged toluene solution in a quartz cuvette and then measured using the UV-vis spectrophotometer.

Optical Modelling for Perovskite-On-Silicon Tandem Subcells

We modelled the optical response of the stack using the generalised transfer matrix method (Appl. Opt. 41, 3978-3987, 2002). All calculations were done in Python with use of the NumPy and SciPy libraries. Transfer matrix calculations take the complex refractive index spectrum and thickness for each layer as input. The calculation provides us with absorptance of each layer, and the transmittance and reflectance of the stack. We assumed perfect internal quantum efficiency and calculated the short circuit current as the overlap integral of the AM1.5 solar spectrum with the absorptance. To vary the band gap of the perovskite, the extinction coefficient was linearly translated. For each translation, the refractive index was recalculated using the Kramer's Kronig relation (Appl. Spectrosc. 42, 952-957, 1988): G

$$n ( λ ) = 1 + \frac{2}{π} {\int }_{0}^{∞} \frac{{E}^{′} k ( E )}{{E}^{′ 2} - {E}^{2}} {dE}^{′} \qquad ( S 2 )$$

The material stacks and optical data used for each material layer as input for the Transfer Matrix Calculations are the same as though which we used in a previous publication, Adv. Energy Mater. 9, 1803241 (2019), including the device stack which we used for modelling the monolithic perovskite-on-silicon tandem solar cells. For the optical model of the tandem solar cells, we used the optical data from the HTL-perovskite absorber layer-ETL stack in our single-junction perovskite cells. In addition, for the tandem cell simulation we used a SnO₂ buffer layer (5 nm), an ITO layer (80 nm) and a final antireflection coating, similar to the stack reported by Bush et al., Nat. Energy 2, 17009 (2017).

Charge extraction, transient photocurrent, transient photovoltage and photoluminescent. To understand the effect of [BMP]⁺[BF₄]⁻ on device performance, particularly on V_{OC}, light-induced charge carriers, charge carrier lifetime and effective charge carrier diffusion mobility were measured by a series of transient optoelectronic measurements, including charge extraction, transient photocurrent and transient photovoltage. These transient optoelectronic techniques have been widely applied to the study of the recombination and transport kinetics in dye-sensitized solar cells, organic solar cells, and perovskite solar cells.

FIG. 14A shows the light intensity-dependent V_{OC}, and the ideality factors extracted for the control and 0.25 mol % [BMP]⁺[BF₄]⁻ modified devices are 2 and 1.55, respectively. The smaller ideality factor suggests that the [BMP]⁺[BF₄]⁻ modified device has less recombination via deeper trap states. Regarding charge extraction, FIG. 14B shows the extracted total charge as a function of light-induced V_{OC}. We found that the obtained charge rises exponentially with the light-induced V_{OC}, with shallow gradients as a function of V_{OC}, indicative of trapped charge carrier distribution n is significantly larger than thermal energy kT. This exponential increase suggests that the photogenerated carriers fill intraband trap states as the quasi-Fermi level splitting increase. At smaller light-induced V_{OC}, the charge in the control device is significantly higher than the [BMP]⁺[BF₄]⁻ modified device, indicative of a higher density of relatively deeply trapped carriers in the control device, with [BMP]⁺[BF₄]⁻ reducing the density of relatively deep traps in the perovskite film.

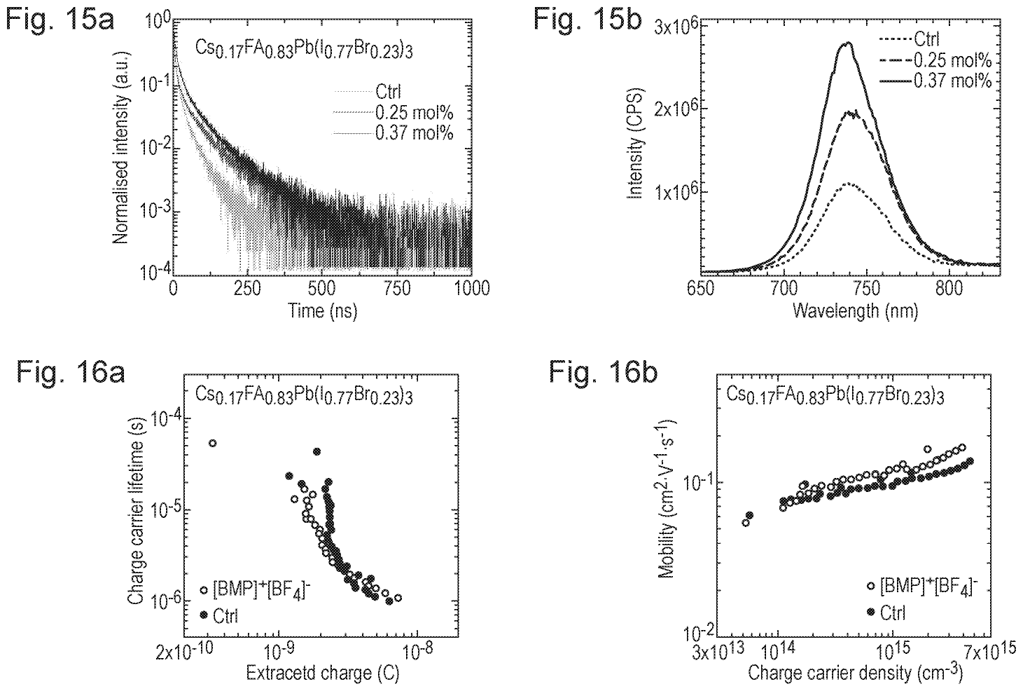

To shed more light on this point, we performed time-resolved and steady-state photoluminescence (i.e. TRPL and SSPL) on the control and [BMP]⁺[BF₄]⁻ modified films made on polyTPD/FTO substrates (FIG. 15). A slower initial TRPL decay and a higher SSPL observed in the [BMP]⁺[BF₄]⁻ device also indicate through the addition of [BMP]⁺[BF₄]⁻ the trap density in the perovskite film can be effectively reduced.

The effective charge carrier lifetime measured as a function of the total charge is shown in FIG. 16A. We found that the control device exhibits longer carrier lifetimes than the [BMP]⁺[BF₄]⁻ modified device at matched carrier densities, particularly in the low charge density regime. These longer carrier lifetimes are also indicative of a higher trap density for the control device as. In FIG. 16B, the effective diffusion mobility of both devices is similar, with the value of ˜10⁻¹ cm²·V⁻¹·s⁻¹ measured at a short circuit condition.

Evaluation of Time-Resolved Microwave Conductivity Figure of Merit

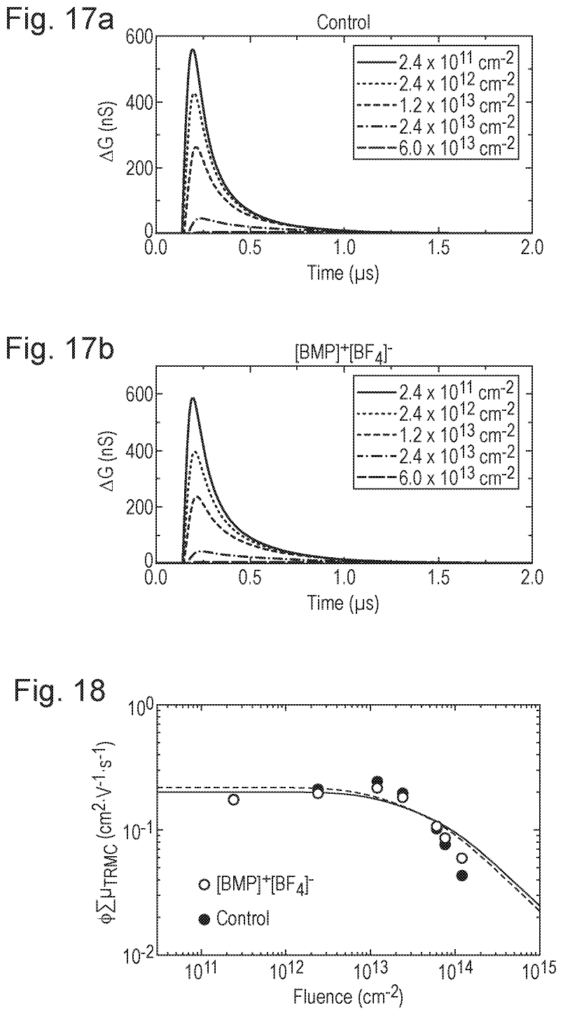

FIGS. 17A and 17B show the time-resolved microwave conductivity (TRMC) transient data (photo-conductance AG as a function of time t) for the control hybrid perovskite sample [i.e. Cs_{0.17}FA_{0.83}Pb(I_{0.77}Br_{0.23})₃] and the sample with 0.25 mol % [BMP]⁺[BF₄]⁻, respectively. The peak observed value of ΔG (i.e. ΔGmax) can then be used to determine the TRMC figure of merit through Eq. S3:

$$ϕ {Σμ}_{TRMC} = \frac{Δ {G}_{max}}{β {eI}_{0} {F}_{A} M} \qquad ( S 3 )$$

Here ϕΣμ_{TRMC}=ϕ(μₑ+μₕ), where ϕ is the carrier generation efficiency, μₑ and μₕ are the electron and hole mobility in the perovskite sample, respectively. e is the fundamental unit of charge. I₀ is the fluence of the incident light, and F_{A} is the fractional absorption of photons of the sample at the excitation wavelength (between 0 and 1). I₀ can be measured by placing a calibrated photodiode/thermopile in the path of the excitation path. F_{A} can be measured using ultraviolet-visible spectroscopy. M is a parameter we define as the “masking parameter” and is the fraction (between 0 and 1) of the cross-sectional area of the cavity that is exposed to the incident light. In our case M=0.25 in all cases.

Equation S3 assumes that no recombination takes place on the response-time of the measurement. At low fluence this is a reasonable assumption, but at high fluence the carrier density could reach a very high number. Under these conditions, bimolecular and Auger recombination can reduce the peak value of ΔG from what one would expect under ideal conditions. This is manifest as a reduction in ϕΣμ_{TRMC} as a function of fluence at high fluence. Using a simple model based on recombination during the finite duration of the laser pulse, this behaviour can be modelled. The specific details of the model can be found in J. Appl. Phys. 122, 065501 (2017).

Time-Resolved Microwave Conductivity and In-Plane Transient Photoconductivity

In order to further investigate the impact of the additive of charge carrier mobility, perovskite films were studied using time-resolved microwave conductivity (TRMC). TRMC was here carried out on isolated thin films on quartz. FIG. 18 shows the TRMC figure of merit ϕΣμ_{TRMC}=ϕ(μₑ+μₕ), for the control sample and the sample with the additive as function of optical fluence, where ϕ is the number of electron hole pairs generated per absorbed photon, and μₑ and μₕ are the average electron and hole mobilities over the sample area, respectively. The parameter #Ely has the same dimensions as the mobility (e.g. cm²V⁻¹s⁻¹) but carrier-type-specific information remains obscured.

At high optical fluence, a significant amount of bimolecular and Auger recombination will occur during the ˜5 ns laser pulse of the TRMC experiment, resulting in a reduction in peak observable photo-conductance, and hence a reduction in extracted ϕΣμ_{TRMC} (as observed in FIG. 18). This is a widely-observed phenomenon in TRMC, and models have been developed to account for it. We have applied this model to our experimental data to approximate representative values of ϕΣμ_{TRMC} for the films studied here. From these fits we evaluate ϕΣμ=0.23 cm²·V⁻¹·s⁻¹ for the control sample and ϕΣμ_{TRMC}=0.21 cm²·V⁻¹·s⁻¹ for the [BMP]⁺[BF₄]⁻ modified sample.

The results from TRMC hence broadly agree with those obtained from our charge extraction characterization. We note that while for charge extraction the measured mobility is in the out-of-plane direction, TRMC is an area-average local probe of electrical properties of the semiconductor in the plane of the sample.

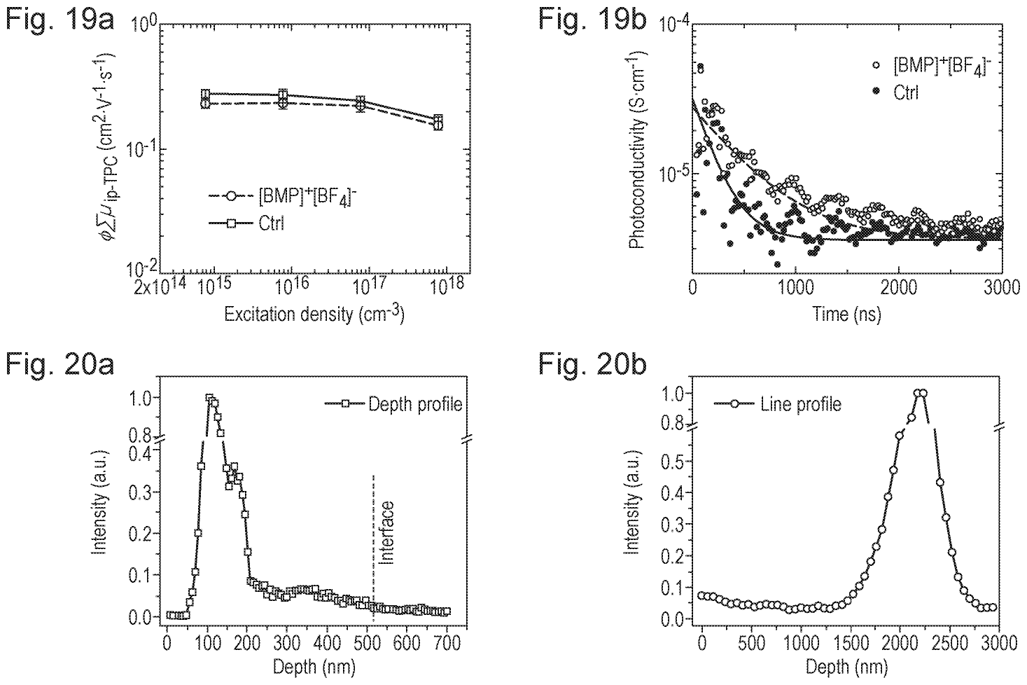

In addition, we have performed in-plane transient photoconductivity (ip-TPC) for both the control and [BMP]⁺[BF₄]⁻ samples (FIG. 19). The obtained mobilities ϕΣμ_{ip-TPC} are 0.26 cm²·V⁻¹·s⁻¹ and 0.23 cm²·V⁻¹·s⁻¹, respectively, as a function of the excitation density across three orders of magnitude (FIG. 19A). We note that these values are in good agreement with those obtained from our TRMC study. For the excitation density below 10¹⁷ cm⁻³ the mobility value is nearly constant as the excited charge carriers mostly exist as the free carrier under the equilibrium due to the low binding energy. From the decay profile of photoconductivity measured as a function of time after excitation (FIG. 19B), we found the [BMP]⁺[BF₄]⁻ modified sample has a much longer decay. The latter suggests a longer lifetime for the charge carriers in the [BMP]⁺[BF₄]⁻ device than the control one.

Solid-State Nuclear Magnetic Resonance

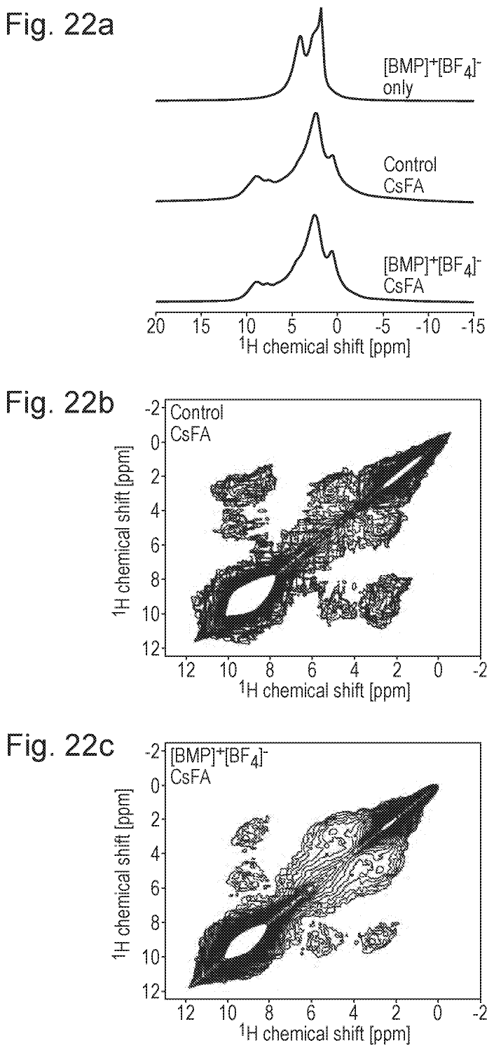

We examined if there was any direct interaction existing between [BMP]⁺[BF₄]⁻ and Cs_{0.17}FA_{0.83}Pb(I_{0.77}Br_{0.23})₃ perovskite through solid-state nuclear magnetic resonance (ssNMR) studies of the modified and control perovskites. The corresponding one-dimensional spectra and the 2D ¹H-¹H spin-diffusion (mixing time of 100 ms) spectra of the control and modified perovskite are shown in FIG. 22. A [BMP]⁺[BF₄]⁻ reference sample was used as the reference. The modified perovskite exhibited similar spectra to the control as most of the signature peaks of [BMP]⁺[BF₄]⁻ overlapped with those of Cs_{0.17}FA_{0.83}Pb(I_{0.77}Br_{0.23})₃(FIG. 22A). We also performed a ¹H-¹H 2D correlation experiment. Since, the cross-peaks obtained in both samples are similar (FIGS. 22B and 22C), this indicates either very weak or even no direct interaction between [BMP]⁺[BF₄]⁻ and perovskites.

X-Ray Photoemission Spectroscopy

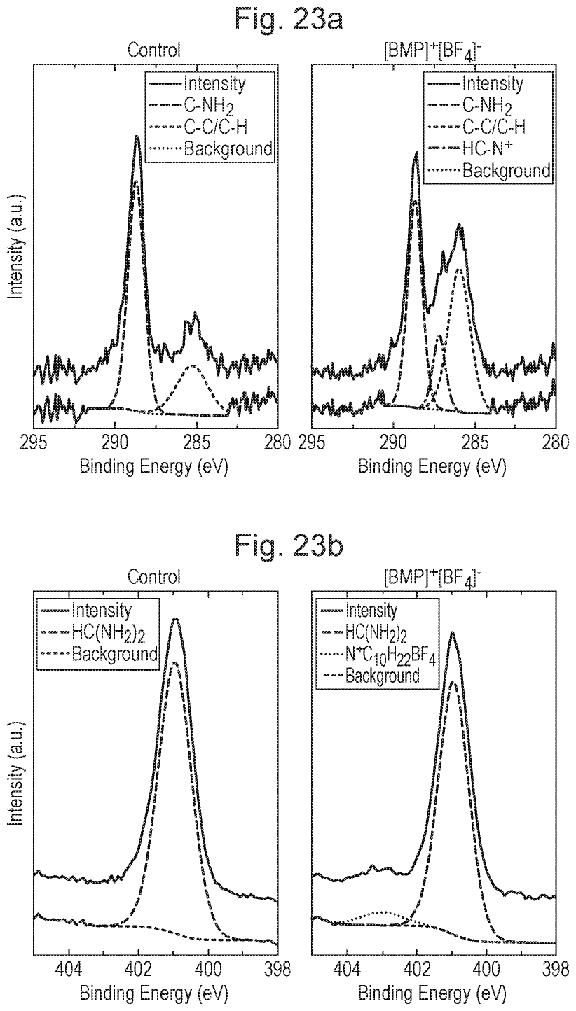

We further confirmed our findings from the ssNMR measurement by characterizing the modified and control samples using X-ray photoemission spectroscopy (XPS). FIGS. 23A and 23B show high-resolution scans of C 1s and N 1s, respectively. When comparing both the modified and control samples, there was barely any change observed in the A-site relevant peaks, i.e. C(NH₂)₂ (288.7 eV, FIG. 23A) and HC(NH₂)₂ (400 eV, FIG. 23B). This result again indicated that the interaction between [BMP]⁺[BF₄]⁻ and Cs_{0.17}FA_{0.83}Pb(I_{0.77}Br_{0.23})₃ was too weak to determine through the change in the chemical shift. However, because the peaks assigned to HC—N⁺ (286 eV, FIG. 23A) and N⁺ C₁₀H₂₂BF₄ (402 eV, FIG. 23B) were only found in the modified sample, this observation confirmed the presence of [BMP]⁺[BF₄]⁻ at the sample surfaces. These results indicated that it was unlikely that [BMP]⁺[BF₄]⁻ grew as part of the perovskite structures and only weakly interacted at the surface of the crystalline material, presumably between the crystalline grains within the film.

Equivalent Solar Illumination Intensity Measurement

The equivalent solar irradiance used to illuminate the PbI₂ films is given by the ratio of absorbed irradiance from the AM1.5G solar spectrum to that from the LED luminaire. The absorbed irradiance (Fᵢ) from illumination source (i) is given by:

Fᵢ=∫₀^{∞}A(λ)Pᵢ(λ)dλ (S4)

where λ is incident wavelength, A(λ) is the spectral absorptance of the material, and Pᵢ(λ) is the spectral irradiance. The equivalent solar irradiance (M) is thus:

M=F_{LED}/F_{AM1.5G} (S5)

To calculate the approximate equivalent intensity of the illumination source the short-circuit current (I_{SC}) from a KG3-filtered certified silicon reference diode (Fraunhofer), placed on top of the hotplate (the diode itself was thus ˜1 cm above the surface of the hotplate) under illumination by the luminaire, was measured using a source-measure unit (Keithley Instruments, 2400). The illumination spectrum from the luminaire was measured using a fiber-coupled spectrograph (Ocean Optics MAYA Pro 2000) with a cosine corrector on the light input aperture of the optical fiber. Dispersion in the optical measurement system was corrected using a calibration lamp of known spectral irradiance (Ocean Optics, HL-3P-CAL). The short circuit current density (J_{SC}) of a solar cell is given by:

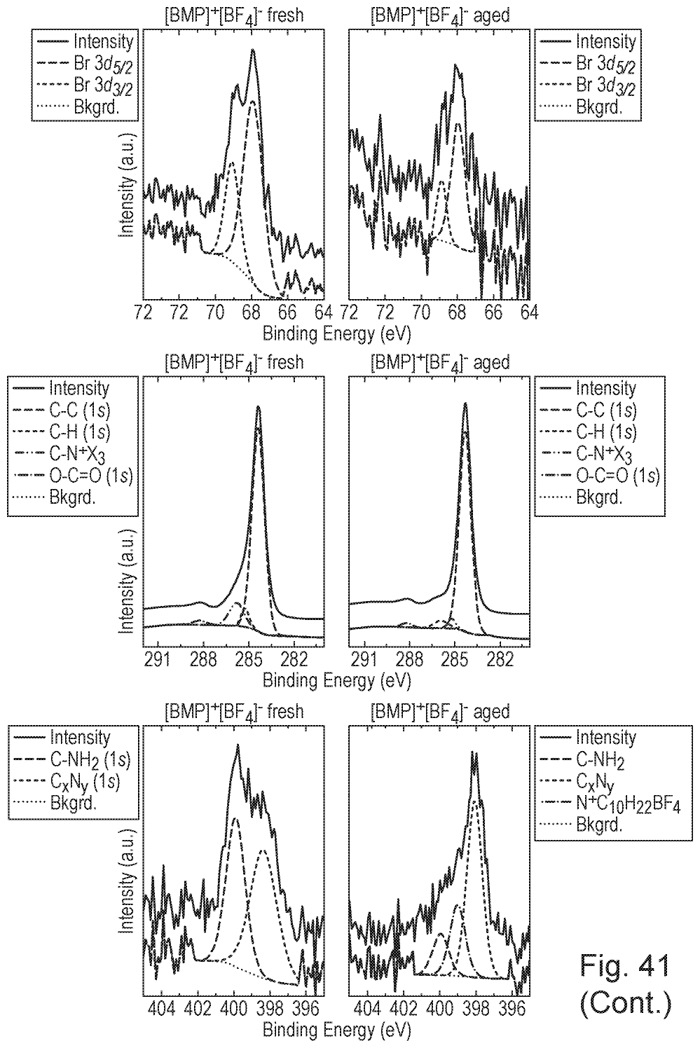

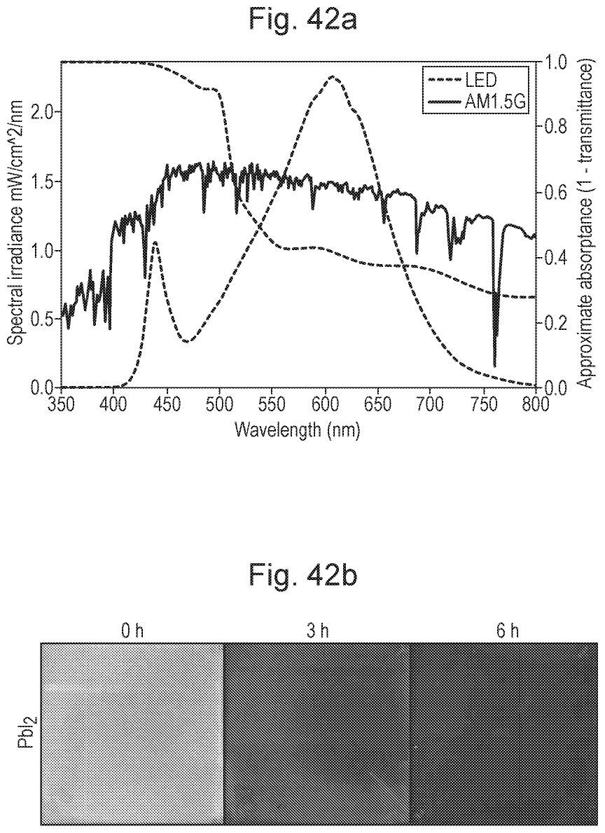

J_{SC}=∫₀^{∞}EQE(λ)ϕ_{i,norm}(λ)dλ (S6)

where q is the charge of an electron, EQE(λ) is the spectral external quantum efficiency, and ϕ_{i,norm} is the normalized incident photon flux from the illumination source, and N is a factor that scales the normalized photon flux spectrum to physical units. Therefore, using the measured J_{SC} and certified EQE of the calibration cell as well as the measured ϕ_{i,norm} from the LED array, it is possible to calculate N, P_{LED} in absolute units, as well as F_{LED} and F_{AM1.5G} using the measured PbI₂ absorptance spectrum (approximated as 1−T, where T is transmittance). Our setup gave equivalent solar intensity ≈0.32 suns (FIG. 42) when aging PbI₂ films in N₂.

Results

The results in this Example further demonstrate that ionic solid additives enable exceptional longevity for perovskite solar cells by effectively inhibiting the generation of degradation products. Longevity has been a long-standing concern for hybrid perovskite photovoltaics. This Example demonstrates high-resilience positive-intrinsic-negative perovskite solar cells by incorporating a piperidinium-based ionic-compound into the formamidinium-cesium lead-trihalide perovskite absorber. With the band gap tuned to be well suited for perovskite-on-silicon tandem cells, this piperidinium additive enhances the open-circuit voltage and cell efficiency. This additive also retards compositional segregation into impurity phases and pinhole formation in the perovskite absorber layer during aggressive aging. Under full-spectrum simulated sunlight in ambient atmosphere, unencapsulated and encapsulated cells retained 80% and 95% of their peak and “post-burn-in” efficiencies for 1010 and 1200 hours at 60 and 85 degree Celsius, respectively. Analysis reveals detailed degradation routes that contribute to the failure of aged cells.

Two-terminal monolithic perovskite-on-silicon tandem cells appear to be one of the most promising photovoltaic technologies for a near-term commercial-scale deployment. They feature a wide band-gap perovskite “top-cell” which absorbs in a complementary region of the solar spectrum, in comparison to the silicon “bottom-cell”, and such solar cells with a certified PCE reaching 29.1% have been demonstrated.

There is often a compromise between achieving high efficiency and long-term stability. The presence of methylammonium (MA) as the A-site cation in the perovskite absorber, which leads to more rapid decomposition under elevated temperature, light exposure, and atmosphere (4), can be alleviated by substitution with formamidinium (FA) or compositions of FA and cesium (Cs). However, the use of MA persists in many recent reports on the highest efficiency perovskite cells in the form of the mixed cation MA/FA/Cs or FA/MA perovskites. Also, the organic hole-conductor 2,2′,7,7′-tetrakis[N,N-di(4-methoxyphenyl)amino]-9,9′-spirobifluorene (Spiro-OMeTAD) and the “additives” required to deliver high efficiency are detrimental to the stability of perovskite cells, but often used in the highest PCE single-junction perovskite cells. Finally, molecular passivation of defects in the perovskite absorber, in order to deliver solar cells approaching the “radiative” efficiency limit, are often thermally unstable. The absorber layers and cells reverting to their “unpassivated” state, after thermal treatment at temperatures as low as 60° to 85° C. Efforts hence are required to deliver efficiency enhancements and improve long-term stability. This Example demonstrates high-performance p-i-n perovskite solar cells using “thermally-stable” Cs/FA-based lead-halide perovskite absorber layers, low-temperature processed organic charge extraction layers, and an organic ionic solid additive, 1-n-butyl-1-methylpiperidinium tetrafluoroborate ([BMP]⁺[BF₄]⁻). The incorporation of [BMP]⁺[BF₄]⁻ into the perovskite absorber suppressed deep trap states improved performance and enhanced the operational stability of cells stressed under full spectrum sunlight at elevated temperatures up to 85° C.

Perovskite Solar Cells with a Piperidinium Additive

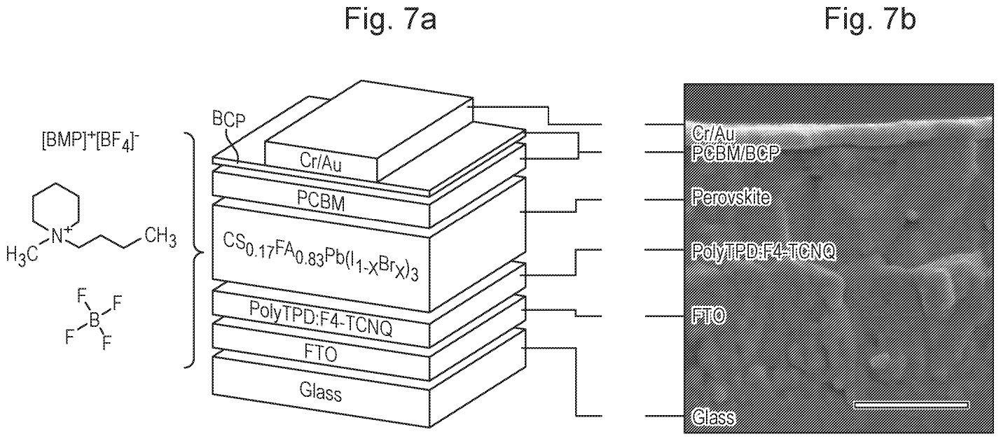

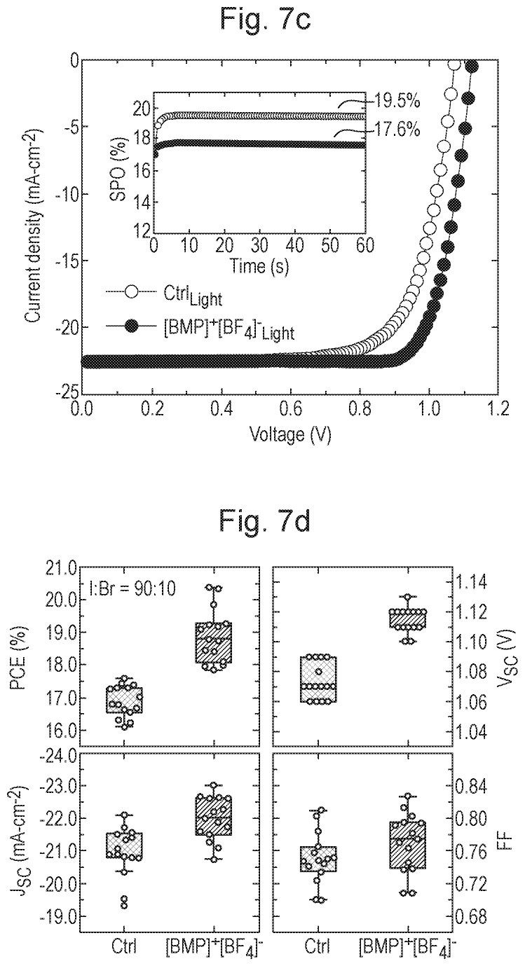

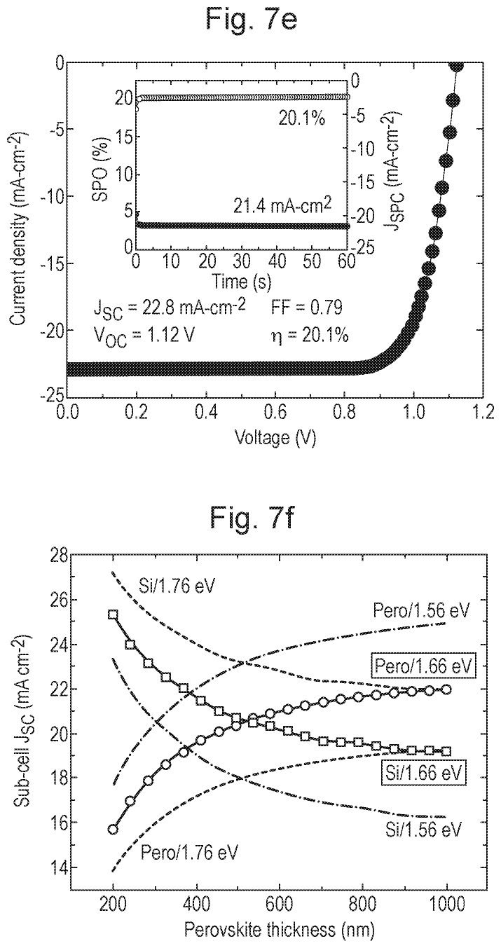

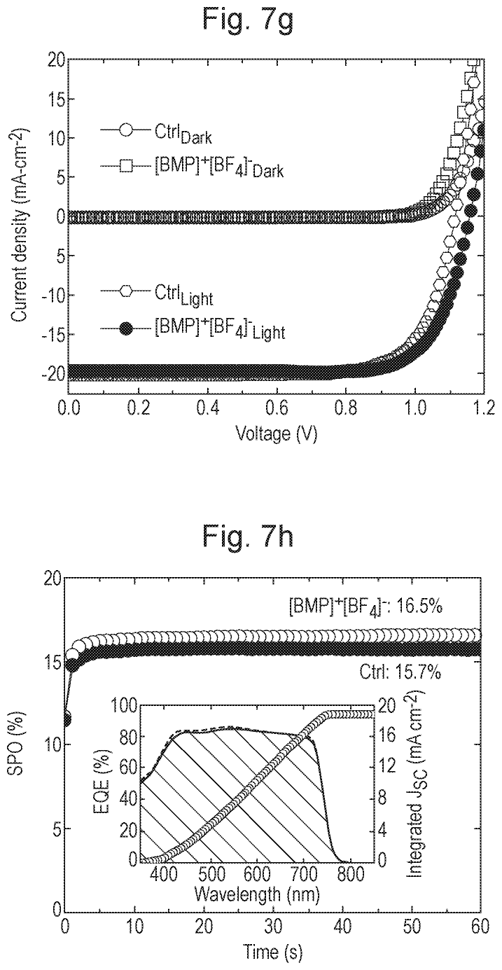

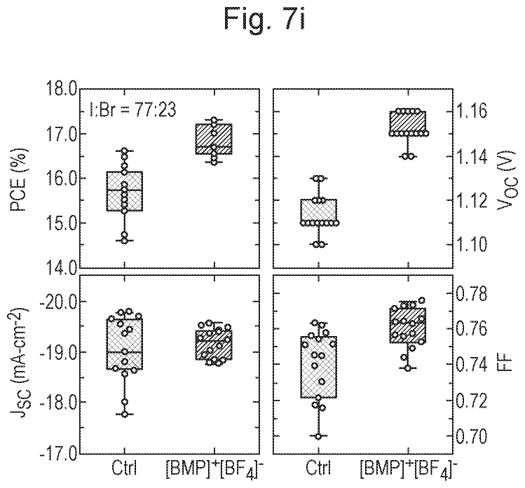

We screened a number of ionic salts as additives for improving the efficiency of perovskite solar cells, with the commonality of having a large chemically stable organic cation and [BF₄]⁻ anion. At low concentrations, [BMP]⁺[BF₄]⁻ (see FIG. 7A for the chemical structure) resulted in a particularly positive influence in photovoltaic performance. We depict the device architecture in FIG. 7A, where polyTPD and [6,6]-phenyl-C₆₁-butyric acid methyl ester (PC₆₁BM) were used as the hole-transporting and electron-transporting layers, respectively. The scanning electron microscopy (SEM) image for a representative p-i-n cell based on a perovskite composition of C_{0.17}FA_{0.83}Pb(I_{0.77}Br_{0.23})₃ and 0.25 mol % [BMP]⁺[BF₄]⁻ (with respect to the Pb content) is shown in FIG. 7B.

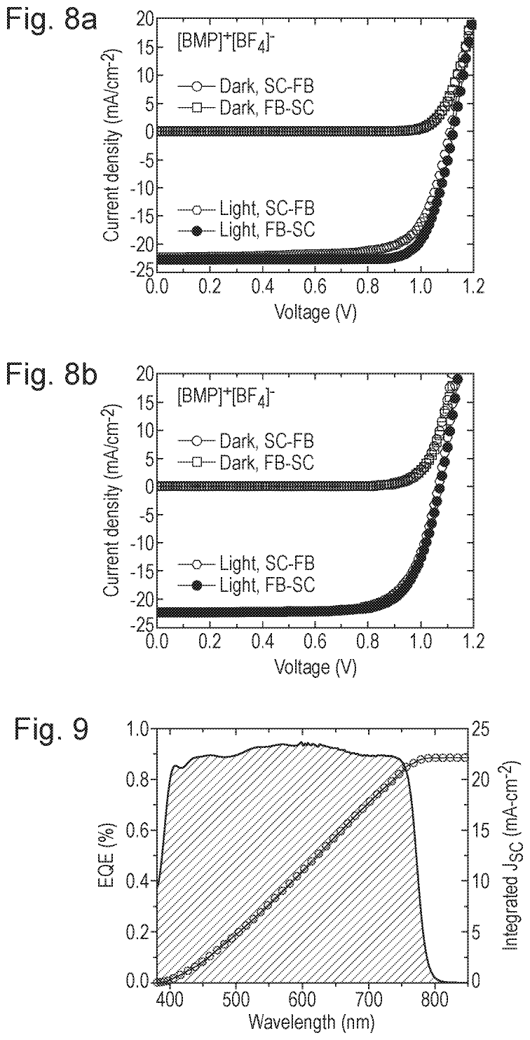

To demonstrate the potential of [BMP]⁺[BF₄]⁻ for the performance enhancement, we fabricated mixed halide perovskites with a low Br content Cs_{0.17}FA_{0.83}Pb(I_{0.90}Br_{0.10})₃, which we have found to be the best composition for maximum efficiency of single-junction cells. In FIG. 7C and FIG. 8 we show typical current density-voltage (J-V) characteristics for the 0.25 mol % [BMP]⁺[BF₄]⁻ modified and control devices with the statistical results of the device performance parameters shown in FIG. 7D. A champion [BMP]⁺[BF₄]⁻ device (FIG. 7E) exhibited an open-circuit voltage (V_{OC}) of 1.12 V, a short-circuit current density (J_{SC}) of 22.8 mA·cm⁻² and a fill factor (FF) of 0.79, resulting in a PCE of 20.1% and a steady-state power output (SPO) of 20.1%. The corresponding external-quantum efficiency (EQE) (FIG. 9) yielded an integrated J_{SC} with a negligible variation (˜2.5%) from the measured J_{SC}. The addition of [BMP]⁺[BF₄]⁻ in the perovskite light absorber led to very high performance for “MA-free” single-junction p-i-n perovskite solar cells compared to reports to date (D. P. McMeekin et al., A mixed-cation lead mixed-halide perovskite absorber for tandem solar cells. Science 351, 151-155 (2016); S. H. Turren-Cruz, A. Hagfeldt, M. Saliba, Methylammonium-free, high-performance, and stable perovskite solar cells on a planar architecture. Science 362, 449-453 (2018)).

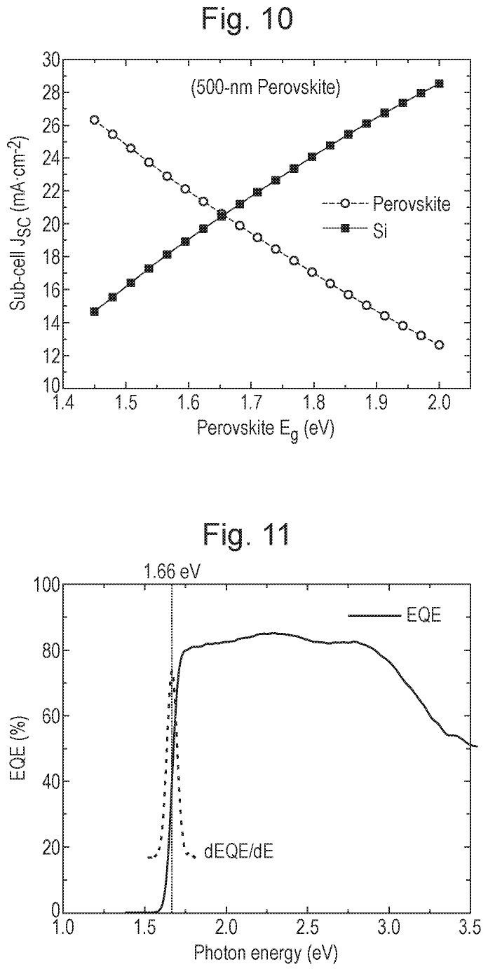

For a perovskite-on-silicon tandem solar cell, balancing the light absorption between the constituent subcells is key to achieve current matching to maximize PCE. Following Mazzarella et al., Adv. Energy Mater. 9, 1803241 (2019) we simulated the evolution of subcell JSC values in perovskite-on-silicon tandem cells as a function of absorber layer thickness for perovskite band gaps of 1.56, 1.66, and 1.76 eV (FIG. 1F). This ideal thickness needed for a 1.66 eV band gap was ˜500 nm, which falls into a common perovskite processing window (Turren-Cruz et al., Science 362, 449-453 (2018)). We also modeled the subcell J_{SC} with various band gaps for a 500-nm perovskite layer (FIG. 10), and a 1.66 eV band gap was also nearly ideal for maximizing energy yield for monolithic perovskite-on-silicon tandem cells deployed in real-world locations (M. T. Hörantner, H. J. Snaith, Energy. Environ. Sci. 10, 1983-1993, 2017).

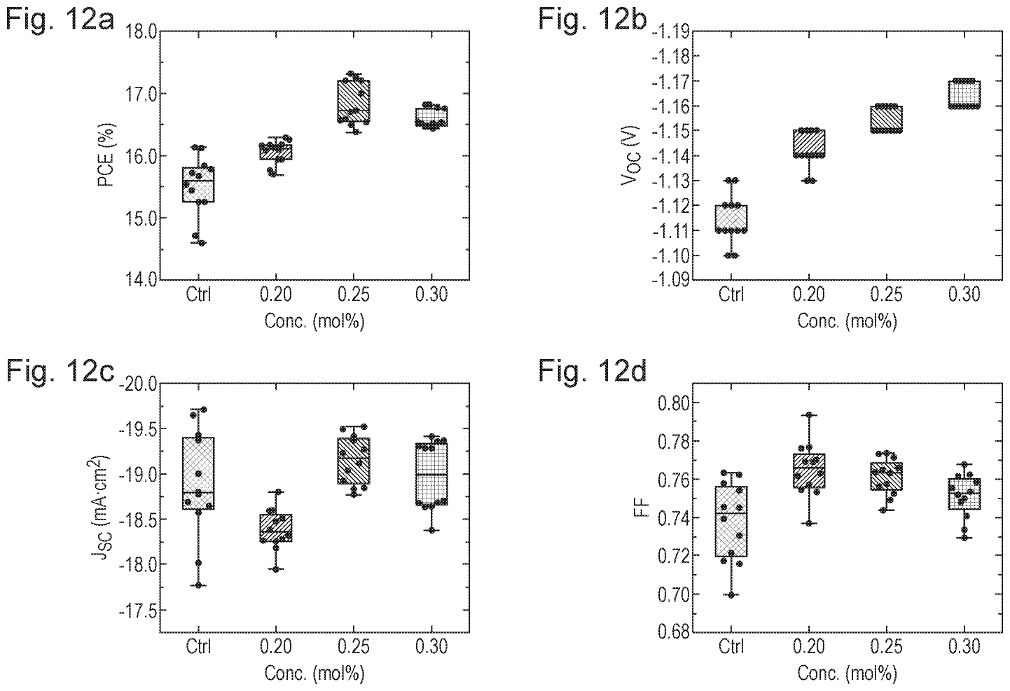

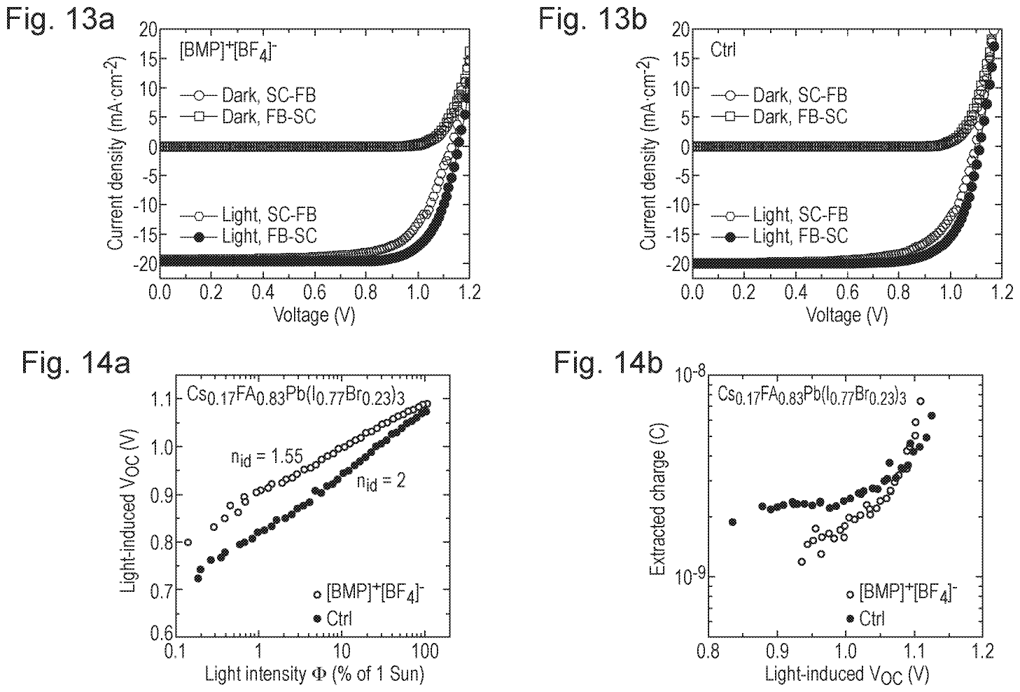

By tuning the I/Br composition, we found that Cs_{0.17}FA_{0.83}Pb(I_{0.77}Br_{0.23})₃ perovskite delivered the desired 1.66 eV band gap, as determined from the derivative of the EQE spectrum, (FIG. 11). We optimized the single-junction cells using different [BMP]⁺[BF₄] concentrations ranging from 0.0 (control) to 0.3 mol %, and summarize the device performance parameters from a large batch of cells in FIG. 12. With increasing concentrations of [BMP]⁺[BF₄]⁻, we observed that V_{OC} rose from an average of 1.11 V for the control device to >1.16 V for the 0.3 mol % [BMP]⁺[BF₄]⁻ modified device; J_{SC} did not vary appreciably relative to the control. However, on average, the FF increased at low concentrations but tended to decrease at higher concentrations of [BMP]⁺[BF₄]⁻. Thus, devices with 0.25 mol % [BMP]⁺[BF₄]⁻ exhibited the highest PCEs. Characteristic J-V curves for an optimized 0.25 mol % [BMP]⁺[BF₄]⁻ modified perovskite solar cell and a control device are shown in FIG. 7G, and the corresponding SPOs are shown in FIG. 7H. The corresponding forward and reverse direction J-V scans are shown in FIG. 13. The device comprising 0.25 mol % [BMP]⁺[BF₄]⁻ exhibited a V_{OC} of 1.16 V, a J_{SC} of 19.5 mA·cm⁻² and a FF of 0.77, yielding a PCE of 17.3%. The control device, which exhibited a lower PCE of 16.6%, had a V_{OC} of 1.11 V and a FF of 0.75. The corresponding SPOs were 16.5% and 15.7% for the modified and control devices, respectively. We show a set of the statistical results obtained from 15 individual cells of each type in FIG. 7I. The external quantum efficiency (EQE) (the inset of FIG. 7H) was in good agreement with the J_{SC} measured from the J-V scans (FIG. 7G). With the addition of [BMP]⁺[BF₄]⁻, the cells generally exhibited an increase in V_{OC}, FF and PCE. The J_{SC} was similar or slightly higher with the optimum piperidinium content for all perovskite compositions.

Optoelectronic and Material Analyses of Fresh and Aged Perovskites

To understand the impact upon the optoelectronic characteristics of the perovskite films with the addition [BMP]⁺[BF₄]⁻, we carried out a series of spectroscopic measurements, including transient photovoltage (TPV, FIG. 14A), charge extraction (FIG. 14B), time-resolved photoluminescence (TRPL, FIG. 15A), steady-state photoluminescence (SSPL, FIG. 15B), and transient photoconductivity (TPC, FIG. 16) on half-complete or complete device structures, and time-resolved microwave conductivity (TRMC, FIGS. 17-18) and in-plane transient photoconductivity (ip-TPC, FIG. 19) on isolated perovskite films. We found that adding [BMP]⁺[BF₄]⁻ did not compromise charge carrier mobilities (FIGS. 16B, 18 and 19A). More importantly, from light-intensity dependent V_{OC} and charge-extraction measurements of complete devices, we observed a reduced ideality factor and capacitance (or reduced total stored charge density) for the [BMP]⁺[BF₄]⁻ modified devices under low-light intensity (FIG. 14B). We also observed a slower TRPL decay and more than doubling of the SSPL intensity in the [BMP]⁺[BF₄]⁻ film (FIG. 15). These results were consistent with a reduced density of deep trap sites in the [BMP]⁺[BF₄]⁻ modified devices. Further analysis of the optoelectronic and spectroscopic characterizations is provided elsewhere in Example 2.

To reveal how [BMP]⁺[BF₄]⁻ was distributed within the perovskite layer, we used high-resolution secondary-ion mass spectrometry (NanoSIMS). We present the secondary electron and elemental mapping for the ¹⁹F⁻ and ¹¹B¹⁶O₂⁻ distributions in a Cs_{0.17}FA_{0.83}Pb(I_{0.77}Br_{0.23})₃ perovskite film in FIG. 21, A to C. In FIG. 21A, the ¹⁹F⁻ signals show agglomeration and despite yielding much lower intensities, the ¹¹B¹⁶O₂⁻ intensity map (FIG. 21B) coincided reasonably well with the ¹⁹F⁻ map. We show the three-dimensional (3D) visualization of the entire ¹⁹F⁻ dataset in FIG. 21D, where we observed that the ¹⁹F⁻ signal originated from roughly spherical regions a few hundred nanometers in diameter that were evenly distributed over the surveyed volume. Both the depth (FIG. 20A) and line (FIG. 20B) profiles revealed that a small amount of F could be detected throughout the perovskite, in addition to the agglomerates.

From this NanoSIMS characterisation we deduce that most of the [BMP]⁺[BF₄]⁻ molecules were localized in isolated aggregates that presumably accumulated between the perovskite domains, but small amounts penetrated the entire volume of the film. This distribution is in contrast to the imidazolium-based ionic liquid, which we have previously used with NiO p-type layers. For that material, the predominant accumulation of [BF₄]⁻ was at the buried NiO-perovskite interface (S. Bai et al., Nature 571, 245-250, 2019). Presumably, the distribution throughout the entire volume of the perovskite film helped the [BMP]⁺[BF₄]⁻ ionic salt enhance the performance of the cells when we used the poly-TPD organic hole-conductor. We attempted to observe interactions between the [BMP]⁺[BF₄]⁻ and the perovskites via solid-state nuclear magnetic resonance (ssNMR) and X-ray photoemission spectroscopy (XPS), which we show in this Example and in FIGS. 22 and 23, respectively. However, we observed no discernible differences.

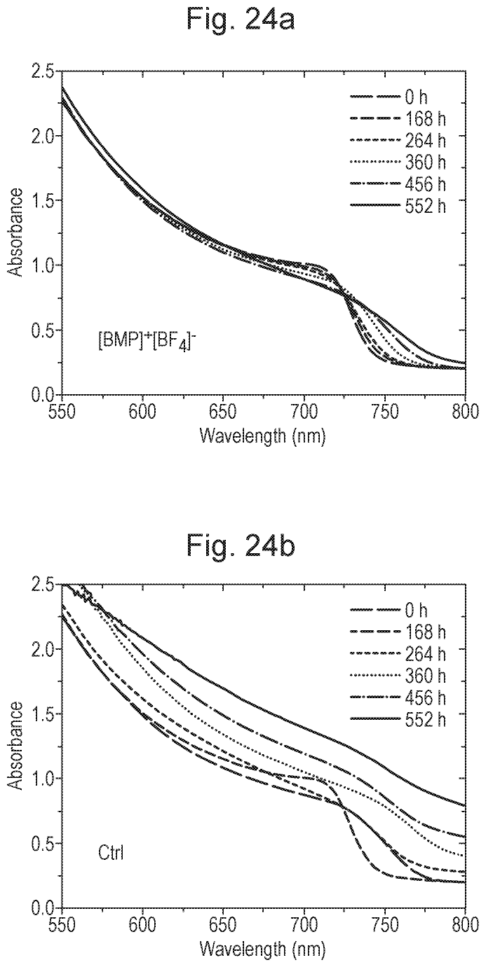

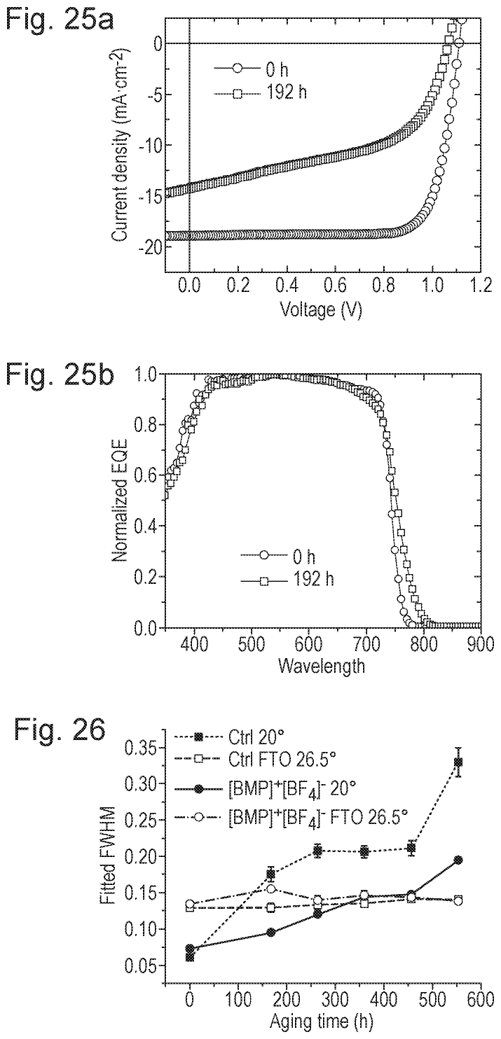

We also carried out characterizations to assess the stability of the Cs/FA perovskite compounds upon the addition of [BMP]⁺[BF₄]⁻. Ultraviolet-visible (UV-vis) absorption spectra (FIG. 24) and X-ray diffraction (XRD) patterns (FIGS. 21, E and F) were obtained of the [BMP]⁺[BF₄]⁻ modified and control Cs_{0.17}FA_{0.83}Pb(I_{0.77}Br_{0.23})₃ perovskite films aged under simulated full-spectrum sunlight at 60° C. in ambient air (relative humidity in the laboratory ˜50%). The absorption edge of the [BMP]⁺[BF₄]⁻ modified sample exhibited a minor change for the first 264 h (FIG. 24A), while the control sample exhibited a clear redshift in the absorption edge, which moved from ˜750 nm to >775 nm (FIG. 24B). This redshift in absorption, also coincides with a redshift in the EQE spectrum of complete solar cells aged in a similar manner (FIG. 25), indicating that a similar change is occurring, albeit at a slower rate, in the complete devices. The XRD measurements did not reveal any noticeable formation of lead halide (˜12.7°) with aging time for both the control (FIG. 21E) and modified (FIG. 21F) samples, which is usually observed during degradation of MA containing perovskites due to the loss of MAI (Habisreutinger et al., APL Mater. 4, 091503, 2016). Conversely, a small PbI₂ peak present at 12.7° in both the control and modified films at early time, disappeared during aging. During the time series, the main perovskite phase peaks broadened (FIG. 26) and decreased in intensity, and additional peaks at 14.6° and 20.7°, as well as a low-angle peak at 11.3°, appeared for long aging in the control film.

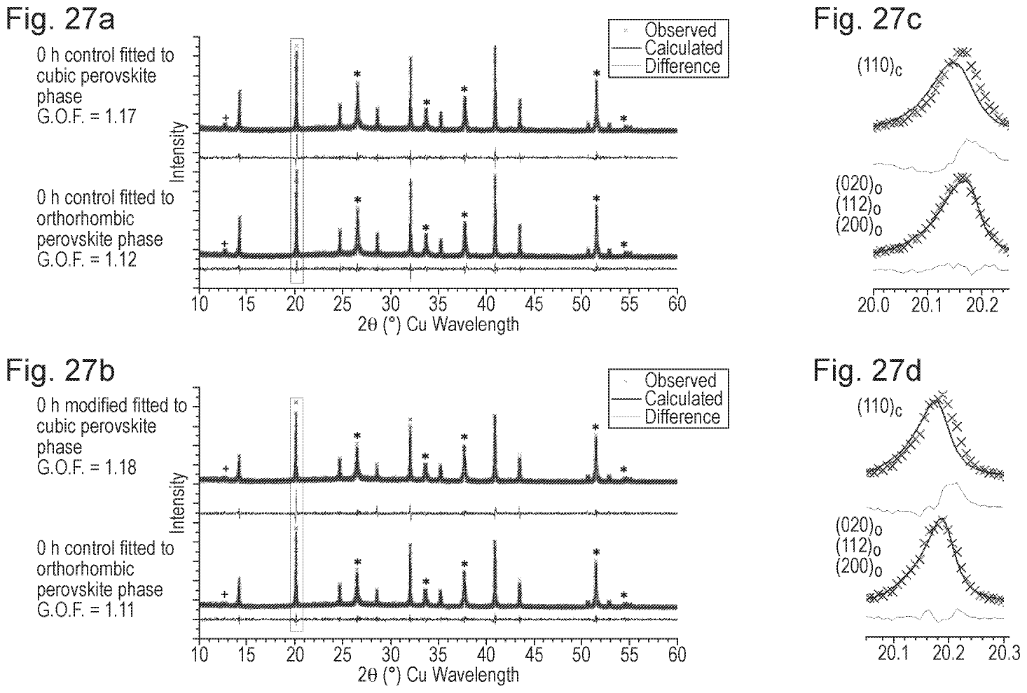

The broadening of the main phase can be explained by orthorhombic strain. Before aging, we fit the main perovskite phase to an orthorhombic cell in space group Pnma, with lattice parameters of a=8.801(1)Å, b=8.8329(3) Å, c=12.4940(5) Å, vol=971.3(2) Å³ for the control film, and a=8.8146(3) Å, b=8.8333(9) Å, c=12.4892(9) Å for the modified film with a larger volume of 972.4(1) Å³. Both are larger than the orthorhombic perovskite γ-CsPbI₃(vol=947.33(5) Å³), indicating the mixed Cs/FA phase. The lowering of symmetry from cubic to orthorhombic was needed to fit the XRD data well (FIG. 27). We refined the orthorhombic unit cell across the aging series, and define the orthorhombic strain as

$$S ( \% ) = 100 \times \sqrt{{( \frac{\sqrt{2} a}{\sqrt{2} b} - 1 )}^{2} + {( \frac{\sqrt{2} b}{c} - 1 )}^{2} + {( \frac{\sqrt{2} a}{c} - 1 )}^{2}} ,$$

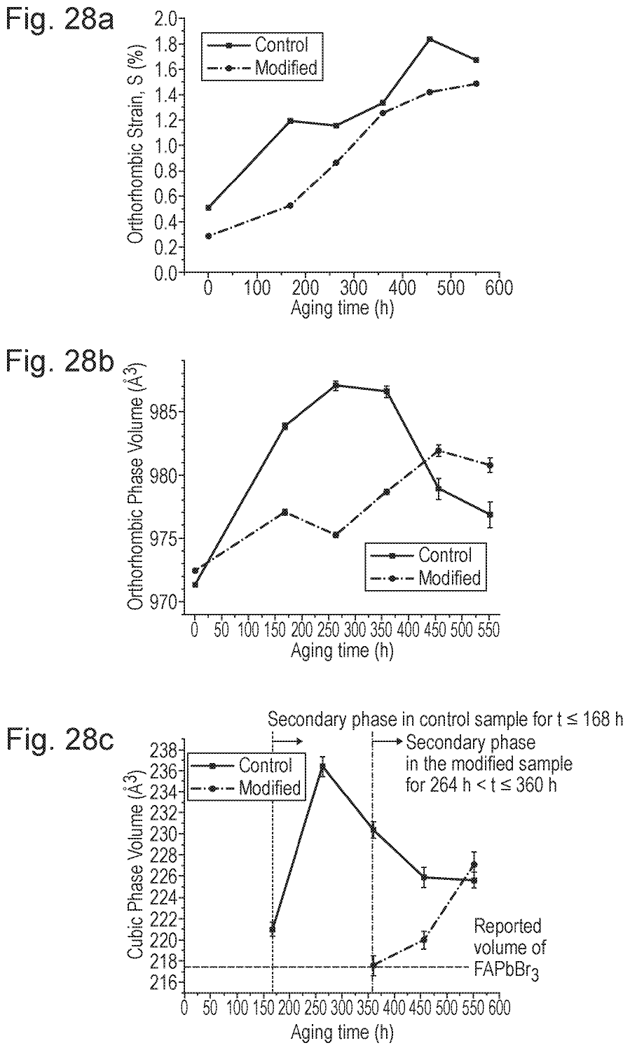

which we show in FIG. 28A. The orthorhombic strain in the control and modified films increased at a similar rate; however, the control sample started with a slightly more orthorhombic phase.

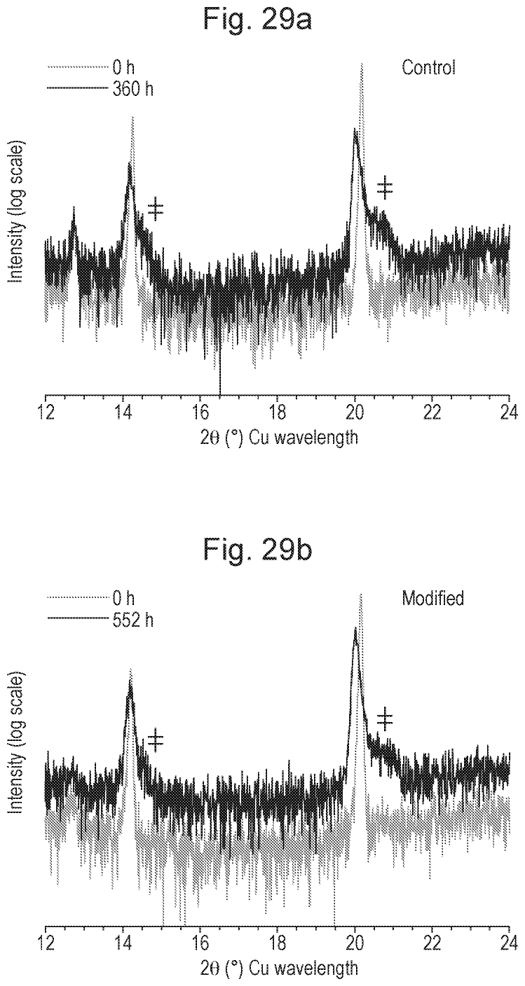

The orthorhombic strain was the only sign of change in the XRD pattern for the [BMP]⁺[BF₄] modified samples at aging times less than 360 h. Comparison of spectra showed that the orthorhombic strain did not have a large effect on the absorption. The additional peaks at 14.6° and 20.7° appeared for the control sample after the first aging step of 168 h and for the modified sample between the 264 and 360 h aging. These peaks were fitted to a cubic unit cell in the Pm3̅m space group and could not be fitted with the unit cells of any of the relevant binary halide salts. For the modified sample aged at 360 h the cubic unit cell has a volume of 217(1) Å³, which is within error of the reported volume of FAPbBr₃ (217.45(2) Å³). We indicate the XRD peaks associated with this second phase by (‡) in FIGS. 21E, 21F and 29.

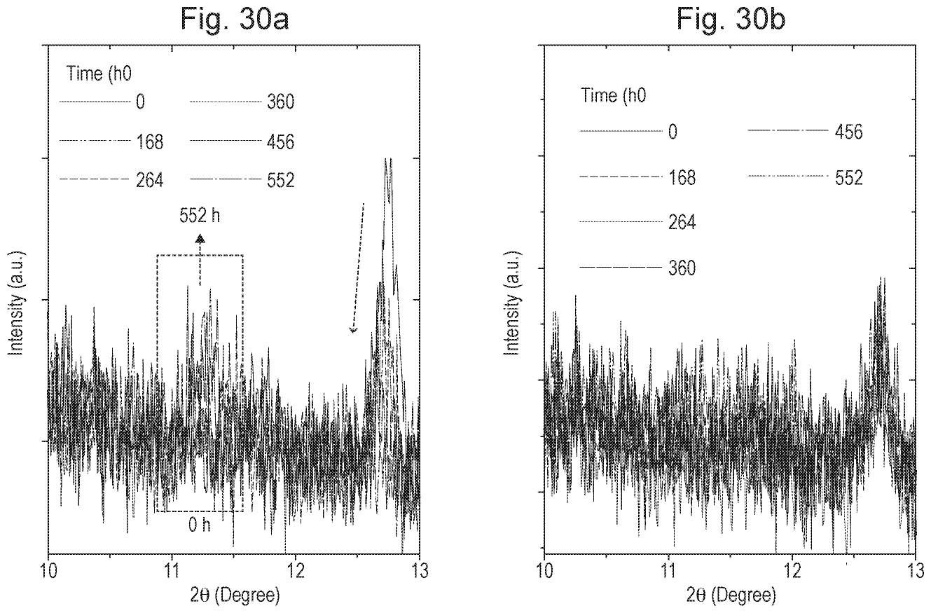

Segregation of FAPbBr₃, would leave the main phase Cs and I rich. Iodide enrichment was consistent with the redshift seen in absorption spectra (FIG. 24), and the time at which these phases emerged in the XRD patterns coincide with the timing for the redshift. The volume of the main orthorhombic perovskite phase and the secondary FAPbBr₃ perovskite phase both initially increased over time but started to decrease for the control after the 264-h aging (FIG. 28, B and C). This decrease suggests that after the initial separation of FAPbBr₃, other compositional changes continued, either because of mixing of the halides or external factors. At the same time, the intensity of the main phase peaks decreased, and the decrease was faster in the control sample. The peak at 11.3°, which appeared in the control sample after 456 h (FIG. 30) but was suppressed in the modified sample, was previously ascribed to the non-perovskite yellow hexagonal δ-FAPbI₃ phase (Energy. Environ. Sci. 10, 361-369 (2017); Chem. Mater. 28, 284-292 (2016)) that can form in the perovskite film when the Cs or FA content is strongly unbalanced (Adv. Energy Mater. 5, 1501310 (2015); Energy. Environ. Sci. 10, 361-369 (2017); Energy. Environ. Sci. 9, 656-662 (2016)).

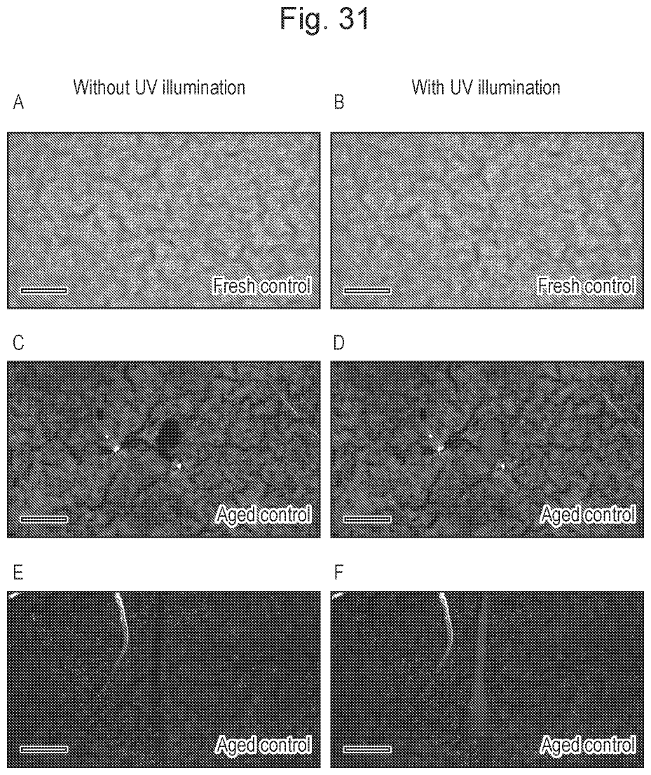

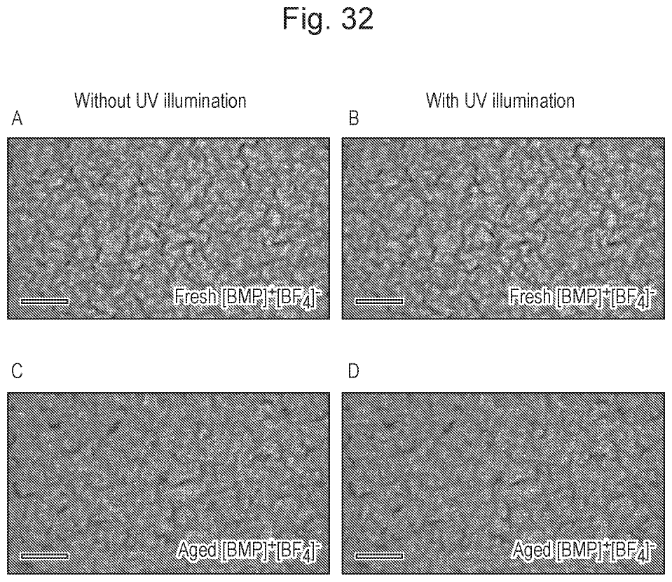

In an attempt to visualize the impurity phases generated during aging, we performed optical microscopy measurements on the fresh and aged control (FIG. 31) and [BMP]⁺[BF₄]⁻ modified (FIG. 32) Cs_{0.17}FA_{0.83}Pb(I_{0.77}Br_{0.23})₃ perovskite films grown on FTO glass. The aged samples were subjected to the same aging environmental parameters as applied to the XRD samples for 500 hours. The microscope was backlit with a halogen lamp, with the optional additional photoexcitation from the front with a 375-nm UV light-emitting diode (LED), in order to induce PL. Both the fresh control and [BMP]⁺[BF₄]⁻ modified films appeared orange-red in color and had wrinkled surface characteristic of the antisolvent quenching spin-coating fabrication method (ACS Energy Lett. 3, 1225-1232, 2018), and showed no clear difference with and without the UV illumination. After aging, the [BMP]⁺[BF₄]⁻ modified films appeared to be predominantly unchanged.

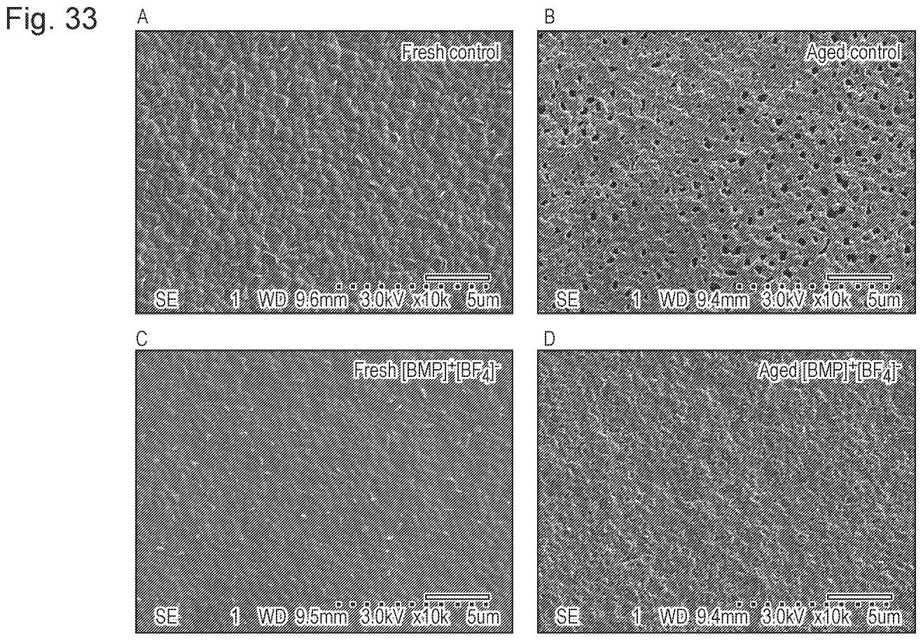

In contrast, for the control films, we observed a strong darkening in color and the appearance of large dark domains. Upon UV illumination, these dark domains emitted blue light. The blue emission was consistent with these regions containing some wider gap impurity phase material, most likely the non-perovskite hexagonal δ-FAPbI₃ phase (Energy. Environ. Sci. 10, 361-369 (2017); Chem. Mater. 28, 284-292 (2016)). In addition to these coarse features, we observed numerous white/yellow bright spots in images of the aged control samples (FIG. 31, C to F). From SEM images on the same samples, which we present in (FIG. 33), we confirmed that these “bright spots” were pinholes in the film. The presence of these pinholes in the aged control films, which were absent from the [BMP]⁺[BF₄]⁻ modified films, was a key difference. The addition of [BMP]⁺[BF₄]⁻ appeared to have inhibited the formation of the blue emitting impurity phase, FAPbBr₃ impurity phase growth, and importantly, strongly suppressed pinhole formation (FIG. 33D).

Long-Term Stability of Perovskite Solar Cells

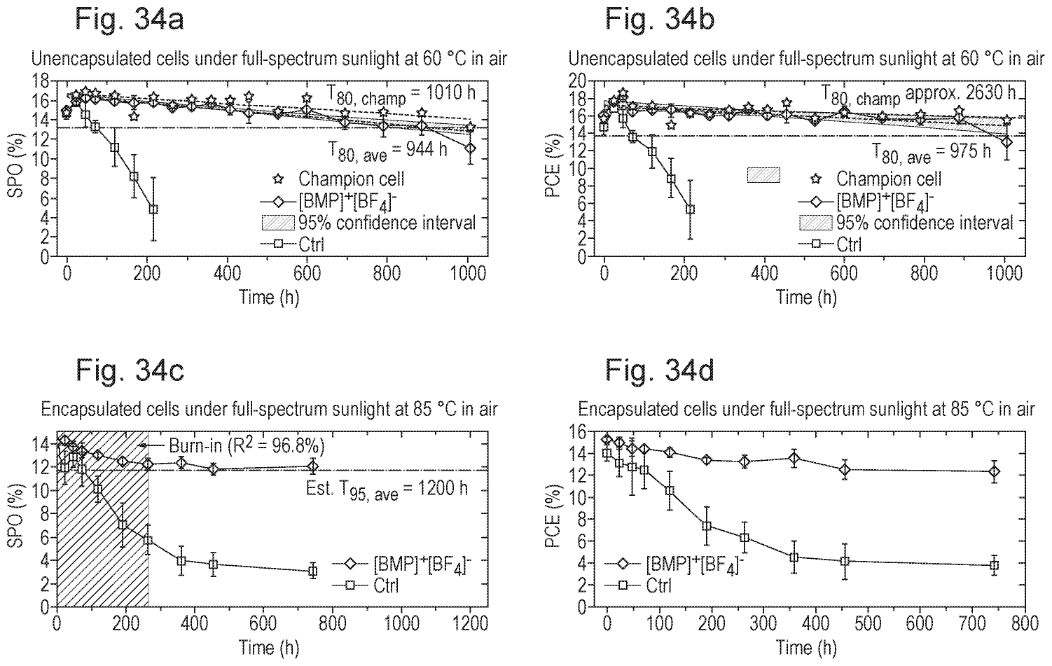

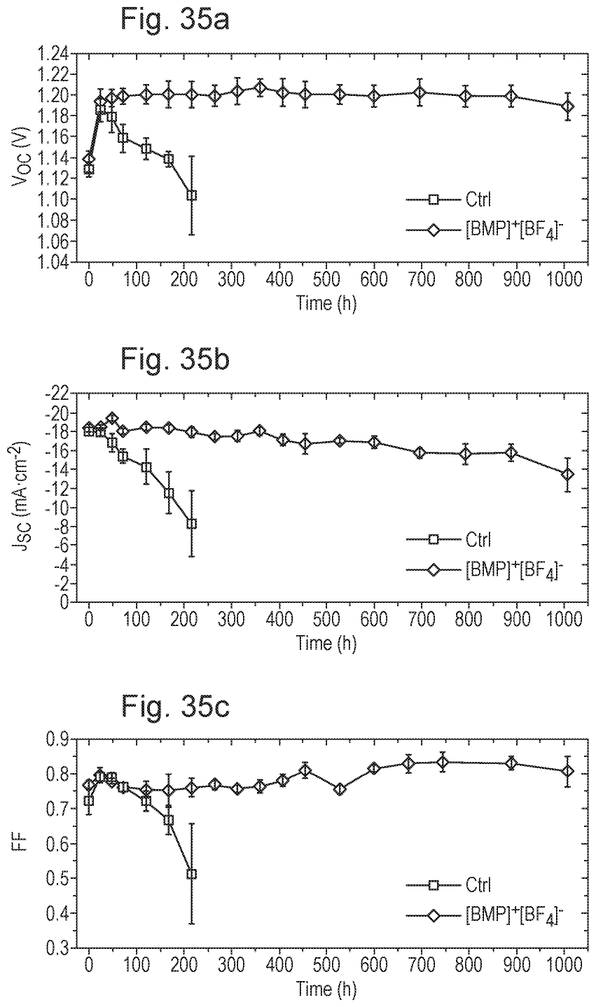

We investigated the operational stability of Cs_{0.17}FA_{0.83}Pb(I_{0.77}Br_{0.23})₃ based perovskite solar cells aged at open-circuit condition under full-spectrum sunlight at elevated temperatures in ambient air (relative humidity in the laboratory ˜50%). We first examined the stability of unencapsulated devices aged at 60° C. The average SPOs and PCEs obtained from 8 individual devices for each condition are shown in FIG. 34, A and B, respectively, and the evolution of the device parameters is plotted in FIG. 35. For both the [BMP]⁺[BF₄]⁻ modified and control devices, we observed a positive light-soaking effect that enhanced the SPO and PCE values by ˜2% absolute during the first few days of aging, whereas the average SPO and PCE of the control devices dropped below the initial performance after 72 h and continuously decrease to ˜5% absolute efficiency after 216 h. The efficiency of the [BMP]⁺[BF₄] modified devices improved over the first few hundred hours, likely to be due to the “photo-brightening” effect resulting from passivation of defects in the perovskite film, via reaction with photo-generated superoxide and peroxide species.

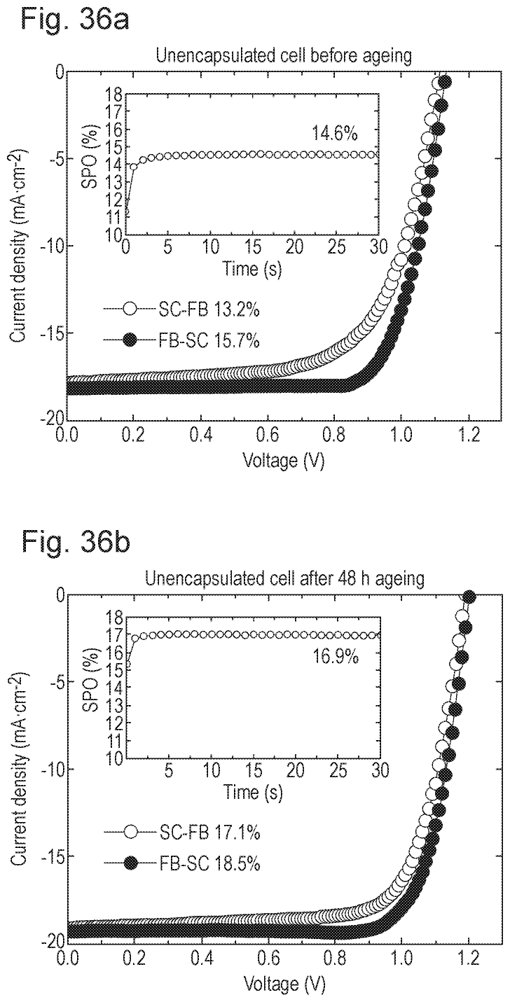

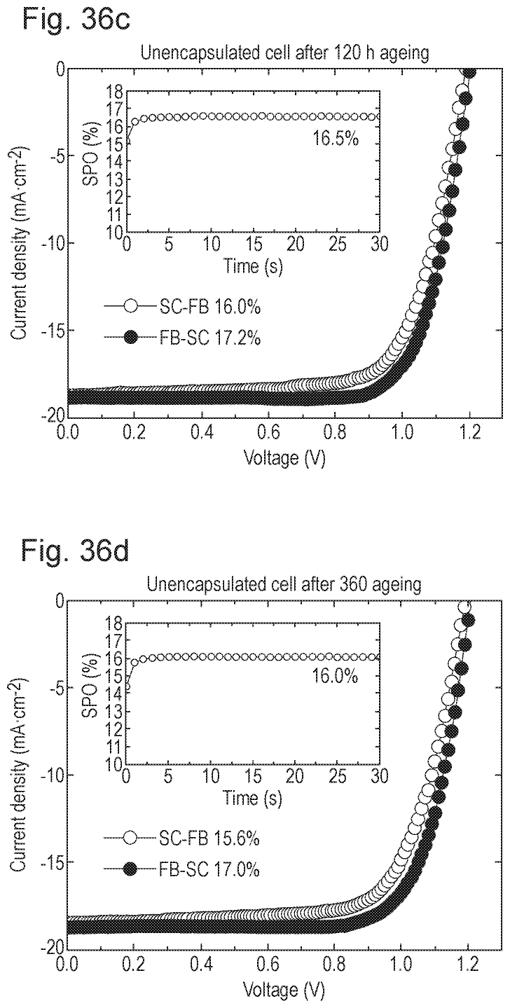

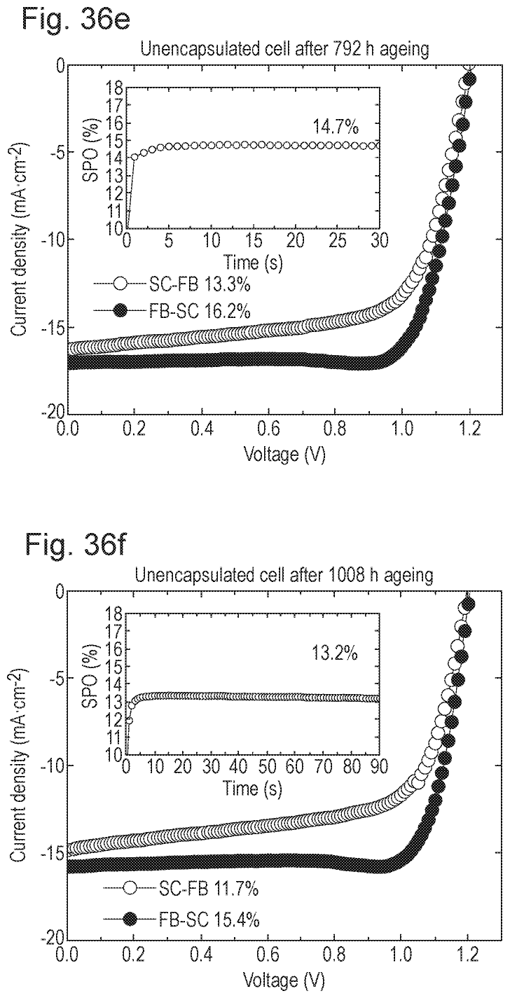

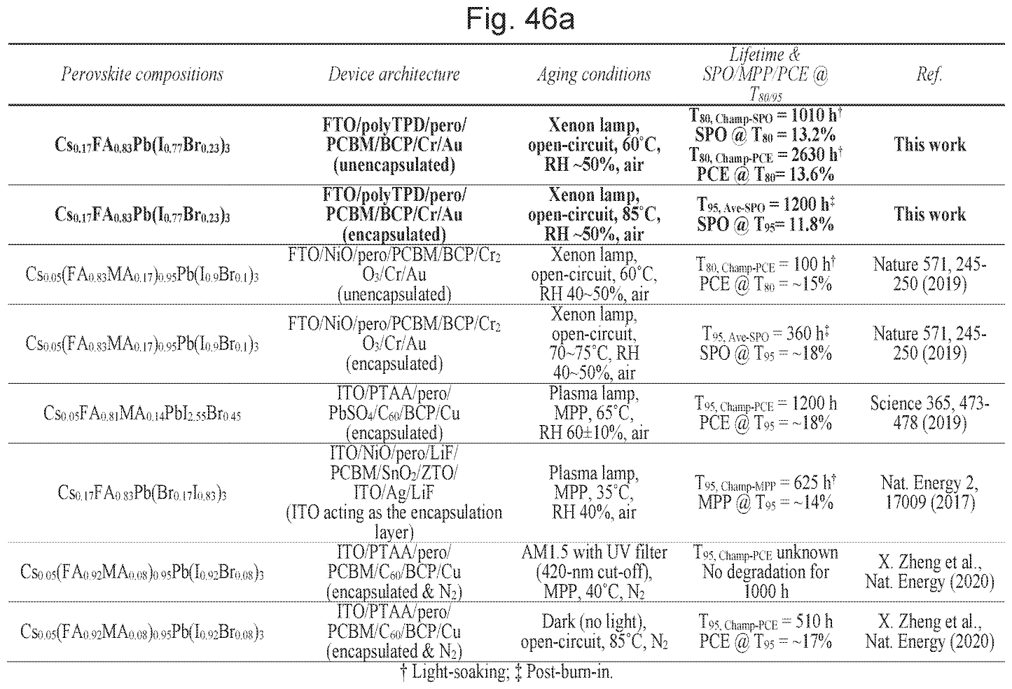

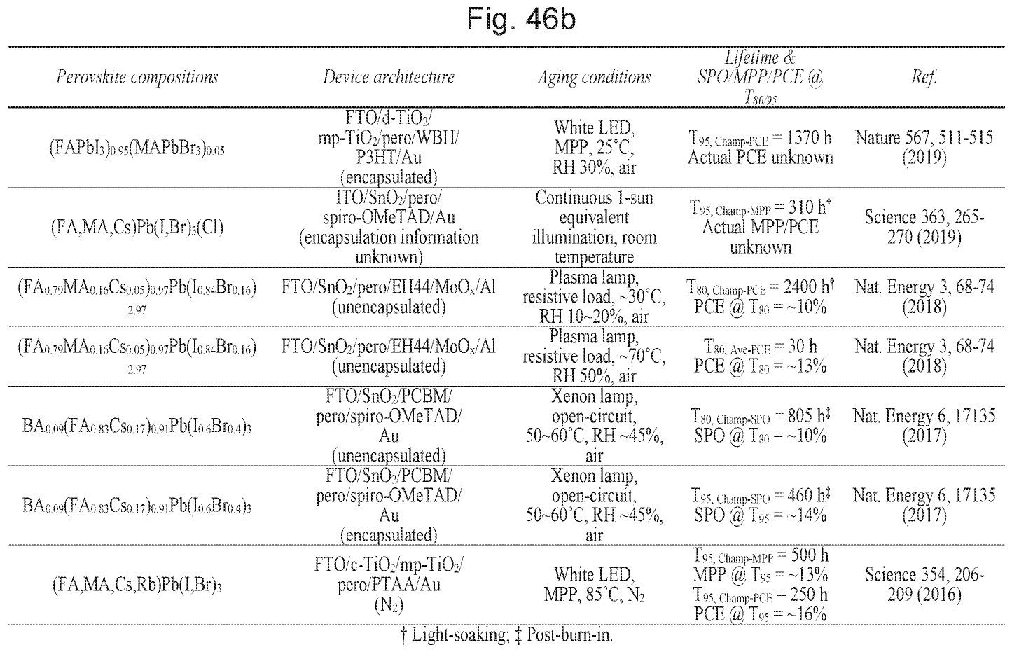

Our [BMP]⁺[BF₄]⁻ modified devices remained highly operational, with average times to decrease to 80% of the peak SPO and PCE (T_{80,ave}) of 944 h and 975 h, respectively. We observed that the V_{OC} remained beyond its initial level for >1000 h at close to 1.2V (FIG. 35A). We note that the unencapsulated devices appear to be much more stable than the isolated perovskite films aged under the same conditions. This is likely to be due to the PC₆₁BM/BCP electron extraction layer and the Cr/Au electrode partially encapsulating the perovskite film, by inhibiting ingress of atmosphere and loss of degradation products (Nano Lett. 14, 5561-5568 (2014), Nat. Energy 2, 17009 (2017), Nat. Mater. 14, 1032-1039 (2015), Nat. Energy 4, 939-947, 2019). To benchmark our stability results against the long-term stability data from the literature (FIGS. 46A and 46B), our champion [BMP]⁺[BF₄]⁻ device exhibited the measured and estimated lifetimes through a linear extrapolation for 80% of the peak SPO and PCE (i.e., T_{80,champ}) of 1010 h and 2630 h, respectively (Nat. Energy 5, 35-49, 2020). The difference in T_{80,champ} between SPO and PCE originates from non-negligible hysteresis in the J-V scans from the aged samples (FIG. 36). Notably, most stability studies are performed on encapsulated cells, or cells in an inert atmosphere. Previous reports from unencapsulated cells in ambient atmosphere have delivered T₈₀ of ˜100 hours under similar aging conditions (Nature 571, 245-250, 2019) or similar T₈₀ lifetimes, but at 25° C. in Colorado at a relative humidity of 15%, dropping to ˜30 hours at 70° C. (Nat. Energy 3, 68-74, 2018).

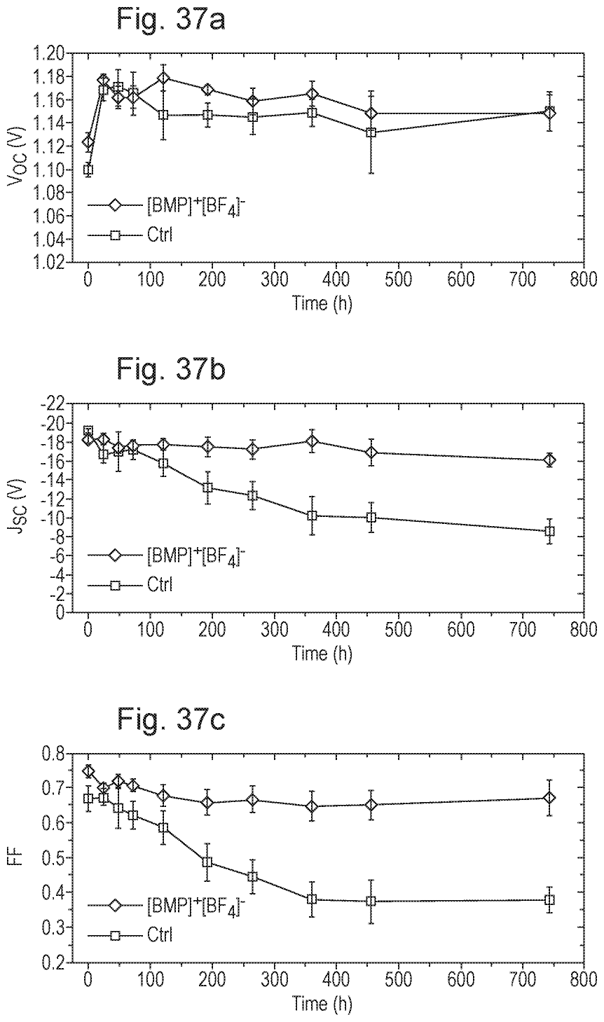

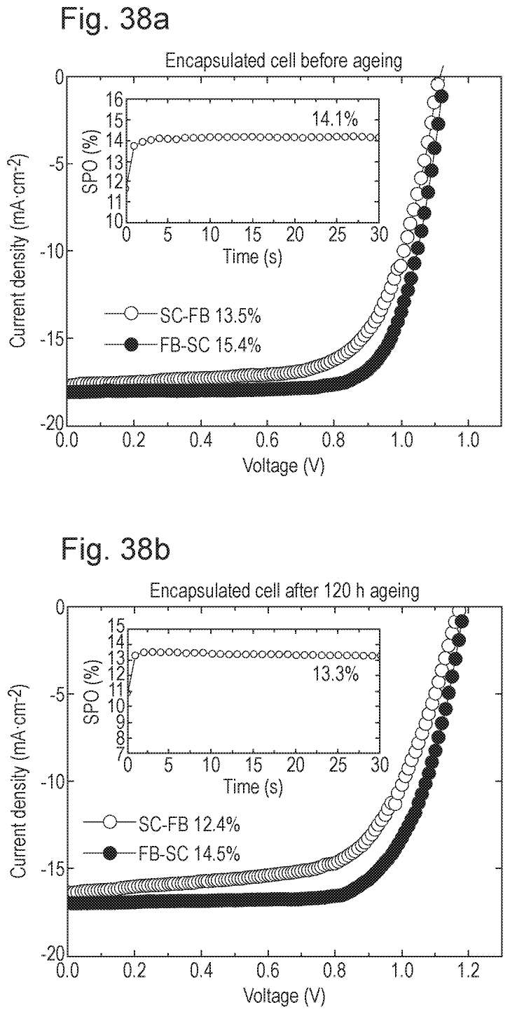

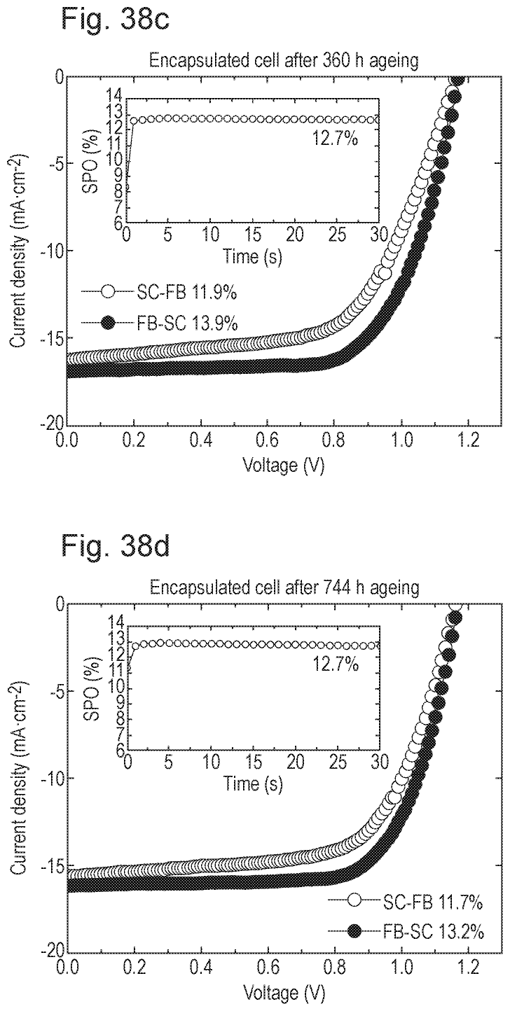

In order to explore the stability of our cells under higher elevated temperatures, we sealed our cells in a nitrogen atmosphere with glass cover slides and UV-cured epoxy resin and aged the encapsulated devices under full spectrum sun light at 85° C. in air. FIG. 37 shows the evolution of the device parameters. The J-V scans for the champion [BMP]⁺[BF₄]⁻ device at different aging stages are shown in FIG. 38. At this temperature, a clear “burn-in” effect was observed in the SPOs (FIG. 34C) and PCEs (FIG. 34D) for both the [BMP]⁺[BF₄]⁻ modified and control devices. The SPO of the control devices decreased rapidly to <6% absolute efficiency after 264 h, while the modified devices retain an operational SPO of ˜12% absolute over the aging period. We estimated the lifetime to 95% of the “post-burn-in” efficiency (T_{95,ave}) of 1200 h, using a linear extrapolation of the post-burn-in SPO (FIG. 34C) (Nature 571, 245-250, 2019; Nat. Energy 5, 35-49, 2020).

Much variation in aging conditions for perovskite solar cells occur between laboratories, so it is not directly feasible to compare results. With respect to our previous “best-in-class”, the T₈₀ lifetime of our unencapsulated cells at 60° C. here is approximately 7 times longer (Nature 571, 245-250, 2019). The post-burn-in T_{95,ave} SPO lifetime of our encapsulated cells at 85° C. was 1200 hours, three times longer than our previous best-in-class cells which were stressed at 75° C. and gave a T_{95,ave} of ˜360 h (Nature 571, 245-250, 2019). Considering that we would expect about a twofold increase in the degradation rate with 10° C. increase in temperature (Nat. Energy 3, 459-465, 2018), our cells here appear to degrade approximately six times as slowly.

Degradation Mechanism and Failure Analysis of Aged Cells

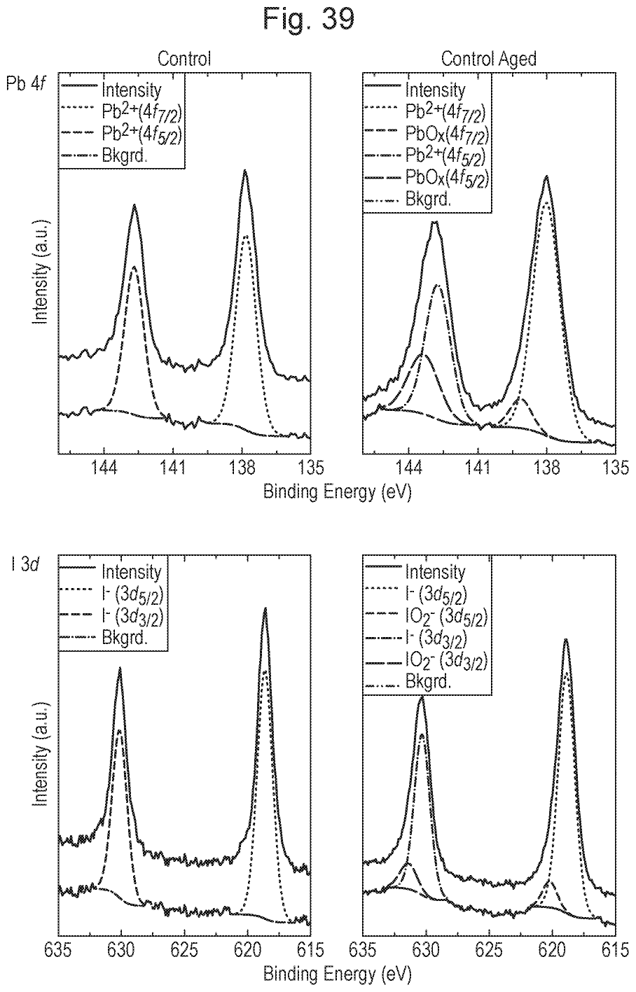

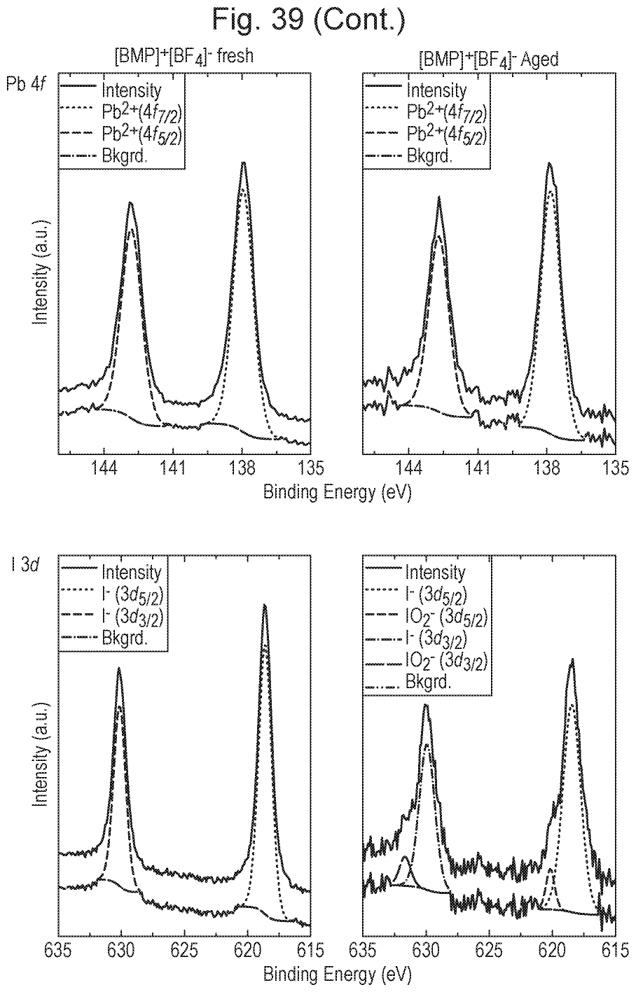

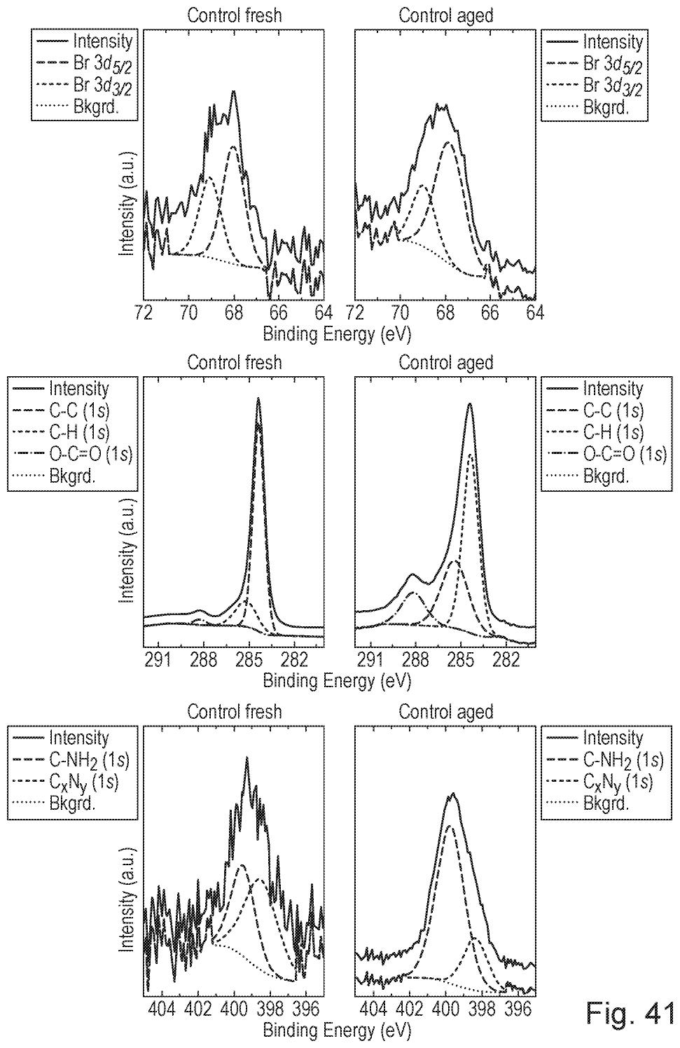

To elucidate the degradation mechanism in complete cells, we carried out XPS analysis on the unencapsulated device stacks, absent of electrodes, before and after a 60° C. light soaking 300 h aging process. The XPS spectra of the core levels relevant to the perovskite elements were measured, and full peak positions, spectra and fittings can be found in FIGS. 39-41 and Table 7. Subtle differences between the [BMP]⁺[BF₄]⁻ and control devices were observed in the C 1s, N 1s core levels, but these additional peaks correspond to the presence of [BMP]⁺[BF₄]⁻. The I 3d_{5/2} core levels for both the [BMP]⁺[BF₄]⁻ and control devices show that aging resulted in the emergence of an additional peak at ˜620 eV next to the main peak at ˜618 eV, which is attributed to I⁻. This peak at the higher binding energy could correspond to either the formation of IO₂⁻, which has been previously observed in methylammonium lead iodide perovskite (Phys. Chem. Chem. Phys. 18, 7284-7292 (2016)), or to the formation of methyl iodide (Surf. Sci. 219, 294-316, 1989).

TABLE 7
Peak positions and full assignments for high
resolution XPS of all fresh and aged devices.

| | Br 3d_{5/2} | Pb 4f_{7/2} | I 3d_{5/2} | C 1s | N 1s |
|---|---|---|---|---|---|
| | (eV) | (eV) | (eV) | (eV) | (eV) |
| Control | 68.0 | 137.8 | 618.6 (I⁻) | 284.4 (C—C) | 398.4 (CₓN_{y}) |
| (fresh) | (Br⁺) | (Pb²⁺) | | 285.2 (C—H) | 399.5 (C—NH₂) |
| | | | | 288.3 | |
| Control | 67.8 | 138.0 | 618.9 (I⁻) | 284.3 (C—C) | 398.4 (CₓN_{y}) |
| (aged) | (Br⁺) | (Pb²⁺) | 620.2 | 285.4 (C—H) | 399.7 (C—NH₂) |
| | | 139.1 | (IO₂⁻)/(ICH₃) | 288.1 (O—C═O) | |
| | | (PbOₓ) | | | |
| Treated | 67.9 | 137.9 | 618.6 (I⁻) | 284.4 (C—C) | 398.3 (CₓN_{y}) |
| (fresh) | (Br⁺) | (Pb²⁺) | | 285.2 (C—H) | 399.9 (C—NH₂) |
| | | | | 285.8 (C—N⁺X₃) | |
| | | | | 288.3 (O—C═O) | |
| Treated | 68.0 | 137.8 | 618.4 (I⁻) | 284.3 (C—C) | 398.3 (CₓN_{y}) |
| (aged) | (Br⁺) | (Pb²⁺) | 620.1 | 285.2 (C—H) | 399.0 (N⁺C₁₀H₂₂BF₄) |
| | | | (IO₂⁻)/(ICH₃) | 285.9 (C—N⁺X₃) | 399.9 (C—NH₂) |
| | | | | 288.2 (O—C═O) | |

The Pb 4f core level spectra demonstrated a clear difference between the aged devices with and without [BMP]⁺[BF₄]⁻. In the aged control devices, we observed two peaks at 138.0 and 139.1 eV (Pb 4f_{7/2}) whereas in the devices with [BMP]⁺[BF₄]⁻ we only observed one peak at 137.8 eV. The peaks at ˜138 eV correspond to the presence of Pb²⁺ while the peak at 139.1 eV corresponds to PbOₓ. This result suggest that when aged, the control devices formed lead oxide, which is a product formed from a photo-oxidative degradation process (J. Mater. Chem. A 7, 2275-2282, 2019). The addition of [BMP]⁺[BF₄]⁻ inhibited lead oxidation.

In order to understand how [BMP]⁺[BF₄]⁻ can improve the stability of the perovskite film, we first review the mechanisms that have been proposed to explain the photoinduced degradation processes of metal halide perovskites. The role of oxygen and moisture has been extensively discussed, in particular for methylammonium containing perovskites (Adv. Mater. 30, 1706208 (2018), Adv. Energy Mater., 1903109 (2019), Nat. Commun. 8, 15218, 2017). However, for perovskite films prepared in inert atmosphere and encapsulated, photodegradation is still observed. A key factor in the photodegradation is the generation of I₂ under illumination, which has been observed experimentally via a range of analytic methods including electrochemistry and optical absorption (Nat. Mater. 17, 445-449, 2018) and mass spectrometry (Sustain. Energy Fuels 2, 2460-2467, 2018). The detrimental role of I₂ has been established for silver electrodes (Adv. Mater. Interfaces 2, 1500195, 2015) but also directly upon the perovskite (Nat. Energy 2, 16195, 2016).

Several mechanisms have been proposed to explain the generation of I₂. They have in common the capture of a hole by an iodide ion (I⁻), with I⁻ being either in its lattice site (I_{I}^{x}+h^{⋅}→I_{I}^{⋅} in Kröger-Vink notation) (Joule 3, 2716-2731, 2019; Physica 35, 386-394, 1967), as an interstitial ion from a Frenkel pair (Iᵢ′+h^{⋅}→Iᵢ^{x}) (Nat. Photon. 13, 532-539, 2019), or becoming interstitial upon hole capture (I_{I}^{x}+h^{⋅}→Iᵢ^{x}+V_{I}^{⋅}) (Nat. Mater. 17, 445-449, 2018). To generate gaseous iodine (I₂), two neutral atoms (Iᵢ^{x} or I_{I}^{⋅}) need to diffuse and combine. Due to the ability to release iodine from the surface of the film, and the likelihood of a higher vacancy density at the surface than in the bulk of the grains, this process is more likely to happen at the surface of the grains, leading to the release of iodine and the generation of a pair of iodide vacancies (2V_{I}^{⋅}).

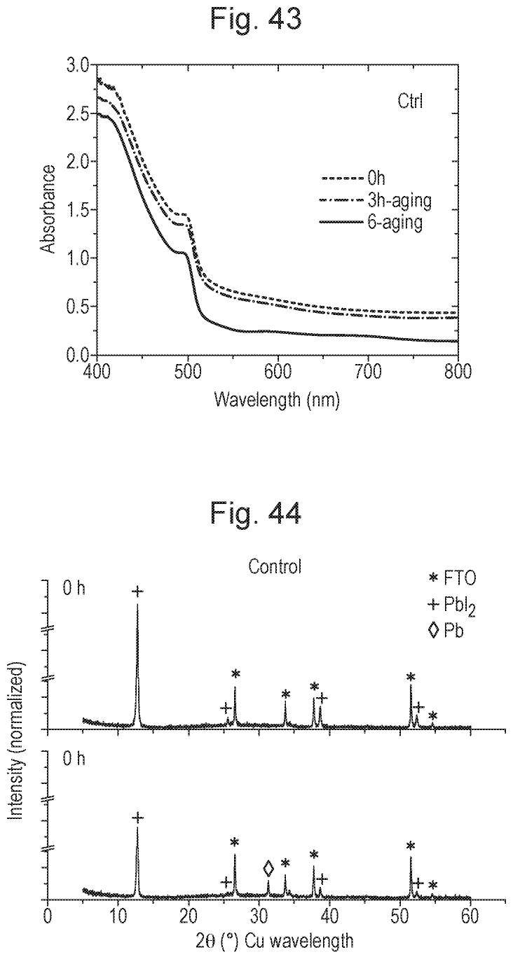

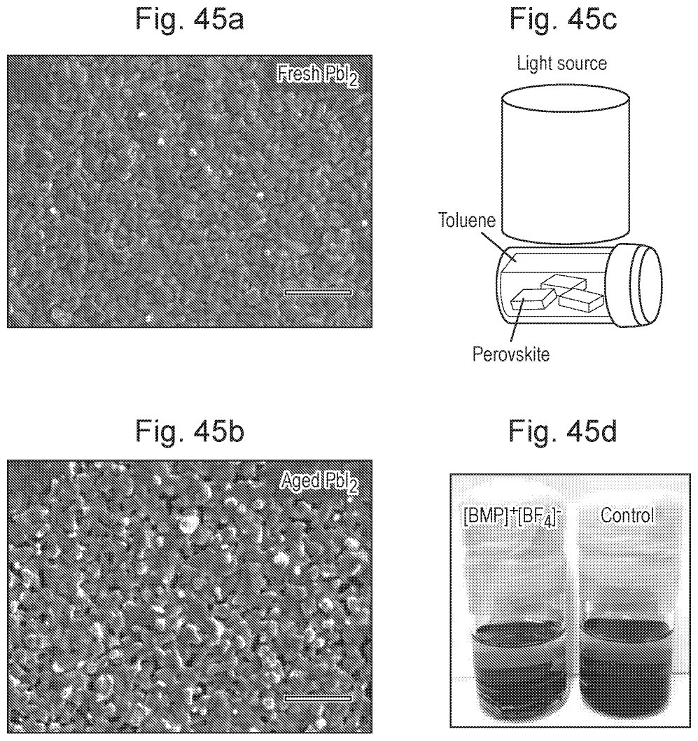

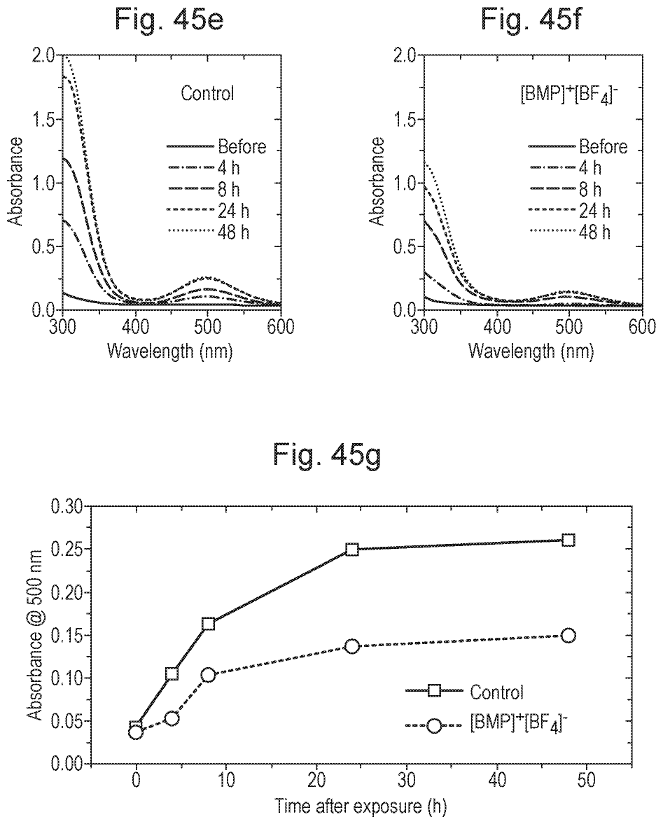

The exact nature of the detrimental effect of I₂ on the perovskite is still under debate (Nat. Energy 2, 16195 (2016). Fu et al. investigated these effects in detail and found that PbI₂ was more prone to degradation than the perovskite itself, and that voids were left in the films of PbI₂ upon prolonged exposure to light and heat (Fu et al., Energy. Environ. Sci. 12, 3074-3088, 2019). We repeated Fu's experiments for the photodegradation of PbI₂ at elevated temperatures under light in a nitrogen atmosphere (FIGS. 42 and 43) and confirmed the observed generation of lead (FIG. 44) and pinholes in the films (FIG. 45, A and B). Although slower than for MA⁺ containing perovskites, this degradation pathway was also observed in FACs perovskites, indicating that the I₂ generation process is related to the lead-halide framework.

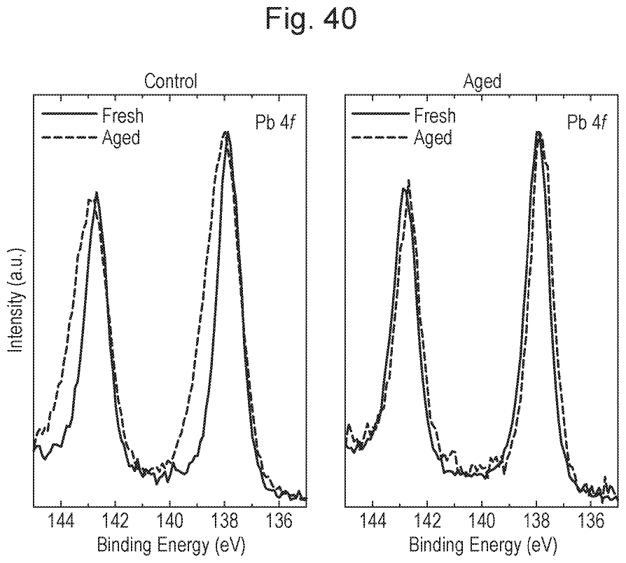

We confirmed a faster release of I₂ from our control perovskite films versus the [BMP]⁺[BF₄]⁻ modified films during light soaking, from UV-vis absorption spectrum of toluene, after exposing toluene-submersed films to light and heat (FIG. 45, C to G) (Nat. Mater. 17, 445-449 (2018), Nat. Energy 2, 16195, 2016). The presence of voids (or pinholes, FIG. 45B) in the PbI₂ films (and control perovskite films, FIG. 33) after aging can be explained by the loss of volume during I₂ release, upon conversion to metallic lead. The presence of PbOₓ, which we observed in the degraded unencapsulated devices (FIG. 40), is also consistent with the formation of metallic lead, which is subsequently oxidized to PbOₓ or Pb(OH)₂ in ambient air, which notably have a much higher density than perovskite.

In the light of this degradation mechanism, we discuss the stabilization induced by the ionic additive. For degradation to occur hole-trapping is likely to require interstitial I⁻, and two neutral interstitial iodine atoms need to diffuse together and combine to form I₂. Therefore, this reaction could be slowed down by either reducing the overall density of Frenkel defects (iodide vacancies/interstitial pairs), or by reducing the diffusivity of interstitials. As with most crystalline materials, defect densities are usually highest on the crystal surface. Therefore, we would expect the interstitials or Frenkel defects to mostly diffuse around the surfaces of the polycrystalline domains, where there exists the highest density of defects.

If the crystallisation in the presence of [BMP]⁺[BF₄]⁻ leads to reduced Frenkel defect densities, then this would have the effect of reducing the number of sites available for iodide oxidation. Furthermore, if the [BMP]⁺[BF₄]⁻ adsorbs to these surface defects, it is likely to block or inhibit the further migration of such defects, slowing down the diffusivity of interstitial iodide or neutral iodine interstitials, reducing the rate of I₂ formation. Finally, [BMP]⁺[BF₄]⁻ does appear to reduce the amount of residual PbI₂ in the films, by improving the crystallisation (as indicated in FIG. 21). Since the I₂ generation occurs preferentially at PbI₂ sites (Energy. Environ. Sci. 12, 3074-3088 (2019)), minimizing the amount of residual PbI₂ may play a crucial role.

The project leading to this application has received funding from the European Union's Horizon 2020 research and innovation programme under grant agreement No. 763977.

1. An optoelectronic device comprising:
(a) a layer comprising a crystalline A/M/X material, wherein the crystalline A/M/X material comprises a compound of formula:
[A]ₐ[M]_{b}[X]_{c}
wherein:
[A] comprises one or more A cations;
[M] comprises one or more M cations which are metal or metalloid cations;
[X] comprises one or more X anions;
a is a number from 1 to 6;
b is a number from 1 to 6; and
c is a number from 1 to 18; and
(b) an ionic solid which is a salt comprising an organic cation and a counter anion, wherein the ionic solid is other than a primary, secondary, tertiary or quaternary ammonium halide salt and other than a formamidinium or guanidinium halide salt.

2. An optoelectronic device according to claim 1 wherein the organic cation is present within the layer comprising the crystalline A/M/X material.

3. An optoelectronic device according to claim 1 or claim 2 wherein the organic cation is present at the surfaces of the layer comprising the crystalline A/M/X material and throughout the bulk of the layer comprising the crystalline A/M/X material.

4. An optoelectronic device according to any one of claims 1 to 3 wherein the crystalline A/M/X material is a polycrystalline A/M/X material comprising crystallites of the A/M/X material and grain boundaries between the crystallites, wherein the organic cation is present at grain boundaries between the crystallites and on an outer surface of the crystalline A/M/X material.

5. An optoelectronic device according to any one of the preceding claims wherein the optoelectronic device further comprises a layer comprising a charge-transporting material, wherein the layer comprising the crystalline A/M/X material is disposed on the layer comprising the charge-transporting material,
optionally wherein:
(A) the charge-transporting material is a hole-transporting (p-type) material, optionally wherein the hole-transporting material comprises (i) an organic hole-transporting material such as poly[N,N′-bis(4-butylphenyl)-N,N′-bisphenylbenzidine] (polyTPD), or polyTPD doped with a p-type dopant such as 2,3,5,6-Tetrafluoro-7,7,8,8-tetracyanoquinodimethane (F4-TCNQ), or (ii) a solid state inorganic hole transporting material such as an oxide of nickel, vanadium, copper or molybdenum;
or
(B) the charge-transporting material is an electron-transporting (n-type) material, optionally wherein the electron-transporting material comprises (i) a solid state inorganic electron transporting material such as titanium dioxide, or (ii) an organic electron-transporting material such as [6,6]-phenyl-C₆₁-butyric acid methyl ester (PCBM).

6. An optoelectronic device according to claim 5 wherein the counter-anion is present (a) within the layer comprising the crystalline A/M/X material, (b) between the layer comprising the crystalline A/M/X material and the layer comprising the charge-transporting material, and/or (c) within the layer comprising the charge-transporting material.

7. An optoelectronic device according to claim 5 or claim 6 wherein the charge-transporting material is a hole-transporting (p-type) material which comprises polyTPD, optionally wherein the polyTPD is doped with a p-type dopant such as 2,3,5,6-Tetrafluoro-7,7,8,8-tetracyanoquinodimethane (F4-TCNQ), and optionally wherein the hole-transporting material further comprises N,N′-bis(1-naphthyl)-N,N′-diphenyl-1,1′-biphenyl-4,4′-diamine (NPD).

8. An optoelectronic device according to any one of the preceding claims wherein the organic cation and the counter anion are present within the layer comprising the crystalline A/M/X material.

9. An optoelectronic device according to any one of the preceding claims wherein the crystalline A/M/X material is a polycrystalline A/M/X material comprising crystallites of the A/M/X material and grain boundaries between the crystallites, wherein the organic cation and the counter anion are present at grain boundaries between the crystallites and on an outer surface of the crystalline A/M/X material.

10. An optoelectronic device according to any one of the preceding claims wherein the organic cation comprises a moiety of formula (I):

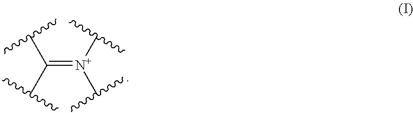 [SMILES: *C(*)=[N+](*)*]

11. An optoelectronic device according to any one of the preceding claims wherein the organic cation is an unsubstituted or substituted imidazolium cation, an unsubstituted or substituted triazolium cation, an unsubstituted or substituted pyrazolium cation, an unsubstituted or substituted iminium cation, or an unsubstituted or substituted pyridinium cation.

12. An optoelectronic device according to any one of the preceding claims wherein the organic cation is:
(a) an imidazolium cation of formula III: 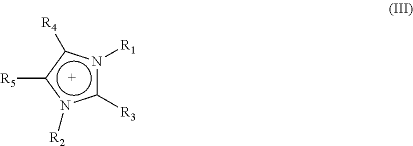
wherein each of R₁, R₂, R₃, R₄ and R₅ is independently selected from hydrogen, unsubstituted or substituted C₁₋₂₀ alkyl, unsubstituted or substituted C₂₋₂₀ alkenyl, unsubstituted or substituted C₂₋₂₀ alkynyl, unsubstituted or substituted aryl, unsubstituted or substituted C₃₋₁₀ cycloalkyl, unsubstituted or substituted heterocyclyl, unsubstituted or substituted amino, unsubstituted or substituted (C₁₋₁₀ alkyl)amino, and unsubstituted or substituted di(C₁₋₁₀ alkyl)amino, provided that R¹ and R⁴, or R⁴ and R⁵, or R⁵ and R², or R² and R³, or R³ and R¹, may together form a C₁₋₁₀ alkylene group; or
(b) a triazolium cation of formula IV: 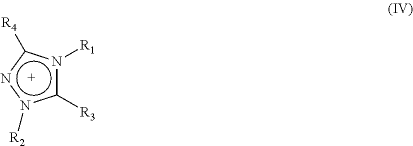
wherein each of R₁, R₂, R₃ and R₄ is independently selected from hydrogen, unsubstituted or substituted C₁₋₂₀ alkyl, unsubstituted or substituted C₂₋₂₀ alkenyl, unsubstituted or substituted C₂₋₂₀ alkynyl, unsubstituted or substituted aryl, unsubstituted or substituted C₃₋₁₀ cycloalkyl, unsubstituted or substituted heterocyclyl, unsubstituted or substituted amino, unsubstituted or substituted (C₁₋₁₀ alkyl)amino, and unsubstituted or substituted di(C₁₋₁₀ alkyl)amino, provided that R¹ and R⁴, or R² and R³, or R¹ and R³, may together form a C₁₋₁₀ alkylene group; or
(c) an iminium cation of formula II:
[R^{x}R^{y}C═NR^{z}R^{w}]⁺ (II)
wherein
R^{x} is hydrogen, unsubstituted or substituted C₁₋₂₀ alkyl, unsubstituted or substituted C₂₋₂₀ alkenyl, unsubstituted or substituted C₂₋₂₀ alkynyl, unsubstituted or substituted aryl, unsubstituted or substituted C₃₋₁₀ cycloalkyl, unsubstituted or substituted heterocyclyl, unsubstituted or substituted amino, unsubstituted or substituted (C₁₋₁₀ alkyl)amino, or unsubstituted or substituted di(C₁₋₁₀ alkyl)amino, provided that R^{x} may together with R^{y} form a C₁₋₂₀ alkylene group;
R^{y} is hydrogen, unsubstituted or substituted C₁₋₂₀ alkyl, unsubstituted or substituted C₂₋₂₀ alkenyl, unsubstituted or substituted C₂₋₂₀ alkynyl, unsubstituted or substituted aryl, unsubstituted or substituted C₃₋₁₀ cycloalkyl, unsubstituted or substituted heterocyclyl, unsubstituted or substituted amino, unsubstituted or substituted (C₁₋₁₀ alkyl)amino, or unsubstituted or substituted di(C₁₋₁₀ alkyl)amino, provided that R^{y} may together with R^{x} form a C₁₋₂₀ alkylene group;
R^{z} is unsubstituted or substituted C₁₋₂₀ alkyl, unsubstituted or substituted C₂₋₂₀ alkenyl, unsubstituted or substituted C₂₋₂₀ alkynyl, unsubstituted or substituted aryl, unsubstituted or substituted C₃₋₁₀ cycloalkyl, unsubstituted or substituted heterocyclyl, unsubstituted or substituted amino, unsubstituted or substituted (C₁₋₁₀ alkyl)amino, or unsubstituted or substituted di(C₁₋₁₀ alkyl)amino, provided that R may together with R^{z} form a C₁₋₂₀ alkylene group; and
R^{w} is unsubstituted or substituted C₁₋₂₀ alkyl, unsubstituted or substituted C₂₋₂₀ alkenyl, unsubstituted or substituted C₂₋₂₀ alkynyl, unsubstituted or substituted aryl, unsubstituted or substituted C₃₋₁₀ cycloalkyl, unsubstituted or substituted heterocyclyl, unsubstituted or substituted amino, unsubstituted or substituted (C₁₋₁₀ alkyl)amino, or unsubstituted or substituted di(C₁₋₁₀ alkyl)amino, provided that R^{w} may together with R^{z} form a C₁₋₂₀ alkylene group.

13. An optoelectronic device according to any one of claims 1 to 9 wherein the organic cation is an unsubstituted or substituted piperidinium cation.

14. An optoelectronic device according to any one of claims 1 to 9 and 13 wherein the organic cation is a piperidinium cation of formula VI: 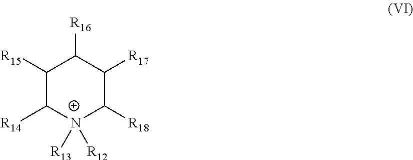
wherein each of R₁₂, R₁₃, R₁₄, R₁₅, R₁₆, R₁₇ and R₁₈ is independently selected from hydrogen, unsubstituted or substituted C₁₋₁₀ alkyl, unsubstituted or substituted C₂₋₁₀ alkenyl, unsubstituted or substituted C₂₋₁₀ alkynyl, unsubstituted or substituted C₆₋₁₂ aryl, unsubstituted or substituted C₃₋₁₀ cycloalkyl, unsubstituted or substituted C₃₋₁₀ cycloalkenyl, amino, unsubstituted or substituted (C₁₋₆ alkyl)amino and unsubstituted or substituted di(C₁₋₆ alkyl)amino.

15. An optoelectronic device according to claim 14 wherein each of R₁₄, R₁₅, R₁₆, R₁₇ and R₁₈ is hydrogen, R₁₂ is n-butyl and R₁₃ is methyl.

16. An optoelectronic device according to any one of claims 1 to 9 wherein the organic cation is an unsubstituted or substituted piperidinium cation as defined in any one of claims 13 to 15 and wherein: (i) the compound of formula [A]ₐ[M]_{b}[X]_{c} does not comprise methylammonium, or (ii) [A] of the compound of formula [A]ₐ[M]_{b}[X]_{c} consists of methylammonium and at least one A cation other than methylammonium, provided that the molar fraction of methylammonium in [A] is less than 15% of [A].

17. An optoelectronic device according to any one of the preceding claims wherein the counter-anion is a polyatomic anion, preferably wherein the polyatomic anion is a non-coordinating anion, optionally wherein the counter-anion is a borate anion.

18. An optoelectronic device according to any one of the preceding claims wherein the counter-anion is BF₄⁻.

19. An optoelectronic device according to any one of claims 1 to 12 wherein the counter-anion is a halide anion.

20. An optoelectronic device according to any one of the preceding claims wherein [A] does not comprise methylammonium, or [A] consists of methylammonium and at least one A cation other than methylammonium, provided that the molar fraction of methylammonium in [A] is less than 15% of [A].

21. An optoelectronic device according to any one of the preceding claims wherein [A] comprises two or more different A cations.

22. An optoelectronic device according to any one of the preceding claims wherein [A] comprises Cs⁺ and formamidinium.

23. An optoelectronic device according to any one of the preceding claims wherein [A] comprises Cs⁺ and formamidinium and:
(i) the compound of formula [A]ₐ[M]_{b}[X]_{c} does not comprise methylammonium, or
(ii) [A] of the compound of formula [A]ₐ[M]_{b}[X]_{c} comprises methylammonium, Cs⁺ and formamidinium, provided that the molar fraction of methylammonium in [A] is less than 15% of [A].

24. An optoelectronic device according to any one of the preceding claims wherein [A] comprises at least two different A cations and [X] comprises at least two different X anions, optionally wherein [A] comprises at least three different A cations and [X] comprises at least two different X anions.

25. An optoelectronic device according to any one of the preceding claims wherein the compound of formula [A]ₐ[M]_{b}[X]_{c} is a compound of formula [(H₂N—C(H)═NH₂)ₓCs₁₋ₓ]Pb[Br_{y}I_{1-y}]₃ wherein x is greater than 0 and less than 1, and y is greater than 0 and less than 1.

26. An optoelectronic device according to any one of the preceding claims wherein [X] comprises I and Br, wherein the molar ratio of I to Br in the compound of formula [A]ₐ[M]_{b}[X]_{c} is less than 9:1, preferably less than 7:1, more preferably equal to or less than 4:1.

27. An optoelectronic device according to any one of the preceding claims wherein the compound of formula [A]ₐ[M]_{b}[X]_{c} is other than [{(H₂N—C(H)═NH₂)_{0.83}(CH₃NH₃)_{0.17}}_{0.95}Cs_{0.05}]Pb[Br_{0.1}I_{0.9}]₃, and preferably wherein the compound is [(H₂N—C(H)═NH₂)_{0.83}Cs_{0.17}]Pb[Br_{0.23}I_{0.77}]₃.

28. An optoelectronic device according to any one of the preceding claims which comprises a layer comprising the hole-transporting (p-type) material as defined in claim 5 or claim 7, wherein the layer comprising the crystalline A/M/X material is disposed on the layer comprising the hole-transporting material, and wherein the optoelectronic device further comprises:
a first electrode comprising a transparent conducting oxide, wherein the layer comprising the hole-transporting material is disposed between the layer comprising the crystalline A/M/X material and the first electrode;
a layer comprising an electron-transporting (n-type) material; and
a second electrode which comprises a metal in elemental form, wherein the layer comprising the electron-transporting material is disposed between the layer comprising the crystalline A/M/X material and the second electrode.

29. An optoelectronic device according to any one of the preceding claims which is a photovoltaic device, optionally wherein the photovoltaic device is a single-junction photovoltaic device, a tandem junction photovoltaic device or a multi-junction photovoltaic device.

30. A process for producing an ionic solid-modified film of a crystalline A/M/X material, wherein the crystalline A/M/X material comprises a compound of formula:
[A]ₐ[M]_{b}[X]_{c}
wherein:
[A] comprises one or more A cations;
[M] comprises one or more M cations which are metal or metalloid cations;
[X] comprises one or more X anions;
wherein
a is a number from 1 to 6;
b is a number from 1 to 6; and
c is a number from 1 to 18,
and the ionic solid is a salt which comprises an organic cation and a counter-anion, wherein the ionic solid is other than a primary, secondary, tertiary or quaternary ammonium halide salt and other than a formamidinium or guanidinium halide salt,
which process comprises:
disposing a film-forming solution on a substrate, wherein the film-forming solution comprises a solvent, the one or more A cations, the one or more M cations, the one or more X anions, the organic cation and the counter-anion.

31. A process for producing an ionic solid-modified film of a crystalline A/M/X material, which crystalline A/M/X material comprises a compound of formula:
[A]ₐ[M]_{b}[X]_{c}
wherein:
[A] comprises one or more A cations;
[M] comprises one or more M cations which are metal or metalloid cations;
[X] comprises one or more X anions;
wherein
a is a number from 1 to 6;
b is a number from 1 to 6; and
c is a number from 1 to 18,
the process comprising:
a) disposing a first solution on a substrate wherein the first solution comprises a solvent and one or more M cations, and optionally removing the solvent, to produce a treated substrate;
b) contacting the treated substrate with a second solution comprising a solvent and one or more A cations or with vapour comprising one or more A cations,
wherein:
one or more X anions are present in one or both of: (i) the first solution employed in step (a), and (ii) the second solution or vapour employed in step (b); and
the first solution employed in step (a) further comprises an organic cation and a counter-anion of an ionic solid or step (b) further comprises contacting the treated substrate with an ionic solid, wherein the ionic solid is a salt which comprises an organic cation and a counter-anion and the ionic solid is other than a primary, secondary, tertiary or quaternary ammonium halide salt and other than a formamidinium or guanidinium halide salt.

32. A process according to claim 31 wherein
the first solution employed in step (a) further comprises the organic cation and the counter-anion of the ionic solid; or
wherein step (b) further comprises contacting the treated substrate with the ionic solid, optionally wherein step (b) comprises:
(i) contacting the treated substrate with said second solution wherein the second solution further comprises the organic cation and the counter-anion of the ionic solid; or
(ii) contacting the treated substrate with said vapour comprising one or more A cations and with vapour comprising the organic cation and the counter-anion of the ionic solid, optionally wherein step (b) comprises:
b1) vapourising a composition, or compositions, which comprise the one or more A cations and the ionic solid, and
b2) depositing the resulting vapour on the treated substrate.

33. A process for producing an ionic solid-modified film of a crystalline A/M/X material,
wherein the crystalline A/M/X material comprises a compound of formula:
[A]ₐ[M]_{b}[X]_{c}
wherein:
[A] comprises one or more A cations;
[M] comprises one or more M cations which are metal or metalloid cations;
[X] comprises one or more X anions;
a is a number from 1 to 6;
b is a number from 1 to 6; and
c is a number from 1 to 18;
and wherein the ionic solid is a salt which comprises an organic cation and a counter-anion, wherein the ionic solid is other than a primary, secondary, tertiary or quaternary ammonium halide salt and other than a formamidinium or guanidinium halide salt,
which process comprises:
exposing a substrate to vapour comprising the one or more A cations, vapour comprising the one or more M cations, vapour comprising the one or more X anions, vapour comprising the organic cation, and vapour comprising the counter anion.

34. A process according to claim 33 wherein the process comprises:
exposing the substrate to vapour which comprises the one or more A cations, the one or more M cations, the one or more X anions, the organic cation, and the counter anion; or
exposing the substrate to two or more different vapour phases, wherein the two or more different vapour phases together comprise the one or more A cations, the one or more M cations, the one or more X anions, the organic cation, and the counter anion.

35. A process according to any one of claims 30 to 34, wherein the substrate comprises a first charge-transporting material which is optionally disposed on a first electrode, which first charge-transporting material is preferably a hole-transporting (p-type) material as defined in claim 5 or claim 7, and wherein the first electrode is preferably a transparent electrode.

36. A process for producing an ionic solid-modified film of a crystalline A/M/X material,
wherein the crystalline A/M/X material comprises a compound of formula:
[A]ₐ[M]_{b}[X]_{c}
wherein:
[A] comprises one or more A cations;
[M] comprises one or more M cations which are metal or metalloid cations;
[X] comprises one or more X anions;
a is a number from 1 to 6;
b is a number from 1 to 6; and
c is a number from 1 to 18;
and wherein the ionic solid is a salt which comprises an organic cation and a counter-anion, wherein the ionic solid is other than a primary, secondary, tertiary or quaternary ammonium halide salt and other than a formamidinium or guanidinium halide salt,
which process comprises treating a film of the crystalline A/M/X material with the organic cation and the counter-anion of the ionic solid.

37. A process according to claim 36 wherein the film of the crystalline A/M/X material is disposed on a substrate, optionally wherein the substrate is as defined in claim 35.

38. A process for producing an optoelectronic device, which process comprises producing, on a substrate, an ionic solid-modified film of a crystalline A/M/X material, by a process as defined in any one of claims 30 to 37, optionally wherein the substrate comprises a first charge-transporting material disposed on a first electrode which is a transparent electrode, and the process optionally further comprises:
disposing a second charge-transporting material on the ionic solid-modified film of a crystalline A/M/X material, and
disposing a second electrode on the second charge-transporting material,
wherein the first charge transporting material is a hole-transporting (p-type) material and the second charge transporting material is an electron-transporting (n-type) material, or the first charge transporting material is an electron-transporting (n-type) material and the second charge transporting material is a hole-transporting (p-type) material,
preferably wherein the first electrode comprises a transparent conducting oxide and the second electrode comprises elemental metal.

39. An ionic solid-modified film of a crystalline A/M/X material which is obtainable by a process as defined in any one of claims 30 to 38.

40. An optoelectronic device which
(a) comprises an ionic solid-modified film of a crystalline A/M/X material as defined in claim 39; or
(b) is obtainable by a process as defined in claim 38.
